SEQUENCE LISTING (48 sequences)

SEQ ID NO: 1 - RNA, 45 nt, synthetic construct
  source 1..45: mol_type = other RNA
gtggaatagtataacaatatgctaaatgttgttatagtatcccac

SEQ ID NO: 2 - RNA, 103 nt, synthetic construct
  source 1..103: mol_type = other RNA
tgcaaagattgctgactacagcattgctcagtactgctgtagaatataggctaaatcggtgttccaggggtactcatcagttccgtgttggcaactcgttgta

SEQ ID NO: 3 - RNA, 77 nt, synthetic construct
  source 1..77: mol_type = other RNA
gaccatcgacaagaaagggactgaagctgctgatatgctaaatgtcccacgccacagtccgttgctcgtcgagggtt

SEQ ID NO: 4 - RNA, 77 nt, synthetic construct
  source 1..77: mol_type = other RNA
gaccatcgacaagaaagggactgaagctgctgatatgctaaatgtcaatcgctacaggccgtggctcgtcgatggct

SEQ ID NO: 5 - RNA, 77 nt, synthetic construct
  source 1..77: mol_type = other RNA
gaccatcgacaagaaagggactgaagctgctgatatgctaaatgtcaactaccacactccgtcgctcgtccatggcg

SEQ ID NO: 6 - RNA, 77 nt, synthetic construct
  source 1..77: mol_type = other RNA
gaccatcgacaagaaagggactgaagctgctgatatgctaaatgtcgcacgtcacattccgttgctcgtcgagggct

SEQ ID NO: 7 - RNA, 77 nt, synthetic construct
  source 1..77: mol_type = other RNA
gaccatcgacaagaaagggactgaagctgctgatatgctaaatgtcagcccgggcagtgcctggctcgtcgccggca

SEQ ID NO: 8 - RNA, 77 nt, synthetic construct
  source 1..77: mol_type = other RNA
gaccatcgacaagaaagggactgaagctgctgatatgctaaatgtgcacagctacagtccgtggctcgtcgctggcc

SEQ ID NO: 9 - RNA, 77 nt, synthetic construct
  source 1..77: mol_type = other RNA
gaccatcgacaagaaagggactgaagctgctgatatgctaaatgtcgcacgctacactccgttactcgtcgaaggtt

SEQ ID NO: 10 - RNA, 77 nt, synthetic construct
  source 1..77: mol_type = other RNA
gaccatcgacaagaaagggactgaagctgctgatatgctaaatgttaaacgccacactccgttactcgtctatgcct

SEQ ID NO: 11 - RNA, 77 nt, synthetic construct
  source 1..77: mol_type = other RNA
gaccatcgacaagaaagggactgaagctgctgatatgctaaatgttagttgtcacggtccattcctcgtcgagggtt

SEQ ID NO: 12 - RNA, 77 nt, synthetic construct
  source 1..77: mol_type = other RNA
gaccatcgacaagaaagggactgaagctgctgatatgctaaatgtcaaccgccacattccgttgctcgtccatgtct

SEQ ID NO: 13 - RNA, 77 nt, synthetic construct
  source 1..77: mol_type = other RNA
gaccatcgacaagaaagggactgaagctgctgatatgctaaatgtaagcagctacaaggtcttactcgtcgaagact

SEQ ID NO: 14 - RNA, 77 nt, synthetic construct
  source 1..77: mol_type = other RNA
gaccatcgacaagaaagggactgaagctgctgatatgctaatgtcgcacgctacactccgtttactcgtcgaaggtt

SEQ ID NO: 15 - RNA, 77 nt, synthetic construct
  source 1..77: mol_type = other RNA
gaccatcgacaagaaagggactgaagctgctgatatgctaaatgtcagcagcttcagtccctttctcgtcgatggtc

SEQ ID NO: 16 - RNA, 77 nt, synthetic construct
  source 1..77: mol_type = other RNA
gaccatcgacaagaaagggactgaagctgctgatatgctaaatgtcagcagcgtcggtccgtttctgctcgatggtc

SEQ ID NO: 17 - RNA, 77 nt, synthetic construct
  source 1..77: mol_type = other RNA
gaccatcgacaagaaagggactgaagctgctgatatgctaaatgtcagcagcctgagtccatttttcgtcgatggtc

SEQ ID NO: 18 - RNA, 77 nt, synthetic construct
  source 1..77: mol_type = other RNA
gaccatcgacaagaaagggactgaagctgctgctatgctaaatgtcagcagcttcagtccgtctctcgacgatggtc

SEQ ID NO: 19 - RNA, 77 nt, synthetic construct
  source 1..77: mol_type = other RNA
gaccatcgacaagaaagggactgaagctgctgatatgctaaatgtcagcagcttcagtccgtttctgatcgatggtc

SEQ ID NO: 20 - RNA, 77 nt, synthetic construct
  source 1..77: mol_type = other RNA
gaccatcgacaagaaagggactgaagctgctgatatgctaaatgtcagcagcttccgtcccttcctcgtcgatggtc

SEQ ID NO: 21 - RNA, 77 nt, synthetic construct
  source 1..77: mol_type = other RNA
gaccatcgacaagaaagggactgaagctgctgatatgctaaatgtcagcagctttagtccgtttctcgtcattggtc

SEQ ID NO: 22 - RNA, 77 nt, synthetic construct
  source 1..77: mol_type = other RNA
gaccatcgacaagaaagggactgaagctgctgatatgctaaatgtcagcagcttcggtccgtttctgttcgaaggtc

SEQ ID NO: 23 - RNA, 77 nt, synthetic construct
  source 1..77: mol_type = other RNA
gaccatcgacaagaaagggactgaagctgctgatatgctaaatgtcagcagctacactccgttactcgtcgttggtc

SEQ ID NO: 24 - RNA, 77 nt, synthetic construct
  source 1..77: mol_type = other RNA
gaccatcgacaagaaagggactgaagctgctgaatagctaaatgtcagcagcttcagtgcgttgctcgtcgaaggtc

SEQ ID NO: 25 - RNA, 107 nt, synthetic construct
  source 1..107: mol_type = other RNA
nactgggagtggccattcgacgacgacagtgtggtgtaaaggaattcattagccatggatgtattcatgaaaggactttcaaagccaaggagggagttgtggctgct

SEQ ID NO: 26 - RNA, 104 nt, synthetic construct
  source 1..104: mol_type = other RNA
agcagccacaactccgggaaccctttgaaagtcctttcccgaatacatccacggctaaacttctcctttacaccacactgtcgtcgattaggccactcccagtn

SEQ ID NO: 27 - RNA, 22 nt, synthetic construct
  source 1..22: mol_type = other RNA
tgcaaagattgctgactacagc

SEQ ID NO: 28 - RNA, 23 nt, synthetic construct
  source 1..23: mol_type = other RNA
attgctcagtactgctgtagaat

SEQ ID NO: 29 - RNA, 36 nt, synthetic construct
  source 1..36: mol_type = other RNA
tgcaaagattgctgactacagcattgctcagtactg

SEQ ID NO: 30 - RNA, 11 nt, synthetic construct
  source 1..11: mol_type = other RNA
ttgagccctgt

SEQ ID NO: 31 - RNA, 13 nt, synthetic construct
  source 1..13: mol_type = other RNA
tgactacagcatt

SEQ ID NO: 32 - RNA, 225 nt, synthetic construct
  source 1..225: mol_type = other RNA
tgctgcttttcacactgtatcccaatgctgccatcattgcaaagattgcgactacagcattgctcagtactgctgtagaatggggataaaaacatcagagggcacaccaggccctggtgtgccctctgatgttttttaggggattctacaggaggactgggcagtcccgtggtcgcccttctttgcatactacgcagcatttgggatacagtgtgaaaagcagca

SEQ ID NO: 33 - RNA, 227 nt, synthetic construct
  source 1..227: mol_type = other RNA
tgctgcttttcacactgtatcccaatgctgccatcatttgcaaagattgctgactacagcattgctcagtactgctgtagaatggggataaaaacatcagagggcacaccagggccctggtgtgccctctgatgttttttaggggattctacagcagtactgtccagtcccgtggtcgtaaatctttgcatactacgcagcattgggatacagtgtgaaaagcagca

SEQ ID NO: 34 - RNA, 226 nt, synthetic construct
  source 1..226: mol_type = other RNA
tgctgcttttcacactgtatcccaatgctgccatcattgcaaagattgctgactacagcattgctcagtactgctgtagaatggggataaaaacatcagagggcacaccagggccctggtgtgccctctgatgttttttaggggattctacagcagtactgagcaatgccgtagtcagcaatctttgcatactacgcagcattgggatacagtgtgaaaagcagca

SEQ ID NO: 35 - RNA, 224 nt, synthetic construct
  source 1..224: mol_type = other RNA
tgctgcttttcacactgtatcccaatgctgccatcattgcaaagattgctgactacagcattgctcagtactgctgtagaatggggataaaaacatcagagggcaccagggccctggtgtgccctctgatgttttttaggggattctacagtaggactgagcactgccgagctgggcaatctttgcatactacgcagcattgggatacagtgtgaaaagcagca

SEQ ID NO: 36 - RNA, 224 nt, synthetic construct
  source 1..224: mol_type = other RNA
tgctgcttttcacactgtatcccaatgctgccatcattgcaaagattgctgactacagcattgctcagtactgctgtagaatggggataaaaacatcagagggcaccagggccctggtgtgccctctgatgttttttaggggcttctacagcagttcggaggaatcccgaggtcagcaatctttgcatactacgcagcattgggatacagtgtgaaaagcagca

SEQ ID NO: 37 - RNA, 224 nt, synthetic construct
  source 1..224: mol_type = other RNA
tgctgcttttcacactgtatcccaatgctgccatcattgcaaagattgctgactacagcattgctcagtactgctgtagaatggggataaaaacatcagagggcacaccagggccctggtgtgccctctgatgttttttaggggattctaccgctgtgctgggcaatcccgtggtcagcaatctttgcatacgcagcaattgggatacagtgtgaaaagcagca

SEQ ID NO: 38 - RNA, 227 nt, synthetic construct
  source 1..227: mol_type = other RNA
tgctgcttttcacactgtatcccaatgctgccatcattgcaaagattgctgactacagcattgctcagtactgctgtagaatggggataaaaacatcagagggcacaccagggccctggtgtgccctctgatgttttttaggggattctacatctgtagtgagcaattccgtgctcagcaatctttgcatactacgcaggcattgggatacagtgtgaaaagcagca

SEQ ID NO: 39 - RNA, 226 nt, synthetic construct
  source 1..226: mol_type = other RNA
tgctgcttttcacactgtatcccaatgctgccatcattgcaaagattgctgactacagcattgctcagtactgctgtagaatggggataaaaacatcagagggcacaccagggccctggtgtgccctctgatgttttttaggggattctacaacagtactgagctatcccgaattcaacaatctttgcatactacgcagcattgggatacagtgtgaaaagcagca

SEQ ID NO: 40 - RNA, 225 nt, synthetic construct
  source 1..225: mol_type = other RNA
tgctgcttttcacactgtatcccaatgctgccatcattgcaaagattgctgactacagcattgctcagtactgctgtagaatggggataaaaacatcagagggcacaccagggccctggtgtgccctctgatgttttttaggggattctacagcacgactgagcagtgcttagtcggcaatctttgcatactacgcagcattgggatacagtgtgaaaagcagca

SEQ ID NO: 41 - RNA, 227 nt, synthetic construct
  source 1..227: mol_type = other RNA
tgctgcttttcacactgtatcccaatgctgccatcattgcaaagattgctgactacagcattgctcagtactgctgtagaatggggataaaaacatcagagggcaacaccagggccctggtgtgccctctgatgttttttaggggattctacgcacgactgagcagtgcgttagtcggcaatctttgcatcatcagcagcaattgggatacagtgtgaaaagcagca

SEQ ID NO: 42 - RNA, 118 nt, synthetic construct
  source 1..118: mol_type = other RNA
ccatcattgcaaagattgctgactacagcattgctcagtactgctgtagaatggggatattaggggattctacagcattactcagcagtgcgttagtcagcactctttgcatactacg

SEQ ID NO: 43 - RNA, 118 nt, synthetic construct
  source 1..118: mol_type = other RNA
ccatcattgcaaagattgctgactacagcattgctcagtactgctgtagaatggggatattaggggattctacagcacgactgagcagtgcgttagtcggcaatctttgcatactacg

SEQ ID NO: 44 - RNA, 118 nt, synthetic construct
  source 1..118: mol_type = other RNA
ccatcattgcaaagattgctgactacagcatttgctcagtactgctgtagaatggggatattaggggattctacacaatactcacgagtgcgtgagtcagcaatctttgcatactacg

SEQ ID NO: 45 - RNA, 118 nt, synthetic construct
  source 1..118: mol_type = other RNA
ccatcattgcaaagattgctgactacagcattgctcagtactgctgtagaatggggatattaggggattctacagcacgactgagcagtgcgctagtcggcaatctttgcatactacg

SEQ ID NO: 46 - RNA, 119 nt, synthetic construct
  source 1..119: mol_type = other RNA
ccatcattgcaaagattgctgactacagcattgctcagtactgctgtagaatggggatattaggggattctacagcacgacctgagcagtgcgctagtcagcaatatttgcatactacg

SEQ ID NO: 47 - RNA, 118 nt, synthetic construct
  source 1..118: mol_type = other RNA
ccatcattgcaaagattgctgactacagcattgctcagtactgctgtagaatggggatattaggggattctacagcaatactcagcagtgcgctagtcagccatctttgcatactacg

SEQ ID NO: 48 - RNA, 118 nt, synthetic construct
  source 1..118: mol_type = other RNA
ccatcattgcaaagattgctgactacagcattgctcagtactgctgtagaatggggatattaggggattctacagcacgactgagcagtgcgttagtcagccatctttgcatactacg

(19) United States
(12) Patent Application Publication
(10) Pub. No.: US 2025/0356949 A1
(43) Pub. Date: Nov. 20, 2025

(54) MACHINE-LEARNING BASED DESIGN OF ENGINEERED GUIDE SYSTEMS FOR ADENOSINE DEAMINASE ACTING ON RNA EDITING

(71) Applicant: Shape Therapeutics Inc., Seattle, WA (US)

(72) Inventors: Lan Guo, Seattle, WA (US); Jason Thaddeus Dean, Seattle, WA (US); Adrian Wrangham Briggs, Seattle, WA (US); Ronald James Hause, JR., Seattle, WA (US); Brian John Booth, Boston, MA (US); Lina Rajili Bagepalli, Cambridge, MA (US); Yue Jiang, Los Angeles, CA (US); Yiannis Savva, Seattle, WA (US); Bora Banjanin, Seattle, WA (US); Katherine Rupp, Edmonds, WA (US); Richard Thomas Sullivan, Seattle, WA (US)

(21) Appl. No.: 18/709,008

(22) PCT Filed: Nov. 10, 2022
(86) PCT No.: PCT/US22/79663

§ 371 Date: May 9, 2024

Related U.S. Application Data
(60) Provisional application No. 63/277,801, filed on Nov. 10, 2021.
(60) Provisional application No. 63/284,857, filed on Dec. 1, 2021.
(60) Provisional application No. 63/342,014, filed on May 13, 2022.
(60) Provisional application No. 63/355,955, filed on Jun. 27, 2022.

(51) Int. Cl. G16B 30/00; G16B 15/10; G16B 15/30; G16B 25/00; G16B 40/20

(52) U.S. Cl. CPC G16B 30/00; G16B 15/10; G16B 15/30; G16B 25/00; G16B 40/20

(57) ABSTRACT

Systems and methods for predicting deamination efficiency or specificity associated with a guide RNA (gRNA) are provided. A nucleic acid sequence for the gRNA is received. Responsive to inputting a data structure into a model, a metric for an efficiency or specificity of deamination by a first Adenosine Deaminase Acting on RNA (ADAR) protein of a target nucleotide position in mRNA transcribed from a target gene is obtained as output from the model. The data structure includes an encoding of the nucleic acid sequence for the gRNA.

### 1. CROSS REFERENCE TO RELATED APPLICATIONS

This application claims priority to U.S. Provisional Patent Application Ser. No. 63/277,801, filed Nov. 10, 2021, U.S. Provisional Patent Application Ser. No. 63/284,857, filed Dec. 1, 2021, U.S. Provisional Patent Application Ser. No. 63/342,014, filed May 13, 2022, and U.S. Provisional Patent Application Ser. No. 63/355,955, filed Jun. 27, 2022, each of which is hereby incorporated by reference in its entirety.

### 2. TECHNICAL FIELD

This specification describes technologies generally relating to generating candidate sequences for guide RNAs and predicting attributes of the same.

### 3. BACKGROUND

RNA editing is a post-transcriptional process that recodes hereditary information by changing the nucleotide sequence of RNA molecules (Rosenthal, J Exp Biol. 2015 June; 218 (12): 1812-1821). One form of post-transcriptional RNA modification is the conversion of adenosine-to-inosine (A-to-I), mediated by adenosine deaminase acting on RNA (ADAR) enzymes. Adenosine-to-inosine (A-to-I) RNA editing alters genetic information at the transcript level and is a biological process commonly conserved in metazoans. A-to-I editing is catalyzed by adenosine deaminase acting on RNA (ADAR) enzymes. Such an intracellular RNA-editing mechanism potentially provides a versatile RNA-mutagenesis method for transcriptome manipulation.

Current systems used to edit RNA have limitations which, in some embodiments, lead to aberrant effector activity, have a delivery barrier, unintended transcriptomic modifications, or immunogenicity. Further methods and systems for improved efficiency, specificity, and safety of targeted RNA editing are needed.

### 4. SUMMARY

Provided herein includes various machine learning approaches to design a guide system for editing a desired target RNA (e.g., a pre-mRNA or an mRNA) by an ADAR enzyme.

The engineered guide system, in some embodiments, includes an engineered guide RNA (gRNA) comprising a sequence that has a predicted percentage of on-target editing of a desired nucleotide and a predicted specificity score (e.g., (sum of on-target edits of the desired nucleotide)/(sum of off-target edits)) as determined by a machine learning model. The machine learning model, in some embodiments, receives various inputs such as a sequence of a gRNA and a sequence of the target RNA comprising the desired nucleotide to be edited. In some embodiments, an input is a sequence of a gRNA and a sequence of the target RNA. In some embodiments, an input is a self-annealing RNA structure comprising a sequence of a gRNA and a sequence of the target RNA linked by a hairpin. In some embodiments, the input additionally comprises one or more of specific structural features of a gRNA, time, the editing enzyme, etc. The target RNA sequence, in some embodiments, is a personalized sequence that is determined based on a patient's biological sample. The target RNA sequence, in some embodiments, comprises a common mutation sequence that is known to cause disease or is associated with a cause of a disease. The target RNA sequence, in some embodiments, comprises a nucleotide that when targeted for editing using the engineered RNA as described herein, relieves symptoms of a disease (e.g., targeting a nucleotide at a splice site for editing, resulting in non-functional version of a disease-causing protein). In some embodiments, the machine learning model outputs a predicted percentage of on-target editing of a desired nucleotide and a predicted specificity score ((sum of on-target edits of the desired nucleotide)/(sum of off-target edits)) based on the input sequence. In some embodiments, the machine learning model further shows the impact of an input on the predicted percentage of on-target editing of a desired nucleotide and a predicted specificity score. For example, if an input is a structural feature, the machine learning model further shows the impact of that structural feature on the predicted percentage of on-target editing of a desired nucleotide and a predicted specificity score.

The engineered guide system, in some embodiments, includes an engineered guide RNA (gRNA) comprising a sequence that is determined by a machine learning model using one or more inputs. The machine learning model, in some embodiments, receives various inputs such as a percentage of on-target editing of a desired nucleotide and a specificity score ((sum of on-target editing of the desired nucleotide)/(sum of editing off-target edits)) for a specific nucleotide of a target RNA. The target RNA sequence, in some embodiments, is a personalized sequence that is determined based on a patient's biological sample or is a common mutation sequence that is known to cause disease or is associated with the cause of a disease. In some embodiments, the machine learning model outputs a sequence of RNA that is, at least in part, a sequence of an engineered gRNA that is specific for the target RNA and is predicted to have the input percentage of on-target editing of a desired nucleotide and the input specificity score (e.g., (sum of on-target editing of the desired nucleotide)/(sum of editing off-target edits)).

The machine learning approaches as described herein, in some embodiments, are applied to drug discovery and therapeutic processes such as personalized therapeutics that generate a personalized system for treating a mutation that is specific to a patient.

One aspect of the present disclosure provides a method for predicting a deamination efficiency or specificity. In some embodiments, the method comprises receiving, in electronic form, information comprising a nucleic acid sequence for a guide RNA (gRNA) that hybridizes to a target mRNA. In some embodiments, the method further comprises inputting the information into a model comprising a plurality of parameters to obtain as output from the model a set of one or more metrics for a deamination efficiency or specificity by an Adenosine Deaminase Acting on RNA (ADAR) protein of a target nucleotide position in the target mRNA when facilitated by hybridization of the gRNA to the target mRNA.

Another aspect of the present disclosure provides a method for generating a candidate sequence for a guide RNA (gRNA). In some embodiments, the method comprises receiving, in electronic form, information comprising a desired set of one or more metrics for an efficiency or specificity of deamination of a target nucleotide position in a target mRNA by an Adenosine Deaminase Acting on RNA (ADAR) protein when facilitated by hybridization of the gRNA to the target mRNA. In some embodiments, the method further comprises receiving, in electronic form, seed information comprising (i) a seed nucleic acid sequence for the gRNA and (ii) a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA. In some embodiments, the method further includes inputting the seed information into a model comprising a plurality of parameters to obtain as output from the model a calculated set of the one or more metrics for the efficiency or specificity of deamination of the target nucleotide position in the target mRNA by the ADAR protein. In some embodiments, the method further includes iteratively updating the seed nucleic acid sequence, while holding the plurality of parameters and the target nucleic acid sequence fixed, to reduce a difference between (i) the desired set of the one or more metrics and (ii) the calculated set of the one or metrics, thereby generating the candidate sequence.

Yet another aspect of the present disclosure provides a system comprising a processor and a memory storing instructions, which when executed by the processor, cause the processor to perform steps comprising any of the methods disclosed above.

Still another aspect of the present disclosure provides a non-transitory computer-readable medium storing computer code comprising instructions, when executed by one or more processors, causing the processors to perform any of the methods disclosed above.

### 5. BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 illustrates an exemplary RNA editing system, in accordance with some embodiments of the present disclosure.

FIG. 2 is an example flowchart depicting an example process 200 for treating a patient, in accordance with some embodiments of the present disclosure.

FIG. 3 is an example flowchart depicting two examples of machine learning processes, in accordance with some embodiments of the present disclosure.

FIG. 4 illustrates an example convolutional neural network, in accordance with an embodiment of the present disclosure.

FIGS. 5A and 5B collectively illustrate example features of nucleic acid sequences for use in machine learning models, in accordance with an embodiment of the present disclosure. FIGS. 5C and 5D illustrate examples of validation of a machine learning model, in accordance with some embodiments of the present disclosure.

FIGS. 6A, 6B, 6C, 6D, 6E, 6F, 6G, 6H, 6I, 6J, 6K, 6L, 6M and 6N provide example graphical illustrations of inputs, outputs, performance, and validation of a convolutional neural network in accordance with some embodiments of the present disclosure.

FIGS. 7A and 7B collectively illustrate example candidate sequences obtained from machine learning having top performance, in accordance with an embodiment of the present disclosure.

FIG. 8 shows a legend of various exemplary structural features present in guide-target RNA scaffolds formed upon hybridization of a latent guide RNA of the present disclosure to a target RNA, in accordance with an embodiment of the present disclosure.

FIGS. 9A, 9B and 9C collectively show a schematic of an example CNN, in accordance with an embodiment of the present disclosure.

FIG. 10 shows a graph of the number of guide RNAs with different numbers of mutations (compared to a perfect duplex) used to train a CNN, in accordance with an embodiment of the present disclosure.

FIG. 11 illustrates the correlation between predicted and experimentally tested on-target editing, in accordance with an embodiment of the present disclosure.

FIG. 12 illustrates the method for using a trained CNN to predict an engineered guide RNA sequence having a desired target editing and specificity score, in accordance with an embodiment of the present disclosure.

FIG. 13 shows a graph of the number of guide RNAs with different numbers of mutations (compared to a perfect duplex) generated by a CNN, in accordance with an embodiment of the present disclosure.

FIG. 14 illustrates correlation in predicted and experimentally tested on-target editing using a machine learning model as disclosed herein, with a Spearman's rank correlation coefficient of 0.74 for on-target editing, in accordance with an embodiment of the present disclosure.

FIG. 15 illustrates correlation in predicted and experimentally tested specificity using a machine learning model as disclosed herein, with a Spearman's rank correlation coefficient of 0.67 for specificity score, in accordance with an embodiment of the present disclosure.

FIG. 16 illustrates correlation in predicted and experimentally tested on-target editing using a machine learning model as disclosed herein, with R² of 0.95, in accordance with an embodiment of the present disclosure.

FIG. 17 illustrates correlation in predicted and experimentally tested specificity using a machine learning model as disclosed herein, with R² of 0.79, in accordance with an embodiment of the present disclosure.

FIG. 18 illustrates important features for predicting on-target editing and specificity as determined using a machine learning model disclosed herein, including length of time for editing, ADAR type, right and left barbell positioning, and nucleotide identity and positioning, in accordance with an embodiment of the present disclosure.

FIGS. 19A and 19B illustrate positioning of a feature (the right barbell) is important for achieving high target editing, relative to a target adenosine to be edited, in accordance with an embodiment of the present disclosure.

FIGS. 20A and 20B illustrate positioning of a feature (the right barbell) is important for achieving a high specificity, relative to a target adenosine to be edited, in accordance with an embodiment of the present disclosure.

FIG. 21 illustrates an example in which machine learning is used to obtain the relative importance of guide nucleotide sequencing influencing editing of the LRRK2 G2019S mutant mRNA, in accordance with an embodiment of the present disclosure.

FIG. 22 is a block diagram illustrating components of an example computing machine, in accordance with an embodiment of the present disclosure.

FIGS. 23A and 23B illustrate a workflow using a high throughput screen (HTS) and Machine Learning (ML) platform and a schematic of an example gRNA design, in accordance with an embodiment of the present disclosure.

FIG. 24 illustrates example outputs from XGBoost models predicting gRNA editing and specificity across several targets, in accordance with an embodiment of the present disclosure.

FIGS. 25A and 25B illustrate prediction performance of CNN and XGBoost model architectures, in accordance with an embodiment of the present disclosure. FIG. 25C illustrates experimentally validated target editing and specificity of a select number of top-performing guide RNAs from a HTS library (HTS top performers), guide RNAs obtained from an exhaustive machine learning strategy (ML exhaustive), and guide RNAs obtained from a generative machine learning strategy (ML generative) that were retested to confirm the predictive ability of the ML models. Guide RNAs were observed in the ML exhaustive and ML generative strategies that exhibited better target editing and specificity than the guide RNAs in the original HTS library.

FIGS. 26A, 26B, 26C, 26D, 26E, 26F, 26G, 26H, 26I, and 26J illustrate prediction performance of CNN or XGBoost model architectures, in accordance with an embodiment of the present disclosure.

FIG. 27 illustrates the performance of gRNAs targeting the LRRK2 G2019S mutation in human cells expressing the LRRK2 G2019S mutation with endogenous ADAR1, wherein some of the tested gRNAs were produced in accordance with an embodiment of the present disclosure.

FIG. 28 illustrates that ML gRNAs predicted to have high editing activity for specific ADAR isoforms are validated to have their predicted isoform specificity when tested in cells. The scatterplot shows ML gRNAs editing activity in cells, comparing activity in cells having ADAR1 (x-axis) or in cells having ADAR1 and ADAR2 (y-axis).

FIGS. 29A and 29B collectively show an example block diagram illustrating a computing device and related data structures used by the computing device in accordance with some implementations of the present disclosure.

FIGS. 30A and 30B collectively show an example block diagram illustrating a computing device and related data structures used by the computing device in accordance with some implementations of the present disclosure.

FIGS. 31A, 31B, 31C, 31D, 31E, 31F, 31G, 31H and 31I collectively illustrate an example method in accordance with an embodiment of the present disclosure, in which optional steps are indicated by broken lines.

FIGS. 32A, 32B, 32C, 32D, 32E, 32F, 32G and 32H collectively illustrate an example method in accordance with an embodiment of the present disclosure, in which optional steps are indicated by broken lines.

FIGS. 33A, 33B, 33C, and 33D collectively illustrate that gRNAs derived from generative ML models can outperform HTS guide RNAs, in accordance with an embodiment of the present disclosure.

FIGS. 34A, 34B, and 34C collectively show ADAR editing profiles for HTS guide RNAs, in accordance with an embodiment of the present disclosure.

FIGS. 35A, 35B, 35C, and 35D collectively illustrate that exhaustive ML gRNA models accurately predict ADAR specificity, in accordance with an embodiment of the present disclosure.

FIG. 36 illustrates A-G mismatches across from the target nucleotide position in gRNAs with ADAR2 preference, in accordance with an embodiment of the present disclosure.

6. DETAILED DESCRIPTION

### INTRODUCTION

Therapeutic RNA editing by redirecting natural ADAR enzymes offers huge promise as a safe method of gene therapy without the risk of DNA damage or requiring the delivery of non-human proteins. However, ADAR enzymes possess inherent promiscuity, and sequence preferences and deterministic rules for how different guide RNA (gRNA) sequences result in various editing performances remain not well understood. Described herein is an application of machine learning coupled with a novel high throughput screening (HTS) and validation platform to dramatically improve the effectiveness of targeted ADAR-mediated RNA editing as a therapeutic modality. This approach allows for the exploration of the enormous gRNA design space to propose highly efficient and specific novel gRNA designs that validate experimentally. Further, machine learning approaches to expand modeling gRNA performances for additional targets are described herein.

In some embodiments, the methods, systems, and platforms described herein generate gRNAs that direct natural ADAR enzymes to therapeutically relevant sites in the transcriptome to correct G→A mutations, control splicing, or modulate protein expression and function. In some embodiments the disclosure describes a HTS platform capable of assessing many structurally unique gRNAs (e.g., hundreds of thousands to millions) against any clinically relevant target sequence. In some embodiments, machine learning models are used to model gRNA performances using primary gRNA sequences as inputs, which results in high predictive accuracy for ADAR1 and/or ADAR2 editing. In some embodiments, input optimization is used to generate novel gRNA designs that outperform gRNA from HTS used, in part, to train the model. Advantageously, in some embodiments, the novel gRNA designs exhibit primary and secondary sequence diversity beyond that of the original HTS screen.

Accordingly, in some embodiments, a pipeline is described for integrating supervised learning into HTS screen design for a variety of ADAR targets. In some embodiments, the pipeline is described for integrating supervised learning into screens for a variety of ADAR in a cell or in multiple different types of cells. In some embodiments, the methods and systems described herein can identify rules that predict gRNA editing outcomes for a specific target. In some embodiments, secondary structural features are generated across gRNAs to model gRNA editing performance, e.g., using gradient boosted decision trees, that can identify important structural features to prioritize for future HTS or future screening in cells. In some embodiments, tertiary structural features are generated across gRNAs to model gRNA editing performance, e.g., using gradient boosted decision trees, that can identify important structural features to prioritize for future HTS or future screening in cells. These developments will help shorten gRNA discovery timelines through in silico guide design for any number of common or orphan genetic diseases.

### Definitions

Unless defined otherwise, all technical and scientific terms used herein have the meaning commonly understood by one of ordinary skill in the art to which the invention pertains.

As used herein, an “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, “messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As used herein, unless otherwise dictated by context “nucleotide” or “nt” refers to ribonucleotide.

As used herein, the terms “patient” and “subject” are used interchangeably, and may be taken to mean any living organism which may be treated with compounds of the present invention. As such, the terms “patient” and “subject” include, but are not limited to, any non-human mammal, primate and human.

The term “stop codon” can refer to a three nucleotide contiguous sequence within messenger RNA that signals a termination of translation. Non-limiting examples include in RNA, UAG (amber), UAA (ochre), UGA (umber, also known as opal) and in DNA TAG, TAA or TGA. Unless otherwise noted, the term can also include nonsense mutations within DNA or RNA that introduce a premature stop codon, causing any resulting protein to be abnormally shortened.

The term “structured motif,” as disclosed herein, comprises two or more features in a guide-target RNA scaffold.

A “therapeutically effective amount” of a composition is an amount sufficient to achieve a desired therapeutic effect, and does not require cure or complete remission.

The terms “treat,” “treated,” “treating”, or “treatment” as used herein have the meanings commonly understood in the medical arts, and therefore does not require cure or complete remission, and therefore includes any beneficial or desired clinical results. Treatment includes eliciting a clinically significant response without excessive levels of side effects. Treatment also includes prolonging survival as compared to expected survival if not receiving treatment.

As used herein, “preventing” a disease refers to inhibiting the full development of a disease.

A double stranded RNA (dsRNA) substrate is formed upon hybridization of an engineered guide RNA of the present disclosure to a target RNA. The resulting dsRNA substrate is also referred to herein as a “guide-target RNA scaffold.” Described herein are structural features that can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

As used herein, the term “latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

As used herein, the term “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “guide-target RNA scaffold” refers to the resulting double-stranded RNA formed upon hybridization of a guide RNA, with latent structure, to a target RNA. A guide-target RNA scaffold has one or more structural features formed within the double-stranded RNA duplex upon hybridization. For example, the guide-target RNA scaffold can have one or more structural features selected from a bulge, mismatch, internal loop, hairpin, or wobble base pair.

As used herein, the term “structured motif” refers to two or more structural features in a guide-target RNA scaffold.

As used herein, the term “double-stranded RNA substrate” or “dsRNA substrate” refers to a guide-target RNA scaffold formed upon hybridization of an engineered guide RNA to a target RNA.

As used herein, the term “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature.

As used herein, the term “bulge” refers to a structure, substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can have from 0 to 4 contiguous nucleotides on the guide RNA side of the guide-target RNA scaffold and 1 to 4 contiguous nucleotides on the target RNA side of the guide-target RNA scaffold or a bulge can have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop.

As used herein, the term “symmetrical bulge” refers to a structure formed when the same number of nucleotides is present on each side of the bulge.

As used herein, the term “asymmetrical bulge” refers to a structure formed when a different number of nucleotides is present on each side of the bulge.

As used herein, the term “internal loop” refers to the structure, substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop.

As used herein, the term “symmetrical internal loop” refers to a structure formed when the same number of nucleotides is present on each side of the internal loop.

As used herein, the term “asymmetrical internal loop” refers to a structure formed when a different number of nucleotides is present on each side of the internal loop.

As used herein, the term “hairpin” refers to an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

As used herein, the term “recruitment hairpin” refers to a hairpin structure capable of recruiting, at least in part, an RNA editing entity, such as ADAR. In some cases, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, can include a naturally occurring ADAR substrate or truncations thereof. Thus, a recruitment hairpin such as GluR2 is a pre-formed structural feature that may be present in constructs comprising an engineered guide RNA, not a structural feature formed by latent structure provided in an engineered latent guide RNA.

As used herein, the term “non-recruitment hairpin” refers to a hairpin structure with a dissociation constant for binding to an RNA editing entity under physiological conditions that is insufficient for binding, e.g., that is not capable of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention.

As used herein, the term “wobble base pair” refers to two bases that weakly base pair. For example, a wobble base pair of the present disclosure can refer to a G paired with a U.

As used herein, the term “macro-footprint” refers to an over-arching structure of a guide RNA. In some embodiments, a macro-footprint flanks a micro-footprint. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint.

As used herein, the term “micro-footprint” refers to a guide structure with latent structures that, when manifested, facilitate editing of the adenosine of a target RNA via an adenosine deaminase enzyme. A macro-footprint can serve to guide an RNA editing entity (e.g., ADAR) and direct its activity towards a micro-footprint. In some embodiments, included within the micro-footprint sequence is a nucleotide that is positioned such that, when the guide RNA is hybridized to the target RNA, the nucleotide opposes the adenosine to be edited by the adenosine deaminase and does not base pair with the adenosine to be edited. This nucleotide is referred to herein as the “mismatched position” or “mismatch” and can be a cytosine. Micro-footprint sequences as described herein have upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA. Engineered guide RNAs selected for their ability to facilitate editing of a specific target are capable of adopting various micro-footprint latent structures, which can vary on a target-by-target basis.

As used herein, the term “barbell” refers to a guide macro-footprint having a pair of internal loop latent structures that manifest upon hybridization of the guide RNA to the target RNA.

As used herein, the term “dumbbell” refers to a macro-footprint having two symmetrical internal loops, wherein the target A to be edited is positioned between the two symmetrical loops for selective editing of the target A. The two symmetrical internal loops are each formed by 6 nucleotides on the guide RNA side of the guide-target RNA scaffold and 6 nucleotides on the target RNA side of the guide-target RNA scaffold. Thus, a dumbbell can be a structural feature formed from latent structure provided by an engineered latent guide RNA.

As used herein, the term “U-deletion” refers to a type of asymmetrical bulge. In some embodiments, a U-deletion is an asymmetrical bulge formed upon binding of an engineered guide RNA to an mRNA transcribed from a target gene. In some embodiments, a U-deletion is formed by 0 nucleotides on the engineered guide RNA side of the guide-target RNA scaffold and 1 nucleotide on the target RNA side of the guide-target RNA scaffold. For instance, in some implementations, a U-deletion is formed by an “A” on the target RNA side of the guide-target RNA scaffold and a deletion of a “U” on the engineered guide RNA side of the guide-target RNA scaffold. In some embodiments, U-deletions are used opposite of a local off-target nucleotide position (e.g., an off-target adenosine) to reduces off-target editing.

As used herein, the term “base paired region” or “bp region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold.

As used interchangeably herein, the term “classifier” or “model” refers to a machine learning model or algorithm.

In some embodiments, a model includes an unsupervised learning algorithm. One example of an unsupervised learning algorithm is cluster analysis. In some embodiments, a model includes supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, Gradient Boosting, mixture models, hidden Markov models, Gaussian NB algorithms, linear discriminant analysis, diffusion models, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). In some embodiments, neural networks are machine learning algorithms that are trained to map an input dataset to an output dataset, where the neural network includes an interconnected group of nodes organized into multiple layers of nodes. For example, in some embodiments, the neural network architecture includes at least an input layer, one or more hidden layers, and an output layer. In some embodiments, the neural network includes any total number of layers, and any number of hidden layers, where the hidden layers function as trainable feature extractors that allow mapping of a set of input data to an output value or set of output values. In some embodiments, a deep learning algorithm is a neural network including a plurality of hidden layers, e.g., two or more hidden layers. In some instances, each layer of the neural network includes a number of nodes (or “neurons”). In some embodiments, a node receives input that comes either directly from the input data or the output of nodes in previous layers, and performs a specific operation, e.g., a summation operation. In some embodiments, a connection from an input to a node is associated with a parameter (e.g., a weight and/or weighting factor). In some embodiments, the node sums up the products of all pairs of inputs, xᵢ, and their associated parameters. In some embodiments, the weighted sum is offset with a bias, b. In some embodiments, the output of a node or neuron is gated using a threshold or activation function, f, which, in some instances, is a linear or non-linear function. In some embodiments, the activation function is, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

In some implementations, the weighting factors, bias values, and threshold values, or other computational parameters of the neural network, are “taught” or “learned” in a training phase using one or more sets of training data. For example, in some implementations, the parameters are trained using the input data from a training dataset and a gradient descent or backward propagation method so that the output value(s) that the ANN computes are consistent with the examples included in the training dataset. In some embodiments, the parameters are obtained from a back propagation neural network training process.

Any of a variety of neural networks are suitable for use in accordance with the present disclosure. Examples include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. In some implementations, convolutional and/or residual neural networks are used, in accordance with the present disclosure.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 50 parameters, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For certain cases in which no linear separation is possible, SVMs work in combination with the technique of ‘kernels’, which automatically realizes a non-linear mapping to a feature space. The hyper-plane found by the SVM in feature space corresponds, in some instances, to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. In some implementations, nearest neighbor models are memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. In some embodiments, the nearest neighbor rule is refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. For example, one specific algorithm is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. In some embodiments, a regression algorithm is any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. In some embodiments, linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. In some embodiments, the resulting combination is used as the model (linear model) in some embodiments of the present disclosure.

Mixture model and Hidden Markov model. In some embodiments, the model is a mixture model, such as that described in McLachlan et al., Bioinformatics 18(3): 413-422, 2002. In some embodiments, in particular, those embodiments including a temporal component, the model is a hidden Markov model such as described by Schliep et al., 2003, Bioinformatics 19(1): i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As an illustrative example, in some embodiments, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to begin a clustering investigation is to define a distance function and to compute the matrix of distances between all pairs of samples in the training set. If distance is a good measure of similarity, then the distance between reference entities in the same cluster is significantly less than the distance between the reference entities in different clusters. However, in some implementations, clustering does not use a distance metric. For example, in some embodiments, a nonmetric similarity function s(x, x′) is used to compare two vectors x and x′. In some such embodiments, s(x, x′) is a symmetric function whose value is large when x and x′ are somehow “similar.” Once a method for measuring “similarity” or “dissimilarity” between points in a dataset has been selected, clustering uses a criterion function that measures the clustering quality of any partition of the data. Partitions of the dataset that extremize the criterion function are used to cluster the data. Particular exemplary clustering techniques contemplated for use in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering includes unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “untrained model” (e.g., “untrained classifier” and/or “untrained neural network”) refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that can be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning is used, in some such embodiments. For instance, consider the case where there is a first auxiliary training dataset and a second auxiliary training dataset in addition to the primary training dataset. In such a case, the parameters learned from the first auxiliary training dataset (by application of a first model to the first auxiliary training dataset) are applied to the second auxiliary training dataset using transfer learning techniques (e.g., a second model that is the same or different from the first model), which in turn results in a trained intermediate model whose parameters are then applied to the primary training dataset and this, in conjunction with the primary training dataset itself, is applied to the untrained model. Alternatively, in another example embodiment, a first set of parameters learned from the first auxiliary training dataset (by application of a first model to the first auxiliary training dataset) and a second set of parameters learned from the second auxiliary training dataset (by application of a second model that is the same or different from the first model to the second auxiliary training dataset) are each individually applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) are then applied to the untrained model in order to train the untrained model.

### RNA Editing System

RNA editing refers to a process by which RNA is, in some embodiments, enzymatically modified post synthesis on specific nucleosides. In some embodiments, RNA editing comprises any one of an insertion, deletion, or substitution of a nucleotide(s). Examples of RNA editing include pseudouridylation (the isomerization of uridine residues) and deamination (removal of an amine group from cytidine to give rise to uridine or C-to-U editing through recruitment of an APOBEC enzyme, or from adenosine to inosine or A-to-I editing through recruitment of an adenosine deaminase such as ADAR) as described herein. Editing of RNA, in some embodiments, is a way to regulate gene translation. RNA editing, in some embodiments, is a mechanism in which to regulate transcript recoding by regulating the triplet codon to introduce silent mutations and/or non-synonymous mutations.

Provided herein, in certain embodiments, are compositions that comprise engineered guide RNAs that facilitate RNA editing via an RNA editing entity or a biologically active fragment thereof and methods of using the same. In an aspect, an RNA editing entity, in some embodiments, comprises an adenosine Deaminase Acting on RNA (ADAR) and biologically active fragments thereof. In some instances, ADARs are enzymes that catalyze the chemical conversion of adenosines to inosines in RNA. Because the properties of inosine mimic those of guanosine (inosine will form two hydrogen bonds with cytosine, for example), inosine, in some embodiments, is recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs, in some embodiments, form homodimer as well as heterodimer with other ADARs when bound to double-stranded RNA; however, and without being limited to any one theory of operation, it is currently inconclusive if dimerization is needed for editing to occur.

Three human ADAR genes have been identified (ADARs 1-3) with ADAR1 (official symbol ADAR) and ADAR2 (ADARB1) proteins having well-characterized adenosine deamination activity. ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain. In some embodiments, different cell types express different ADAR isoforms. For example, neurons mainly express ADAR2. Many cell types, such as liver cells, express ADAR1 and ADAR2.

Specific RNA editing, in some embodiments, leads to transcript recoding. Because inosine shares the base pairing properties of guanosine, the translational machinery interprets edited adenosines as guanosine, altering the triplet codon, which, in some embodiments, results in amino acid substitutions in protein products. More than half the triplet codons in the genetic code could be reassigned through RNA editing. Due to the degeneracy of the genetic code, in some implementations, RNA editing causes both silent and non-synonymous amino acid substitutions.

In some cases, targeting an RNA affects splicing. Adenosines targeted for editing, in some embodiments, are disproportionately localized near splice junctions in pre-mRNA. Therefore, in some instances, during formation of a dsRNA ADAR substrate, intronic cis-acting sequences form RNA duplexes encompassing splicing sites and potentially obscuring them from the splicing machinery. Furthermore, in some instances, through modification of select adenosines, ADARs create or eliminate splicing sites, broadly affecting later splicing of the transcript. Similar to the translational machinery, the spliceosome interprets inosine as guanosine, and therefore, in some cases, a canonical GU 5′ splice site and AG 3′ acceptor site is created via the deamination of AU (IU=GU) and AA (AI=AG), respectively. Correspondingly, in some implementations, RNA editing destroys a canonical AG 3′ splice site (IG=GG).

Adenosines in a target RNA that are targeted for editing, in some embodiments, are in a coding sequence or a non-coding sequence of an RNA. For example, an adenosine targeted for editing in a coding sequence of an RNA can be part of a translation initiation site (TIS). The subsequent ADAR-mediated RNA editing of the TIS (AUG) to GUG facilitated by an engineered guide RNA, in some embodiments, results in inhibition of RNA translation and, thereby, protein knockdown. Protein knockdown can also be referred to as reduced expression of wild-type protein. In some embodiments, an adenosine targeted for editing in a non-coding sequence of an RNA can be part of a polyadenylation (polyA) signal sequence in a 3′UTR. The subsequent ADAR-mediated RNA editing of an adenosine in a polyA signal sequence facilitated by an engineered guide RNA, in some embodiments, results in disruption of RNA processing and degradation of the target mRNA and, thereby, protein knockdown.

In some cases, targeting an RNA affects microRNA (miRNA) production and function. For example, in various embodiments, RNA editing of a pre-miRNA precursor affects the abundance of a miRNA, RNA editing in the seed of the miRNA redirects it to another target for translational repression, and/or RNA editing of a miRNA binding site in an RNA interferes with miRNA complementarity, and thus interferes with suppression via RNAi.

In an aspect, an RNA editing entity is recruited by a guide RNA of the present disclosure. In some examples, a guide RNA recruits an RNA editing entity that, when associated with the guide RNA and a target RNA as described herein, facilitates: an editing of a base of a nucleotide of the target RNA, a modulation of the expression of a polypeptide encoded by a subject target RNA; or a combination thereof. A guide RNA, in some embodiments, optionally contains an RNA editing entity recruiting domain capable of recruiting an RNA editing entity. In some embodiments, a guide RNA lacks an RNA editing entity recruiting domain and is still capable of binding an RNA editing entity, or of being bound by it.

Disclosed herein are engineered guide RNAs for site-specific, selective editing of a target RNA via an RNA editing entity or a biologically active fragment thereof. An engineered guide RNA of the present disclosure, in some embodiments, comprises latent structures, such that when the engineered guide RNA is hybridized to the target RNA to form a guide-target RNA scaffold, at least a portion of the latent structure manifests as at least a portion of a structural feature as described herein.

In some embodiments, an engineered guide RNA as described herein comprises a targeting domain with complementarity to a target RNA described herein. As such, in some implementations, a guide RNA is engineered to site-specifically/selectively target and hybridize to a particular target RNA, thus facilitating editing of a specific target RNA via an RNA editing entity or a biologically active fragment thereof. The targeting domain, in some embodiments, includes a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch, in some embodiments, helps to localize editing of the RNA editing entity to the desired base of the target RNA. In some embodiments, this mismatch is absent and other latent structures help localize editing of the RNA editing entity to the desired base of the target RNA. However, in some instances there are some, and in some cases significant, off-target editing in addition to the desired edit.

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. A micro-footprint sequence of a guide RNA comprising latent structures (e.g., a “latent structure guide RNA”), in some embodiments, comprises a portion of sequence that, upon hybridization to a target RNA, forms at least a portion of a structural feature, other than a single A/C mismatch feature at the target adenosine to be edited. Further, the structural features in combination generally facilitate an increased amount of editing of a target adenosine, fewer off target edits, or both, as compared to a construct comprising the mismatch alone or a construct having perfect complementarity to a target RNA. In some embodiments, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold, in some embodiments, is a powerful tool to promote editing of a target RNA with high specificity, selectivity, and robust activity.

In some embodiments, hybridization of the target RNA and the targeting domain of the guide RNA also produces specific tertiary structures in the guide-target RNA scaffold that manifest upon hybridization. Tertiary structures when manifested become features described herein, including coaxial stacking, A-platforms, interhelical packing motifs, triplexes, major groove triples, minor groove triples, tetraloop motifs, metal-core motifs, ribose zippers, kissing loops, and pseudoknots. Without wishing to be bound by theory, the presence of tertiary structures features described herein that are produced upon hybridization of the guide RNA with the target RNA configure the guide RNA aid in a specific, or selective, targeted edit of the target RNA via the RNA editing entity or biologically active fragment thereof.

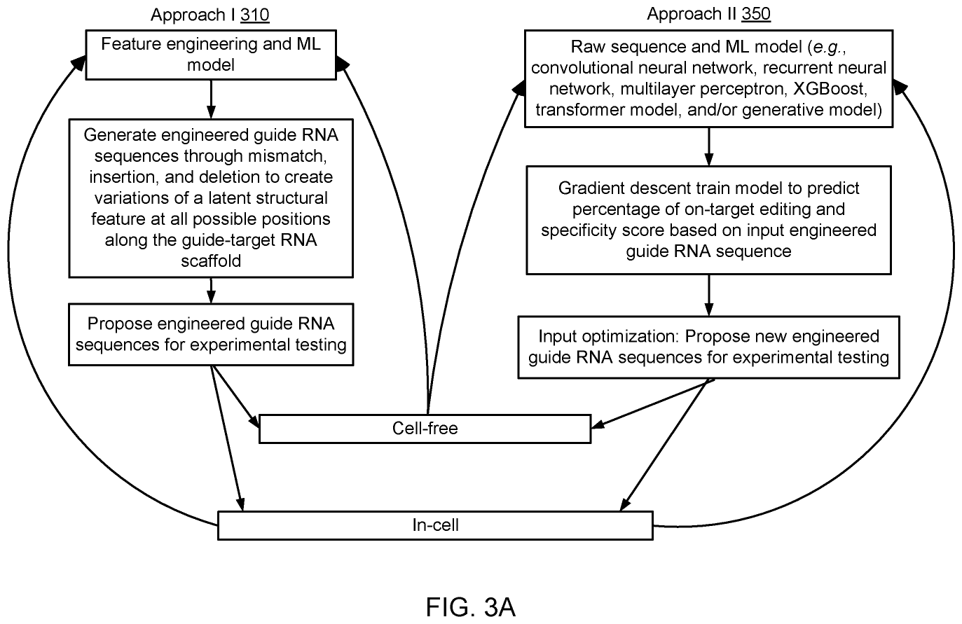

However, the theoretical guide design space for a target RNA (e.g., the number of possible of permutations of latent structural features, secondary structural features, tertiary structures, and/or ADAR recruiting domains in an engineered guide RNA for a target RNA) that requires experimental testing to determine if the engineered guide RNA has the desired on-target editing and specificity score is extremely large. For example, for an engineered guide RNA that is 100 nt in length, a pool of about 10⁶⁰ engineered guide RNAs (comprising only latent structural features) that would need to be tested. Furthermore, even if about two-thirds of the engineered guide RNA needs to be kept constant (e.g., two-thirds of the sequence needs to maintain complementarity to the target RNA to form a stable guide-target RNA scaffold), this assumes a constraint of 30 nt mutation window, there is still a pool of about 10⁴³ engineered guide RNAs (comprising only latent structural features) that would need to be tested. As described herein, in some implementations, various machine learning approaches are used to aid in reducing the pool of engineered guide RNAs for experimental testing. For instance, in some example embodiments, machine learning as described herein is used to predict the on-target editing and specificity score of an engineered guide RNA for a target RNA. Machine learning, in some embodiments, is also used to generate engineered guide RNA sequences that have a specified on-target editing and specificity score for a target RNA. Furthermore, machine learning, in some embodiments, is used to determine key features (e.g., latent structural features) that impact on-target editing and specificity score for a target RNA. Therefore, using these machine learning models alone or in any combination, in some embodiments, aids in narrowing the pool of engineered guide RNAs to be tested for having the desired on-target editing and specificity score (see, e.g., FIG. 3).

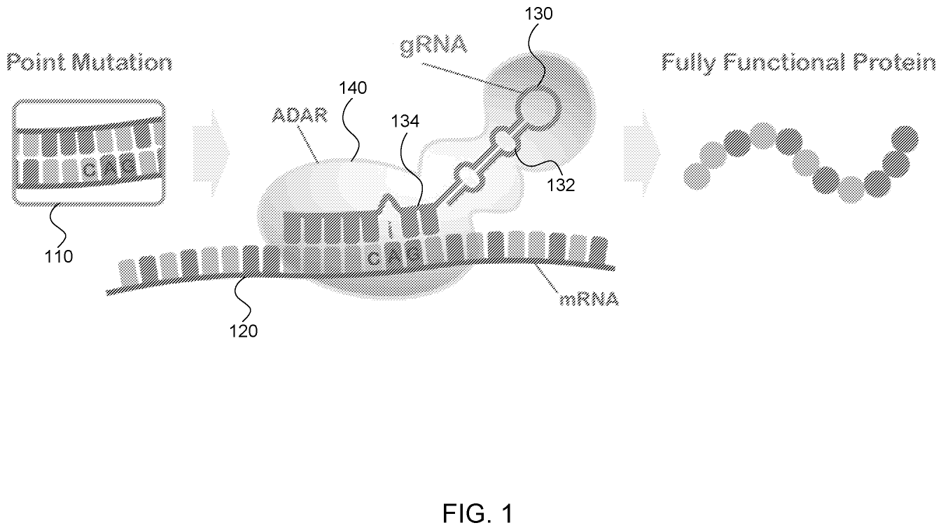

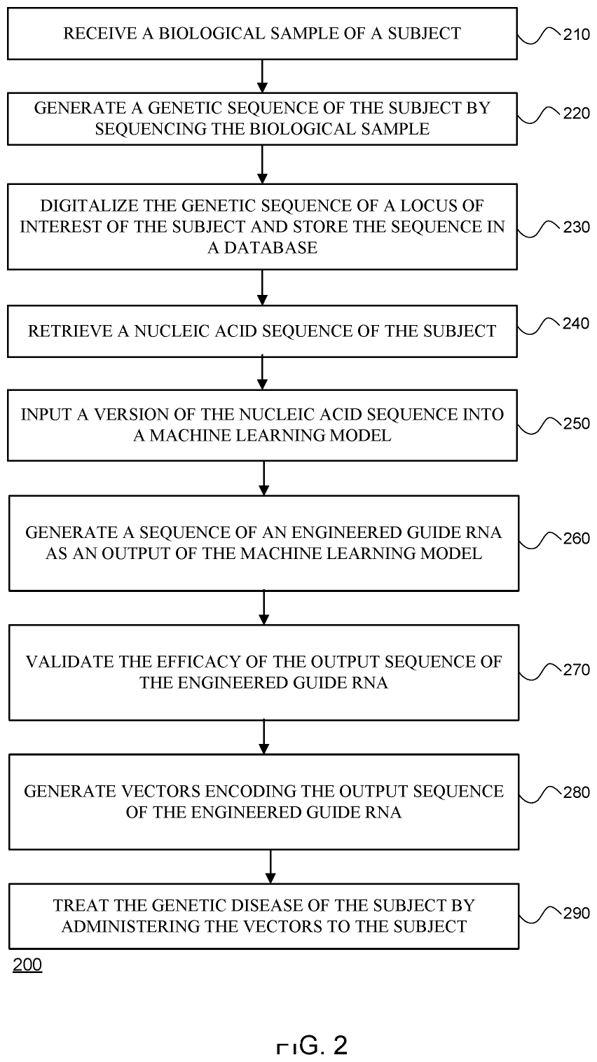

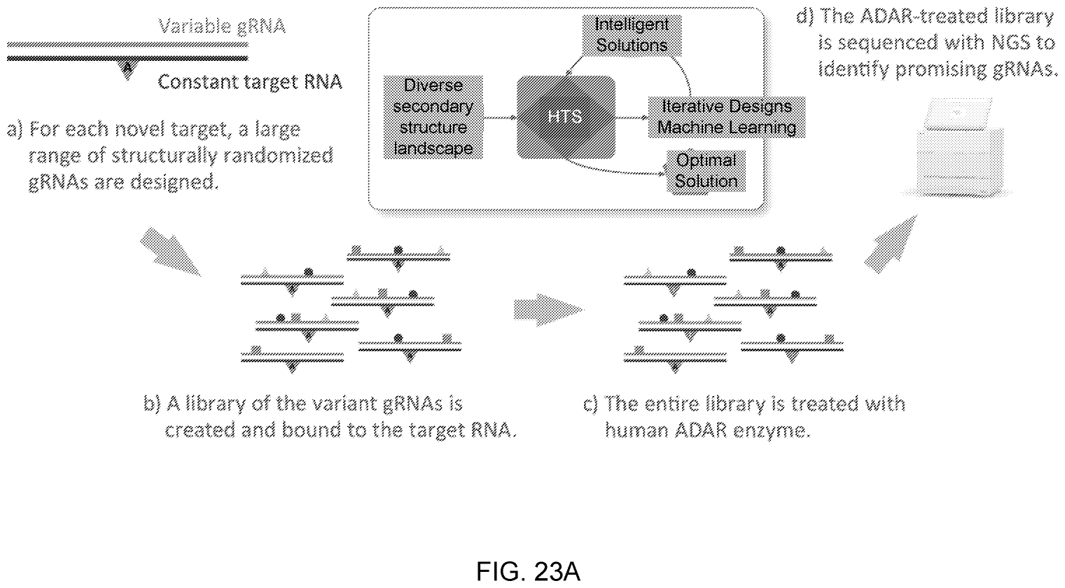

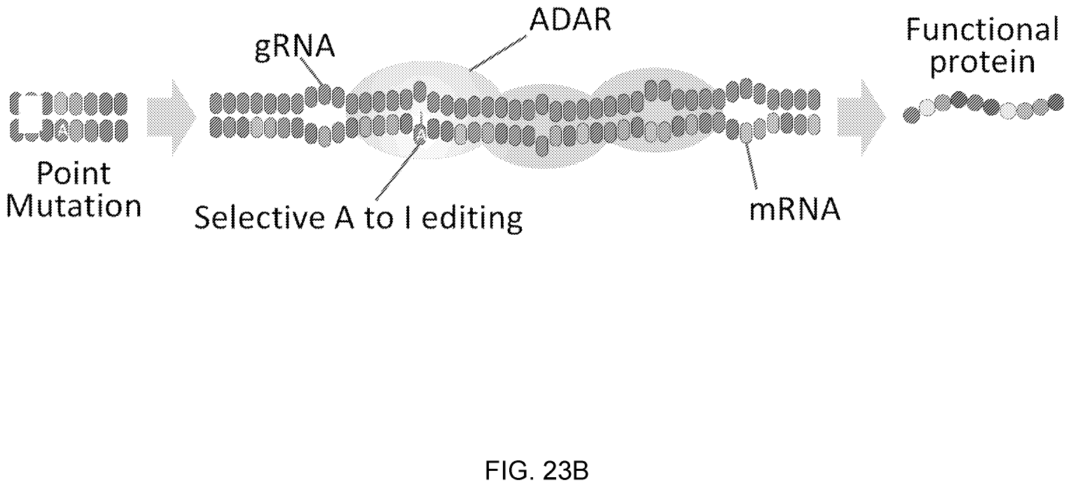

FIG. 1 is a conceptual diagram illustrating an exemplary RNA editing system as described herein, in accordance with some embodiments. The patient's DNA sequence 110, in some embodiments, suffers from a mutation, such as a point mutation. In the example shown, the patient suffers from a G>A substitution that renders the gene non-functional. The mutation in DNA is carried into a mutated RNA sequence after the DNA sequence 110 is transcribed into a messenger RNA (mRNA) 120. The mutation at the site of interest from G>A, in some embodiments, leads to dysfunctional or toxic protein products, thereby causing a genetic disease. In some embodiments, an engineered guide agent 130, such as a guide RNA (gRNA), is used to guide an adenosine deaminase acting on RNA (ADAR) enzyme 140 (simply referred to as ADAR) to edit the mutated mRNA 120. The ADAR 140, in some embodiments, is a naturally occurring enzyme editor that is found in most, if not all, human cells. A portion of the engineered guide RNA (gRNA) 130, in some embodiments, is hybridized to the target mRNA 120 to form a guide-target RNA scaffold. The engineered gRNA 130 recruits the ADAR 140 to catalyze a formation of RNA editing complex that includes the mRNA 120, the engineered gRNA 130, and the ADAR 140. The ADAR 140 catalyzes editing that substitutes the site of interest from adenosine to inosine (A>I). Inosine is read by the ribosome as guanosine (G), which causes an amino acid change in a protein. As a result, in some embodiments, a fully functional protein 150 is translated from the edited mRNA 120 and the patient's genetic disease is treated. As an additional example, an engineered RNA guide of this RNA editing system, in some embodiments, is an engineered RNA guide of FIG. 23B.

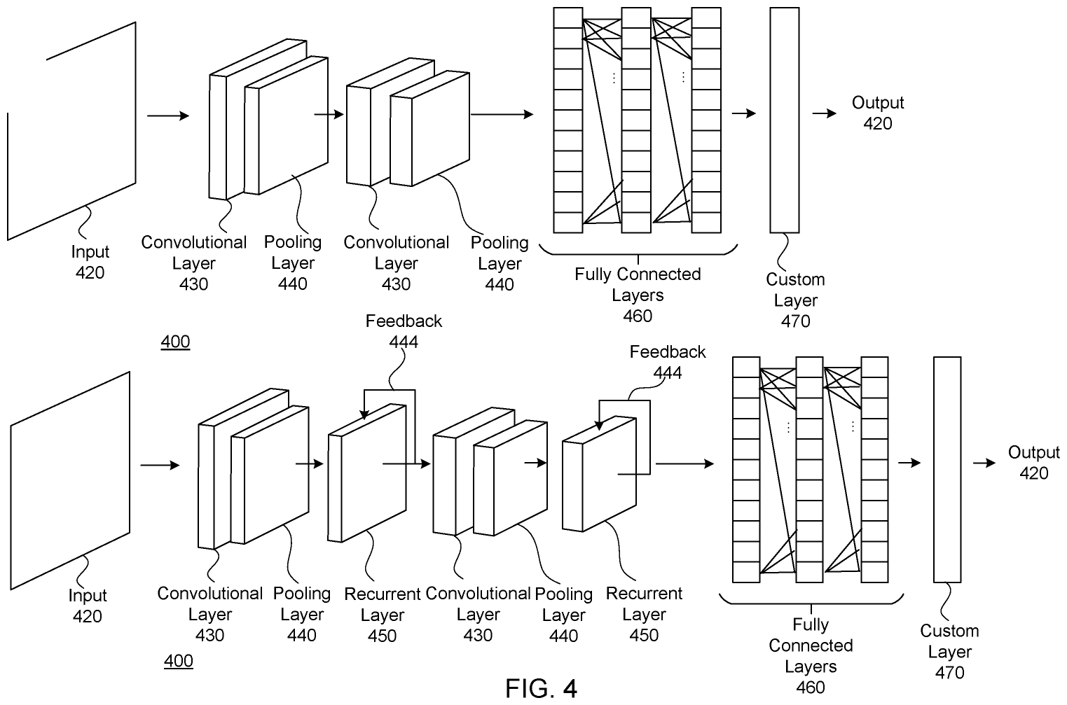

In some embodiments, the percentage of on-target editing of the mutation at the site of interest and the specificity score of the engineered gRNA 130 or of FIG. 23B is determined by one or more machine learning models. In some embodiments, the precise sequence of the engineered gRNA 130 or of FIG. 23B is determined by one or more machine learning models. The precise sequence, in some embodiments, is generated for a high percentage of on-target editing and a high specificity score to improve or optimize the RNA editing system. The sequence of the engineered gRNA 130 or of FIG. 23B, in some embodiments, is determined based on the sequence of the target mRNA and the nucleotide of interest for editing. This machine learning based sequence determination process is discussed in further detail with references to FIGS. 3 through 4.

The engineered gRNA 130 comprises one or more specific RNA targeting domains 134. In some embodiments, at least one RNA targeting domain 134 has a sequence that is only partially complementary to the sequence of a segment of the target RNA. The one or more specific RNA targeting domains 134, in some embodiments, further comprises one or more latent structural features. Binding of the engineered guide RNA 130 to the target mRNA 120 generates a double stranded substrate (also referred to as a guide-target RNA scaffold) for ADAR 140, which when ADAR is bound to the guide-target RNA scaffold, deaminates one or more mismatched adenosine residues in target mRNA 120. The engineered guide RNA 130 thus serves, in typical embodiments, to facilitate ADAR editing. In certain embodiments, the engineered guide RNA 130 facilitates editing by ADAR2. In certain embodiments, the engineered guide RNA 130 facilitates editing by ADAR1. Another exemplary engineered guide RNA is shown in FIG. 23B.

In some embodiments, the RNA targeting domain 134 is at least partially complementary to a target RNA. In some embodiments, RNA targeting domain 134 has a sequence that is complementary to the sequence of a segment of the target RNA 120 except for a mismatch corresponding to a target editing site for modifying/changing a specific adenosine to inosine in the target RNA 120. In some embodiments, the RNA targeting domain 134 is an antisense oligonucleotide sequence. In some embodiments, the engineered guide RNA of FIG. 23B also has a targeting domain corresponding to RNA targeting domain 134.

In some embodiments, the engineered gRNA 130 optionally further comprises an ADAR recruiting domain 132. The ADAR recruiting domain 132, in some embodiments, mimics the ADAR recruiting portion of a mammalian pre-mRNA. The RNA targeting domain 134 is at 5′ and/or 3′ end of the ADAR recruiting domain 132. For example, even though, in the particular example shown in FIG. 1, the RNA targeting domain 134 is present at only one end of the ADAR recruiting domain 132, in other embodiments, a second RNA targeting domain 134 is present at the other end of the ADAR recruiting domain 132. Binding of the target mRNA 120 to the engineered guide RNA comprising an ADAR recruiting domain 134 generates a guide-target RNA scaffold for ADAR 140 and recruits ADAR 140, which when bound to the guide-target RNA scaffold, ADAR deaminates one or more mismatched adenosine residues in target mRNA 120.

The optional ADAR recruiting domain 132 of the gRNA 130 mimics certain aspects of the ADAR-recruiting portion of a mammalian RNA. The recruiting domain 132 thus serves, in typical embodiments, to recruit ADAR 1, ADAR2, and/or ADAR3, or any combination thereof, to the target sequence, and facilitates subsequent editing. In certain embodiments, the ADAR recruiting domain 132 facilitates editing by ADAR2. In certain embodiments, the ADAR recruiting domain 132 facilitates editing by ADAR1.

The ADAR recruiting domain 132, in some embodiments, includes one or more recruitment hairpins. For example, the ADAR recruiting domain is a GluR2 domain or a Alu-based domain. In some embodiments, the ADAR recruiting domain 132 forms a contiguous sequence with the targeting domain 134. In other embodiments, the ADAR recruiting domain 132 is separate from the targeting domain 134, but will form a complex when both are transcribed within a cell at the same time.

In various embodiments, the engineered guide agent 130 or engineered guide RNA of FIG. 23B promotes both ADAR recruitment and target recognition by target-RNA hybridization. In some embodiments, site-directed RNA editing is achieved by guiding ADAR onto the target site.

In various embodiments, the ADAR 140 or of FIG. 23B targets adenosine located in double-stranded RNA (dsRNA) generated by the engineered guide RNA 130 or of FIG. 23B hybridizing to the target mRNA 120 or of FIG. 23B (also referred to as the “guide-target RNA scaffold”). In some embodiments, binding of the ADAR 140 or of FIG. 23B to guide-target RNA scaffold facilitates site-direct A-to-I editing of the target mRNA 120 or of FIG. 23B, resulting in translation of a functional protein from edited target mRNA in a cell.

In various embodiments, the ADAR recruiting domain 132 is between 40-90 ribonucleotides in length. In some embodiments, the recruiting domain 132 is between 50-80 ribonucleotides in length, or 60-70 ribonucleotides in length. In certain embodiments, the recruiting domain 132 is 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt, 81 nt, 82 nt, 83 nt, 84 nt, 85 nt, 86 nt, 87 nt, 88 nt, 89 nt, or 90 nt in length.

In various embodiments, the ADAR recruiting domain 132 comprises the ADAR-recruiting portion of a mammalian mRNA with one or more substitutions, insertions and/or deletions of nucleotides, so long as the ADAR recruiting activity is not lost. In some embodiments, the one or more substitutions, insertions and deletions of nucleotides improve a desired property of the engineered guide RNA 130. In some embodiments, the sequence of the recruiting domain 132 is modified (e.g., substitution, deletion, insertion) by one or more machine learning models so that the recruiting throughput, on-target activity, and specificity of the engineered guide agent 130 are improved. The modification, in some embodiments, is performed based on a base sequence such as an engineered sequence or a wild-type gRNA.

In some embodiments, the engineered guide RNA 130 or of FIG. 23B recruits any one of or a combination of ADAR1 and ADAR2. In some embodiments, the engineered guide agent 130 or engineered guide RNA of FIG. 23B has a preferential binding to ADAR1. In some embodiments, the engineered guide agent 130 or engineered guide RNA of FIG. 23B has a preferential binding to ADAR2.

In some embodiments, the engineered guide RNA 130 lacks an ADAR recruiting domain 132, for example, see the engineered guide RNA of FIG. 23B. In some embodiments, the engineered guide RNA 130 has one ADAR recruiting domain 132. In some embodiments, the engineered guide agent 130 has two ADAR recruiting domains 132. In some embodiments, the engineered guide RNA 130 includes a plurality of ADAR recruiting domains 132.

### 1. Engineered Guide RNAs

Disclosed herein are engineered guide RNAs and engineered polynucleotides encoding the same for site-specific, selective editing of a target RNA via an RNA editing entity or a biologically active fragment thereof. An engineered guide RNA of the present disclosure, in some embodiments, comprises latent structures, such that when the engineered guide RNA is hybridized to the target RNA to form a guide-target RNA scaffold, at least a portion of the latent structure manifests as at least a portion of a structural feature as described herein. In some embodiments, an engineered guide RNA of the present disclosure comprises tertiary structures, such that when the engineered guide RNA is hybridized to the target RNA to form a guide-target RNA scaffold, at least a portion of the tertiary structure manifests.

An engineered guide RNA as described herein comprises a targeting domain with complementarity to a target RNA described herein. As such, a guide RNA, in some embodiments, is engineered to site-specifically/selectively target and hybridize to a particular target RNA, thus facilitating editing of specific nucleotide in the target RNA via an RNA editing entity or a biologically active fragment thereof. The targeting domain, in some embodiments, includes a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch, in some embodiments, helps to localize editing of the RNA editing entity to the desired base of the target RNA. However, in some instances there are some, and in some cases significant, off-target editing in addition to the desired edit.

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. A micro-footprint sequence of a guide RNA comprising latent structures (e.g., a “latent structure guide RNA”), in some embodiments, comprises a portion of sequence that, upon hybridization to a target RNA, forms at least a portion of a structural feature, other than a single A/C mismatch feature at the target adenosine to be edited. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination generally facilitate an increased amount of editing of a target adenosine, fewer off target edits, or both, as compared to a construct comprising the mismatch alone or a construct having perfect complementarity to a target RNA. In some embodiments, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold, in some embodiments, is a powerful tool to promote editing of the target RNA with high specificity, selectivity, and robust activity.

In some embodiments, hybridization of the target RNA and the targeting domain of the guide RNA also produces specific tertiary structures in the guide-target RNA scaffold that manifest upon hybridization. Tertiary structures when manifested become features described herein, including coaxial stacking, A-platforms, interhelical packing motifs, triplexes, major groove triples, minor groove triples, tetraloop motifs, metal-core motifs, ribose zippers, kissing loops, and pseudoknots. Without wishing to be bound by theory, the presence of tertiary structures features described herein that are produced upon hybridization of the guide RNA with the target RNA configure the guide RNA aid in a specific, or selective, targeted edit of the target RNA via the RNA editing entity or biologically active fragment thereof.

Provided herein are engineered guides and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

In some examples, the engineered guides provided herein comprise an engineered guide that is configured, upon hybridization to a target RNA molecule, to form, at least in part, a guide-target RNA scaffold with at least a portion of the target RNA molecule, where the guide-target RNA scaffold comprises at least one structural feature, and where the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure is a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA. The part of the engineered guide RNA that hybridizes to the target RNA is of sufficient complementary to the sequence of the target RNA for hybridization to occur.

### A. Targeting Domain

Engineered guide RNAs disclosed herein, in some embodiments, are engineered in any way suitable for RNA editing. In some examples, an engineered guide RNA generally comprises at least a targeting sequence that allows it to hybridize to a region of a target RNA molecule. A targeting sequence is also referred to herein as a “targeting domain” or a “targeting region”.

In some cases, a targeting domain of an engineered guide allows the engineered guide to target an RNA sequence through base pairing, such as Watson Crick base pairing. In some examples, the targeting sequence is located at either the N-terminus or C-terminus of the engineered guide. In some cases, the targeting sequence is located at both termini. The targeting sequence, in some embodiments, is of any length. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence is no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide comprises a targeting sequence that is from about 60 to about 500, from about 60 to about 200, from about 75 to about 100, from about 80 to about 200, from about 90 to about 120, or from about 95 to about 115 nucleotides in length. In some examples, an engineered guide RNA comprises a targeting sequence that is about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence, in some embodiments, have a single base mismatch.

The targeting sequence, in some embodiments, has sufficient complementarity to a target RNA to allow for hybridization of the targeting sequence to the target RNA. In some embodiments, the targeting sequence has a minimum antisense complementarity of about 50 nucleotides or more to the target RNA. In some embodiments, the targeting sequence has a minimum antisense complementarity of about 60 nucleotides or more to the target RNA. In some embodiments, the targeting sequence has a minimum antisense complementarity of about 70 nucleotides or more to the target RNA. In some embodiments, the targeting sequence has a minimum antisense complementarity of about 80 nucleotides or more to the target RNA. In some embodiments, the targeting sequence has a minimum antisense complementarity of about 90 nucleotides or more to the target RNA. In some embodiments, the targeting sequence has a minimum antisense complementarity of about 100 nucleotides or more to the target RNA. In some embodiments, antisense complementarity refers to non-contiguous stretches of sequence. In some embodiments, antisense complementarity refers to contiguous stretches of sequence.

### B. Engineered Guide RNAs Having a Recruiting Domain

In some examples, a subject engineered guide RNA comprises a recruiting domain that recruits an RNA editing entity (e.g., ADAR), where in some instances, the recruiting domain is formed and present in the absence of binding to the target RNA. A “recruiting domain” is also referred to herein as a “recruiting sequence” or a “recruiting region”. In some examples, a subject engineered guide facilitates editing of a base of a nucleotide of in a target sequence of a target RNA that results in modulating the expression of a polypeptide encoded by the target RNA. Said modulation, in some embodiments, refers to increased expression of the polypeptide or decreased expression of the polypeptide. In some cases, an engineered guide is configured to facilitate an editing of a base of a nucleotide or polynucleotide of a region of an RNA by an RNA editing entity (e.g., ADAR). In order to facilitate editing, an engineered polynucleotide of the disclosure, in some embodiments, recruits an RNA editing entity (e.g., ADAR). Various RNA editing entity recruiting domains can be utilized. In some examples, a recruiting domain comprises: Glutamate ionotropic receptor AMPA type subunit 2 (GluR2), or an Alu sequence.

In some examples, more than one recruiting domain is included in an engineered guide of the disclosure. In examples where a recruiting domain is present, the recruiting domain is utilized to position the RNA editing entity to effectively react with a subject target RNA after the targeting sequence hybridizes to a target sequence of a target RNA. In some cases, a recruiting domain allows for transient binding of the RNA editing entity to the engineered guide. In some examples, the recruiting domain allows for permanent binding of the RNA editing entity to the engineered guide. A recruiting domain can be of any length. In some cases, a recruiting domain is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain is no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain is about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In an embodiments, a recruiting domain comprises a GluR2 sequence or functional fragment thereof. In some cases, a GluR2 sequence is recognized by an RNA editing entity, such as an ADAR or biologically active fragment thereof. In some embodiments, a GluR2 sequence comprises a non-naturally occurring sequence. In some cases, a GluR2 sequence is modified, for example for enhanced recruitment. In some embodiments, a GluR2 sequence comprises a portion of a naturally occurring GluR2 sequence and a synthetic sequence.

In some embodiments, a recruiting domain comprises a recruitment hairpin. A “recruitment hairpin,” as disclosed herein, in some embodiments, recruits at least in part an RNA editing entity, such as ADAR. In some cases, a recruitment hairpin is formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, in some embodiments, includes a naturally occurring ADAR substrate or truncations thereof. Thus, in some embodiments, a recruitment hairpin such as GluR2 is a pre-formed structural feature that is present in constructs comprising an engineered guide RNA, not a structural feature formed by latent structure provided in an engineered latent guide RNA.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 1). In some cases, a recruiting domain comprises at least about 80% sequence homology to at least about 10, 15, 20, 25, or 30 nucleotides of SEQ ID NO: 1. In some examples, a recruiting domain comprises at least about 90%, 95%, 96%, 97%, 98%, or 99% sequence homology and/or length to SEQ ID NO: 1.

Any number of recruiting domains can be found in an engineered guide of the present disclosure. In some examples, at least about 1, 2, 3, 4, 5, 6, 7, 8, 9, or up to about 10 recruiting domains are included in an engineered guide. Recruiting domains, in some embodiments, are located at any position of engineered guide RNAs. In some cases, a recruiting domain is on an N-terminus, middle, or C-terminus of an engineered guide RNA. A recruiting domain, in some embodiments, is upstream or downstream of a targeting sequence. In some cases, a recruiting domain flanks a targeting sequence of a subject guide. In some embodiments, a recruiting sequence comprises all ribonucleotides or deoxyribonucleotides, although a recruiting domain comprising both ribo- and deoxyribonucleotides in some cases is not excluded.

C. Engineered Guide RNAs with Latent Structure

In some examples, an engineered guide disclosed herein useful for facilitating editing of a target RNA via an RNA editing entity is an engineered latent guide RNA. An “engineered latent guide RNA” refers to an engineered guide RNA that comprises latent structure. A micro-footprint sequence of a guide RNA comprising latent structures (e.g., a “latent structure guide RNA”), in some embodiments, comprises a portion of sequence that, upon hybridization to a target RNA, forms at least a portion of a structural feature, other than a single A/C mismatch feature at the target adenosine to be edited. A micro-footprint, in some embodiments, serves to guide an RNA editing enzyme and direct its activity towards the target adenosine to be edited. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

A double stranded RNA (dsRNA) substrate is formed upon hybridization of an engineered guide RNA of the present disclosure to a target RNA. The resulting dsRNA substrate is also referred to herein as a “guide-target RNA scaffold.”

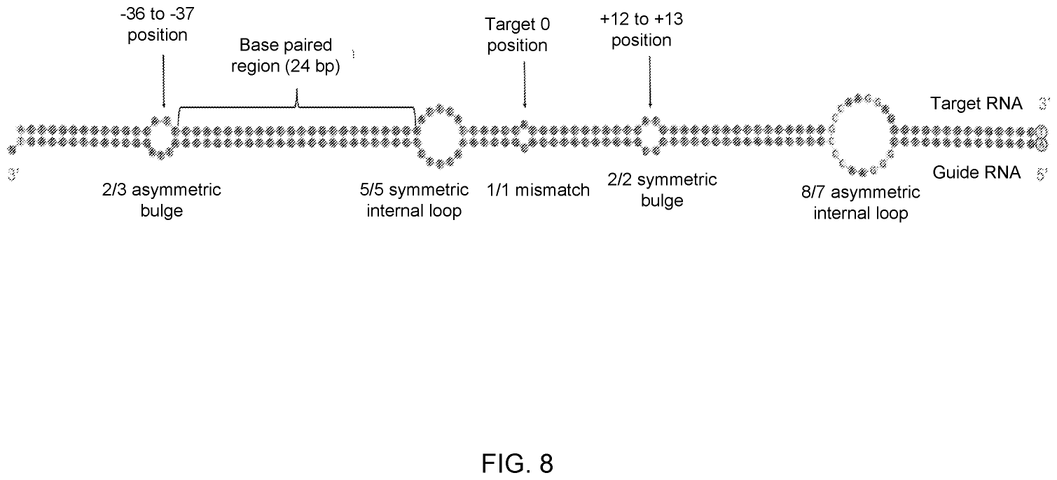

FIG. 8 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity is effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR). The engineered guide RNA, upon hybridization to a target RNA, form with the target RNA molecule, one or more structural features that recruits an RNA editing entity (e.g., ADAR).

In cases where a recruiting sequence is absent, an engineered guide RNA is still capable of associating with a subject RNA editing entity (e.g., ADAR) to facilitate editing of a target RNA and/or modulate expression of a polypeptide encoded by a subject target RNA. This is achieved, in some embodiments, through structural features formed in the guide-target RNA scaffold formed upon hybridization of the engineered guide RNA and the target RNA. Structural features, in some embodiments, comprise any one of a: mismatch, symmetrical bulge, asymmetrical bulge, symmetrical internal loop, asymmetrical internal loop, hairpins, wobble base pairs, or any combination thereof.

Described herein are structural features which, in some embodiments, are present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). In some embodiments, a structural feature further comprises a U-deletion formed upon binding of the gRNA to the mRNA transcribed from the target gene. In some embodiments, structural features (e.g., mismatches, bulges, internal loops) are formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA). Engineered guide RNAs of the present disclosure, in some embodiments, have from 1 to 50 features. Engineered guide RNAs of the present disclosure, in some embodiments, have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features.

Structural features, in some embodiments, are separated by a base paired region in an engineered guide. As disclosed herein, a “base paired (bp) region” refers to a region of the guide-target RNA scaffold in which bases in the guide RNA are paired with opposing bases in the target RNA. Base paired regions, in some embodiments, extend from one end or proximal to one end of the guide-target RNA scaffold to or proximal to the other end of the guide-target RNA scaffold. Base paired regions, in some embodiments, extend between two structural features. Base paired regions, in some embodiments, extend from one end or proximal to one end of the guide-target RNA scaffold to or proximal to a structural feature. Base paired regions, in some embodiments, extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

A guide-target RNA scaffold is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch, in some embodiments, comprises any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch, in some embodiments, comprises a C in an engineered guide RNA of the present disclosure opposite an A in a target RNA. An A/C mismatch, in some embodiments, comprises an A in an engineered guide RNA of the present disclosure opposite a C in a target RNA. A G/G mismatch, in some embodiments, comprises a G in an engineered guide RNA of the present disclosure opposite a G in a target RNA.

In some embodiments, a mismatch positioned 5′ of the edit site facilitates base-flipping of the target A to be edited. A mismatch, in some embodiments, also helps confer sequence specificity. Thus, a mismatch, in some embodiments, is a structural feature formed from latent structure provided by an engineered latent guide RNA.

In another aspect, a structural feature comprises a wobble base. A wobble base pair refers to two bases that weakly base pair. For instance, in an example embodiment, a wobble base pair of the present disclosure refers to a G paired with a U. Thus, a wobble base pair, in some embodiments, is a structural feature formed from latent structure provided by an engineered latent guide RNA.

In some cases, a structural feature is a hairpin. A hairpin, in some embodiments, refers to a recruitment hairpin (as described above), a non-recruitment hairpin, or any combination thereof. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin, in some embodiments, has from 10 to 500 nucleotides in length of the entire duplex structure. The loop portion of a hairpin, in some embodiments, is from 3 to 15 nucleotides long. A hairpin, in some embodiments, is present in any of the engineered guide RNAs disclosed herein. The engineered guide RNAs disclosed herein, in some embodiments, have from 1 to 10 hairpins. In some embodiments, the engineered guide RNAs disclosed herein have 1 or 2 hairpins. As disclosed herein, a hairpin, in some embodiments, includes a recruitment hairpin or a non-recruitment hairpin. A hairpin, in some embodiments, is located anywhere within the engineered guide RNAs of the present disclosure. In some embodiments, one or more hairpins is proximal to or present at 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin binds an RNA editing entity when present at 25° C. with a dissociation constant greater than about 1 mM, 10 mM, 100 mM, or 1 M, as determined in an in vitro assay. A non-recruitment hairpin, in some embodiments, exhibits functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that, in some embodiments, is present in constructs comprising engineered guide RNA constructs, not a structural feature formed by latent structure provided in an engineered latent guide RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that, in some embodiments, is present in constructs comprising engineered guide RNA constructs, not a structural feature formed by latent structure provided in an engineered latent guide RNA.

A hairpin of the present disclosure, in some embodiments, is of any length. In an aspect, a hairpin is from about 10-500 or more nucleotides. In some cases, a hairpin comprises at least about 10, 20, 30, 40, 50, 100, 150, 200, 300, 400, 500 or more nucleotides. In other cases, a hairpin comprises 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

In some aspects, a structural feature of an engineered guide RNA is a bulge. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge, in some embodiments, changes the secondary or tertiary structure of the guide-target RNA scaffold. In some embodiments, a bulge independently has from 0 to 4 contiguous nucleotides on the guide RNA side of the guide-target RNA scaffold and 1 to 4 contiguous nucleotides on the target RNA side of the guide-target RNA scaffold or a bulge independently has from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair-a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, in some embodiments, a bulge is a structural feature formed from latent structure provided by an engineered latent guide RNA.

In some embodiments, the presence of a bulge in a guide-target RNA scaffold positions or helps to position ADAR to selectively edit the target A in the target RNA and reduce off-target editing of non-target A(s) in the target RNA. In some embodiments, the presence of a bulge in a guide-target RNA scaffold recruits or helps recruit additional amounts of ADAR. Bulges in guide-target RNA scaffolds disclosed herein, in some embodiments, recruit other proteins, such as other RNA editing entities. In some embodiments, a bulge positioned 5′ of the edit site facilitates base-flipping of the target A to be edited. A bulge, in some embodiments, also helps confer sequence specificity for the A of the target RNA to be edited, relative to other A(s) present in the target RNA. For example, in some implementations, a bulge helps direct ADAR editing by constraining it in an orientation that yields selective editing of the target A.

A bulge, in some embodiments, is a symmetrical bulge or an asymmetrical bulge. A symmetrical bulge is formed when the same number of nucleotides is present on each side of the bulge. For example, in some implementations, a symmetrical bulge in a guide-target RNA scaffold of the present disclosure has the same number of nucleotides on the engineered guide RNA side and the target RNA side of the guide-target RNA scaffold. A symmetrical bulge of the present disclosure, in some embodiments, is formed by 2 nucleotides on the engineered guide RNA side of the guide-target RNA scaffold target and 2 nucleotides on the target RNA side of the guide-target RNA scaffold. A symmetrical bulge of the present disclosure, in some embodiments, is formed by 3 nucleotides on the engineered guide RNA side of the guide-target RNA scaffold target and 3 nucleotides on the target RNA side of the guide-target RNA scaffold. A symmetrical bulge of the present disclosure, in some embodiments, is formed by 4 nucleotides on the engineered guide RNA side of the guide-target RNA scaffold target and 4 nucleotides on the target RNA side of the guide-target RNA scaffold. Thus, a symmetrical bulge, in some embodiments, is a structural feature formed from latent structure provided by an engineered latent guide RNA.

An asymmetrical bulge is formed when a different number of nucleotides is present on each side of the bulge. For example, in some implementations, an asymmetrical bulge in a guide-target RNA scaffold of the present disclosure has different numbers of nucleotides on the engineered guide RNA side and the target RNA side of the guide-target RNA scaffold. An asymmetrical bulge of the present disclosure, in some embodiments, is formed by 0 nucleotides on the engineered guide RNA side of the guide-target RNA scaffold and 1, 2, 3, or 4 nucleotides on the target RNA side of the guide-target RNA scaffold. In some implementations, an asymmetrical bulge of the present disclosure is formed by 0 nucleotides on the target RNA side of the guide-target RNA scaffold and 1, 2, 3, or 4 nucleotides on the engineered guide RNA side of the guide-target RNA scaffold. An asymmetrical bulge of the present disclosure, in some embodiments, is formed by 1, 2, 3, or 4 nucleotides on the engineered guide RNA side of the guide-target RNA scaffold and 1, 2, 3, or 4 nucleotides on the target RNA side of the guide-target RNA scaffold, where the engineered guide RNA side and the target RNA side of the guide-target RNA scaffold have different numbers of nucleotides. Thus, an asymmetrical bulge, in some embodiments, is a structural feature formed from latent structure provided by an engineered latent guide RNA.

In some cases, a structural feature is an internal loop. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop, in some embodiments, is a symmetrical internal loop or an asymmetrical internal loop. Internal loops present in the vicinity of the edit site, in some embodiments, help with base flipping of the target A in the target RNA to be edited.

In some implementations, one side of the internal loop, either on the target RNA side or the engineered guide RNA side of the guide-target RNA scaffold, is formed by from 5 to 150 nucleotides. One side of the internal loop, in some embodiments, is formed by at least 5, 10, 15, 20, 50, 100, 200, 300, 400, 500, or 1000 nucleotides, or any number of nucleotides therebetween. Thus, an internal loop, in some embodiments, is a structural feature formed from latent structure provided by an engineered latent guide RNA.

An internal loop, in some embodiments, is a symmetrical internal loop or an asymmetrical internal loop. A symmetrical internal loop is formed when the same number of nucleotides is present on each side of the internal loop. For example, in some implementations, a symmetrical internal loop in a guide-target RNA scaffold of the present disclosure has the same number of nucleotides on the engineered guide RNA side and the target RNA side of the guide-target RNA scaffold. A symmetrical internal loop of the present disclosure, in some embodiments, is formed by at least 5, 6, 7, 8, 9, 10, 10, 15, 20, 50, 100, 200, 300, 400, 500, or 1000 nucleotides on the engineered guide RNA side of the guide-target RNA scaffold target and at least 5, 6, 7, 8, 9, 10, 10, 15, 20, 50, 100, 200, 300, 400, 500, or 1000 nucleotides on the target RNA side of the guide-target RNA scaffold, where the number of nucleotides on the engineered side of the guide-target RNA scaffold target is the same as the number of nucleotides on the target RNA side of the guide-target RNA scaffold. Thus, a symmetrical internal loop, in some embodiments, is a structural feature formed from latent structure provided by an engineered latent guide RNA.

An asymmetrical internal loop is formed when a different number of nucleotides is present on each side of the internal loop. For example, in some implementations, an asymmetrical internal loop in a guide-target RNA scaffold of the present disclosure has different numbers of nucleotides on the engineered guide RNA side and the target RNA side of the guide-target RNA scaffold.

An asymmetrical internal loop of the present disclosure, in some embodiments, is formed by from 5 to 150 nucleotides on the engineered guide RNA side of the guide-target RNA scaffold and from 5 to 150 nucleotides on the target RNA side of the guide-target RNA scaffold, wherein the number of nucleotides is different on the engineered side of the guide-target RNA scaffold target than the number of nucleotides on the target RNA side of the guide-target RNA scaffold. An asymmetrical internal loop of the present disclosure, in some embodiments, is formed by from 5 to 1000 nucleotides on the engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure, in some embodiments, is formed by at least 5, 6, 7, 8, 9, 10, 10, 15, 20, 50, 100, 200, 300, 400, 500, or 1000 nucleotides on the engineered guide RNA side of the guide-target RNA scaffold and at least 5, 6, 7, 8, 9, 10, 10, 15, 20, 50, 100, 200, 300, 400, 500, or 1000 nucleotides on the target RNA side of the guide-target RNA scaffold, where the number of nucleotides is different on the engineered side of the guide-target RNA scaffold target than the number of nucleotides on the target RNA side of the guide-target RNA scaffold. Thus, an asymmetrical internal loop, in some embodiments, is a structural feature formed from latent structure provided by an engineered latent guide RNA.

In some embodiments, a structural feature is a U-deletion formed upon binding of the gRNA to the mRNA transcribed from the target gene.

### 2. Barbell Macro-Footprints

In some embodiments, an engineered guide RNA targeting a target RNA further comprises a macro-footprint sequence such as a barbell macro-footprint. As disclosed herein, a barbell macro-footprint sequence, upon hybridization to a target RNA, produces a pair of internal loop structural features that improve one or more aspects of editing, as compared to an otherwise comparable guide RNA lacking the pair of internal loop structural features. In some instances, inclusion of a barbell macro-footprint sequence improves an amount of editing of an adenosine of interest (e.g., an on-target adenosine), relative to an amount of editing of on-target adenosine in a comparable guide RNA lacking the barbell macro-footprint sequence. In some instances, inclusion of a barbell macro-footprint sequence decreases an amount of editing of adenosines other than the adenosine of interest (e.g., decreases off-target adenosine), relative to an amount of off-target adenosine in a comparable guide RNA lacking the barbell macro-footprint sequence.

A macro-footprint sequence, in some embodiments, is positioned such that it flanks a micro-footprint sequence. Further, while in some cases a macro-footprint sequence flanks a micro-footprint sequence, in some implementations additional latent structures are incorporated that flank either end of the macro-footprint as well. In some embodiments, such additional latent structures are included as part of the macro-footprint. In some embodiments, such additional latent structures are separate, distinct, or both separate and distinct from the macro-footprint.

In some embodiments, each internal loop is positioned towards 5′ end or 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the target RNA, results in formation of barbell internal loops on opposing sides of the micro-footprint, which in turn comprises at least one structural feature that facilitates editing of a specific target RNA. The present disclosure demonstrates that, in some implementations, the presence of barbells flanking the micro-footprint improves one or more aspects of editing. For instance, in an example embodiment, the presence of a barbell macro-footprint in addition to a micro-footprint results in a higher amount of on target adenosine editing, relative to an otherwise comparable guide RNA lacking the barbells. Additionally, and or alternatively, in another example embodiment, the presence of a barbell macro-footprint in addition to a micro-footprint results in a lower amount of local off-target adenosine editing, relative to an otherwise comparable guide RNA lacking the barbells. Further, while the effect of various micro-footprint structural features varies, in some instances, on a target-by-target basis based on selection in a high throughput screen, the present disclosure demonstrates that the increase in the one or more aspects of editing provided by the barbell macro-footprint structures is independent, in certain embodiments, of the particular target RNA. Thus, the present disclosure provides a facile method of improving editing of guide RNAs previously selected to facilitate editing of a target RNA of interest. For example, in some embodiments, the barbell macro-footprint and the micro-footprint of the disclosure provides an increased amount of on target adenosine editing relative to an otherwise comparable guide RNA lacking the barbells. In other embodiments, the presence of the barbell macro-footprint in addition to the micro-footprint described here results in a lower amount of local off-target adenosine editing, relative to an otherwise comparable guide RNA, upon hybridization of the guide RNA and target RNA to form a guide-target RNA scaffold lacking the barbells.

In some embodiments, a macro-footprint sequence comprises a barbell macro-footprint sequence comprising latent structures that, when manifested, produce a first internal loop and a second internal loop.

In some examples, a first internal loop is positioned “near 5′ end of the guide-target RNA scaffold” and a second internal loop is positioned near 3′ end of the guide-target RNA scaffold. The length of the dsRNA comprises a 5′ end and a 3′ end, where up to half of the length of the guide-target RNA scaffold at the 5′ end is considered to be “near the 5′ end” while up to half of the length of the guide-target RNA scaffold at 3′ end is considered “near 3′ end.” Non-limiting examples of 5′ end include about 50% or less of the total length of the dsRNA at the 5′ end, about 45%, about 40%, about 35%, about 30%, about 25%, about 20%, about 15%, about 10%, or about 5%. Non-limiting examples of 3′ end include about 50% or less of the total length of the dsRNA at 3′ end about 45%, about 40%, about 35%, about 30%, about 25%, about 20%, about 15%, about 10%, or about 5%.

In some embodiments, the engineered guide RNAs of the disclosure comprising a barbell macro-footprint sequence (that manifests as a first internal loop and a second internal loop) improve RNA editing efficiency of a target RNA, and/or increase the amount or percentage of RNA editing generally, as well as for on-target nucleotide editing, such as on-target adenosine. In some embodiments, the engineered guide RNAs of the disclosure comprising a first internal loop and a second internal loop also facilitate a decrease in the amount of or reduce off-target nucleotide editing, such as off-target adenosine or unintended adenosine editing. The decrease or reduction in some examples is of the number of off-target edits or the percentage of off-target edits.

Each of the first and second internal loops of the barbell macro-footprint is, in some embodiments, independently symmetrical or asymmetrical, where symmetry is determined by the number of bases or nucleotides of the engineered guide RNA and the number of bases or nucleotides of the target RNA, that together form each of the first and second internal loops.

As described herein, a double stranded RNA (dsRNA) substrate (a guide-target RNA scaffold) is formed upon hybridization of an engineered guide RNA of the present disclosure to a target RNA. An internal loop, in some embodiments, is a symmetrical internal loop or an asymmetrical internal loop. Symmetrical and asymmetrical internal loops contemplated for use in barbell macro-footprints are described in further detail elsewhere herein (see, e.g., the section entitled “Engineered Guide RNAs,” above).

In some embodiments, a first internal loop or a second internal loop independently comprises a number of bases of at least about 5 bases or greater (e.g., at least 6, 7, 8, 9, 10, 15, 20, 30, 40, 50, 100, 150 or more); about 150 bases or fewer (e.g., 145, 105, 55, 25, 15, 10, 9, 8, 7, 6, 5 or fewer); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the engineered guide RNA and a number of bases of at least about 5 bases or greater (e.g., at least 6, 7, 8, 9, 10, 15, 20, 30, 40, 50, 100, 150 or more); about 150 bases or fewer (e.g., 145, 105, 55, 25, 15, 10, 9, 8, 7, 6, 5 or fewer); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

In some embodiments, an engineered guide RNA comprising a barbell macro-footprint (e.g., a latent structure that manifests as a first internal loop and a second internal loop) comprises a cytosine in a micro-footprint sequence in between the macro-footprint sequence that, when the engineered guide RNA is hybridized to the target RNA, is present in the guide-target RNA scaffold opposite an adenosine that is edited by the RNA editing entity (e.g., an on-target adenosine). In such embodiments, the cytosine of the micro-footprint is comprised in an A/C mismatch with the on-target adenosine of the target RNA in the guide-target RNA scaffold.

A first internal loop and a second internal loop of the barbell macro-footprint, in some embodiments, are positioned a certain distance from the A/C mismatch, with respect to the base of the first internal loop and the base of the second internal loop that is the most proximal to the A/C mismatch. In some embodiments, the first internal loop and the second internal loop are positioned the same number of bases from the A/C mismatch, with respect to the base of the first internal loop and the base of the second internal loop that is the most proximal to the A/C mismatch. In some embodiments, the first internal loop and the second internal loop are positioned a different number of bases from the A/C mismatch, with respect to the base of the first internal loop and the base of the second internal loop that is the most proximal to the A/C mismatch.

In some embodiments, the first internal loop of the barbell or the second internal loop of the barbell is positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell is positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, the first internal loop is positioned from about 5 bases away from the A/C mismatch to about 15 bases away from the A/C mismatch (e.g., 6-14, 7-13, 8-12, 9-11) with respect to the base of the first internal loop that is most proximal to the A/C mismatch. In some examples, the first internal loop is positioned from about 9 bases away from the A/C mismatch to about 15 bases away from the A/C mismatch (e.g., 10-14, 11-13) with respect to the base of the first internal loop that is the most proximal to the A/C mismatch.

In some embodiments, the second internal loop is positioned from about 12 bases away from the A/C mismatch to about 40 bases away from the A/C mismatch (e.g., 13-39, 14-38, 15-37, 16-36, 17-35, 18-34, 19-33, 20-32, 21-31, 22-30, 23-29, 24-28, 25-27) with respect to the base of the second internal loop that is the most proximal to the A/C mismatch. In some embodiments, the second internal loop is positioned from about 20 bases away from the A/C mismatch to about 33 bases away from the A/C mismatch with respect to the base of the second internal loop that is most proximal to the A/C mismatch.

3. Engineered Guide RNAs with Tertiary Structure

In some embodiments, hybridization of the target RNA and the targeting domain of the guide RNA also produces specific tertiary structures in the guide-target RNA scaffold that manifest upon hybridization. Tertiary structures when manifested become features described herein, including coaxial stacking, A-platforms, interhelical packing motifs, triplexes, major groove triples, minor groove triples, tetraloop motifs, metal-core motifs, ribose zippers, kissing loops, and pseudoknots. Without wishing to be bound by theory, the presence of tertiary structures features described herein that are produced upon hybridization of the guide RNA with the target RNA configure the guide RNA aid in a specific, or selective, targeted edit of the target RNA via the RNA editing entity or biologically active fragment thereof. Further, the tertiary structures in combination generally facilitate an increased amount of editing of a target adenosine, fewer off target edits, or both, as compared to a construct comprising the mismatch alone or a construct having perfect complementarity to a target RNA. In some embodiments, the tertiary structures in combination with the mismatch described above generally facilitate an increased amount of editing of a target adenosine, fewer off target edits, or both, as compared to a construct comprising the mismatch alone or a construct having perfect complementarity to a target RNA. Accordingly, in some implementations, rational design taking the effects of tertiary structures into account in engineered guide RNAs of the present disclosure to produce specific structural features in a guide-target RNA scaffold is a powerful tool to promote editing of the target RNA with high specificity, selectivity, and robust activity.

Generally, tertiary structures are structures involved in interactions between distinct secondary structures, such as the structural features described herein, and determine the three-dimension structure of the guide-target RNA scaffold. In some embodiments, a tertiary structure involves interactions between two double-stranded helical regions, and includes, for example, coaxial stacking, an adenosine platform, or an interhelical packing motif. In some embodiments, a tertiary structure involves interactions between a helical region and a non-double-stranded region, and includes, for example, a triplex, a major groove triple, a minor groove triple, a tetraloop motif, a metal-core motif, or a ribose zipper. In some embodiments, a tertiary structure involves interactions between two non-helical regions, and includes, for example, a kissing loop or a pseudoknot. In some embodiments, a guide-target RNA scaffold as described herein has one or more tertiary structures. In some implementations, different biophysical forces are involved in a forming a tertiary structure, including, but not limited to, torsion, hydrogen bonding, Van der Waals, base-pair interactions, hydrophobicity, and Hoogsteen interactions.

### Machine Learning for Engineered Guide RNA

The theoretical engineered guide RNA design space for editing a target RNA (e.g., the number of possible of permutations of latent structural features, secondary structural features, tertiary structures, and/or ADAR recruiting domains in an engineered guide RNA for a target RNA) that requires experimental testing to determine if the engineered guide RNA has the desired on-target editing and specificity score is extremely large. For example, for an engineered guide RNA comprising 30 nt mutation window, there is a pool of about 10⁴³ engineered guide RNAs (comprising latent structural features) that would need to be tested. The approaches described in FIG. 3 have the potential to identify a subspace of engineered guide RNAs having the desired on-target editing and specificity score much faster than a non-ML-based approach. Unlike target-specific screening methods, the ML-based approaches have the potential to distill knowledge from complex ADAR-guide interactions, which, in some embodiments, are transferrable to unknown targets in the future. In some implementations, the ML-based approaches disclosed herein significantly shorten the screening cycle. The laboratory results, in some embodiments, are fed back to the machine learning models (e.g., as additional training samples) to further iteratively train the machine learning models, as illustrated by the arrows in FIG. 3.

FIG. 3 is a flowchart depicting two examples of machine learning processes (further described below) that, in some embodiments, are used for identifying a subspace of engineered guide RNAs having the desired on-target editing and specificity score. The machine learning approaches, in some embodiments, include iterative processes of screening, modeling, and in some embodiments, generating new guide RNAs for screening. In some embodiments, the machine learning predicts a percentage of on-target editing and a specificity score of an engineered guide RNA and a target RNA. In some embodiments, this machine learning model is end-to-end differentiable, which allows it to generate a potential engineered guide RNA sequence for a specified percentage of on-target editing and specificity score. In some embodiments, this machine learning model allows for identification of key feature determinates that impact the percentage of on-target editing and/or specificity score.

### 4. Machine Learning Model Structure and Training

In various embodiments, a wide variety of machine learning techniques are applicable for performing the methods disclosed herein. Non-limiting examples include different forms of supervised learning, unsupervised learning, and semi-supervised learning such as decision trees, support vector machines (SVMs), linear regression, logistic regression, Bayesian networks, and boosted gradient algorithms. Deep learning techniques such as neural networks, including convolutional neural networks (CNN), recurrent neural networks (RNN), and attention-based models (such as Transformers), are also contemplated. The processes discussed in FIGS. 2 and 3, in some embodiments, apply one or more machine learning and deep learning techniques.

In various embodiments, training techniques for a machine learning model include, but are not limited to, supervised, semi-supervised, and/or unsupervised training. In supervised learning, the machine learning models, in some embodiments, are trained with a set of training samples that are labeled. For instance, in an example embodiment, for a machine learning model that is iteratively trained to predict the binding catalyst performance of an engineered guide RNA 130 or the engineered guide RNA of FIG. 23B, the training samples are versions of sequences of known engineered guide RNA 130 or the engineered guide RNA of FIG. 23B and those engineered guide RNAs' associated metrics (e.g., percentage of on-target editing, specificity score, etc.) that are determined experimentally (e.g., in vitro in an HTS, in vitro in one or more cell types, or in vivo). The labels for each training sample, in some embodiments, are binary or multi-class. For instance, in another example embodiment, in training a machine learning model using the first approach 310, the training samples are mathematical vectors that include various extracted features of the sequences expressed in different dimensions of the vectors. In some embodiments, the label is binary (e.g., enhancing editing or not enhancing editing) or a series of scores (e.g., experimental values of metrics associated with the engineered guide RNA 130 or an engineered guide RNA of FIG. 23B). In training a machine learning model using the second approach 320, in yet another example embodiment, the training samples are the sequences of the engineered guide RNA 130 or an engineered guide RNA of FIG. 23B. In some embodiments, the label is a series of scores. In some cases, an unsupervised learning technique is used. In such a case, the samples used in training are not labeled. In some implementations, various unsupervised learning techniques such as clustering are used. In some cases, the training is semi-supervised with the training set having a mix of labeled samples and unlabeled samples.

A machine learning model, in some embodiments, is associated with an objective function, which generates a value that describes the objective goal of the training process. For example, in some embodiments, the training intends to reduce the error rate of the model in generating a prediction of the performance metrics of the engineered guide RNAs 130 or engineered guide RNAs of FIG. 23B in the training set. In such a case, the objective function monitors the error rate of the machine learning model. Such an objective function, in some embodiments, is called a loss function. In some embodiments, other forms of objective functions are also used, particularly for unsupervised learning models whose error rates are not easily determined due to the lack of labels. In the second approach 350, the loss function determines the difference between ensemble output (predicted) and desired (predefined) values, and the gradient with regard to the input was calculated and back-propagated to update the input (random seed). In various embodiments, the error rate is measured as cross-entropy loss, L1 loss (e.g., the sum of absolute differences between the predicted values and the actual value), L2 loss (e.g., the sum of squared distances).

Referring to FIG. 4, a structure of an example CNN is illustrated, in accordance with some embodiments. In some embodiments, machine learning models as disclosed herein have different architectures based on the desired target mRNA for which outputs are generated. For instance, in some implementations, different model architectures are selected in order to generate one or more metrics for a deamination efficiency or specificity by an ADAR protein of a target nucleotide position in different corresponding target mRNAs, based on input information for a given gRNA. In some implementations, different model architectures are selected in order to generate a candidate sequence for a gRNA (e.g., input optimization), for different corresponding mRNA targets. As another example, the example CNN illustrated in FIG. 4 is used, in some implementations, for obtaining outputs using SERPINA1 and/or LRRK2 datasets as input. Different model architectures are contemplated for use in the present disclosure, for instance for metric prediction and/or input optimization for different gene targets. Examples of gene targets suitable for use in the present disclosure include, but are not limited to, ABCA4, SERPINA1, LRRK2, DUX4, GRN, MAPT, and/or SNCA. In some embodiments, the CNN 400 receives inputs 410 and generate outputs 420. Although inputs 410 are graphically illustrated as having two dimensions in FIG. 4, in some embodiments, the inputs 410 are in any dimension. For example, in some implementations, the CNN 400 is a one-dimensional convolutional network. In one embodiment, the inputs 410 are an RNA sequence discussed in FIG. 2 and FIG. 3.

In some embodiments, the model 400 includes different kinds of layers, such as convolutional layers 430, pooling layers 440, recurrent layers 450, fully connected layers 460, and custom layers 470. A convolutional layer 430 convolves the input of the layer (e.g., an RNA sequence) with one or more weight kernels to generate different types of sequences that are filtered by the kernels to generate feature sequences. Each convolution result, in some embodiments, is associated with an activation function. A convolutional layer 430, in some embodiments, is followed by a pooling layer 440 that selects the maximum value (max pooling) or average value (average pooling) from the portion of the input covered by the kernel size. The pooling layer 440 reduces the spatial size of the extracted features. Optionally, in some embodiments, a pair of convolutional layer 430 and pooling layer 440 is followed by an optional recurrent layer 450 that includes one or more feedback loops 455. The feedback 455, in some embodiments, is used to account for spatial relationships of the features in an image or temporal relationships in sequences. In some embodiments, the layers 430, 440, and optionally, 450, are followed in multiple fully connected layers 460 that have nodes (represented by squares in FIG. 4) connected to each other. The fully connected layers 460 are, in some embodiments, used for classification and regression. In one embodiment, one or more custom layers 470 are also presented for the generation of a specific format of output 420.

The order of layers and the number of layers of the CNN 400 in FIG. 4 is for example only. In various embodiments, a CNN 400 includes one or more convolutional layer 430 but does not include any pooling layer 440 or recurrent layer 450. In various embodiments, a CNN 400 includes one or more convolutional layer 430, one or more pooling layer 440, one or more recurrent layer 450, or any combination thereof. If a pooling layer 440 is present, not all convolutional layers 430 are always followed by a pooling layer 440. A recurrent layer, in some embodiments, is also positioned differently at other locations of the CNN. For each convolutional layer 430, the sizes of kernels (e.g., 1×3, 1×5, 1×7, etc.) and the numbers of kernels allowed to be learned, in some embodiments, are different from other convolutional layers 430.

In some embodiments, a machine learning model includes certain layers, nodes, kernels and/or coefficients. Training of a neural network, such as the CNN 400, in some embodiments, includes multiple iterations of forward propagation and backpropagation. Each layer in a neural network, in some embodiments, includes one or more nodes, which are fully or partially connected to other nodes in adjacent layers. In forward propagation, the neural network performs the computation in the forward direction based on outputs of a preceding layer. The operation of a node, in some embodiments, is defined by one or more functions. The functions that define the operation of a node, in some embodiments, include various computation operations such as convolution of data with one or more kernels, pooling, recurrent loop in RNN, various gates in LSTM, etc. The functions, in some embodiments, also include an activation function that adjusts the weight of the output of the node. Nodes in different layers, in some embodiments, are associated with different functions.

Each of the functions in the neural network, in some embodiments, is associated with different coefficients (e.g., weights and kernel coefficients) that are adjustable during training. In addition, some of the nodes in a neural network, in some embodiments, are also associated with an activation function that decides the weight of the output of the node in forward propagation. Common activation functions include, but are not limited to, step functions, linear functions, sigmoid functions, hyperbolic tangent functions (tanh), and rectified linear unit functions (ReLU). After an input is provided into the neural network and passes through a neural network in the forward direction, in some implementations, the results are compared to the training labels or other values in the training set to determine the neural network's performance. In some embodiments, the process of prediction is repeated for other inputs in the training sets to compute the value of the objective function in a particular training round. In turn, the neural network performs backpropagation by using gradient descent such as stochastic gradient descent (SGD) to adjust the coefficients in various functions to improve the value of the objective function.

In some embodiments, multiple iterations of forward propagation and backpropagation are performed and the machine learning model is iteratively trained. In some embodiments, training is completed when the objective function has become sufficiently stable (e.g., the machine learning model has converged) or after a predetermined number of rounds for a particular set of training samples. In some implementations, the trained machine learning model is used for performing various machine learning tasks as discussed in this disclosure.

For example, in some embodiments, the machine learning model includes convolutional neural networks, recurrent neural networks, multilayer perceptron, XGBoost (e.g., eXtreme Gradient Boosting), transformer models, and/or generative modeling, optionally for methods of generating candidate sequences for a gRNAs (e.g., input optimization).

As another example, in some embodiments, the machine learning model includes bagging architectures (e.g., random forest, extra tree algorithms) and boosting architectures (e.g., gradient boosting, XGBoost, etc.). In some embodiments, the machine learning model is an extreme gradient boost (XGBoost) model. Description of XGBoost models is found, for example, in Chen T. and Guestrin C, “XGBoost: A Scalable Tree Boosting System,” arXiv:1603.02754v3 [cs.LG] 10 Jun. 2016, the disclosure of which is hereby incorporated by reference, in its entirety, for all purposes, and specifically for its teaching of training and using XGBoost models.

In some embodiments, the machine learning model includes random forest, decision tree, and boosted tree algorithms. In some embodiments, the model is a decision tree. Decision trees suitable for use as model are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm contemplated for use in the present disclosure is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

### 5. Example Machine Learning Processes-Feature Study

An exemplary machine learning model is shown in FIG. 3. In some embodiments, in a first example approach 310, an RNA sequence and secondary structure feature-based ensemble model is used. In some implementations, this approach 310 is driven by domain-knowledge-guided featurization. In some implementations, the output of this approach is easily interpretable and is useful for guiding human experts in highlighting important factors to consider when designing engineered guide agents 130 or engineered guide RNAs of FIG. 23B. In some embodiments, the approach 310 is used to design engineered guide agents 130 or engineered guide RNAs of FIG. 23B based on features predicted by a machine learning model to be important for good performance (e.g., high on-target editing, high specificity).

In some embodiments, in the first approach 310, feature engineering and a ML-based predictor, such as a regression model, a random forests model, a support vector machine (SVM), etc., are used. Inputs include, but are not limited to, sequence-related and/or secondary-structure-related features of an editing site (e.g., A>I editing site). In some embodiments, the ML-based predictor includes convolutional neural networks, recurrent neural networks, multilayer perceptron, XGBoost (e.g., eXtreme Gradient Boosting), transformer models, and/or generative modeling.

The sequence related inputs, in some embodiments, are of the engineered guide agent 130 or engineered guide RNAs of FIG. 23B and the target RNA. In some embodiments, the input is a self-annealing engineered guide RNA and target RNA linked by a hairpin.

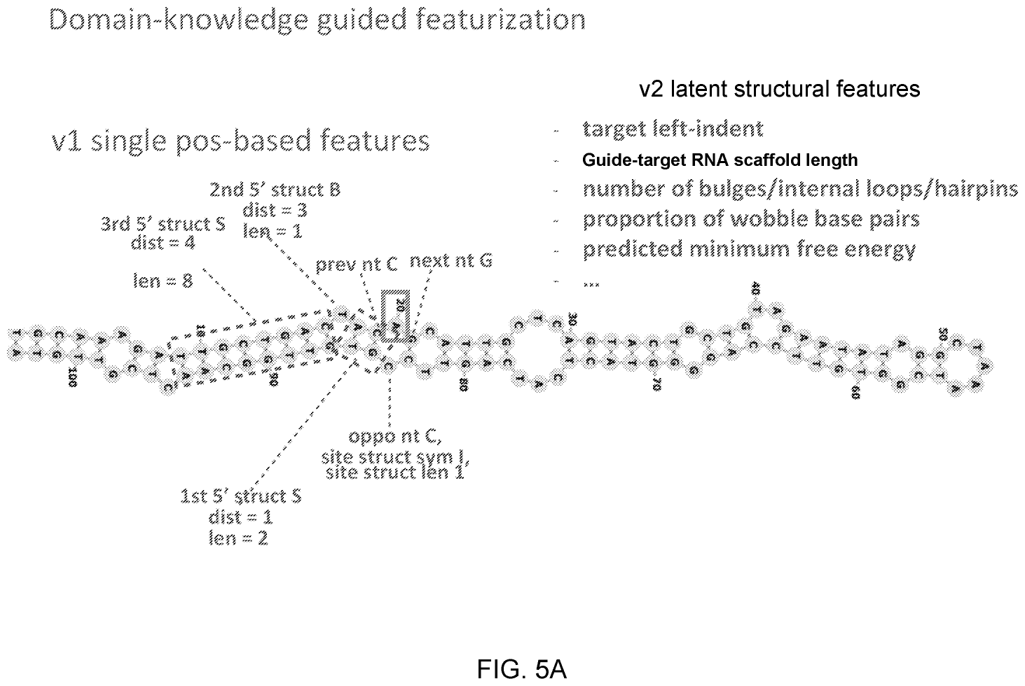

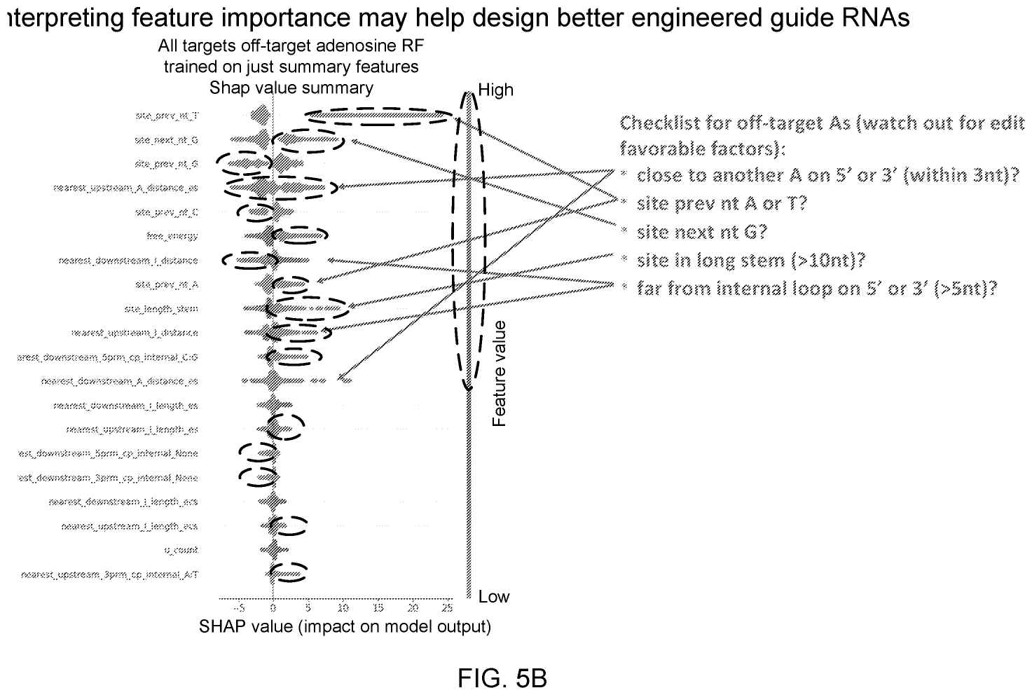

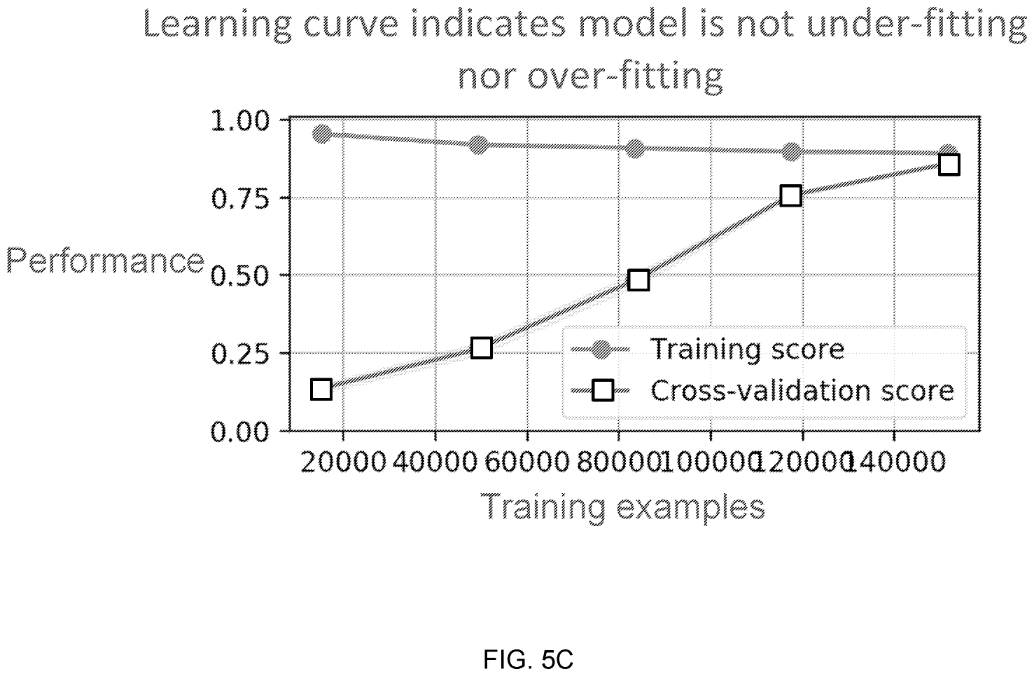

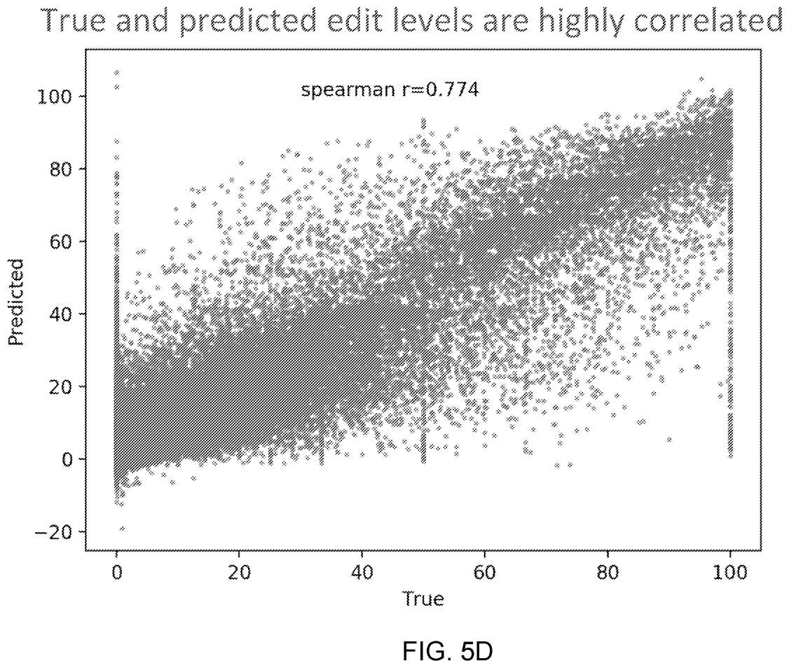

In some embodiments, features are extracted from a nucleic acid sequence. For example, in some embodiments, the RNA secondary structure prediction is one of the features extracted. The prediction of the secondary structure, in some embodiments, is performed via an open-source software package ViennaRNA. In some embodiments, features are sequence-level features, domain-level features, and site-level features. Example features contemplated for extraction from the nucleic acid sequence include, but are not limited to, structural features, thermodynamics features, number of mutations, sequence features (e.g., position), mutation sites value and features, the presence or absence of structural features such as hairpin, bulge, internal loop, stem, multiloop, nucleotide values at the site of interest, nucleotide values at other relevant sites, properties and values of nucleotides within a threshold nucleotide (e.g., 3nt or 5nt) from the editing site or target editing site, properties and values of the editing site or target editing site, properties and values of sequences upstream or downstream of the editing site or target editing site, ratios of two or more features, time of editing, and/or editing enzyme (e.g., ADAR1, ADAR2, or ADAR1 and ADAR2). FIG. 5A is a graphical illustration of some of the example features that are extracted, in some embodiments, from the nucleic acid sequence.

The machine learning model's outputs include, in some embodiments, individual editing levels (e.g., A>I editing) at a specified edit site and/or other metrics that predict the performance of the engineered guide agent 130 or the engineered guide RNA of FIG. 23B. Alternatively or additionally, the machine learning model, in some embodiments, outputs a combined on-target edit score and specificity score corresponding to the candidate sequence of gRNA (e.g., for editing by an ADAR protein on a target nucleotide position in a target mRNA sequence facilitated by the guide RNA, as determined using a plurality of sequence reads obtained from a plurality of target mRNAs). Specificity score is defined, in some embodiments, as the target edit percentage divided by the sum of all nonsynonymous off-target edits. In some embodiments, a specificity score is determined as the (sum of on-target editing of the desired nucleotide)/(sum of off-target editing). In some embodiments, a specificity score is determined as 1−(# of reads with only on-target edits)−(# of reads with zero edits). Additional predicted variables contemplated for use in the present disclosure include, but are not limited to, minimum free energy, e.g., of the double-stranded self-editing hairpin structure or the guide-target RNA scaffold. The machine learning model, in some embodiments, simultaneously predicts target adenosine editing and off-target editing (or specificity). Additionally or alternatively, the output, in some embodiments, includes a prediction of certain features likely to affect the editing performance for further laboratory studies. In some embodiments, a computing device generates an engineered guide RNA through mismatch, insertion, and deletion for a structural feature to create variants of the structural feature (e.g., various lengths) at various possible positions along the engineered guide agent 130: target mRNA 120 duplex or the engineered guide RNA: target mRNA duplex of FIG. 23B.

Alternatively or additionally, in some embodiments, the machine learning model generates a prediction of one or more metrics that measure the deamination ability of an ADAR protein on a target nucleotide position in a target mRNA when facilitated by hybridization of a gRNA having the respective candidate sequence. In some embodiments, the one or more metrics are selected from the group consisting of any on-target editing, specificity, target-only editing, no editing, and normalized specificity, for one or more ADAR proteins in a plurality of different ADAR proteins. For instance, in some embodiments, any on-target editing is determined as a proportion of sequence reads with any on-target edits. In some embodiments, specificity is determined as a (proportion of sequence reads with on-target edits+1)/(proportion of sequence reads with off-target edits+1). In some embodiments, target-only editing is determined as a proportion of sequence reads with only on-target edits. In some embodiments, no editing is determined as a proportion of sequence reads without any edits. In some embodiments, normalized specificity is determined as 1−(proportion of sequence reads with any off-target edits). In some embodiments, the one or more metrics further includes a difference in editing preference between a first ADAR protein and a second ADAR protein, in the plurality of different ADAR proteins. In some embodiments, the difference in editing preference is determined as (target-only editing of the first ADAR protein)−(target-only editing of the second ADAR protein). In some embodiments, the one or more metrics are obtained for ADAR1, ADAR2, or ADAR1/2. Alternatively or additionally, in some embodiments, the one or more metrics further includes editability, where editability is a measure of central tendency of the any on-target editing and target-only editing scores. In some embodiments, editability is the average of the any on-target editing and target-only editing scores.

The machine learning model used, in some embodiments, is a regression model, a random forests model, a support vector machine (SVM), a gradient boosting model, a clustering model, etc., whether the model is supervised, unsupervised, or semi-supervised. Examples of training of model will be discussed in further detail with reference to FIG. 4. Model and hyperparameter selection, in some embodiments, is done prior to training the selected model on the training set and evaluated on the validation set. Model performance (e.g., for regression), in some embodiments, is measured by percent variance explained and correlation between predicted and true values in the hold-out test set. In various embodiments, different models have been iteratively trained and evaluated on different datasets. In some embodiments, trained models have reached 80% variance in data explained. In some embodiments, the models are gradient boosted tree ensemble models.

In some embodiments, a computing device is used to study the importance or attribution of features using the trained model to discover features (e.g., structural features), rules, and patterns likely to influence model prediction with the assumption that such discovered features, rules or patterns accurately describe the underlying biology of ADAR deamination. In some embodiments, the Shapley value (SHAP value) for each extracted feature (e.g., a structural feature, time of editing, editing enzyme, etc.) is generated to determine the impact of each feature on model output. FIG. 5B is a graphical illustration of an example output of SHAP values associated with various features. The graphical illustration identifies key features that have a strong impact on the machine learning output (legend: features circled by dashed lines indicate degrees of high value; features not circled indicate degrees of low value). The features that are identified, in some embodiments, are used by scientists to conduct laboratory experiments on various candidates of engineered guide agent 130 or engineered guide RNAs of FIG. 23B that includes one or more identified features. In FIG. 5B, for example, “site next nt G” refers to a feature that the nucleotide succeeding the editing site is G.

FIG. 5C is a plot illustrating the performance of an example machine learning model using the first approach 310, in accordance with some embodiments. The plot demonstrates the training score and the cross-validation score of the model and indicates that the model is not under-fit or over-fit. FIG. 5D is a plot illustrating true (via experimental testing) and predicted edit levels. The spearman correlation coefficient of the model's predictions (Predictions) compared with observed on-target editing percentages (True) is 0.774, demonstrating that the true and predicted edit levels are highly correlated.

In some implementations, after training and validation of such models, models are used to score randomly and/or algorithmically generated novel nucleic acid sequences of candidates of engineered guide agents 130 or of engineered guide RNAs of FIG. 23B. In some such embodiments, scores are aggregated and used to rank and select new sequences for experimental testing.

In some embodiments, a computing server generates algorithmically nucleic acid sequences of candidates of engineered guide agents 130 or of engineered guide RNAs of FIG. 23B based on a specified secondary structure (e.g., a structural feature). For instance, in an example embodiment, given a set of desired secondary structure features in a gRNA sequence, an algorithm exhaustively generates all possible combinations of the positions and lengths of the desired secondary structure features (the base structure set), given the duplex length of the gRNA sequence and the location of the target adenosine. For each structure in the base structure set, a dot-bracket notation of its secondary structure is given to ViennaRNA, with the target strand sequence fixed to be the same, to generate a diverse set of guide strand sequences given the constraint that the entire gRNA sequence will fold into the desired secondary structure dictated by the given dot-bracket notation.

### 6. Example Machine Learning Processes-Deep Learning

Referring back to FIG. 3, in some embodiments, a second example approach 350 is used to both predict a percentage of on-target editing and specificity score as well as propose new candidate sequences of engineered guide agents 130 or of engineered guide RNAs of FIG. 23B. The second approach, in some embodiments, is a deep learning approach that uses a model, such as a convolutional neural network (CNN), which receives raw sequences as inputs. In some embodiments, the second approach uses a convolutional neural network, a recurrent neural network, a multilayer perceptron, XGBoost (e.g., eXtreme Gradient Boosting), transformer models, and/or generative modeling. The model, in some embodiments, is iteratively trained by a gradient descent process to predict target editing level and specificity score based on input guide sequence. The model, in some embodiments, directly takes RNA primary sequence (instead of extracted features of the sequence as an input). The model is a high capacity model and is end-to-end differentiable. In some embodiments, the operations of this model are differentiable, which allows for propagating the gradients to update either the weights or back to the input. As a result, in some implementations, after training a predictor model, the model is used to optimize an input sequence and generate new and novel guide RNAs for testing.

In some implementations, for the second approach 350, inputs are one hot-encoded sequences of candidates of engineered guide agents 130 or of engineered guide RNAs of FIG. 23B that include an RNA targeting domains 134 (with the site to be edited in a disease-related gene) and the target RNA 120 or the RNA targeting domains and the target RNA of FIG. 23B. The engineered guide 130 or an engineered guide RNA of FIG. 23B, in some embodiments, is connected by a short hairpin loop to the target RNA 120 or the target RNA of FIG. 23B. In addition to or alternative to the one hot-encoded sequence, in some implementations, inputs include positional encodings. The positional encodings serve to transfer coordinate information to the model.

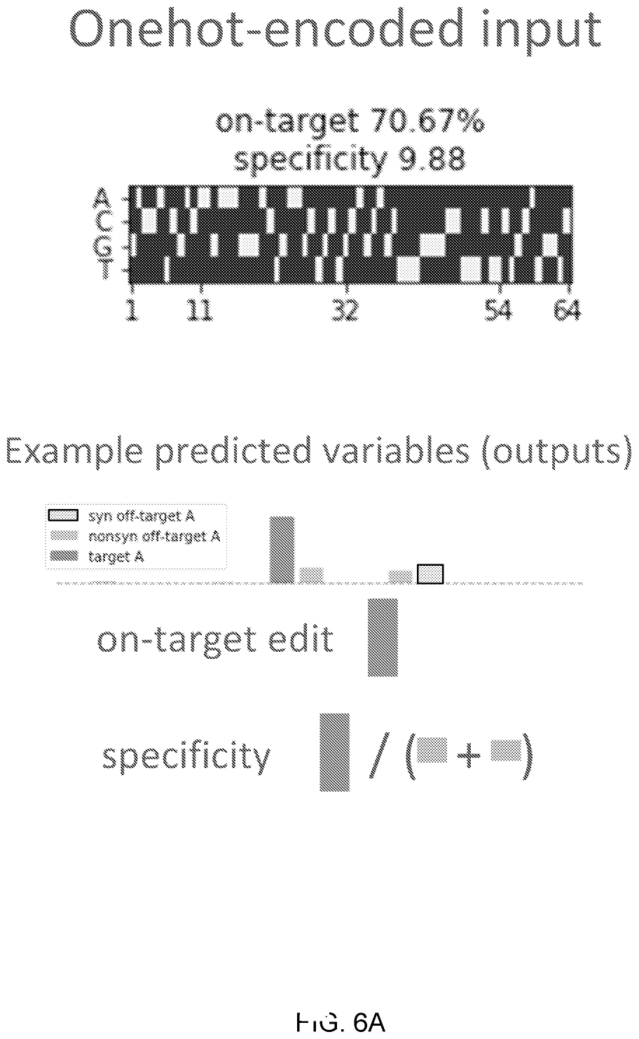

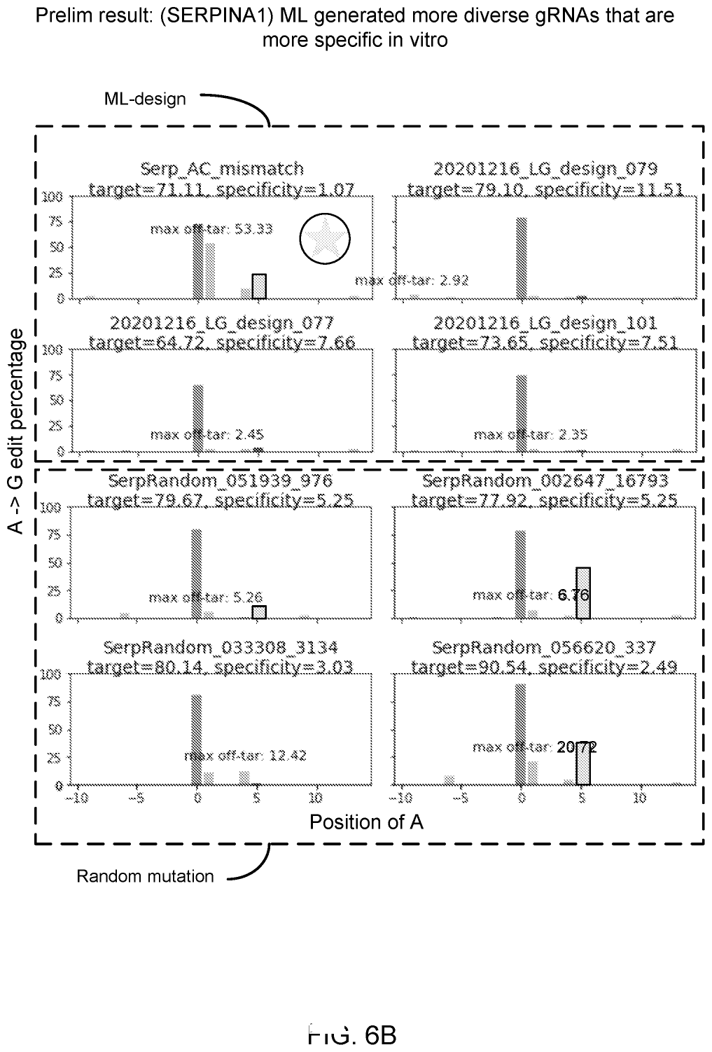

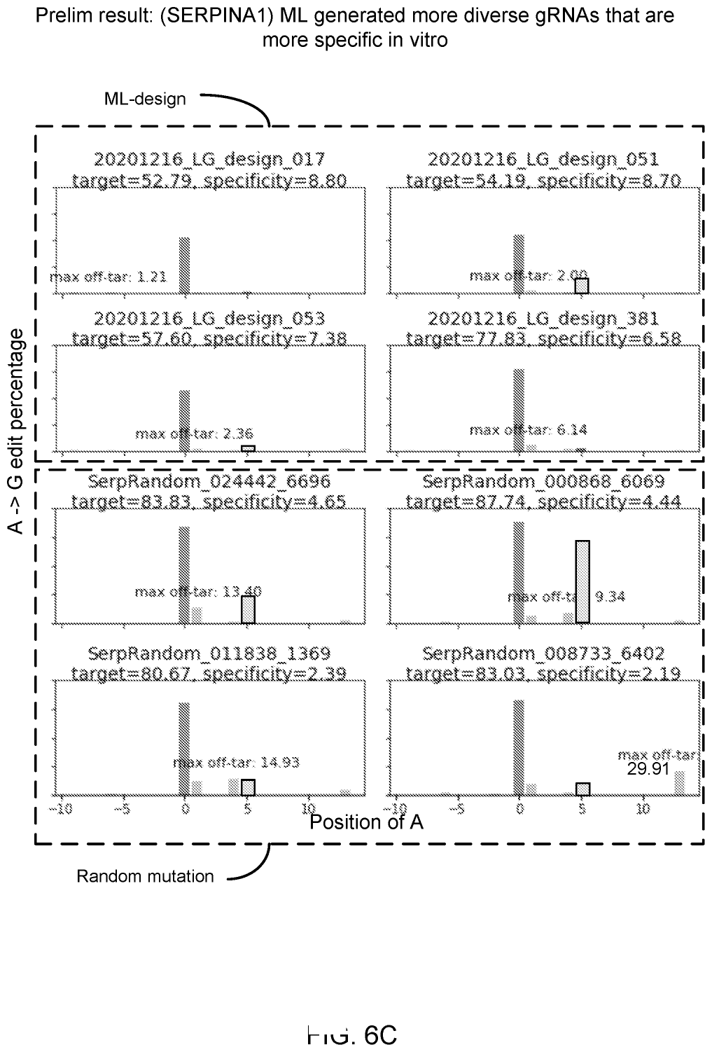

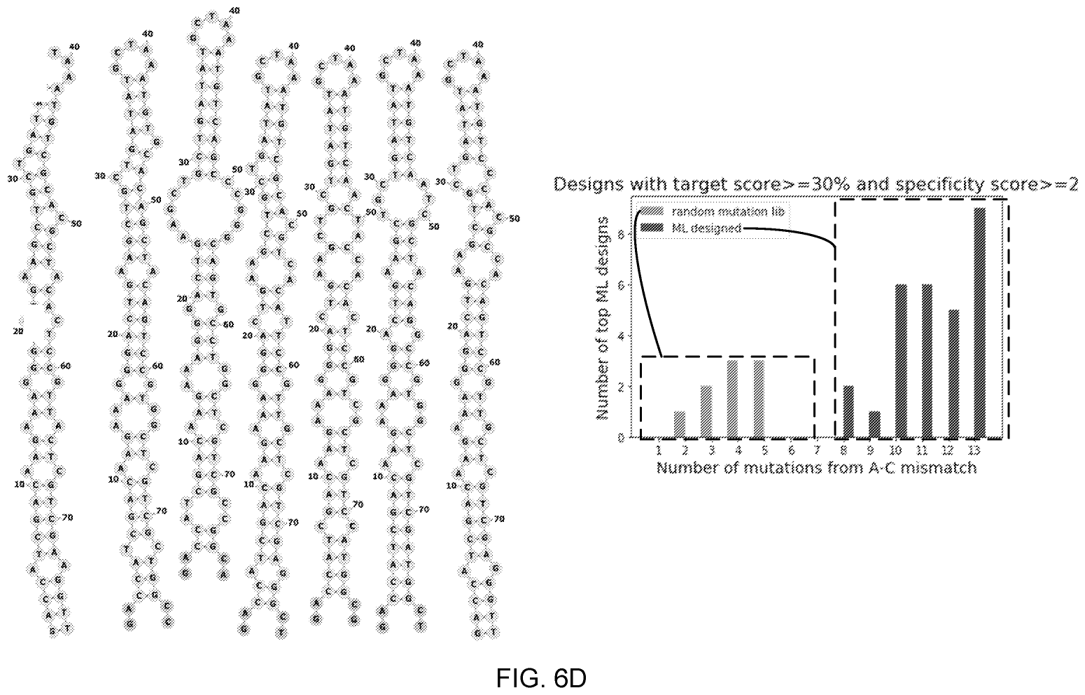

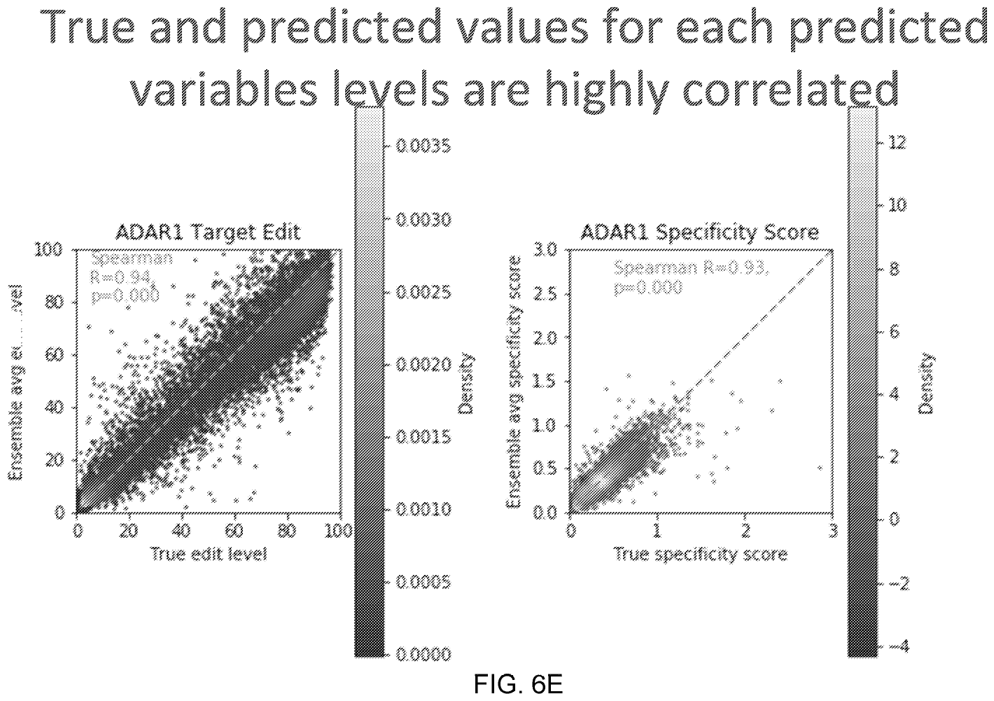

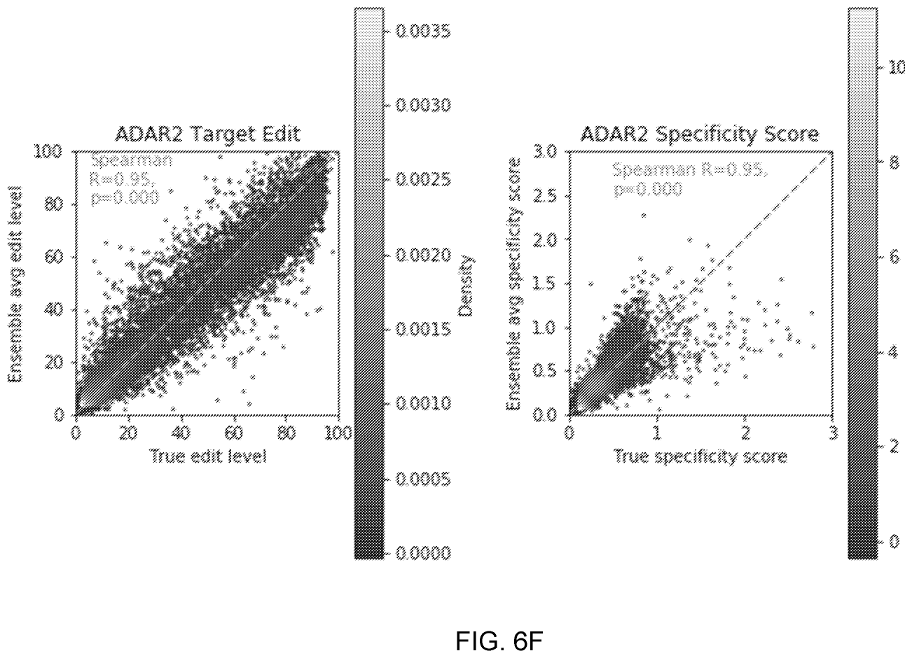

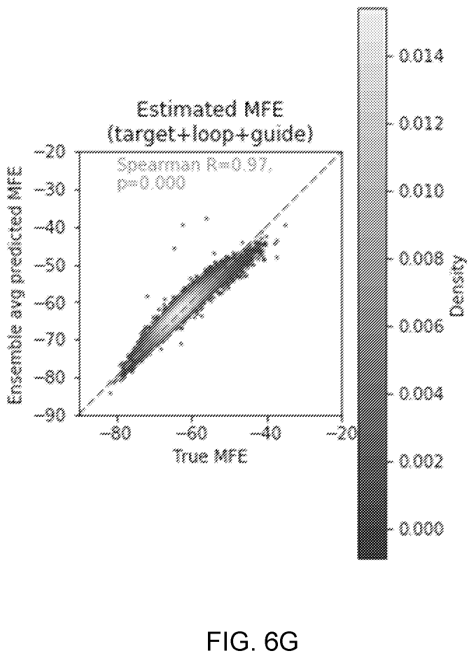

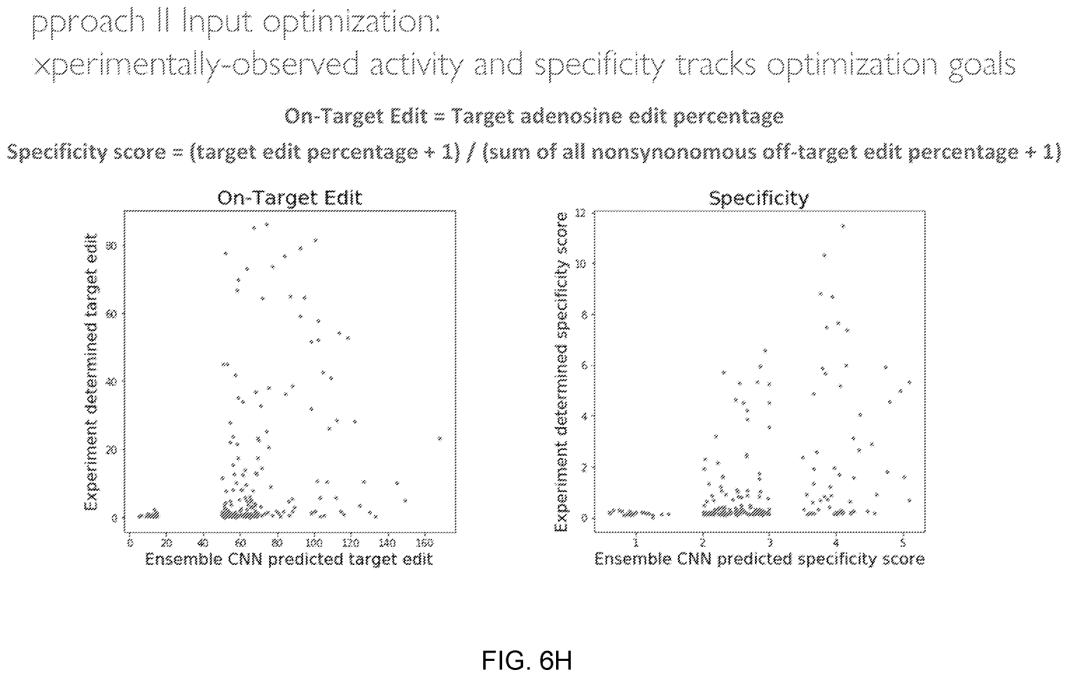

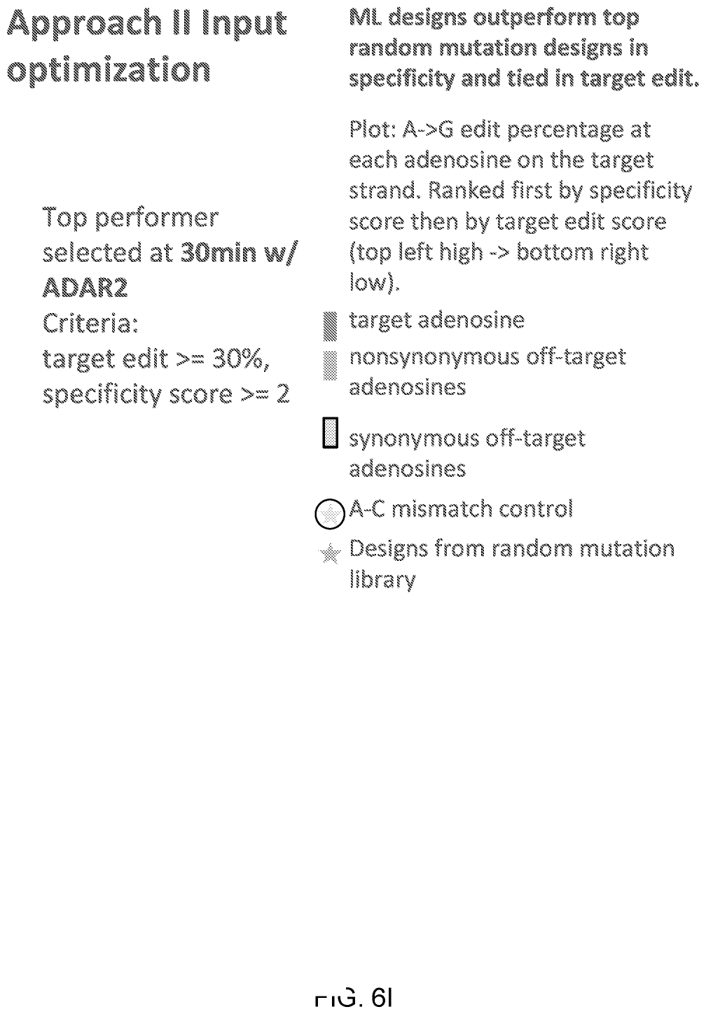

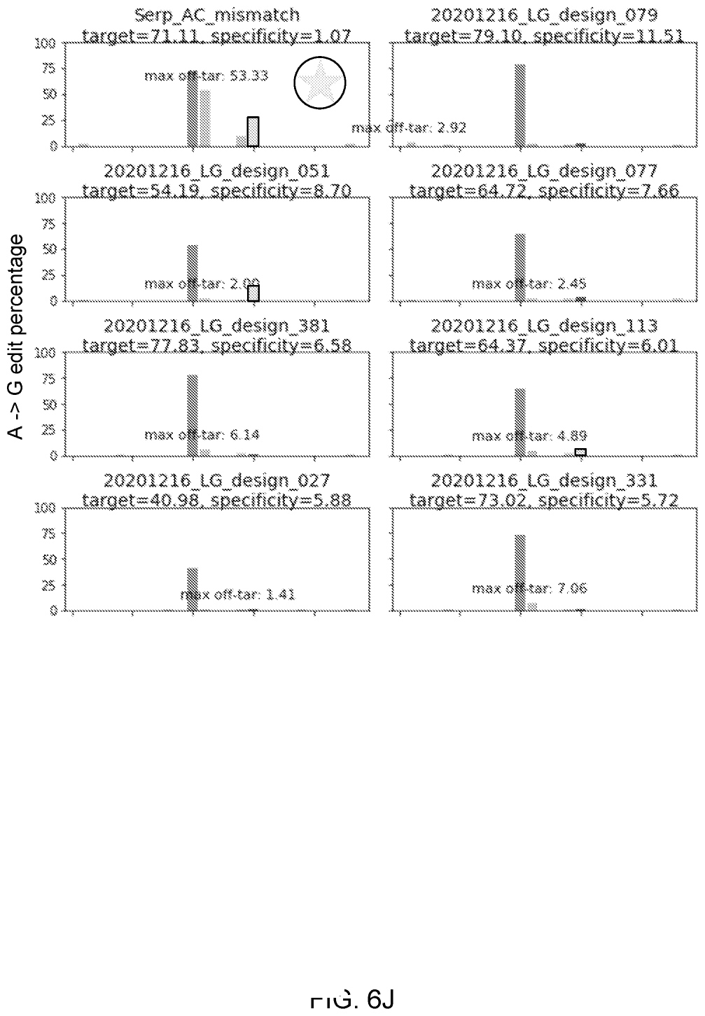

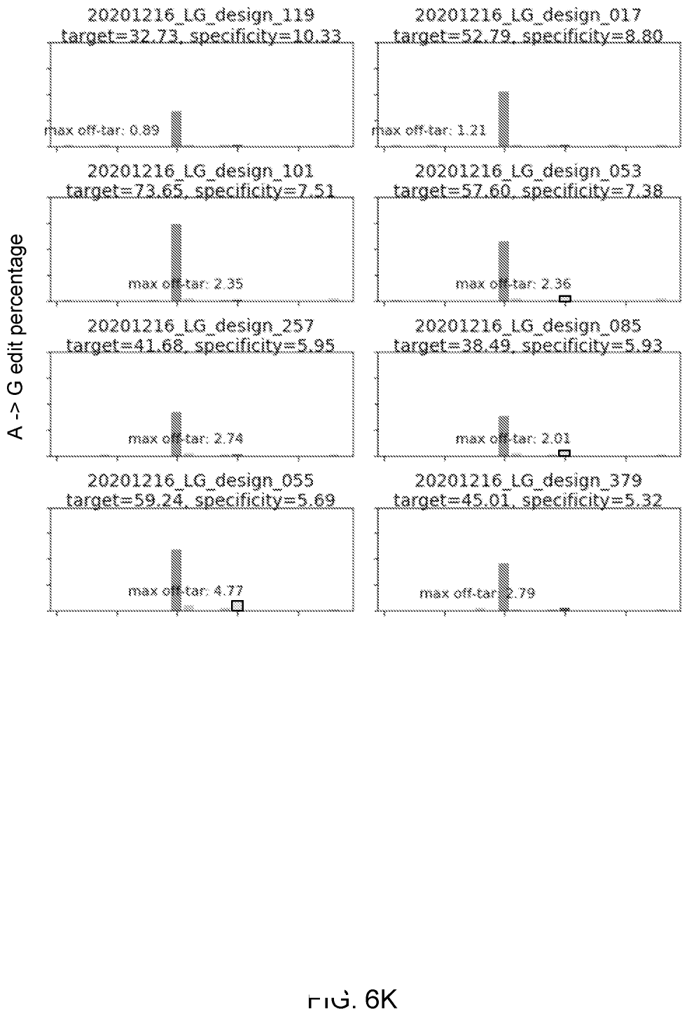

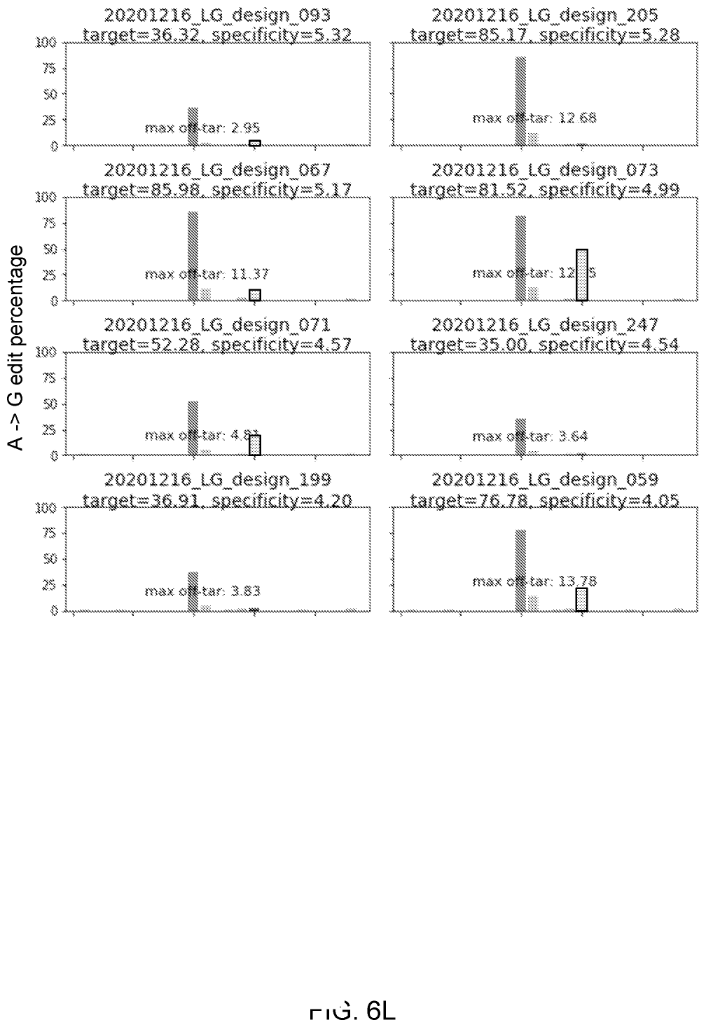

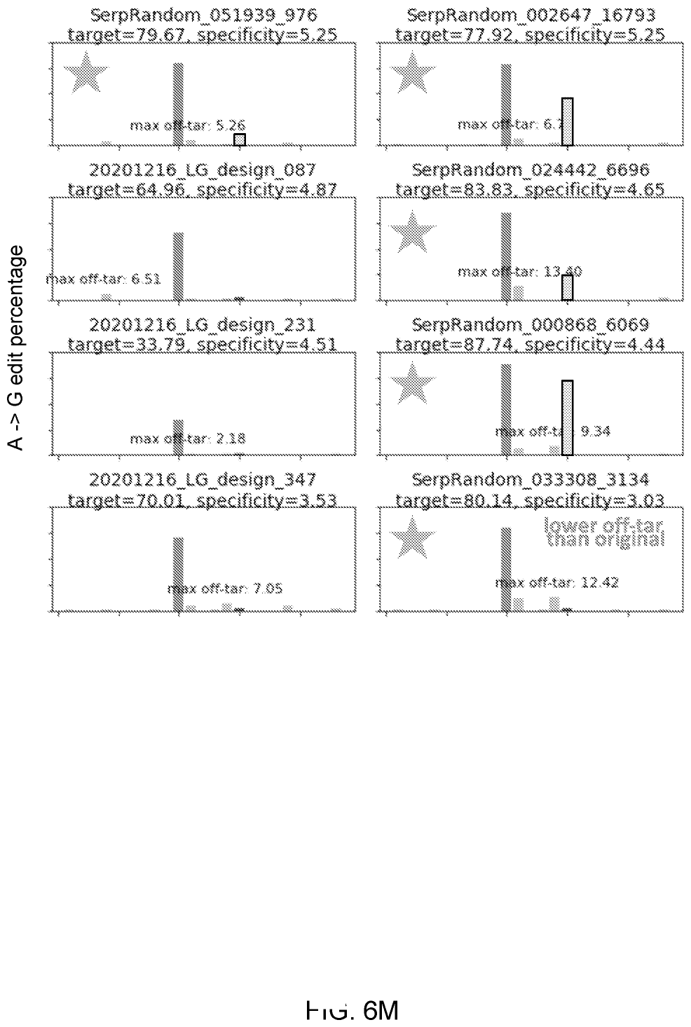

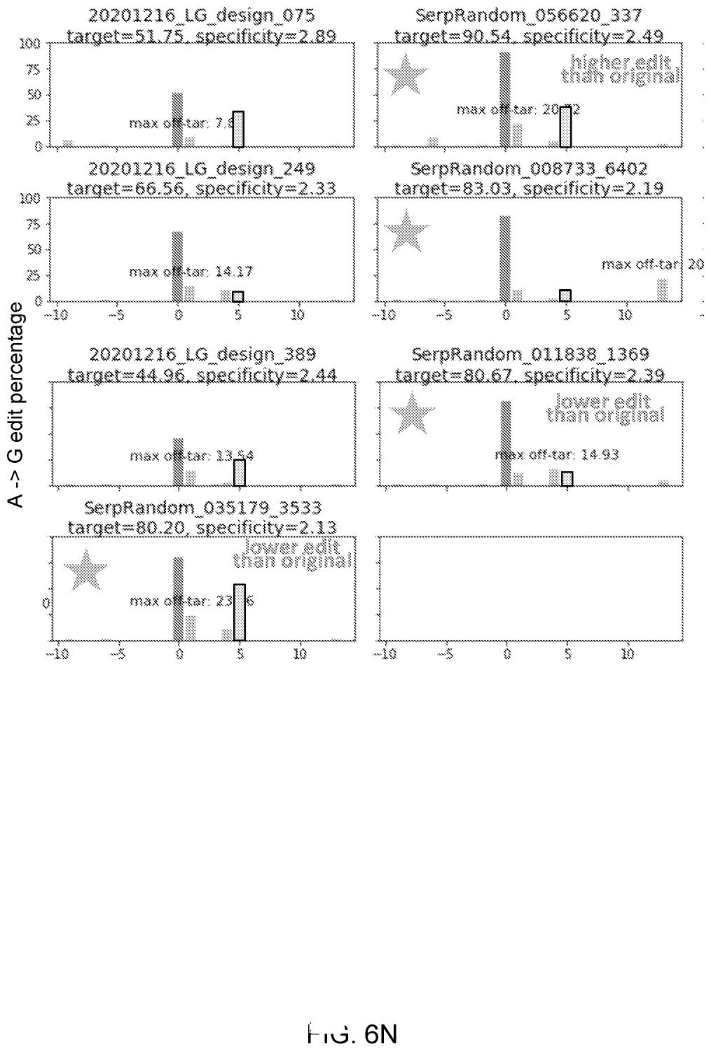

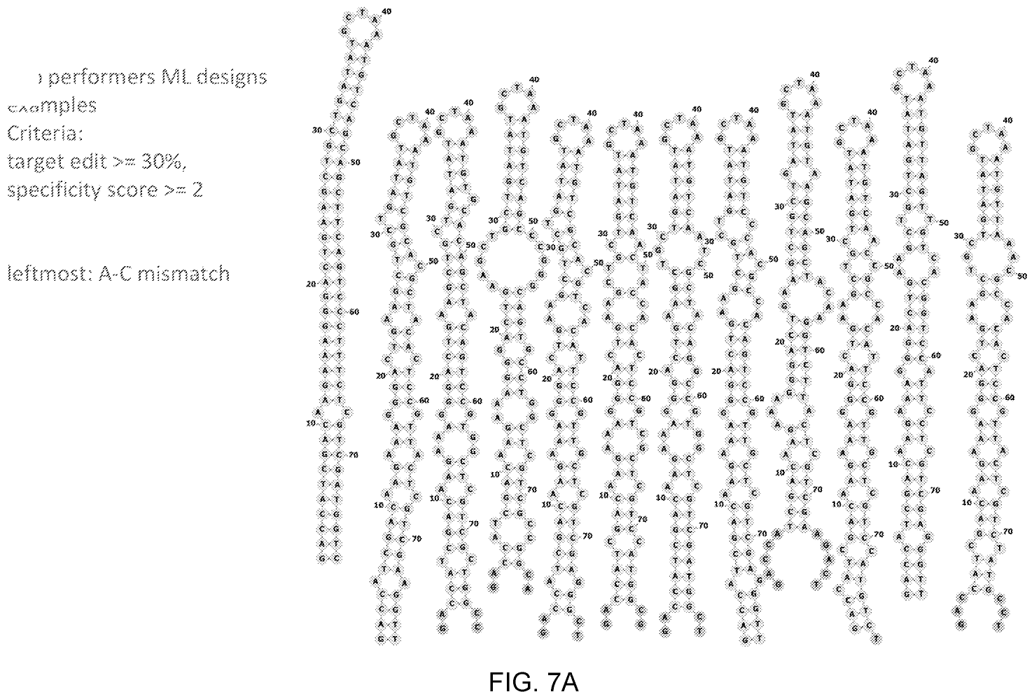

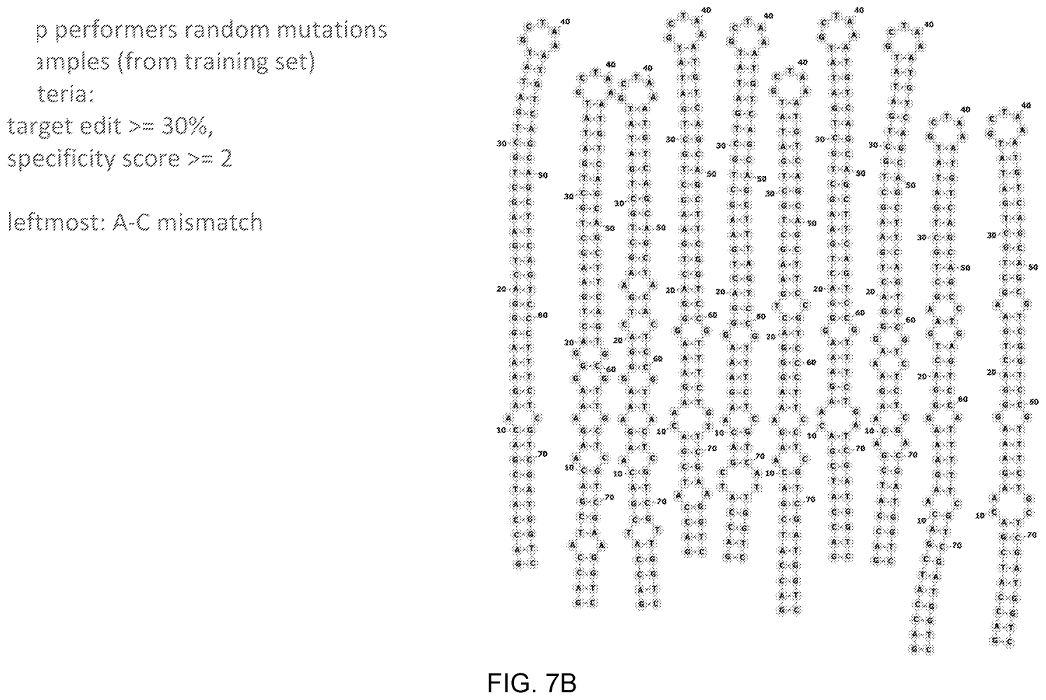

In some embodiments, the model predicts variables such as target editing (e.g., A>I editing) percentage by the ADAR 140 or ADAR of FIG. 23B and editing specificity score (e.g., one or more metrics for deamination by the ADAR protein of a target nucleotide position in a target mRNA sequence, as determined using a plurality of sequence reads obtained from a plurality of target mRNAs). In some embodiments, a specificity score is defined as the target edit percentage divided by the sum of all nonsynonymous off-target edits. In some embodiments, a specificity score is determined as the (sum of on-target editing of the desired nucleotide)/(sum of off-target editing). In some embodiments, a specificity score is determined as 1−(# of reads with only on-target edits)−(# of reads with zero edits). Additional predicted variables contemplated for use in the present disclosure include, but are not limited to, minimum free energy of the double-stranded self-editing hairpin structure or minimum free energy of the guide-target RNA scaffold. The machine learning model, in some embodiments, simultaneously predicts target adenosine edit and off-target edit (or specificity). FIG. 6A is a graphical illustration of example inputs and outputs of a CNN, in accordance with some embodiments. FIGS. 6B-D is a graphical illustration of the results of some of the example sequences generated by a CNN, in accordance with some embodiments.

Alternatively or additionally, in some embodiments, the machine learning model generates a prediction of one or more metrics that measure the deamination ability of an ADAR protein on a target nucleotide position in a target mRNA when facilitated by hybridization of a gRNA having the respective candidate sequence. In some embodiments, the one or more metrics are selected from the group consisting of any on-target editing, specificity, target-only editing, no editing, and normalized specificity, for one or more ADAR proteins in a plurality of different ADAR proteins. For instance, in some embodiments, any on-target editing is determined as a proportion of sequence reads with any on-target edits. In some embodiments, specificity is determined as a (proportion of sequence reads with on-target edits+1)/(proportion of sequence reads with off-target edits+1). In some embodiments, target-only editing is determined as a proportion of sequence reads with only on-target edits. In some embodiments, no editing is determined as a proportion of sequence reads without any edits. In some embodiments, normalized specificity is determined as 1−(proportion of sequence reads with any off-target edits). In some embodiments, the one or more metrics further includes a difference in editing preference between a first ADAR protein and a second ADAR protein, in the plurality of different ADAR proteins. In some embodiments, the difference in editing preference is determined as (target-only editing of the first ADAR protein)−(target-only editing of the second ADAR protein). In some embodiments, the one or more metrics are obtained for ADAR1, ADAR2, or ADAR1/2. Alternatively or additionally, in some embodiments, the one or more metrics further includes editability, where editability is a measure of central tendency of the any on-target editing and target-only editing scores. In some embodiments, editability is the average of the any on-target editing and target-only editing scores.

In some embodiments, model architectures are selected using hyperband early-stop algorithm on training and validation sets. In various embodiments, model architectures differ in the number of layers, number of convolutional filters, size of convolution filter kernel, stride, dilation, padding, number of fully-connected layers, number of neurons in each fully-connected layers, drop-out parameters after convolution or after fully-connected layers, batch size, learning rate, weight decay. In some embodiments, the model is trained by stochastic gradient descent. Detail of training and an exemplary structure of such a neural network machine learning model is illustrated in FIG. 4. For instance, in some implementations, neural network machine learning as disclosed herein has different architectures based on performance of the data set. Additionally, in some implementations, different ensemble models have different numbers of convolutional layers and fully connected layers. In some embodiments, an ensemble of models are trained using random subsets of the whole training set to minimize the risk of overfitting and cover a different part of the pace of the known sequence with a diverse set of architectures (these model architectures are selected as described above).

In some embodiments, models are validated with a holdout test set that is not used in training and validation. Model performance for regression is measured by percent variance explained and correlation between predicted and true values. In some example embodiments, model ensembles reach above 0.9 correlation coefficient on a predicted variable. FIG. 6E-G is a graphical illustration of the model performance that includes plots of correlations between true values and predicted values.

In some embodiments, the approach also generates a list of mutations (exhaustive list or not) in the nucleic acid sequences of candidates of engineered guide agents 130 or of engineered guide RNAs of FIG. 23B. The trained model ensembles, in some embodiments, are used to score and rank the list. For example, given the desired number and lengths of mutations with regard to perfectly complementary target and guide strands (perfect duplex) in an engineered guide RNA 130 or an engineered guide RNA of FIG. 23B, an algorithm exhaustively generates all possible candidate engineered guide RNA 130 or candidate engineered guide RNA of FIG. 23B, such as all mutated engineered guide RNA. These candidates are fed into the model ensembles trained on existing engineered guide RNA data to predict the candidates' target editing score and specificity score when edited by ADAR1 and/or ADAR2, as well as the minimum free energy of the folded structure or of the guide-target RNA scaffold. These mutated sequences are then ranked by their predicted scores, effectively eliminating poorly performed sequences and narrowing down the vast sequence space to be tested experimentally.

For input optimization, in some implementations, a random seed of the same shape as the input between (0,1) with channels summing to one is fed into the ensemble of model networks. In some implementations, the random seed includes positional encoding. In some implementations, the model parameters (e.g., weights) are fixed. In some embodiments, a loss function is used to determine the difference between ensemble output (predicted) and desired (predefined) values, and gradient with regard to the input is calculated and back-propagated to update the input (random seed). Gradients on certain predefined portions of the input, in some embodiments, are masked to prevent changing the target domain. In some embodiments, gradients are clipped to within certain bounds. In some implementations, the input being optimized is clamped to within certain bounds. In some embodiments, the input is projected from continuous space (for example, taking a value between 0 and 1) to one hot encoded space (taking a value of either 0 or 1, only one value is 1 per channel). In some embodiments, iterations are stopped before the predefined number of iterations are reached if the loss of one hot projected sequence stopped improving (e.g., convergence). FIGS. 6H and 6I are graphical illustrations of experimental validations showing that engineered guide RNAs generated by the input optimization of the second approach 350, in certain embodiments, perform better than the original training data inputs. FIG. 6J-N is an enlarged view of plots obtained as described in FIG. 6I.

### 7. Further Discussion on Machine Learning Processes

For input optimization, in some embodiments, the whole procedure generates new (e.g., not in the training set) nucleic acid sequences of candidates of engineered guide RNA 130 or of engineered guide RNA of FIG. 23B that the ensemble model predicts to have predefined scores. This also yields the variability (measured by standard deviation) of the ensemble of networks on their predictions.

Self-supervised models used to learn the distribution of the editing of double-stranded ADAR substrates are used, in some embodiments, as a pre-trained model to transfer information about sequence space constraints to downstream supervised models. This has the advantage of fully utilizing even the “unlabeled” data (data for which experimental measurements have not been obtained).

In some embodiments, different predicted variables are engineered from observed data, and models are trained to predict them, such as ADAR 1, ADAR2, or ADAR 1 and ADAR 2 (ADAR1/2) editing kinetics (e.g., the time course of A>I editing at a particular site or multiple sites).

In some embodiments, the discovery of ADAR editing rules and patterns in the first approach 310 are used to guide the human expert design of guide RNA sequences or used in machine-given generation of novel guide RNA sequences. In some embodiments, proposed engineered guide RNA sequences are used for experimental testing in one or more of in vitro cell-free experiments, in vitro cell experiments, or in vivo experiments, as shown in approach 310 in FIG. 3. In some embodiments, proposed engineered guide RNA sequences are used for experimental testing in one or more of in vitro cell-free experiments, in vitro cell experiments, or in vivo experiments, as shown in approach 350 in FIG. 3. Moreover, in some embodiments, one or more experimental values are obtained from the in vitro cell-free experiments, in vitro cell experiments, or in vivo experiments, which are used to refine the generation of engineered guide RNA sequences in subsequent iterations of approaches 310 and/or 350. In some embodiments, one or more cell types are used in the in vitro cell experiments or in vivo experiments. In some embodiments, the one or more experimental values obtained from the in vitro cell-free experiments, in vitro cell experiments, or in vivo experiments are subsequently used to further train the model in approaches 310 or 350.

### 8. Example Machine Learning Models for Attribute Prediction

Another aspect of the present disclosure provides a method for predicting deamination efficiency by Adenosine Deaminases Acting on RNA (ADAR) that can be associated with a guide RNA (gRNA) comprising, at a computer system comprising at least one processor and a memory storing at least one program for execution by the at least one processor, receiving a nucleic acid sequence for the gRNA. Another aspect of the present disclosure provides a method for predicting deamination specificity score by Adenosine Deaminases Acting on RNA (ADAR) that can be associated with a guide RNA (gRNA) comprising, at a computer system comprising at least one processor and a memory storing at least one program for execution by the at least one processor, receiving a nucleic acid sequence for the gRNA.

Responsive to inputting a data structure into a model, the method includes obtaining as output from the model a metric for an efficiency of deamination of a target nucleotide position by a first ADAR protein in mRNA transcribed from a target gene (e.g., where the model comprises at least 10,000 parameters). Responsive to inputting a data structure into a model, the method includes obtaining as output from the model a metric for an efficiency of deamination of one or more nucleotide positions other than the target nucleotide position (also referred to, in some implementations, as a specificity score herein) by the first ADAR protein (e.g., where the model comprises at least 10,000 parameters). In some embodiments, the data structure comprises a two-dimensional matrix encoding the nucleic acid sequence for the gRNA, where the two-dimensional matrix has a first dimension and a second dimension, and where the first dimension represents nucleotide position within the gRNA and the second dimension represents nucleotide identity within the gRNA.

In some embodiments, the metric for the efficiency of deamination of the target nucleotide position in mRNA transcribed from the target gene by the first ADAR protein is normalized by a metric for an efficiency of deamination of one or more nucleotide positions other than the target nucleotide position by the first ADAR protein in the mRNA transcribed from the target gene estimated by at least a subset of the plurality of (e.g., at least 100,000) parameters responsive to the inputting the representation of the gRNA into the model.

In some embodiments, the metric for the efficiency of deamination of the target nucleotide position in mRNA transcribed from the target gene by the first ADAR protein is normalized by a metric for an efficiency of deamination of a target nucleotide position by a first ADAR protein in mRNA transcribed from a target gene estimated by at least a subset of the plurality of (e.g., at least 100,000) parameters responsive to the inputting the representation of the gRNA into the model.

In some embodiments, the model further outputs, responsive to the inputting the data structure into the model, a metric for an efficiency of deamination of one or more nucleotide positions other than the target nucleotide position by the first ADAR protein in the mRNA transcribed from the target gene.

In some embodiments, the model further outputs, responsive to the inputting the data structure into the model, a metric for an efficiency of deamination of a target nucleotide position by a first ADAR protein in mRNA transcribed from a target gene.

In some embodiments, the first ADAR protein is ADAR1 or ADAR2. In some embodiments, the first ADAR protein comprises both ADAR1 and ADAR2. In some embodiments, the first ADAR protein is human ADAR1 or human ADAR2. In some embodiments, the first ADAR protein comprises both human ADAR1 and human ADAR2. In some embodiments, the first ADAR protein is ADAR2 and the guide RNA targets neurons for editing. In some embodiments, the first ADAR protein is ADAR1 and the guide RNA targets liver for editing. In some embodiments, the first ADAR protein is ADAR1 and the guide RNA de-targets neurons for editing.

In some embodiments, the model further outputs, responsive to the inputting the data structure into the model, a metric for an efficiency of deamination of the target nucleotide position by a second ADAR protein.

In some embodiments, the model further outputs, responsive to the inputting the data structure into the model, a metric for an efficiency of deamination of one or more nucleotide positions other than the target nucleotide position in the mRNA transcribed from the target gene by a second ADAR protein.

In some embodiments, the second ADAR protein is ADAR2 or ADAR1. In some embodiments, the second ADAR protein is human ADAR2 or human ADAR1. In some embodiments, the second ADAR protein is ADAR2 and the guide RNA targets neurons for editing. In some embodiments, the second ADAR protein is ADAR1 and the guide RNA targets liver for editing. In some embodiments, the second ADAR protein is ADAR1 and the guide RNA de-targets neurons for editing.

In some embodiments, the model further generates, responsive to the inputting the data structure into the model, an estimation of a minimum free energy (MFE) for the gRNA. In some embodiments, the model further generates, responsive to the inputting the data structure into the model, an estimation of a minimum free energy (MFE) for the self-annealing hairpin comprising a gRNA linked to the target RNA by a hairpin. In some embodiments, the model further generates, responsive to the inputting the data structure into the model, an estimation of a minimum free energy (MFE) for the guide-target RNA scaffold.

In some embodiments, the model is a neural network, a support vector machine, a Naive Bayes model, a nearest neighbor model, a boosted trees model, a random forest model, a decision tree, or a clustering model. In some implementations, the model is a convolutional or graph-based neural network.

In some embodiments, the model comprises a plurality of parameters. In some embodiments, the model comprises at least 100,000, at least 250,000, at least 500,000, or at least 1,000,000 parameters.

In some embodiments, the plurality of parameters for the model comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million parameters. In some embodiments, the plurality of parameters comprises no more than 8 million, no more than 5 million, no more than 4 million, no more than 1 million, no more than 500,000, no more than 100,000, no more than 50,000, no more than 10,000, no more than 5000, no more than 1000, or no more than 500 parameters. In some embodiments, the plurality of parameters consists of from 10 to 5000, from 500 to 10,000, from 10,000 to 500,000, from 20,000 to 1 million, or from 1 million to 5 million parameters. In some embodiments, the plurality of parameters falls within another range starting no lower than 10 parameters and ending no higher than 8 million parameters.

In some embodiments, the data structure further comprises indications of a plurality of secondary structure features of the gRNA (e.g., in the guide-target RNA scaffold). In some embodiments, the plurality of secondary structure features comprises indications for at least five types of secondary structure features of the gRNA.

In some embodiments, the plurality of secondary structure features comprises indications for at least 1, at least 2, at least 3, at least 4, at least 5, at least 6, at least 7, at least 8, at least 9, at least 10, at least 15, at least 20, at least 25, at least 30, at least 40, at least 50, at least 60, at least 80, or at least 100 types of secondary structure features of the gRNA (e.g., in the guide-target RNA scaffold). In some embodiments, the plurality of secondary structure features comprises indications for no more than 100, indications for no more than 80, indications for no more than 60, indications for no more than 50, indications for no more than 40, no more than 25, no more than 15, no more than 10, or no more than 5 types of secondary structure features of the gRNA. In some embodiments, the plurality of secondary structure features comprises indications for from 1 to 5, from 4 to 10, from 5 to 20, from 10 to 40, from 2 to 100, from 2 to 50, from 1 to 100, from 5 to 100, or from 10 to 100 types of secondary structure features of the gRNA. In some embodiments, the plurality of secondary structure features comprises indications that fall within another range starting no lower than 1 and ending no higher than 100 types of secondary structure features of the gRNA.

In some embodiments, the plurality of secondary structure features comprises one or more secondary structure features selected from the group consisting of a structural motif comprising two or more secondary structure features; a presence or absence of a mismatch formed when binding to the mRNA transcribed from the target gene; a position of a mismatch formed when binding to the mRNA transcribed from the target gene; a presence or absence of a bulge formed when binding to the mRNA transcribed from the target gene; a position of a bulge formed when binding to the mRNA transcribed from the target gene; a size of a bulge formed when binding to the mRNA transcribed from the target gene; a presence or absence of an internal loop in the gRNA when binding to the mRNA transcribed from the target gene; a position of an internal loop in the gRNA when binding to the mRNA transcribed from the target gene; a size of an internal loop in the gRNA when binding to the mRNA transcribed from the target gene; a presence or absence of an internal loop in the mRNA transcribed from the target gene when binding to the gRNA; a position of an internal loop in the mRNA transcribed from the target gene when binding to the gRNA; a size of an internal loop in the mRNA transcribed from the target gene when binding to the gRNA; a presence or absence of a hairpin in the gRNA when binding to the mRNA transcribed from the target gene; a position of a hairpin in the gRNA when binding to the mRNA transcribed from the target gene; a size of a hairpin in the gRNA when binding to the mRNA transcribed from the target gene; a presence or absence of a hairpin in the mRNA transcribed from the target gene when binding to the gRNA; a position of a hairpin in the mRNA transcribed from the target gene when binding to the gRNA; a size of a hairpin in the mRNA transcribed from the target gene when binding to the gRNA; a presence or absence of a wobble base pair formed when binding to the mRNA transcribed from the target gene; a position of a wobble base pair formed when binding to the mRNA transcribed from the target gene; a presence or absence of a barbell when binding to the mRNA transcribed from the target gene; a position of a barbell when binding to the mRNA transcribed from the target gene; a size of a barbell when binding to the mRNA transcribed from the target gene; a presence or absence of a dumbbell when binding to the mRNA transcribed from the target gene; a position of a dumbbell when binding to the mRNA transcribed from the target gene; a size of a dumbbell when binding to the mRNA transcribed from the target gene; a presence or absence of a base paired region formed when binding to the mRNA transcribed from the target gene; a position of a base paired region formed when binding to the mRNA transcribed from the target gene; and a size of a base paired region formed when binding to the mRNA transcribed from the target gene.

In some embodiments, the data structure further comprises indications of a plurality of tertiary structures of the gRNA (e.g., in the guide-target RNA scaffold). In some embodiments, the plurality of tertiary structures comprises indications for at least five types of tertiary structures of the gRNA.

In some embodiments, the plurality of tertiary structures comprises indications for at least 1, at least 2, at least 3, at least 4, at least 5, at least 6, at least 7, at least 8, at least 9, at least 10, at least 15, at least 20, at least 25, at least 30, at least 40, at least 50, at least 60, at least 80, or at least 100 types of tertiary structures of the gRNA (e.g., in the guide-target RNA scaffold). In some embodiments, the plurality of tertiary structures comprises indications for no more than 100, indications for no more than 80, indications for no more than 60, indications for no more than 50, indications for no more than 40, no more than 25, no more than 15, no more than 10, or no more than 5 types of tertiary structures of the gRNA. In some embodiments, the plurality tertiary structures comprises indications for from 1 to 5, from 4 to 10, from 5 to 20, from 10 to 40, from 2 to 100, from 2 to 50, from 1 to 100, from 5 to 100, or from 10 to 100 types of tertiary structures of the gRNA. In some embodiments, the plurality of tertiary structures comprises indications that fall within another range starting no lower than 1 and ending no higher than 100 types of tertiary structures of the gRNA.

In some embodiments, the plurality of tertiary structures comprises one or more tertiary structures selected from the group consisting of a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the gRNA comprises at least 25 nucleotides. In other embodiments, the gRNA comprises at least 5 nucleotides. In some embodiments, the gRNA comprises at least 45 nucleotides. In some embodiments, the gRNA comprises at least 5, 10, 15, 20, 25, 30, 35, 40, 45, 50, 55, 60, 65, 70, 75, 80, 85, 90, 95, 100, 200, 300, 400, 500, 600, 700, 800, 900, or 1000 nucleotides, or any number of nucleotides therebetween. In some embodiments, the gRNA comprises at least 5 nucleotides and no more than 1000 nucleotides. In some embodiments, the gRNA comprises from 5 to 1000, from 20 to 100, from 35 to 60, or from 35 to 50 nucleotides.

In some embodiments, the gRNA facilitates adenosine to inosine editing of a target nucleotide at the target nucleotide position in mRNA transcribed from the target gene by ADAR.

In some embodiments, the data structure further comprises a first polynucleotide sequence flanking a 5′ side of the target nucleotide position in mRNA transcribed from the target gene and a second polynucleotide sequence flanking a 3′ side of the target nucleotide position in mRNA transcribed from the target gene.

### 9. Example Machine Learning Models for Guide Prediction

Another aspect of the present disclosure provides a method for generating a candidate sequence for a guide RNA (gRNA) that guides deamination of a target nucleotide position by an Adenosine Deaminase Acting on RNA (ADAR) protein in mRNA transcribed from a target gene, comprising, at a computer system comprising at least one processor and a memory storing at least one program for execution by the at least one processor, receiving a set of desired values comprising an enumerated value for each property in a set of properties for gRNA, where the set of properties includes a metric for an efficiency and/or specificity score of deamination of a target nucleotide position in mRNA transcribed from a target gene by a first ADAR protein.

The method includes receiving a data structure comprising a seed sequence for the gRNA, and performing an input optimization operation using a model, where the model comprises a plurality of (e.g., at least 100,000) parameters, the model comprises an input layer configured to accept the data structure, the model is configured to output predicted values for each property in the set of properties, and the set of properties comprises a metric for an efficiency and/or specificity score of deamination of a target nucleotide position in mRNA transcribed from a target gene by a first ADAR protein.

The input optimization operation comprises i) responsive to inputting the data structure comprising the seed sequence for the gRNA, obtaining a set of calculated values for the set of properties for gRNA, and ii) back-propagating through the model, while holding the plurality of parameters fixed, a difference between the set of calculated values and the set of desired values to modify the seed sequence for the gRNA responsive to the difference, thereby generating the candidate sequence.

In some embodiments, the model is configured to output predicted values for a specific ADAR isoform to allow for editing specificity in a target cell. For example, in some implementations, configuring for ADAR2 preference limits editing activity to neurons. In some embodiments, configuring for ADAR1 preference avoids editing activity in neurons, and promotes, for example, editing activity in liver cells. In some embodiments, configuring for ADAR1 and ADAR2 preference ensures editing activity in multiple tissues.

In some embodiments, the method further includes determining, using a gRNA having the candidate sequence, a set of experimental values for the set of properties for gRNA; and training a model using a data structure comprising the candidate sequence and a difference between the set experimental values and the set of calculated values. In some embodiments, a set of experimental values is from in vitro cell-free experiments, in vitro cell experiments, or in vivo experiments. In some embodiments, one or more cell types are used in the in vitro experiments and subsequently used to train the model.

### Target RNA

In some embodiments, the target RNA to be edited is a pre-mRNA. In some embodiments, the target RNA is a mature mRNA. In some embodiments, the target RNA is a miRNA or siRNA.

In some embodiments, the target RNA is coding sequence. In some embodiments, the target RNA is a non-coding sequence. In certain embodiments, the target RNA is a splice acceptor or donor site. In certain additional embodiments, the target RNA is a transcriptional start site. In certain embodiments, the target RNA is in a polyA signal sequence.

In some embodiments, the target RNA is an mRNA and/or pre-mRNA. In some embodiments, the mRNA and/or pre-mRNA comprises a mutation that results in loss of wild-type protein expression, and editing effected by contacting the target RNA with the gRNA increases expression of the protein encoded by the RNA. In some embodiments, a full expression of the protein is restored. In some embodiments, partial expression is restored. In particular embodiments, sufficient expression is restored to improve signs or symptoms of a disease or disorder. In select embodiments, the target RNA is expressed from a mutated gene that causes one or more genetic diseases.

In some embodiments, the mRNA and/or pre-mRNA comprises a mutation that results in an increase of protein expression (e.g., a protein associated with a disease phenotype), and editing effected by contacting the target RNA with the gRNA decreases or inhibits expression of the protein encoded by the RNA. In some embodiments, a full inhibition of the protein expression is achieved. In some embodiments, partial inhibition of expression is achieved. In particular embodiments, sufficient expression is inhibited to improve signs or symptoms of a disease or disorder. In select embodiments, the target RNA is expressed from a mutated gene that causes one or more genetic diseases.

In certain embodiments, the target RNA comprises a point mutation. In particular embodiments, the point mutation results in a missense mutation, splice site alteration, or a premature stop codon.

In some embodiments, the target RNA is expressed in one or more cell types. In some embodiments, the cell type is a neuron. In some embodiments, the cell type is a liver cell. In some embodiments, target RNA is expressed in both a neuron and a liver cell.

### Compositions Comprising Engineered Guide RNA

The engineered guide RNA 130 or engineered guide RNA of FIG. 23B, in some embodiments, takes the form of recombinant guide nucleic acid molecules. In some embodiments, the recombinant guide nucleic acid molecules are provided in any number of suitable forms, including in naked form, in complexed form, or in a delivery vehicle.

In certain embodiments, an engineered guide RNA 130 or engineered guide RNA of FIG. 23B is in naked form. In particular embodiments, the engineered guide RNA 130 or engineered guide RNA of FIG. 23B is in a fluid composition without any other carrier proteins or delivery vehicles. In certain embodiments, the engineered guide RNA 130 or engineered guide RNA of FIG. 23B is in complex form, bound to other nucleic acid or amino acids that assist in maintaining stability, such as by reducing exonuclease or endonuclease digestion.

In some embodiments, the engineered guide RNA 130 or engineered guide RNA of FIG. 23B is formulated into a composition that comprises the engineered guide RNA 130 or engineered guide RNA of FIG. 23B and at least one carrier or excipient. In some embodiments intended for direct administration of the engineered guide RNA 130 or engineered guide RNA of FIG. 23B to a patient, the engineered guide RNA 130 or engineered guide RNA of FIG. 23B is formulated in a pharmaceutical composition that comprises the engineered guide RNA 130 or engineered guide RNA of FIG. 23B and at least one pharmaceutically acceptable carrier or excipient. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients, in some embodiments, are further incorporated into the compositions.

Delivery vehicles such as liposomes, nanocapsules, microparticles, microspheres, lipid particles, vesicles, and the like, in some embodiments, are used for the introduction of any of the recombinant nucleic acids or compositions described herein into suitable host cells. In particular, the compositions or recombinant nucleic acids, in some embodiments, are formulated for delivery either encapsulated in a lipid particle, a liposome, a vesicle, a nanosphere, a nanoparticle, or the like.

Methods to deliver recombinant guide nucleic acid molecules and related compositions described herein include any suitable method including: via nanoparticles including using liposomes, synthetic polymeric materials, naturally occurring polymers and/or inorganic materials to form nanoparticles.

Examples of lipid-based materials for delivery of the DNA or RNA molecules include: polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and/or Eufectins (JBL, San Luis Obispo, Calif.). In some implementations, exemplary cationic liposomes are made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl]cholesterol (DC-Chol), 2,3,-dioleyloxy-N-[2 (sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and/or dimethyldioctadecylammonium bromide (DDAB). In some embodiments, nucleic acids (e.g., ceDNA) are also complexed with, e.g., poly (L-lysine) or avidin, with or without the presence of lipids in this mixture, e.g., steryl-poly (L-lysine).

Naturally occurring polymers contemplated for use in the present disclosure include, but are not limited to, chitosan, protamine, atelocollagen and/or peptides.

Non-limiting examples of inorganic materials also contemplated for use in the present disclosure include gold nanoparticles, silica-based, and/or magnetic nanoparticles, which are produced, in some implementations, by methods known to the person skilled in the art.

### Vectors Encoding Engineered Guide Agent

In some embodiments, vectors encoding engineered guide RNA 130 or engineered guide RNA of FIG. 23B are provided.

In some embodiments, the vector does not express the engineered guide RNA 130 or engineered guide RNA of FIG. 23B and is used to propagate polynucleotides that encode the engineered guide RNA 130 or engineered guide RNA of FIG. 23B. In some embodiments, the encoding polynucleotide is DNA. In some embodiments, the vector is a plasmid. In some embodiments, the vector is a phage. In some embodiments, the vector is a phagemid. In some embodiments, the vector is a cosmid.

In some embodiments, the vector is capable of expressing the engineered guide RNA 130 or engineered guide RNA of FIG. 23B. In some embodiments, expression vectors are used to introduce the engineered guide RNA 130 or engineered guide RNA of FIG. 23B into cells in vitro or in vivo.

In typical expression vector embodiments, the vector comprises a coding region, wherein the coding region encodes at least one engineered guide RNA 130 or engineered guide RNA of FIG. 23B as described herein. The coding region is operably linked to expression control elements that direct transcription. In some embodiments, the expression vector is an adenoviral vector, an adeno-associated virus (AAV) vector, a retroviral vector, or a lentiviral vector. In certain preferred embodiments, the vector is an AAV vector, and the expression control elements and engineered guide agent 130 coding region or engineered guide RNA of FIG. 23B coding region are together flanked by 5′ and 3′ AAV inverted terminal repeats (ITR).

In some embodiments, the vector is packaged into a recombinant virion. In particular embodiments, the vector is packaged into a recombinant AAV virion.

### Compositions Comprising Engineered Guide Agent Vectors

In another aspect, compositions comprising the engineered guide RNA vectors are provided.

In some embodiments, the compositions are suitable for administration to a patient, and the composition is a pharmaceutical composition comprising a recombinant virion and at least one pharmaceutically acceptable carrier or excipient. In typical embodiments, the pharmaceutical composition is adapted for parenteral administration. In certain embodiments, the pharmaceutical composition is adapted for intravenous administration, intravitreal administration, posterior retinal administration, intrathecal administration, or intra-cisterna magna (ICM) administration.

### Methods for Editing RNA

To effect editing of RNA, the engineered guide RNA 130 or engineered guide RNA of FIG. 23B is contacted to the target RNA in the presence of ADAR enzymes. Typically, contact is within a cell. In certain embodiments, the contacting is performed in vitro. In some embodiments, in vitro is cell-free. In some embodiments, in vitro is in cells. In certain embodiments, the contacting is performed in vivo.

Thus, in another aspect, methods are provided for editing target RNAs. The methods comprise contacting the target RNA with at least one engineered guide agent 130 or engineered guide RNA of FIG. 23B as described herein. In some embodiments, the contacting is performed in vitro. In some embodiments, the contacting is performed in vivo.

In some embodiments, the method comprises the preceding step of introducing one or more engineered guide RNAs 130 or engineered guide RNAs of FIG. 23B into a cell comprising the target RNA. In some embodiments, the method comprises the preceding step of introducing one or more recombinant expression vectors that are capable of expressing the one or more recombinant engineered guide RNA 130 or engineered guide RNA of FIG. 23B into the cell. In some embodiments, the methods further comprise delivering an ADAR enzyme, or ADAR-encoding polynucleotide, into the cell.

In some embodiments, the engineered guide RNA 130 or engineered guide RNA of FIG. 23B takes the form of a recombinant guide nucleic acid molecule. The recombinant guide nucleic acid molecules and vectors disclosed herein are, in some embodiments, introduced into desired or target cells by any techniques known in the art, such as liposomal transfection, chemical transfection, micro-injection, electroporation, gene-gun penetration, viral infection or transduction, transposon insertion, jumping gene insertion, and/or a combination thereof.

In some embodiments, the recombinant guide nucleic acid molecules and related compositions disclosed herein are delivered by any suitable system, including by using any gene delivery vectors, such as adenoviral vector, adeno-associated vector, retroviral vector, lentiviral vector, or a combination thereof. In some embodiments, a recombinant adenoviral vector, a recombinant adeno-associated vector, a recombinant retroviral vector, a recombinant lentiviral vector, or a combination thereof, is used to introduce any of the recombinant guide molecules or nucleic acid molecules described herein.

In some embodiments, the recombinant guide nucleic acid molecules disclosed herein are present in a composition comprising physiologically acceptable carriers, excipients, adjuvants, or diluents. Neutral buffered saline or saline mixed with serum albumin are exemplary appropriate diluents. Suitable carriers include aqueous isotonic sterile injection solutions, including those that contain antioxidants, buffers, bacteriostats, and solutes that render the formulation isotonic with the blood of the intended recipient, and aqueous and non-aqueous sterile suspensions, including those that comprise suspending agents, solubilizers, thickening agents, stabilizers, and preservatives.

The pharmaceutically acceptable carriers (vehicles) useful in this disclosure are conventional. In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers include, in some implementations, pharmaceutical grades of mannitol, lactose, starch, or magnesium stearate. In addition to biologically-neutral carriers, pharmaceutical compositions to be administered contain, in some embodiments, minor amounts of non-toxic auxiliary substances, such as wetting or emulsifying agents, preservatives, and pH buffering agents and the like, for example, sodium acetate or sorbitan monolaurate.

In some embodiments, compositions, whether they be solutions, suspensions or other like form, include one or more of the following: DMSO, sterile diluents such as water for injection, saline solution, preferably physiological saline, Ringer's solution, isotonic sodium chloride, fixed oils such as synthetic mono or diglycerides for serving as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

### Methods of Treating Diseases Caused by Altering of Protein Expression

In another aspect, methods are provided for treating diseases caused by the loss of wild-type expression. The method comprises delivering an effective amount of at least one engineered guide RNA 130 or engineered guide RNA of FIG. 23B to a patient having a disease or disorder resulting from the loss of wild-type expression of a protein, wherein the engineered guide RNA 130 or engineered guide RNA of FIG. 23B is capable of recruiting ADAR to edit an RNA target, thereby increasing or restoring expression of the wild-type protein whose expression was decreased or lost in the diseased state.

In another aspect, methods are provided for treating diseases associated with expression of a protein. The method comprises delivering an effective amount of at least one engineered guide RNA 130 or engineered guide RNA of FIG. 23B to a patient having a disease or disorder resulting from the expression of a protein, wherein the engineered guide RNA 130 or engineered guide RNA of FIG. 23B is capable of recruiting ADAR to edit an RNA target, thereby decreasing or inhibiting expression of the protein whose expression is associated with the diseased state.

There are numerous examples of diseases or conditions caused by aberrant protein expression, or loss of wild-type protein expression (i.e., either increased or decreased from wild-type expression) that would be suitable for treatment using the methods described herein relating to ADAR editing. There are numerous examples of diseases or conditions caused by protein expression associated with a diseased state (e.g., expression leading to protein aggregates that cause disease, which in some embodiments, is seen in adults or older adults) that would be suitable for treatment using the methods described herein relating to ADAR editing.

An example includes conditions caused by missense mutations that render the resulting protein nonfunctional. Examples of such mutations that are responsible for human diseases including Epidermolysis bullosa, sickle-cell disease, and SOD1 mediated amyotrophic lateral sclerosis (ALS). Another example is cystic fibrosis (Human Molecular Genetics, Vol. 7, Issue 11, October 1998, Pages 1761-1769.).

The RNA editing techniques and methods described herein are likely to be most beneficial during the newborn or infant stages for certain diseases, but, in some embodiments, provide benefits at any stage of life. The term pediatric typically refers to anyone under 15 years of age, and less than 35 kg. A neonate typically refers to a newborn up to the first 28 days of life. The term infant typically refers to an individual from the neonatal period up to 12 months. The term toddler typically refers to an individual from 1-3 years of age. Teenagers are typically considered to be 13-19 years of age. Young adults are typically considered to be from 19-24 years of age.

### Kits

Additionally, the present disclosure provides a kit comprising certain components or embodiments of the heterologous and/or recombinant engineered guide nucleic acid molecule compositions. For example, in some implementations, any of the heterologous/recombinant engineered guide nucleic acid compositions, as well as the related buffers or other components related to administration are provided frozen and packaged as a kit, alone or along with separate containers of any of the other agents from the pre-conditioning or post-conditioning steps, and optional instructions for use. In some embodiments, the kit comprises ampoules, disposable syringes, capsules, vials, tubes, or the like. In some embodiments, the kit comprises a single dose container or multiple-dose containers comprising the embodiments herein. In some embodiments, each dose container contains one or more unit doses. In some embodiments, the kit includes an applicator. In some embodiments, the kits include all components needed for the stages of conditioning/treatment. In some embodiments, the compositions have preservatives or are preservative-free (for example, in a single-use container).

Methods of Treatment using an RNA Editing System

FIG. 2 is a flowchart depicting an example process 200 for treating a patient, in accordance with some embodiments. In some embodiments, the treatment is or is not a personalized treatment. In some embodiments, one or more steps in the process 200 are performed as an engineered guide RNA discovery process or a drug discovery process. In some embodiments, one or more steps are computer-implemented steps that are performed by a computing device. The computer-implemented steps, in some embodiments, are part of a software algorithm that is stored as computer instructions executable by one or more general processors (e.g., CPUs, GPUs). The instructions, when executed by the processors, cause the processors to perform the computer-implemented steps described in the process 200. In various embodiments, one or more steps in the process 200 are skipped or changed.

In accordance with some embodiments, a biological sample of a subject is received 210. In some embodiments, the subject suffers from one or more genetic diseases. The biological sample, in some embodiments, is any suitable biological sample such as saliva, hair, tissue biopsy, blood, whole blood, plasma, serum, urine, cerebrospinal fluid, fecal, saliva, tears, a tissue biopsy, pleural fluid, pericardial fluid, or peritoneal fluid of the subject. In some implementations, a genetic sequence of the subject is generated 220 by sequencing the biological sample. In some embodiments, sequencing includes sequencing of deoxyribonucleic acid (DNA) sequencing, ribonucleic acid (RNA) sequencing, etc. Suitable sequencing techniques contemplated for use in the present disclosure include Sanger sequencing and massively parallel sequencing such as various next-generation sequencing (NGS) techniques including whole genome sequencing, whole transcriptome sequencing, exome sequencing, pyrosequencing, sequencing by synthesis, sequencing by ligation, and ion semiconductor sequencing. The genetic sequence of a locus of interest of the subject, in some embodiments, is determined. The locus of interest, in some embodiments, contains one or more mutations that cause the genetic diseases.

In some embodiments, the genetic sequence of the locus of interest of the subject is digitalized 230 and stored in a database. A computing device, in some embodiments, retrieves 240 a nucleic acid sequence. The nucleic acid sequence, in some embodiments, is the DNA sequence or an mRNA sequence of interest. For example, the mutation in a DNA sequence is carried over to the mRNA through transcription. Thus, the mRNA digital sequence corresponds to the DNA sequence in the coding regions. In some embodiments, the digitalized nucleic acid sequence is an mRNA sequence or a portion of the mRNA sequence that includes one or more mutations. In other embodiments, the digitalized nucleic acid sequence is a DNA sequence that contains the mutations. Other suitable ways to store the mutation information are also possible.

In some embodiments, the computing device inputs 250 a version of the nucleic acid sequence into a machine learning model. A version of the nucleic acid sequence refers to a representation of the nucleic acid sequence that, in some embodiments, takes various forms. For example, in one version, the nucleic acid sequence is in a raw form that is represented by nucleotides such as A, T, C, G, U, and I. In another version, the nucleic acid sequence is converted into bits (e.g., 10101111) with each nucleotide being represented by one or more bits. In yet another version, the nucleic acid sequence is encoded as a mathematical vector through one or more signal processing schemes, encoding schemes, feature extraction techniques, and mappings. The features that are extracted from the nucleic acid sequence, in some embodiments, include, but are not limited to, the length of the sequence, physical properties of the sequences, chemical properties of the sequence, numbers of a particular nucleotide, the nucleotide values at one or more key sites, secondary structure prediction of the nucleic acid sequence, and structural features. Suitable encoding schemes, in some embodiments, include one hot encoding and positional encoding.

In some engineered guide RNA discovery processes or drug discovery processes, the nucleic acid sequence that is inputted 250 to a machine learning model is a common sequence that includes a known mutation that commonly causes a genetic disease, instead of a personalized nucleic acid sequence determined based on the sequencing of the subject's biological sample. In some such cases, one or more of steps 210 through 230 are performed or are skipped.

In some embodiments, the computing device also inputs a version of the nucleic acid sequence of a candidate engineered guide RNA 130 or a candidate engineered guide of FIG. 23B to the machine learning model. Similar to the DNA/mRNA of interest, the version of the sequence of a candidate engineered guide agent 130, in some embodiments, is the raw sequence, sequence that is converted into bits, a sequence that is encoded, or a mathematical vector that includes extracted features of the sequence.

In some embodiments, the computing device, executing the machine learning model, generates 260 an output associated with a sequence of an engineered guide RNA. The output, in some embodiments, is a predicted score of the sequence that predicts the editing performance of an editing system using the engineered guide RNA. In some embodiments, the score is a specificity score such as a ratio of on-target editing to off-target editing. In some embodiments, the specificity score is determined as the target edit percentage divided by the sum of all nonsynonymous off-target edits. In some embodiments, a specificity score is determined as the (sum of on-target editing of the desired nucleotide)/(sum of off-target editing). In some embodiments, a specificity score is determined as 1−(# of reads with only on-target edits)−(# of reads with zero edits). In some embodiments, the score also includes another metric that measures the performance of the engineered guide RNA, such as the throughput. In some embodiments, the output also includes a candidate sequence of the engineered guide RNA. In some embodiments, the sequence of the engineered guide RNA is a portion of the engineered guide RNA or the entirety of the engineered guide RNA. For example, in one embodiment, the output sequence is only the sequence of the ADAR recruiting domain. In some embodiments, the output sequence also includes the sequence of the RNA targeting domain 134. In some embodiments, the output sequence is a modification of a base sequence at one or more specific sites. In some embodiments, the output sequence is selected from multiple sequence candidates. For instance, in an example embodiment, a scientist pre-determines a list of potential sequence candidates that are likely to perform well in the RNA level editing of the mutated mRNA. The machine learning model produces an output that selects one of the sequence candidates that is predicted to provide the best performance. In some embodiments, instead of having a selection of candidates, the machine learning model outputs a new sequence that is predicted to perform well in RNA level editing. Training, structure, and detailed implementation of various examples of machine learning models are further discussed above, and in FIG. 3 through FIG. 4. In some embodiments, training data is from in vitro cell-free experiments, in vitro cell experiments, or in vivo experiments. In some embodiments, one or more cell types are used in the in vitro experiments and subsequently used to train the model.

In some embodiments, the efficacy of the output sequence of the engineered guide RNA 130 or engineered guide RNA of FIG. 23B is validated 270. In some embodiments, the validation is carried in silico through one or more cross-validation machine learning processes. Additionally or alternatively, in some embodiments, the validation is conducted in a wet laboratory. For example, in some implementations, the recruiting throughput, on-target activity, and specificity (e.g., a ratio of on-target editing to off-target editing) of the RNA level sequence editing system using the output sequence of the engineered guide RNA 130 or engineered guide RNA of FIG. 23B, an ADAR, and a target mRNA with the mutation(s) is studied in vitro to confirm the prediction of performance by the machine learning model. In some implementations, additional in vivo studies using biological entities are also conducted.

In some embodiments, the engineered guide RNAs 130 or engineered guide RNAs of FIG. 23B are manufactured. For example, the vectors (biological vectors instead of mathematical vectors) encoding the output sequence of the engineered guide RNA 130 or engineered guide RNA of FIG. 23B are generated 280. In some implementations, the vectors are administered to the subject to treat 290 the genetic disease based on a clinically approved dosage. Alternatively, or additionally, in some embodiments, the engineered guide RNAs 130 or engineered guide RNAs of FIG. 23B are administered directly to the subject to treat the genetic disease. Detail of some example vectors, techniques for manufacturing those vectors, and example treatment processes are discussed below.

### Computer Machine Architecture

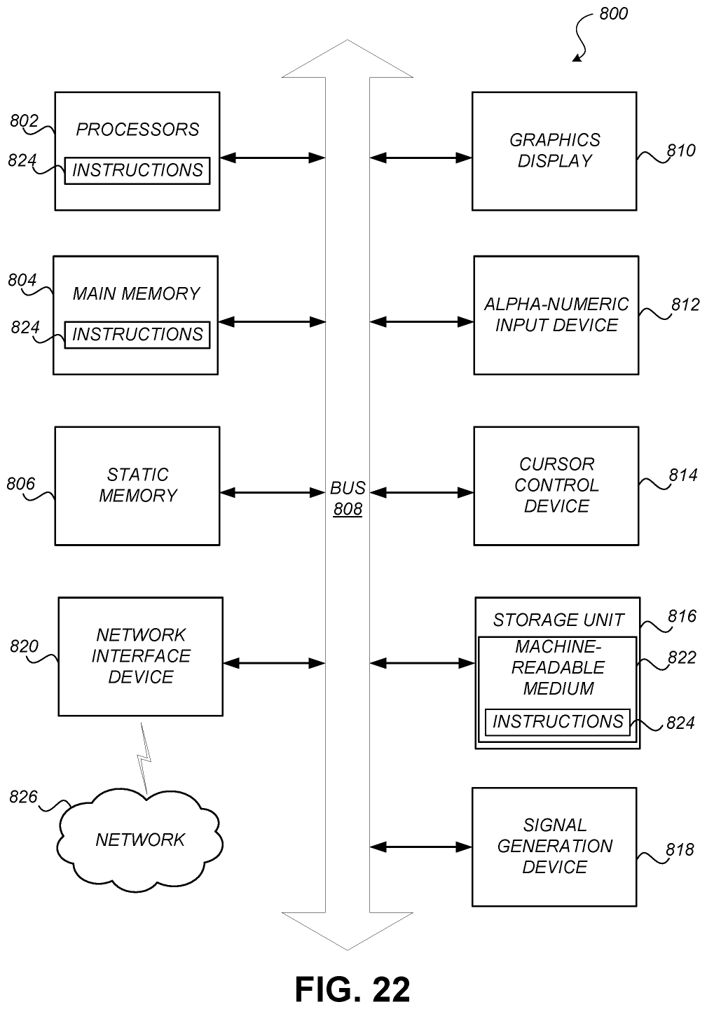

FIG. 22 is a block diagram illustrating components of an example computing machine that is capable of reading instructions from a computer-readable medium and execute them in a processor (or controller). A computer described herein, in some embodiments, includes a single computing machine shown in FIG. 22, a virtual machine, a distributed computing system that includes multiples nodes of computing machines shown in FIG. 22, or any other suitable arrangement of computing devices.

By way of example, FIG. 22 shows a diagrammatic representation of a computing machine in the example form of a computer system 800 within which instructions 824 (e.g., software, source code, program code, expanded code, object code, assembly code, or machine code), which are stored in a computer-readable medium for causing the machine to perform any one or more of the processes discussed herein are executed. In some embodiments, the computing machine operates as a standalone device or is connected (e.g., networked) to other machines. In a networked deployment, in some implementations, the machine operates in the capacity of a server machine or a client machine in a server-client network environment, or as a peer machine in a peer-to-peer (or distributed) network environment.

The structure of a computing machine described in FIG. 22, in some embodiments, corresponds to any software, hardware, or combined components of a computing device that analyzes various genetic sequences and runs one or more machine learning models described herein. While FIG. 22 shows various hardware and software elements, an example computing device, in some embodiments, includes additional or fewer elements.

By way of example, a computing machine, in some embodiments, is a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 824 that specify actions to be taken by that machine. Further, while only a single machine is illustrated, in some embodiments, the term “machine” and “computer” will also be taken to include any collection of machines that individually or jointly execute instructions 824 to perform any one or more of the methodologies discussed herein.

The example computer system 800 includes one or more processors 802 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 800, in some embodiments, include a memory 804 that store computer code including instructions 824 that cause the processors 802 to perform certain actions when the instructions are executed, directly or indirectly by the processors 802. In some embodiments, instructions include any directions, commands, or orders that may be stored in different forms, such as equipment-readable instructions, programming instructions including source code, and other communication signals and orders. In some embodiments, instructions are used in a general sense and are not limited to machine-readable codes. One or more steps in various processes described, in some embodiments, are performed by passing through instructions to one or more multiply-accumulate (MAC) units of the processors.

In some implementations, one or more methods described herein improve the operation speed of the processors 802 and/or reduce the space required for the memory 804. For example, in some embodiments, the database processing techniques and machine learning methods described herein reduce the complexity of the computation of the processors 802 by applying one or more novel techniques that simplify the steps in training, reaching convergence, and generating results of the processors 802. In some embodiments, the algorithms described herein also reduce the size of the models and datasets to reduce the storage space requirement for memory 804.

In some embodiments, the performance of certain of the operations are distributed among the more than processors, not only residing within a single machine, but deployed across a number of machines. In some example embodiments, the one or more processors or processor-implemented modules are located in a single geographic location (e.g., within a home environment, an office environment, or a server farm). In other example embodiments, the one or more processors or processor-implemented modules are distributed across a number of geographic locations. In some instances where the present disclosure refers to processes performed by a processor, this will also be construed to include a joint operation of multiple distributed processors.

In some embodiments, the computer system 800 includes a main memory 804, and a static memory 806, which are configured to communicate with each other via a bus 808. The computer system 800, in some embodiments, further includes a graphics display unit 810 (e.g., a plasma display panel (PDP), a liquid crystal display (LCD), a projector, or a cathode ray tube (CRT)). The graphics display unit 810, controlled by the processors 802, displays a graphical user interface (GUI) to display one or more results and data generated by the processes described herein. The computer system 800, in some embodiments, also includes alphanumeric input device 812 (e.g., a keyboard), a cursor control device 814 (e.g., a mouse, a trackball, a joystick, a motion sensor, or other pointing instrument), a storage unit 816 (a hard drive, a solid state drive, a hybrid drive, a memory disk, etc.), a signal generation device 818 (e.g., a speaker), and a network interface device 820, which also are configured to communicate via the bus 808.

In some implementations, the storage unit 816 includes a computer-readable medium 822 on which is stored instructions 824 embodying any one or more of the methodologies or functions described herein. The instructions 824, in some embodiments, also reside, completely or at least partially, within the main memory 804 or within the processor 802 (e.g., within a processor's cache memory) during execution thereof by the computer system 800, the main memory 804 and the processor 802 also constituting computer-readable media. The instructions 824, in some embodiments, are transmitted or received over a network 826 via the network interface device 820.

While computer-readable medium 822 is shown in an example embodiment to be a single medium, the term “computer-readable medium” should be taken to include a single medium or multiple media (e.g., a centralized or distributed database, or associated caches and servers) able to store instructions (e.g., instructions 824). The computer-readable medium, in some embodiments, includes any medium that is capable of storing instructions (e.g., instructions 824) for execution by the processors (e.g., processors 802) and that cause the processors to perform any one or more of the methodologies disclosed herein. The computer-readable medium, in some embodiments, includes, but is not limited to, data repositories in the form of solid-state memories, optical media, and magnetic media. In some implementations, the computer-readable medium does not include a transitory medium such as a propagating signal or a carrier wave.

### EXEMPLARY SYSTEM EMBODIMENTS

### First Aspect.

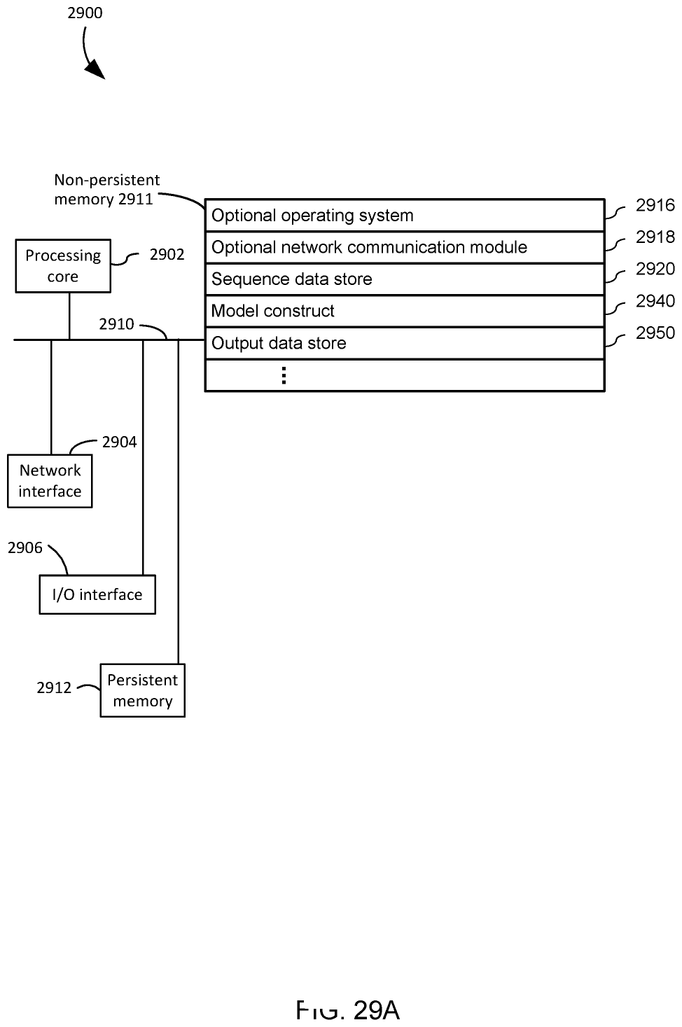

FIGS. 29A-B collectively show a block diagram illustrating a system 2900 for predicting a deamination efficiency (also referred to herein as edit efficiency, editing efficiency, or on-target editing efficiency or score, which can be used interchangeably) or specificity, in accordance with some implementations. The device 2900 in some implementations includes one or more central processing units (CPU(s)) 2902 (also referred to as processors), one or more network interfaces 2904, a user interface 2906, a non-persistent memory 2911, a persistent memory 2912, and one or more communication buses 2910 for interconnecting these components. The one or more communication buses 2910 optionally include circuitry (sometimes called a chipset) that interconnects and controls communications between system components. The non-persistent memory 2911 typically includes high-speed random access memory, such as DRAM, SRAM, DDR RAM, ROM, EEPROM, flash memory, whereas the persistent memory 2912 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 2912 optionally includes one or more storage devices remotely located from the CPU(s) 2902. The persistent memory 2912, and the non-volatile memory device(s) within the non-persistent memory 2912, comprises non-transitory computer readable storage medium. In some implementations, the non-persistent memory 2911 or alternatively the non-transitory computer readable storage medium stores the following programs, modules and data structures, or a subset thereof, sometimes in conjunction with the persistent memory 2912:
an optional operating system 2916, which includes procedures for handling various basic system services and for performing hardware dependent tasks;
an optional network communication module (or instructions) 2918 for connecting the system 2900 with other devices, or a communication network;
a sequence data store 2920, optionally comprising, for a guide RNA (gRNA) 2922 (e.g., 2922-1, . . . 2922-G) that hybridizes to a target mRNA, information 2924 (e.g., 2924-1) comprising a nucleic acid sequence 2926 (e.g., 2926-1) for the gRNA;
a model construct 2940, optionally comprising a plurality of parameters 2942 (e.g., 2942-1, . . . 2942-F); and
an output data store 2950, optionally comprising, as output from the model construct 2940, a set of one or more output metrics 2952 (e.g., 2952-1, . . . 2952-H) for a deamination efficiency or specificity by an Adenosine Deaminase Acting on RNA (ADAR) protein of a target nucleotide position in the target mRNA when facilitated by hybridization of the gRNA to the target mRNA.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, data sets, or modules, and thus various subsets of these modules and data, in some embodiments, are combined or otherwise re-arranged in various implementations. In some implementations, the non-persistent memory 2911 optionally stores a subset of the modules and data structures identified above. Furthermore, in some embodiments, the memory stores additional modules and data structures not described above. In some embodiments, one or more of the above identified elements is stored in a computer system, other than that of system 2900, that is addressable by system 2900 so that system 2900, in some embodiments, retrieves all or a portion of such data when needed.

Although FIGS. 29A-B depict a “system 2900,” the figures are intended more as a functional description of the various features which, in some embodiments, are present in computer systems than as a structural schematic of the implementations described herein. In practice, and as recognized by those of ordinary skill in the art, items shown separately could be combined and some items could be separated. Moreover, although FIGS. 29A-B depict certain data and modules in non-persistent memory 2911, some or all of these data and modules, in some embodiments, are in persistent memory 2912.

### Second Aspect.

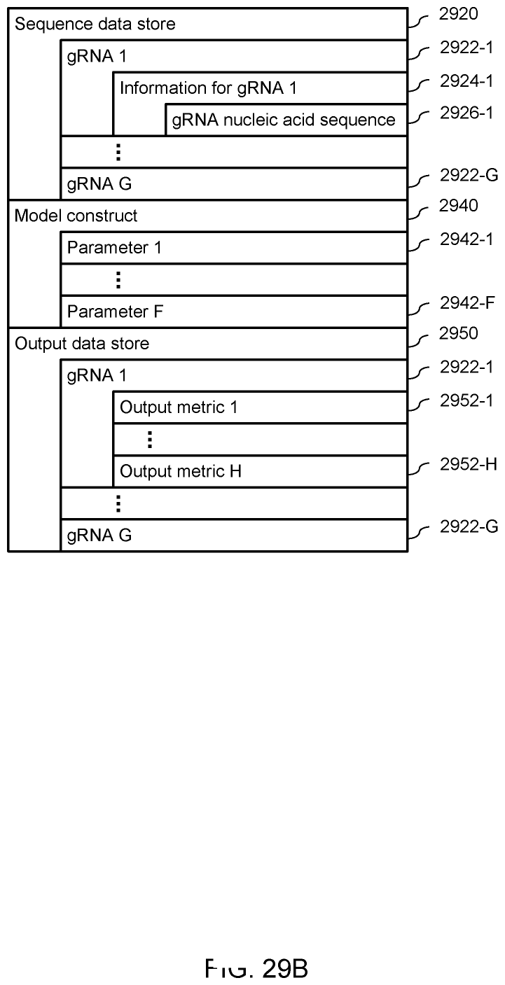

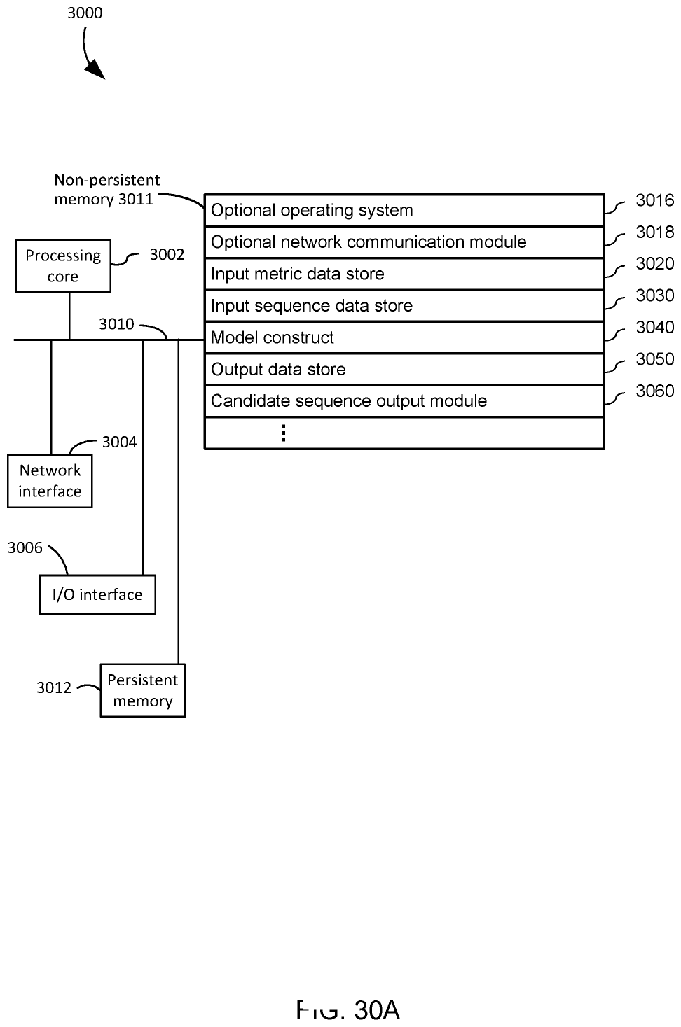

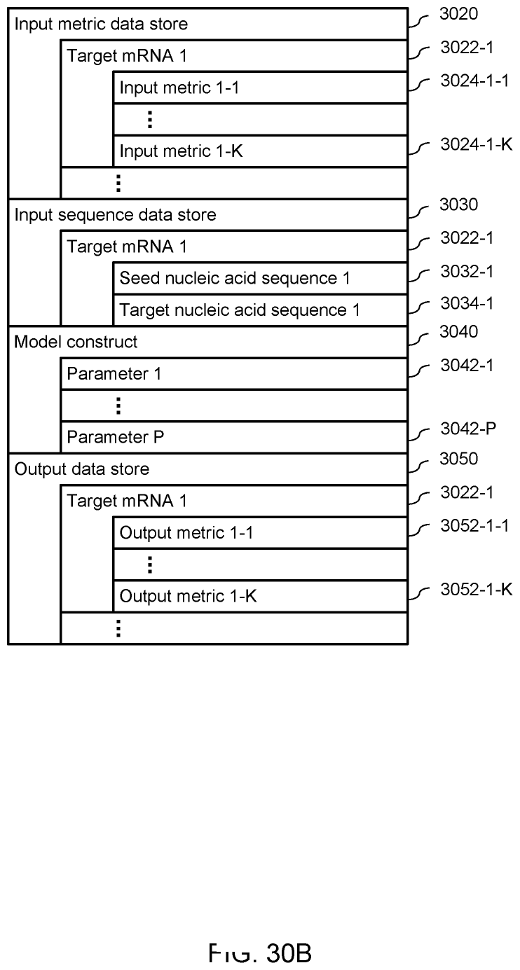

FIGS. 30A-B collectively show a block diagram illustrating a system 3000 for generating a candidate sequence for a guide RNA (gRNA), in accordance with some implementations. The device 3000 in some implementations includes one or more central processing units (CPU(s)) 3002 (also referred to as processors), one or more network interfaces 3004, a user interface 3006, a non-persistent memory 3011, a persistent memory 3012, and one or more communication buses 3010 for interconnecting these components. The one or more communication buses 3010 optionally include circuitry (sometimes called a chipset) that interconnects and controls communications between system components. The non-persistent memory 3011 typically includes high-speed random access memory, such as DRAM, SRAM, DDR RAM, ROM, EEPROM, flash memory, whereas the persistent memory 3012 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 3012 optionally includes one or more storage devices remotely located from the CPU(s) 3002. The persistent memory 3012, and the non-volatile memory device(s) within the non-persistent memory 3012, comprises non-transitory computer readable storage medium. In some implementations, the non-persistent memory 3011 or alternatively the non-transitory computer readable storage medium stores the following programs, modules and data structures, or a subset thereof, sometimes in conjunction with the persistent memory 3012:
an optional operating system 3016, which includes procedures for handling various basic system services and for performing hardware dependent tasks;
an optional network communication module (or instructions) 3018 for connecting the system 3000 with other devices, or a communication network;
an input metric data store 3020, optionally comprising, for a target mRNA 3022, information comprising a desired set of one or more input metrics 3024 (e.g., 3024-1-1, . . . 3024-1-K) for an efficiency or specificity of deamination of a target nucleotide position in the target mRNA by an Adenosine Deaminase Acting on RNA (ADAR) protein when facilitated by hybridization of a gRNA to the target mRNA;
an input sequence data store 3030, optionally comprising, for the target mRNA 3022, (i) a seed nucleic acid sequence for the gRNA 3032 (e.g., 3032-1) and (ii) a target nucleic acid sequence for the target mRNA 3034 (e.g., 3034-1);
a model construct 3040, optionally comprising a plurality of parameters 3042 (e.g., 3042-1, . . . 3042-P);
an output data store 3050, optionally comprising, as output from the model construct 3040, for the target mRNA 3022, a calculated set of one or more output metrics 3052 (e.g., 3052-1-1, . . . 3052-1-K) for the efficiency or specificity of deamination of the target nucleotide position in the target mRNA by the ADAR protein; and
a candidate sequence output module 3060, that optionally generates a candidate gRNA sequence by iteratively updating the seed nucleic acid sequence 3032, while holding the plurality of parameters 3042 and the target nucleic acid sequence 3034 fixed, to reduce a difference between (i) the desired set of the one or more metrics 3024 and (ii) the calculated set of the one or metrics 3052.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, data sets, or modules, and thus various subsets of these modules and data, in some embodiments, are combined or otherwise re-arranged in various implementations. In some implementations, the non-persistent memory 3011 optionally stores a subset of the modules and data structures identified above. Furthermore, in some embodiments, the memory stores additional modules and data structures not described above. In some embodiments, one or more of the above identified elements is stored in a computer system, other than that of system 3000, that is addressable by system 3000 so that system 3000, in some embodiments, retrieves all or a portion of such data when needed.

Although FIGS. 30A-B depict a “system 3000,” the figures are intended more as a functional description of the various features which, in some embodiments, are present in computer systems than as a structural schematic of the implementations described herein. In practice, and as recognized by those of ordinary skill in the art, items shown separately could be combined and some items could be separated. Moreover, although FIGS. 30A-B depict certain data and modules in non-persistent memory 3011, some or all of these data and modules, in some embodiments, are in persistent memory 3012.

### SPECIFIC EMBODIMENTS

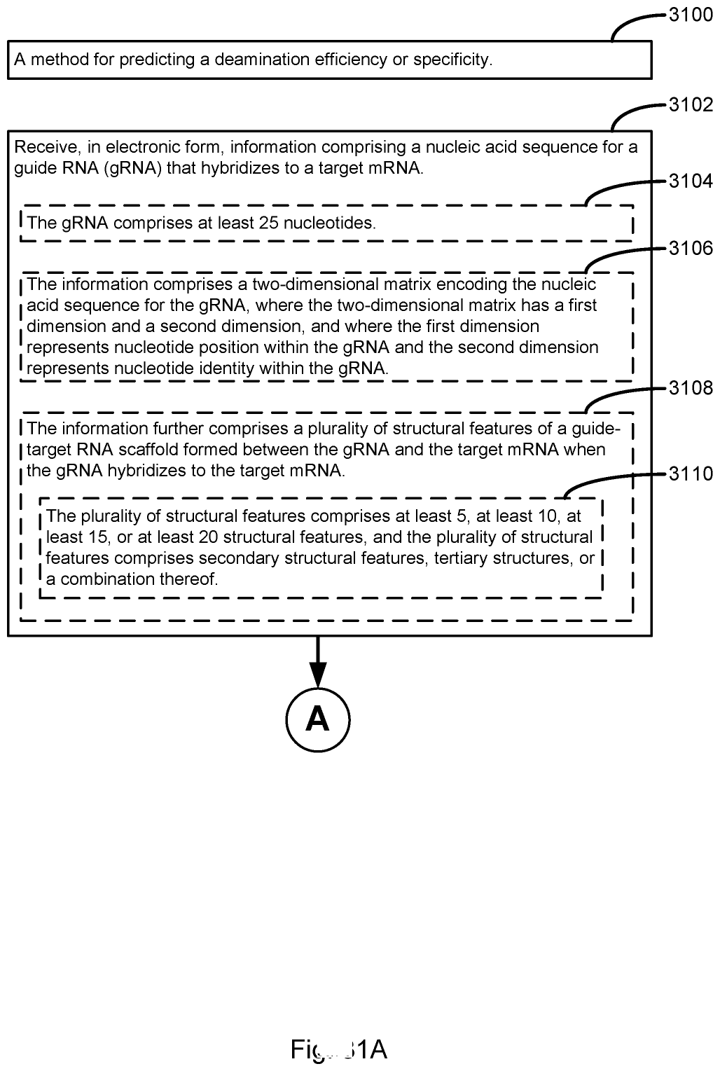

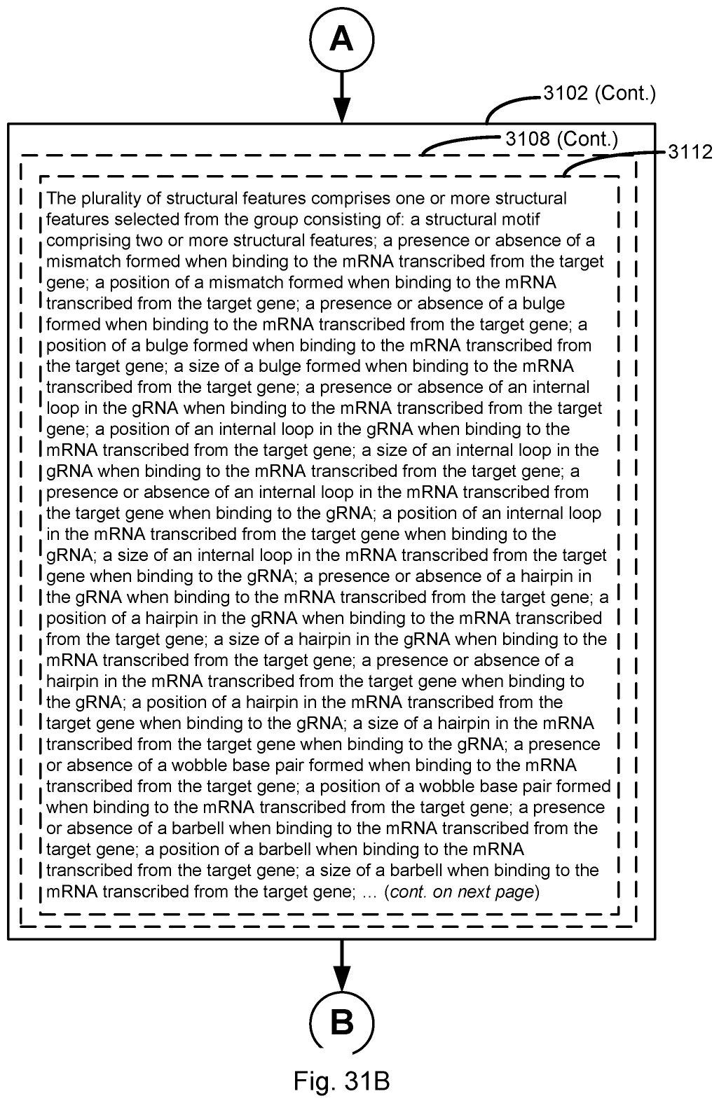

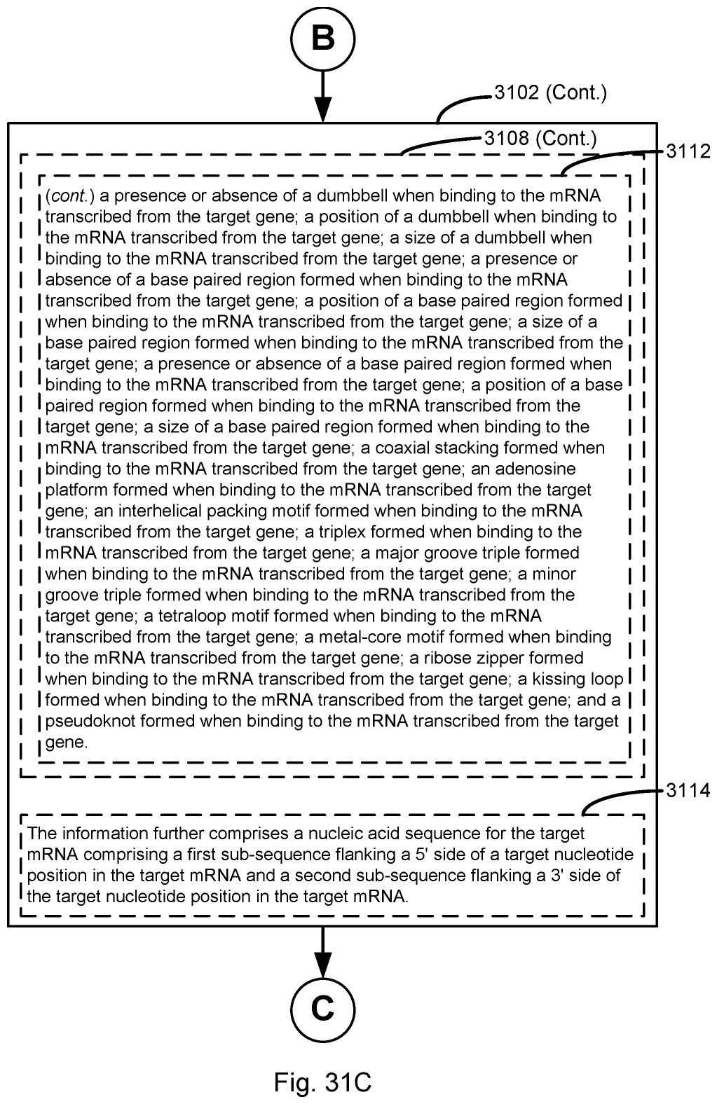

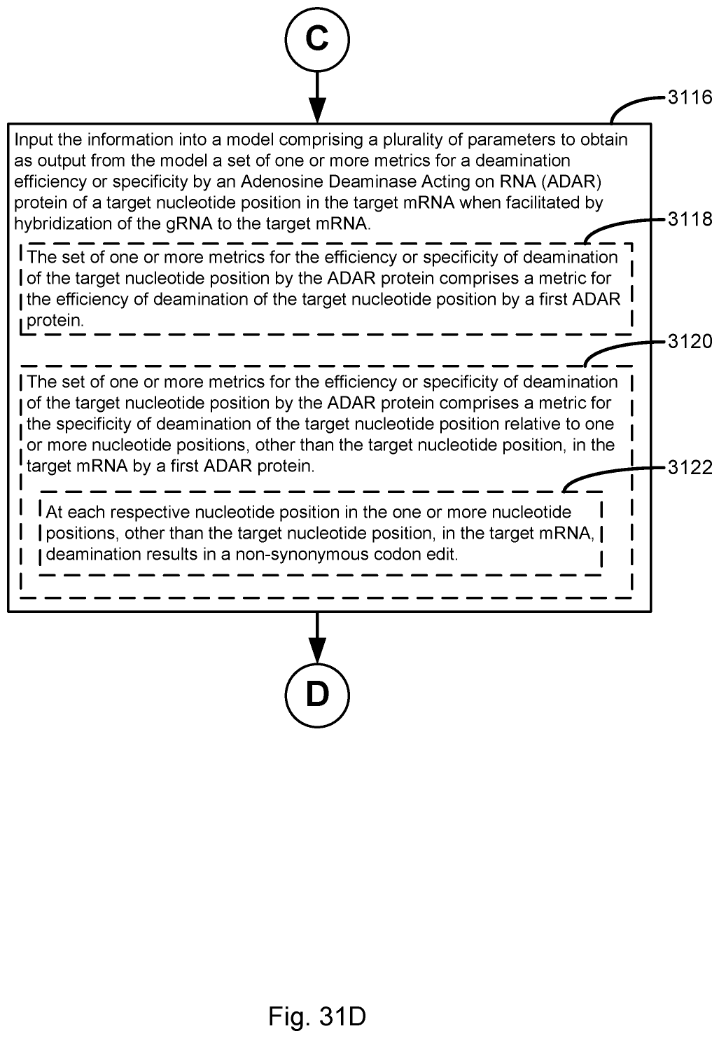

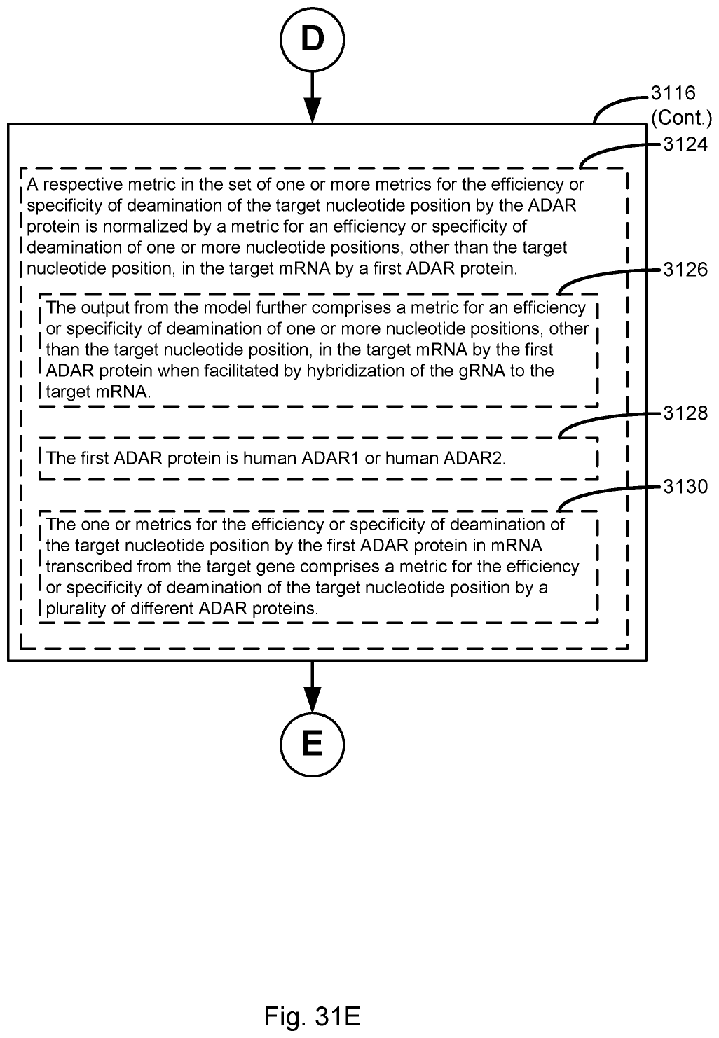

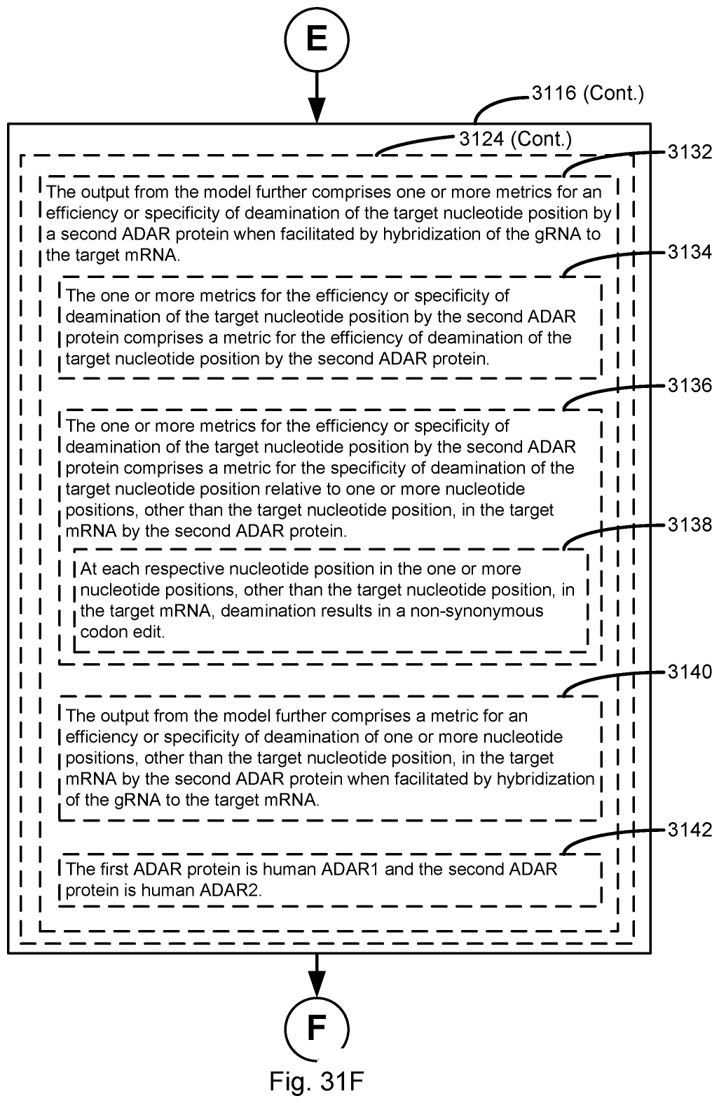

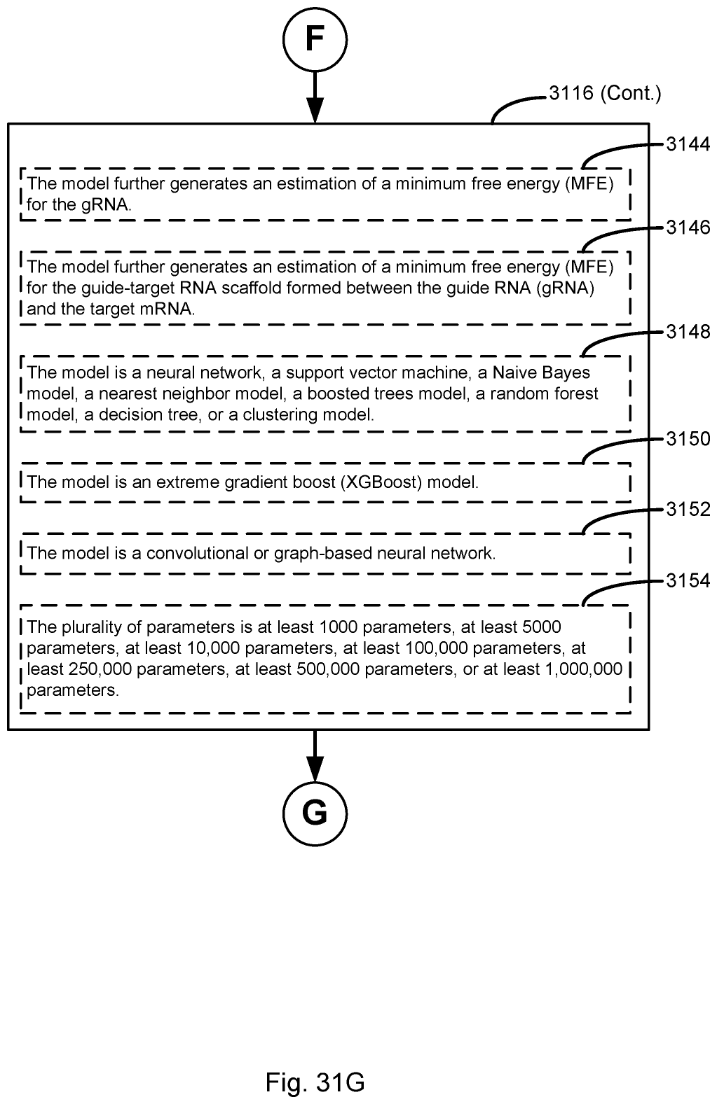

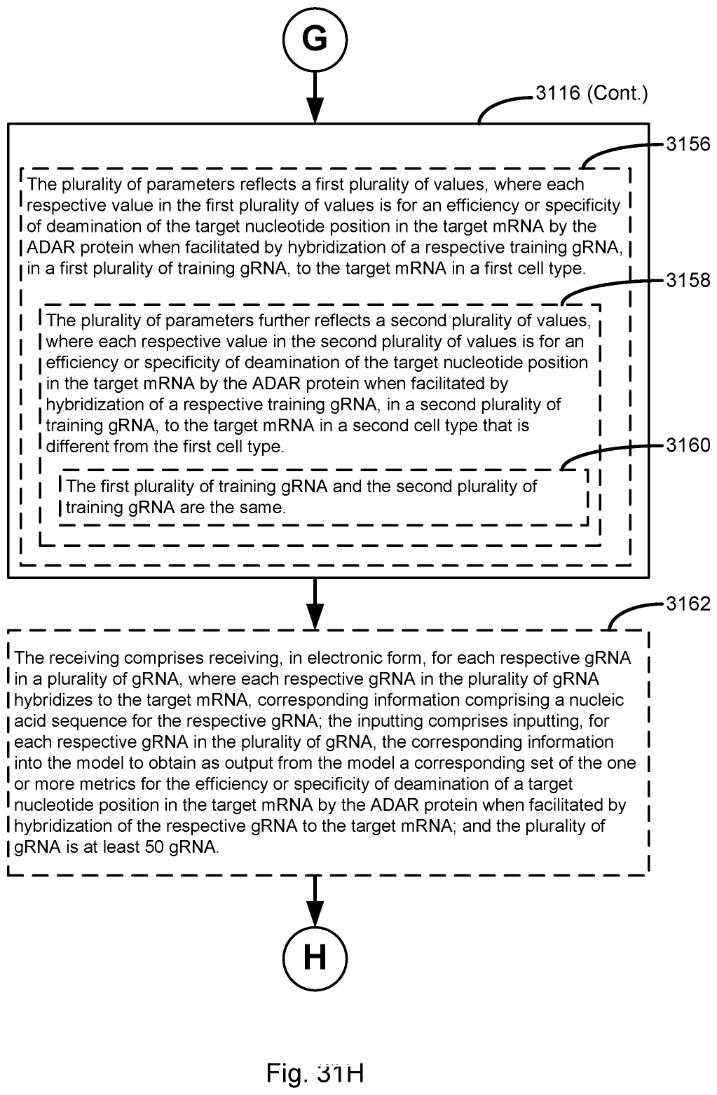

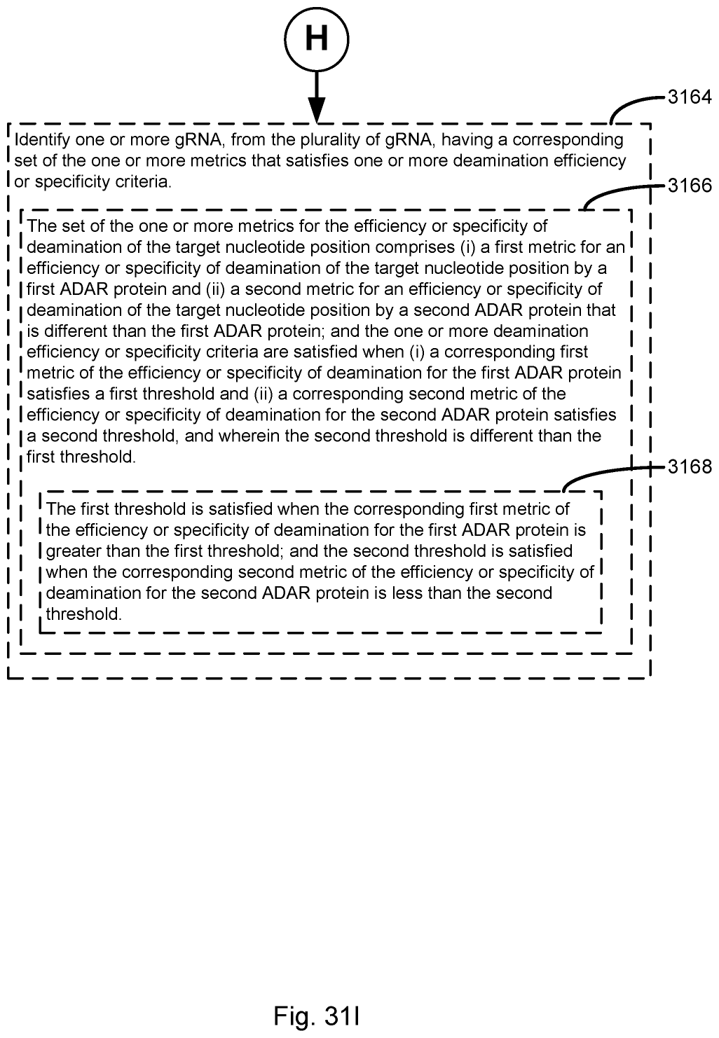

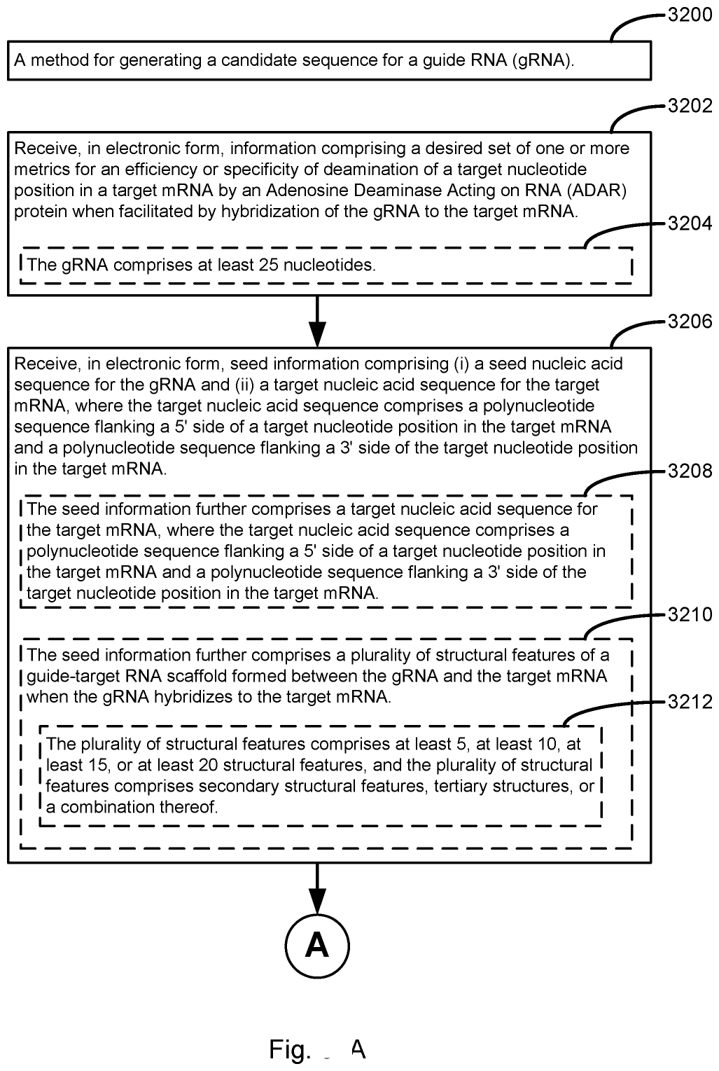

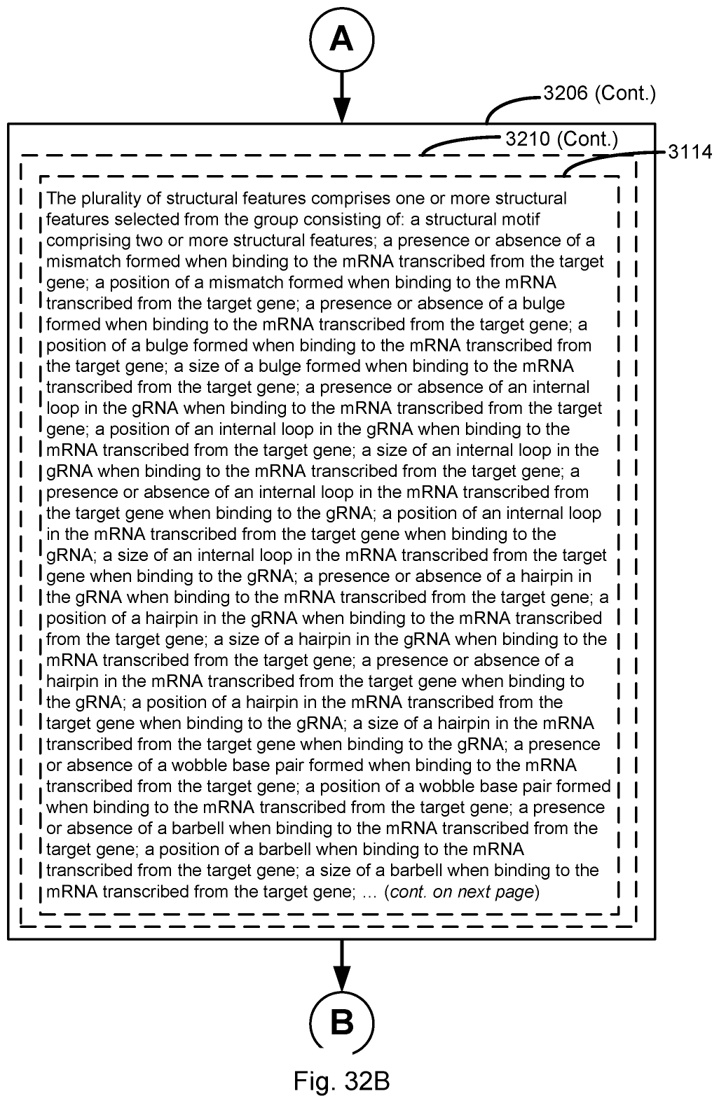

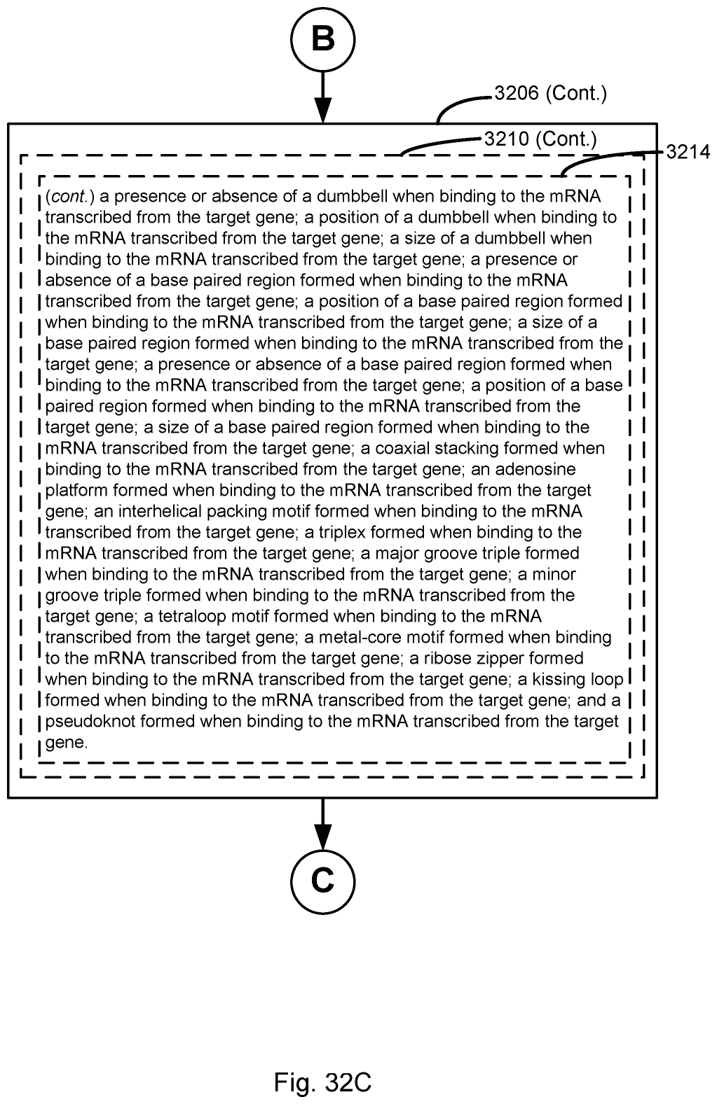

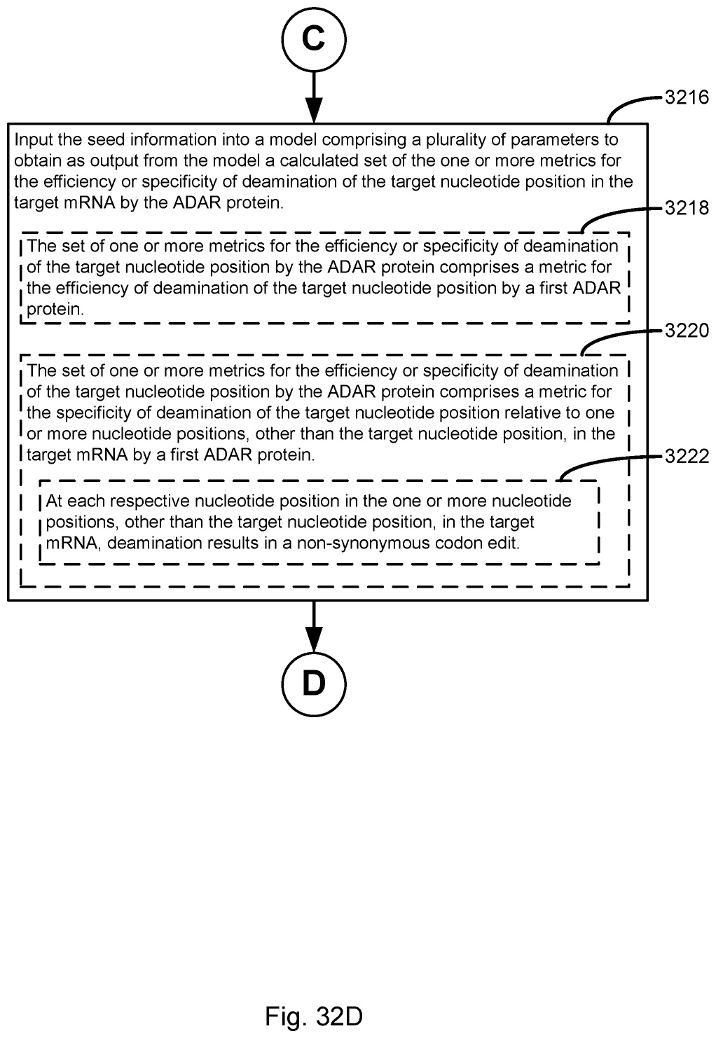

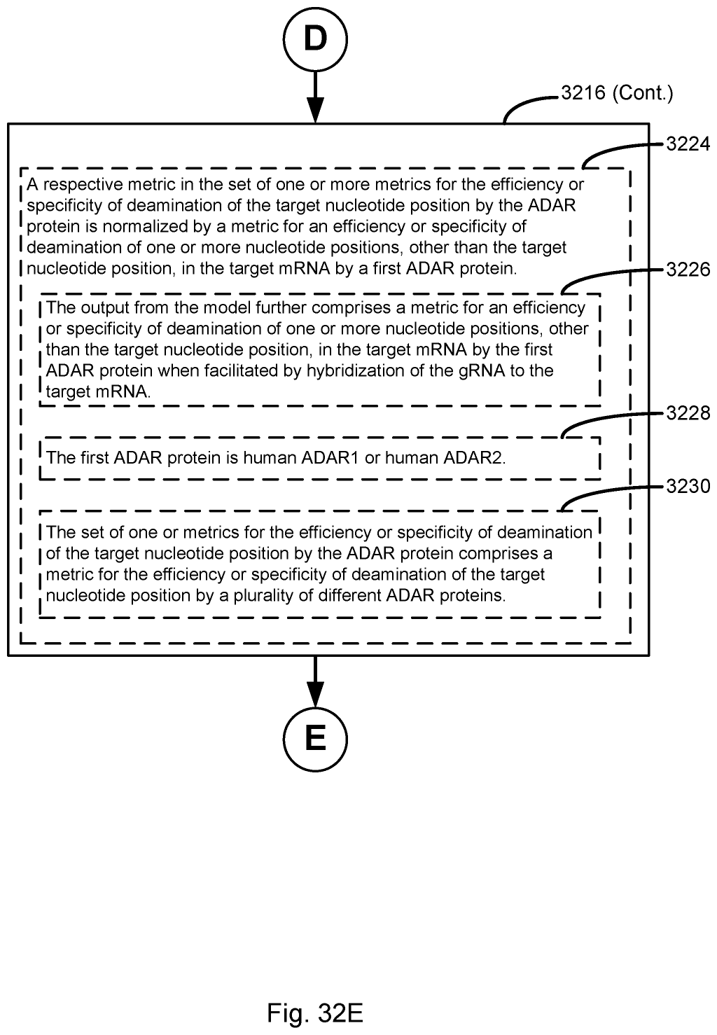

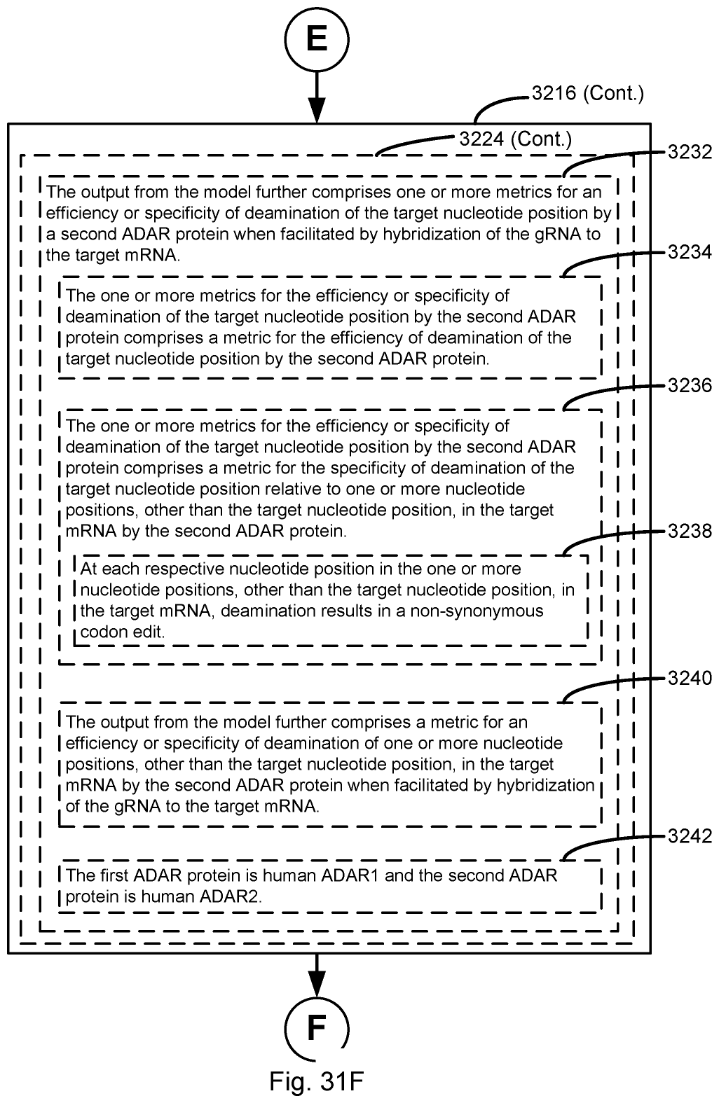

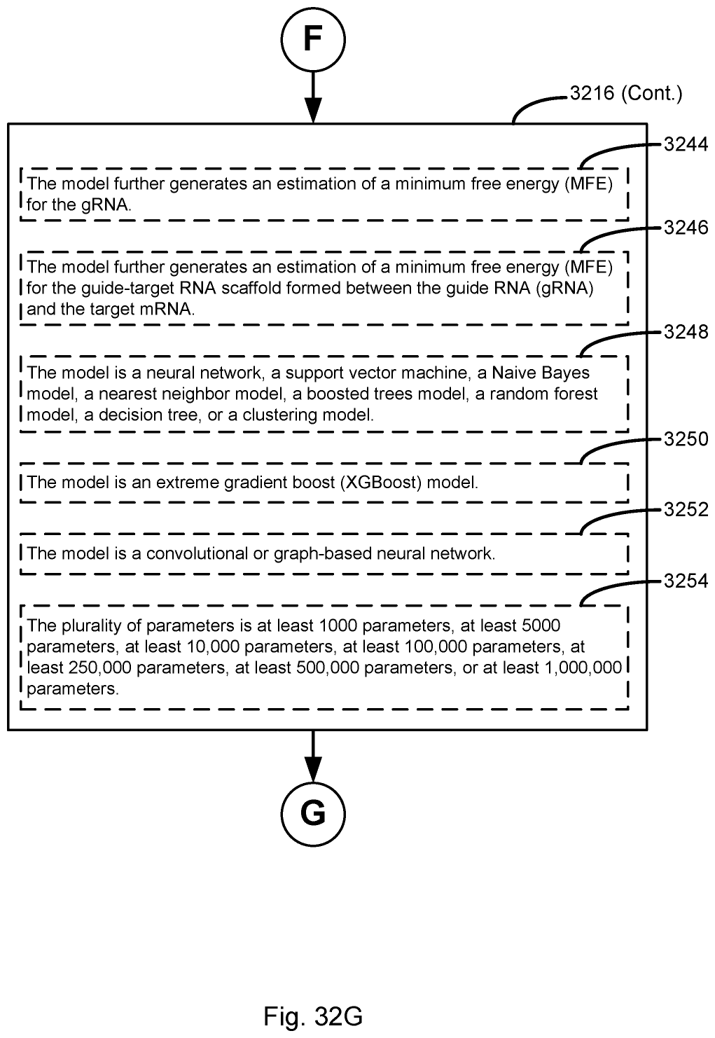

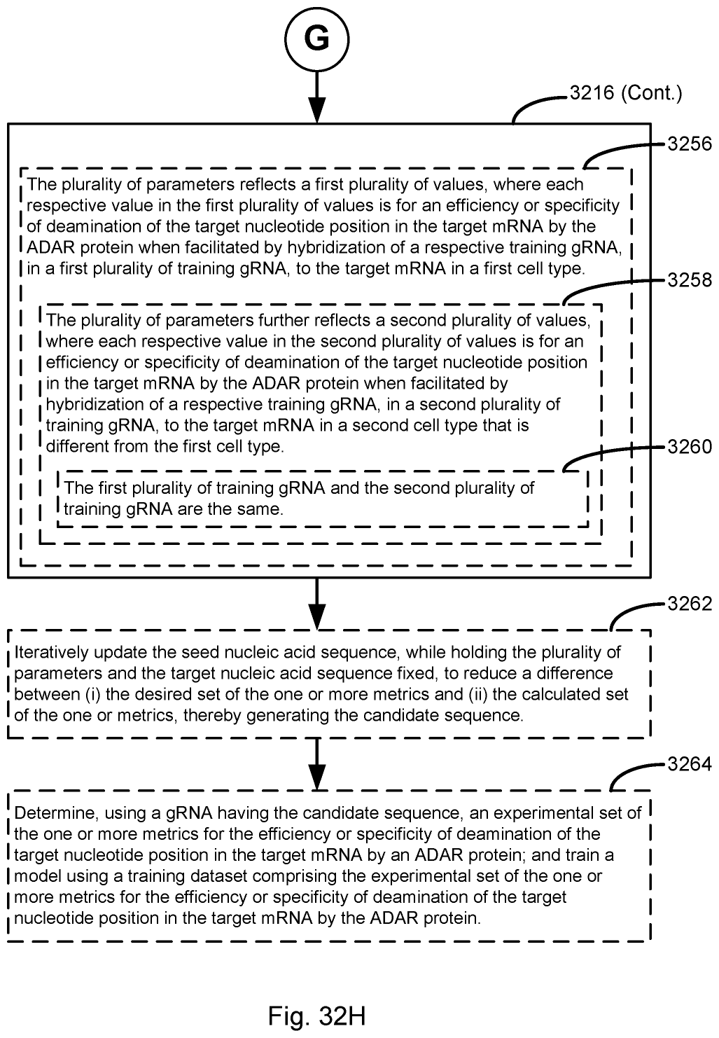

While systems in accordance with the present disclosure have been disclosed with reference to FIGS. 29A-B and 30A-B, methods in accordance with the present disclosure are now detailed with reference to FIGS. 31A-I and 32A-H.

### First Aspect.

One aspect of the present disclosure provides a method 3100 for predicting a deamination efficiency or specificity. In some embodiments, the method 3100 is performed at a computer system comprising at least one processor and a memory storing at least one program for execution by the at least one processor.

Referring to block 3102, in some embodiments, the method includes receiving, in electronic form, information 2924 comprising a nucleic acid sequence 2926 for a guide RNA (gRNA) 2922 that hybridizes to a target mRNA.

Referring to block 3104, in some embodiments, the gRNA comprises at least 25 nucleotides.

In some embodiments, the gRNA comprises at least 5, at least 10, at least 15, at least 20, at least 25, at least 30, at least 40, at least 50, at least 80, at least 100, at least 120, at least 150, at least 180, or at least 200 nucleotides. In some embodiments, the gRNA comprises no more than 300, no more than 200, no more than 180, no more than 150, no more than 100, no more than 80, no more than 50, no more than 40, no more than 30, no more than 20, or no more than 10 nucleotides. In some embodiments, the gRNA consists of from 5 to 20, from 10 to 40, from 15 to 30, from 25 to 50, from 50 to 100, from 80 to 200, from 100 to 250, or from 120 to 300 nucleotides. In some embodiments, the gRNA falls within another range starting no lower than 5 nucleotides and ending no higher than 300 nucleotides.

Referring to block 3106, in some embodiments, the information comprises a two-dimensional matrix encoding the nucleic acid sequence for the gRNA, where the two-dimensional matrix has a first dimension and a second dimension, and where the first dimension represents nucleotide position within the gRNA and the second dimension represents nucleotide identity within the gRNA.

Referring to block 3108, in some embodiments, the information further comprises a plurality of structural features of a guide-target RNA scaffold formed between the gRNA and the target mRNA when the gRNA hybridizes to the target mRNA.

Referring to block 3110, in some embodiments, the plurality of structural features comprises at least 5, at least 10, at least 15, or at least 20 structural features, and the plurality of structural features comprises secondary structural features, tertiary structures, or a combination thereof.

In some embodiments, the plurality of structural features comprises at least 3, at least 5, at least 10, at least 15, at least 20, at least 30, at least 40, at least 50, at least 80, at least 100, or at least 200 structural features. In some embodiments, the plurality of structural features comprises no more than 500, no more than 200, no more than 100, no more than 50, no more than 30, no more than 20, or no more than 10 structural features. In some embodiments, the plurality of structural features consists of from 3 to 20, from 5 to 50, from 20 to 100, from 15 to 80, from 50 to 200, or from 100 to 500 structural features. In some embodiments, the plurality of structural features falls within another range starting no lower than 3 structural features and ending no higher than 500 structural features.

In some embodiments, the plurality of structural features comprises one or more structural features selected from the group consisting of: a structural motif comprising two or more structural features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding to the mRNA transcribed from the target gene; a position of an internal loop in the gRNA upon binding to the mRNA transcribed from the target gene; a size of an internal loop in the gRNA upon binding to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding to the mRNA transcribed from the target gene; a position of a hairpin in the gRNA upon binding to the mRNA transcribed from the target gene; a size of a hairpin in the gRNA upon binding to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a presence or absence of a U-deletion formed upon binding of the gRNA to the mRNA transcribed from the target gene; a position of a U-deletion formed upon binding of the gRNA to the mRNA transcribed from the target gene; a size of U-deletion formed upon binding of the gRNA to the mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features comprises a disruption to a micro-footprint in a guide-target RNA scaffold formed between the gRNA and the target mRNA when the gRNA hybridizes to the target mRNA. In some embodiments, the plurality of structural features comprises a disruption to a macro-footprint in a guide-target RNA scaffold formed between the gRNA and the target mRNA when the gRNA hybridizes to the target mRNA. In some embodiments, the plurality of structural features comprises a disruption to a barbell in a guide-target RNA scaffold formed between the gRNA and the target mRNA when the gRNA hybridizes to the target mRNA. In some embodiments, the plurality of structural features comprises a disruption to a macro-footprint, other than a barbell, in a guide-target RNA scaffold formed between the gRNA and the target mRNA when the gRNA hybridizes to the target mRNA.

In some embodiments, the plurality of structural features further comprises a U-deletion formed upon binding of the gRNA to the mRNA transcribed from the target gene.

In some embodiments, the plurality of structural features further comprises indications of a plurality of tertiary structures of the gRNA (e.g., in the guide-target RNA scaffold). In some embodiments, the plurality of tertiary structures comprises indications for at least five types of tertiary structures of the gRNA.

In some embodiments, the plurality of tertiary structures comprises indications for at least 1, at least 2, at least 3, at least 4, at least 5, at least 6, at least 7, at least 8, at least 9, at least 10, at least 15, at least 20, at least 25, at least 30, at least 40, at least 50, at least 60, at least 80, or at least 100 types of tertiary structures of the gRNA (e.g., in the guide-target RNA scaffold). In some embodiments, the plurality of tertiary structures comprises indications for no more than 100, indications for no more than 80, indications for no more than 60, indications for no more than 50, indications for no more than 40, no more than 25, no more than 15, no more than 10, or no more than 5 types of tertiary structures of the gRNA. In some embodiments, the plurality tertiary structures comprises indications for from 1 to 5, from 4 to 10, from 5 to 20, from 10 to 40, from 2 to 100, from 2 to 50, from 1 to 100, from 5 to 100, or from 10 to 100 types of tertiary structures of the gRNA. In some embodiments, the plurality of tertiary structures comprises indications that fall within another range starting no lower than 1 and ending no higher than 100 types of tertiary structures of the gRNA.

In some embodiments, the plurality of tertiary structures comprises one or more tertiary structures selected from the group consisting of a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

Referring to block 3116, in some embodiments, the information further comprises a nucleic acid sequence for the target mRNA comprising a first sub-sequence flanking a 5′ side of a target nucleotide position in the target mRNA and a second sub-sequence flanking a 3′ side of the target nucleotide position in the target mRNA. In some embodiments, the first sub-sequence flanking a 5′ side of a target nucleotide position in the target mRNA is no more than 150 nucleotides. In some embodiments, the first sub-sequence flanking a 5′ side of a target nucleotide position in the target mRNA is at least 5, at least 10, at least 15, at least 20, at least 25, at least 30, at least 40, at least 50, at least 80, at least 100, at least 120, at least 150, at least 180, or at least 200 nucleotides. In some embodiments, the first sub-sequence flanking a 5′ side of a target nucleotide position in the target mRNA is no more than 300, no more than 200, no more than 180, no more than 150, no more than 100, no more than 80, no more than 50, no more than 40, no more than 30, no more than 20, or no more than 10 nucleotides. In some embodiments, the first sub-sequence flanking a 5′ side of a target nucleotide position in the target mRNA consists of from 5 to 20, from 10 to 40, from 15 to 30, from 25 to 50, from 50 to 100, from 80 to 200, from 100 to 250, or from 120 to 300 nucleotides. In some embodiments, the first sub-sequence flanking a 5′ side of a target nucleotide position in the target mRNA falls within another range starting no lower than 5 nucleotides and ending no higher than 300 nucleotides. In some embodiments, the second sub-sequence flanking a 3′ side of a target nucleotide position in the target mRNA is no more than 150 nucleotides. In some embodiments, the second sub-sequence flanking a 3′ side of a target nucleotide position in the target mRNA is at least 5, at least 10, at least 15, at least 20, at least 25, at least 30, at least 40, at least 50, at least 80, at least 100, at least 120, at least 150, at least 180, or at least 200 nucleotides. In some embodiments, the second sub-sequence flanking a 3′ side of a target nucleotide position in the target mRNA is no more than 300, no more than 200, no more than 180, no more than 150, no more than 100, no more than 80, no more than 50, no more than 40, no more than 30, no more than 20, or no more than 10 nucleotides. In some embodiments, the second sub-sequence flanking a 3′ side of a target nucleotide position in the target mRNA consists of from 5 to 20, from 10 to 40, from 15 to 30, from 25 to 50, from 50 to 100, from 80 to 200, from 100 to 250, or from 120 to 300 nucleotides. In some embodiments, the second sub-sequence flanking a 3′ side of a target nucleotide position in the target mRNA falls within another range starting no lower than 5 nucleotides and ending no higher than 300 nucleotides.

Alternatively or additionally, in some embodiments, the information further comprises a nucleic acid sequence for the target mRNA comprising a first sub-sequence flanking a 5′ side of an off-target nucleotide position in the target mRNA and a second sub-sequence flanking a 3′ side of the off-target nucleotide position in the target mRNA. In some embodiments, the first sub-sequence flanking a 5′ side of an off-target nucleotide position in the target mRNA is no more than 150 nucleotides. In some embodiments, the first sub-sequence flanking a 5′ side of an off-target nucleotide position in the target mRNA is at least 5, at least 10, at least 15, at least 20, at least 25, at least 30, at least 40, at least 50, at least 80, at least 100, at least 120, at least 150, at least 180, or at least 200 nucleotides. In some embodiments, the first sub-sequence flanking a 5′ side of an off-target nucleotide position in the target mRNA is no more than 300, no more than 200, no more than 180, no more than 150, no more than 100, no more than 80, no more than 50, no more than 40, no more than 30, no more than 20, or no more than 10 nucleotides. In some embodiments, the first sub-sequence flanking a 5′ side of an off-target nucleotide position in the target mRNA consists of from 5 to 20, from 10 to 40, from 15 to 30, from 25 to 50, from 50 to 100, from 80 to 200, from 100 to 250, or from 120 to 300 nucleotides. In some embodiments, the first sub-sequence flanking a 5′ side of an off-target nucleotide position in the target mRNA falls within another range starting no lower than 5 nucleotides and ending no higher than 300 nucleotides. In some embodiments, the second sub-sequence flanking a 3′ side of the off-target nucleotide position in the target mRNA is no more than 150 nucleotides. In some embodiments, the second sub-sequence flanking a 3′ side of the off-target nucleotide position in the target mRNA is at least 5, at least 10, at least 15, at least 20, at least 25, at least 30, at least 40, at least 50, at least 80, at least 100, at least 120, at least 150, at least 180, or at least 200 nucleotides. In some embodiments, the second sub-sequence flanking a 3′ side of the off-target nucleotide position in the target mRNA is no more than 300, no more than 200, no more than 180, no more than 150, no more than 100, no more than 80, no more than 50, no more than 40, no more than 30, no more than 20, or no more than 10 nucleotides. In some embodiments, the second sub-sequence flanking a 3′ side of the off-target nucleotide position in the target mRNA consists of from 5 to 20, from 10 to 40, from 15 to 30, from 25 to 50, from 50 to 100, from 80 to 200, from 100 to 250, or from 120 to 300 nucleotides. In some embodiments, the second sub-sequence flanking a 3′ side of the off-target nucleotide position in the target mRNA falls within another range starting no lower than 5 nucleotides and ending no higher than 300 nucleotides.

In some embodiments, the information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding.

As an illustrative example, in some embodiments, the plurality of structural features comprises a set of secondary structural features, each respective secondary structural feature including one or more components selected from the group consisting of: a location of the structural feature relative to the target nucleotide position (e.g., a target adenosine); a dimension of the feature; a name of the secondary structure; and the primary sequence on the gRNA and target mRNA strands. In some embodiments, each respective secondary structural feature in the set of secondary structural features comprises the location of the structural feature relative to the target nucleotide position (e.g., a target adenosine); the dimension of the feature; the name of the secondary structure; and the primary sequence on the gRNA and target mRNA strands. This method of featurization encompasses a large amount of information, such that the plurality of structural features represents a high-dimensional feature space. Without being limited to any one theory of operation, if the coverage of the feature space is too sparse, certain issues can arise when training machine learning models (e.g., overfitting).

Accordingly, in some embodiments, the encoding does not generate, for each respective secondary structural feature in the set of secondary structural features, a corresponding feature vector that includes an encoding of the various components of the respective secondary structural feature (e.g., location, dimension, loop type, and primary sequence). Rather, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Advantageously, in some implementations, encoding dimension and position separately drastically reduces the dimensionality of the feature space, enabling machine learning models to learn the effects of having a certain secondary structure at any given position. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

Referring to block 3116, in some embodiments, the method further includes inputting the information 2924 into a model comprising a plurality of parameters 2942 to obtain as output from the model a set of one or more metrics 2952 for a deamination efficiency or specificity by an Adenosine Deaminase Acting on RNA (ADAR) protein of a target nucleotide position in the target mRNA when facilitated by hybridization of the gRNA 2922 to the target mRNA.

In some embodiments, the set of one or more metrics comprises at least 1 metric, at least 2 metrics, at least 3 metrics, at least 4 metrics, at least 5 metrics, at least 10 metrics, or at least 20 metrics. In some embodiments, the set of one or more metrics comprises no more than 50, no more than 20, no more than 10, no more than 5, or no more than 3 metrics. In some embodiments, the set of one or more metrics consists of from 1 to 5, from 1 to 10, from 3 to 15, from 5 to 20, from 8 to 30, or from 20 to 50 metrics. In some embodiments, the set of one or more metrics falls within another range starting no lower than 1 metric and ending no higher than 50 metrics.

In some embodiments, the set of one or more metrics for the efficiency or specificity of deamination of the target nucleotide position by the ADAR protein is determined using a plurality of instances of the target mRNA, or a plurality of sequence reads obtained therefrom. For example, in some embodiments, the set of one or more metrics for the efficiency or specificity of deamination of the target nucleotide position by the ADAR protein is determined using a plurality of sequence reads obtained from a sequencing of a plurality of target mRNAs.

Referring to block 3118, in some embodiments, the set of one or more metrics for the efficiency or specificity of deamination of the target nucleotide position by the ADAR protein comprises a metric for the efficiency of deamination of the target nucleotide position by a first ADAR protein. In some embodiments, the metric for the efficiency of deamination of the target nucleotide position by a respective ADAR protein is also referred to interchangeably herein as edit efficiency, editing efficiency, or on-target editing efficiency or score.

In some embodiments, the metric for the efficiency of deamination of the target nucleotide position by the first ADAR protein is (i) a prevalence of deamination of the target nucleotide position in a plurality of instances of the target mRNA or (ii) a prevalence of the absence of deamination of any nucleotide position in a respective instance of a target mRNA in a plurality of instances of the target mRNA.

For example, in some embodiments, the prevalence of deamination of the target nucleotide position in a plurality of instances of the target mRNA is determined as the proportion of reads with any on-target edits (e.g., an “any on-target editing” metric). In some embodiments, the prevalence of the absence of deamination of any nucleotide position in a respective instance of a target mRNA in a plurality of instances of the target mRNA is determined as the proportion of reads without any edits (e.g., a “no editing” metric).

Referring to block 3120, in some embodiments, the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

In some embodiments, the metric for the specificity of deamination of the target nucleotide position relative to one or more nucleotide positions, other than the target nucleotide position, in the target mRNA by the first ADAR protein is (i) a comparison of (a) a prevalence of deamination of the target nucleotide position in a plurality of instances of the target mRNA and (b) a prevalence of deamination of at least one nucleotide position, other than the target nucleotide position, in a respective instance of the target mRNA in a plurality of instances of the target mRNA, (ii) a prevalence of deamination of the target nucleotide position, without coincident deamination of one or more nucleotide positions other than the target nucleotide position, in a respective instance of the target mRNA in a plurality of instances of the target mRNA, or (iii) a prevalence of deamination of at least one nucleotide position, other than the target nucleotide position, in a respective instance of the target mRNA in a plurality of instances of the target mRNA.

For example, in some embodiments, the comparison of (a) a prevalence of deamination of the target nucleotide position in a plurality of instances of the target mRNA and (b) a prevalence of deamination of at least one nucleotide position, other than the target nucleotide position, in a respective instance of the target mRNA in a plurality of instances of the target mRNA is determined as (the proportion of reads with on-target edits+1)/(the proportion of reads with off-target edits+1) (e.g., a “specificity” metric). In some embodiments, the prevalence of deamination of the target nucleotide position, without coincident deamination of one or more nucleotide positions other than the target nucleotide position, in a respective instance of the target mRNA in a plurality of instances of the target mRNA is determined as the proportion of reads with only on-target edits (e.g., a “target-only editing” metric). In some embodiments, the prevalence of deamination of at least one nucleotide position, other than the target nucleotide position, in a respective instance of the target mRNA in a plurality of instances of the target mRNA is determined as (1-proportion of reads with any off-target edits) (e.g., a “normalized specificity” metric).

Referring to block 3122, in some embodiments, at each respective nucleotide position in the one or more nucleotide positions, other than the target nucleotide position, in the target mRNA, deamination results in a non-synonymous codon edit. Referring to block 3122, in some embodiments, at the one or more nucleotide positions, other than the target nucleotide position, in the target mRNA, deamination results in a non-synonymous codon edit.

Referring to block 3124, in some embodiments, a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Referring to block 3126, in some embodiments, the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Referring to block 3128, in some embodiments, the first ADAR protein is ADAR1 or ADAR2. Referring to block 3128, in some embodiments, the first ADAR protein is human ADAR1 or human ADAR2.

Referring to block 3130, in some embodiments, the one or metrics for the efficiency or specificity of deamination of the target nucleotide position by the first ADAR protein in mRNA transcribed from the target gene comprises a metric for the efficiency or specificity of deamination of the target nucleotide position by a plurality of different ADAR proteins.

Referring to block 3132, in some embodiments, the output from the model further comprises one or more metrics for an efficiency or specificity of deamination of the target nucleotide position by a second ADAR protein when facilitated by hybridization of the gRNA to the target mRNA.

Referring to block 3134, in some embodiments, the one or more metrics for the efficiency or specificity of deamination of the target nucleotide position by the second ADAR protein comprises a metric for the efficiency of deamination of the target nucleotide position by the second ADAR protein.

In some embodiments, the metric for the efficiency of deamination of the target nucleotide position by the second ADAR protein is (i) a prevalence of deamination of the target nucleotide position in a plurality of instances of the target mRNA or (ii) a prevalence of the absence of deamination of any nucleotide position in a respective instance of a target mRNA in a plurality of instances of the target mRNA.

Referring to block 3136, in some embodiments, the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

In some embodiments, the metric for the specificity of deamination of the target nucleotide position relative to one or more nucleotide positions, other than the target nucleotide position, in the target mRNA by the second ADAR protein is (i) a comparison of (a) a prevalence of deamination of the target nucleotide position in a plurality of instances of the target mRNA and (b) a prevalence of deamination of at least one nucleotide position, other than the target nucleotide position, in a respective instance of the target mRNA in a plurality of instances of the target mRNA, (ii) a prevalence of deamination of the target nucleotide position, without coincident deamination of one or more nucleotide positions other than the target nucleotide position, in a respective instance of the target mRNA in a plurality of instances of the target mRNA, or (iii) a prevalence of deamination of at least one nucleotide position, other than the target nucleotide position, in a respective instance of the target mRNA in a plurality of instances of the target mRNA.

Referring to block 3138, in some embodiments, at each respective nucleotide position in the one or more nucleotide positions, other than the target nucleotide position, in the target mRNA, deamination results in a non-synonymous codon edit. Referring to block 3138, in some embodiments, at the one or more nucleotide positions, other than the target nucleotide position, in the target mRNA, deamination results in a non-synonymous codon edit.

Referring to block 3140, in some embodiments, the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Referring to block 3142, in some embodiments, the first ADAR protein is ADAR1 and the second ADAR protein is ADAR2. Referring to block 3142, in some embodiments, the first ADAR protein is human ADAR1 and the second ADAR protein is human ADAR2.

In some embodiments, the metric for the efficiency of deamination of the target nucleotide position by the first ADAR protein is a comparison of (a) a prevalence of deamination of the target nucleotide position by a first ADAR protein in a plurality of instances of the target mRNA and (b) a prevalence of deamination of the target nucleotide position by a second ADAR protein in the plurality of instances of the target mRNA. For example, in some embodiments, the comparison of (a) a prevalence of deamination of the target nucleotide position by a first ADAR protein in a plurality of instances of the target mRNA and (b) a prevalence of deamination of the target nucleotide position by a second ADAR protein in the plurality of instances of the target mRNA is determined as (target-only editing of the first ADAR protein)−(target-only editing of the second ADAR protein).

In some embodiments, the specificity is determined as the target edit percentage divided by the sum of all nonsynonymous off-target edits. In some embodiments, the specificity is determined as the (sum of on-target editing of the desired nucleotide)/(sum of off-target editing). In some embodiments, the specificity is determined as 1−(# of reads with only on-target edits)−(# of reads with zero edits).

Alternatively or additionally, as described above, in some embodiments, the one or more metrics comprises any on-target editing, specificity, target-only editing, no editing, and/or normalized specificity, for one or more ADAR proteins in a plurality of different ADAR proteins. For instance, in some embodiments, any on-target editing is determined as a proportion of sequence reads with any on-target edits. In some embodiments, specificity is determined as a (proportion of sequence reads with on-target edits+1)/(proportion of sequence reads with off-target edits+1). In some embodiments, target-only editing is determined as a proportion of sequence reads with only on-target edits. In some embodiments, no editing is determined as a proportion of sequence reads without any edits. In some embodiments, normalized specificity is determined as 1−(proportion of sequence reads with any off-target edits). In some embodiments, the one or more metrics further includes a difference in editing preference between a first ADAR protein and a second ADAR protein, in the plurality of different ADAR proteins. In some embodiments, the difference in editing preference is determined as (target-only editing of the first ADAR protein)−(target-only editing of the second ADAR protein). In some embodiments, the one or more metrics are obtained for ADAR1, ADAR2, or ADAR1/2. Alternatively or additionally, in some embodiments, the one or more metrics further includes editability, where editability is a measure of central tendency of the any on-target editing and target-only editing scores. In some embodiments, editability is the average of the any on-target editing and target-only editing scores.

In some embodiments, the output from the model further comprises a determination of a free energy for the gRNA and/or for the guide-target RNA scaffold formed between the guide RNA (gRNA) and the target mRNA.

Referring to block 3144, in some embodiments, the model further generates an estimation of a minimum free energy (MFE) for the gRNA.

Referring to block 3146, in some embodiments, the model further generates an estimation of a minimum free energy (MFE) for the guide-target RNA scaffold formed between the guide RNA (gRNA) and the target mRNA.

Referring to block 3148, in some embodiments, the model is a neural network, a support vector machine, a Naive Bayes model, a nearest neighbor model, a boosted trees model, a random forest model, a decision tree, or a clustering model.

Referring to block 3150, in some embodiments, the model is an extreme gradient boost (XGBoost) model.

Referring to block 3152, in some embodiments, the model is a convolutional or graph-based neural network.

Referring to block 3154, in some embodiments, the plurality of parameters is at least 1000 parameters, at least 5000 parameters, at least 10,000 parameters, at least 100,000 parameters, at least 250,000 parameters, at least 500,000 parameters, or at least 1,000,000 parameters.

In some embodiments, the plurality of parameters comprises at least 200, at least 500, at least 1000, at least 5000, at least 10,000, at least 100,000, at least 250,000, at least 500,000, at least 1,000,000, or at least 5,000,000 parameters. In some embodiments, the plurality of parameters comprises no more than 10,000,000, no more than 5,000,000, no more than 1,000,000, no more than 100,000, no more than 10,000, or no more than 1000 parameters.

In some embodiments, the plurality of parameters consists of from 200 to 10,000, from 1000 to 100,000, from 5000 from 200,000, from 100,000 to 500,000, from 500,000 to 1,000,000, from 1,000,000 to 5,000,000, or from 5,000,000 to 10,000,000 parameters. In some embodiments, the plurality of parameters falls within another range starting no lower than 200 parameters and ending no higher than 10,000,000 parameters.

Referring to block 3156, in some embodiments, the plurality of parameters reflects a first plurality of values, where each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

In some embodiments, the first plurality of values comprises at least 100, at least 1000 values, at least 5000 values, at least 10,000 values, at least 100,000 values, at least 250,000 values, at least 500,000 values, or at least 1,000,000 values. In some embodiments, the first plurality of values comprises no more than 5,000,000, no more than 1,000,000, no more than 100,000, no more than 10,000, or no more than 1000 values. In some embodiments, the first plurality of values consists of from 200 to 10,000, from 1000 to 100,000, from 5000 from 200,000, from 100,000 to 500,000, from 500,000 to 1,000,000, or from 1,000,000 to 5,000,000 values. In some embodiments, the first plurality of values falls within another range starting no lower than 100 values and ending no higher than 5,000,000 values.

In some embodiments, the first plurality of training gRNA comprises at least 1000 gRNAs, at least 5000 gRNAs, at least 10,000 gRNAs, at least 100,000 gRNAs, at least 250,000 gRNAs, at least 500,000 gRNAs, or at least 1,000,000 gRNAs. In some embodiments, the first plurality of training gRNA comprises no more than 5,000,000, no more than 1,000,000, no more than 100,000, no more than 10,000, or no more than 1000 gRNAs. In some embodiments, the first plurality of training gRNA consists of from 200 to 10,000, from 1000 to 100,000, from 5000 from 200,000, from 100,000 to 500,000, from 500,000 to 1,000,000, or from 1,000,000 to 5,000,000 gRNAs. In some embodiments, the first plurality of training gRNA falls within another range starting no lower than 100 gRNAs and ending no higher than 5,000,000 gRNAs.

Referring to block 3158, in some embodiments, the plurality of parameters further reflects a second plurality of values, where each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

In some embodiments, the second plurality of values comprises at least 100, at least 1000 values, at least 5000 values, at least 10,000 values, at least 100,000 values, at least 250,000 values, at least 500,000 values, or at least 1,000,000 values. In some embodiments, the second plurality of values comprises no more than 5,000,000, no more than 1,000,000, no more than 100,000, no more than 10,000, or no more than 1000 values. In some embodiments, the second plurality of values consists of from 200 to 10,000, from 1000 to 100,000, from 5000 from 200,000, from 100,000 to 500,000, from 500,000 to 1,000,000, or from 1,000,000 to 5,000,000 values. In some embodiments, the second plurality of values falls within another range starting no lower than 100 values and ending no higher than 5,000,000 values.

In some embodiments, the second plurality of training gRNAs comprises at least 1000 gRNAs, at least 5000 gRNAs, at least 10,000 gRNAs, at least 100,000 gRNAs, at least 250,000 gRNAs, at least 500,000 gRNAs, or at least 1,000,000 gRNAs. In some embodiments, the second plurality of training gRNA comprises no more than 5,000,000, no more than 1,000,000, no more than 100,000, no more than 10,000, or no more than 1000 gRNAs. In some embodiments, the second plurality of training gRNA consists of from 200 to 10,000, from 1000 to 100,000, from 5000 from 200,000, from 100,000 to 500,000, from 500,000 to 1,000,000, or from 1,000,000 to 5,000,000 gRNAs. In some embodiments, the second plurality of training gRNA falls within another range starting no lower than 100 gRNAs and ending no higher than 5,000,000 gRNAs.

Referring to block 3160, in some embodiments, the first plurality of training gRNA and the second plurality of training gRNA are the same.

Referring to block 3162, in some embodiments, the receiving comprises receiving, in electronic form, for each respective gRNA in a plurality of gRNA, where each respective gRNA in the plurality of gRNA hybridizes to the target mRNA, corresponding information comprising a nucleic acid sequence for the respective gRNA; the inputting comprises inputting, for each respective gRNA in the plurality of gRNA, the corresponding information into the model to obtain as output from the model a corresponding set of the one or more metrics for the efficiency or specificity of deamination of a target nucleotide position in the target mRNA by the ADAR protein when facilitated by hybridization of the respective gRNA to the target mRNA; and the plurality of gRNA is at least 50 gRNA.

Referring to block 3162, in some embodiments, identify one or more gRNA, from the plurality of gRNA, having a corresponding set of the one or more metrics that satisfies one or more deamination efficiency or specificity criteria.

Referring to block 3164, in some embodiments, the set of the one or more metrics for the efficiency or specificity of deamination of the target nucleotide position comprises (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and where the second threshold is different than the first threshold.

Referring to block 3166, in some embodiments, the first threshold is satisfied when the corresponding first metric of the efficiency or specificity of deamination for the first ADAR protein is greater than the first threshold; and the second threshold is satisfied when the corresponding second metric of the efficiency or specificity of deamination for the second ADAR protein is less than the second threshold.

### Second Aspect.

Another aspect of the present disclosure provides a method 3200 for generating a candidate sequence for a guide RNA (gRNA). In some embodiments, the method 3200 is performed at a computer system comprising at least one processor and a memory storing at least one program for execution by the at least one processor.

Referring to block 3202, in some embodiments, the method includes receiving, in electronic form, information comprising a desired set of one or more metrics 3024 for an efficiency or specificity of deamination of a target nucleotide position in a target mRNA 3022 by an Adenosine Deaminase Acting on RNA (ADAR) protein when facilitated by hybridization of the gRNA to the target mRNA.

Referring to block 3204, in some embodiments, the gRNA comprises at least 25 nucleotides.

In some embodiments, the gRNA comprises at least 5, at least 10, at least 15, at least 20, at least 25, at least 30, at least 40, at least 50, at least 80, at least 100, at least 120, at least 150, at least 180, or at least 200 nucleotides. In some embodiments, the gRNA comprises no more than 300, no more than 200, no more than 180, no more than 150, no more than 100, no more than 80, no more than 50, no more than 40, no more than 30, no more than 20, or no more than 10 nucleotides. In some embodiments, the gRNA consists of from 5 to 20, from 10 to 40, from 15 to 30, from 25 to 50, from 50 to 100, from 80 to 200, from 100 to 250, or from 120 to 300 nucleotides. In some embodiments, the gRNA falls within another range starting no lower than 5 nucleotides and ending no higher than 300 nucleotides.

Referring to block 3206, in some embodiments, the method further includes receiving, in electronic form, seed information comprising (i) a seed nucleic acid sequence for the gRNA 3032 and (ii) a target nucleic acid sequence 3034 for the target mRNA 3022, where the target nucleic acid sequence comprises a polynucleotide sequence flanking a 5′ side of a target nucleotide position in the target mRNA and a polynucleotide sequence flanking a 3′ side of the target nucleotide position in the target mRNA.

Referring to block 3208, in some embodiments, the seed information further comprises a target nucleic acid sequence for the target mRNA, where the target nucleic acid sequence comprises a polynucleotide sequence flanking a 5′ side of a target nucleotide position in the target mRNA and a polynucleotide sequence flanking a 3′ side of the target nucleotide position in the target mRNA. In some embodiments, the first sub-sequence flanking a 5′ side of a target nucleotide position in the target mRNA is no more than 150 nucleotides. In some embodiments, the first sub-sequence flanking a 5′ side of a target nucleotide position in the target mRNA is at least 5, at least 10, at least 15, at least 20, at least 25, at least 30, at least 40, at least 50, at least 80, at least 100, at least 120, at least 150, at least 180, or at least 200 nucleotides. In some embodiments, the first sub-sequence flanking a 5′ side of a target nucleotide position in the target mRNA is no more than 300, no more than 200, no more than 180, no more than 150, no more than 100, no more than 80, no more than 50, no more than 40, no more than 30, no more than 20, or no more than 10 nucleotides. In some embodiments, the first sub-sequence flanking a 5′ side of a target nucleotide position in the target mRNA consists of from 5 to 20, from 10 to 40, from 15 to 30, from 25 to 50, from 50 to 100, from 80 to 200, from 100 to 250, or from 120 to 300 nucleotides. In some embodiments, the first sub-sequence flanking a 5′ side of a target nucleotide position in the target mRNA falls within another range starting no lower than 5 nucleotides and ending no higher than 300 nucleotides. In some embodiments, the second sub-sequence flanking a 3′ side of a target nucleotide position in the target mRNA is no more than 150 nucleotides. In some embodiments, the second sub-sequence flanking a 3′ side of a target nucleotide position in the target mRNA is at least 5, at least 10, at least 15, at least 20, at least 25, at least 30, at least 40, at least 50, at least 80, at least 100, at least 120, at least 150, at least 180, or at least 200 nucleotides. In some embodiments, the second sub-sequence flanking a 3′ side of a target nucleotide position in the target mRNA is no more than 300, no more than 200, no more than 180, no more than 150, no more than 100, no more than 80, no more than 50, no more than 40, no more than 30, no more than 20, or no more than 10 nucleotides. In some embodiments, the second sub-sequence flanking a 3′ side of a target nucleotide position in the target mRNA consists of from 5 to 20, from 10 to 40, from 15 to 30, from 25 to 50, from 50 to 100, from 80 to 200, from 100 to 250, or from 120 to 300 nucleotides. In some embodiments, the second sub-sequence flanking a 3′ side of a target nucleotide position in the target mRNA falls within another range starting no lower than 5 nucleotides and ending no higher than 300 nucleotides.

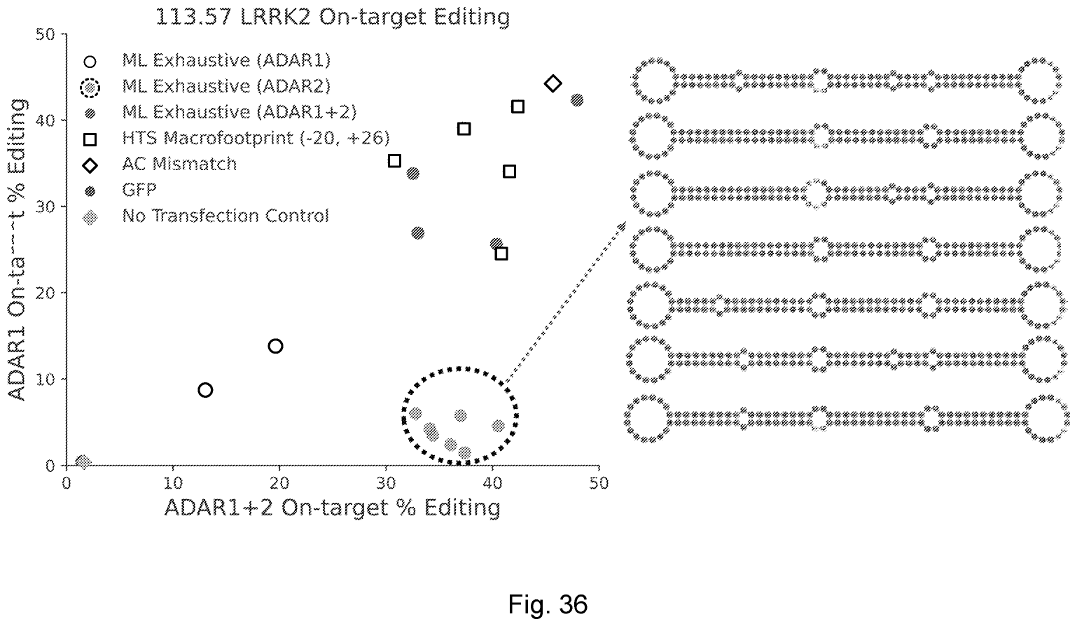

In some embodiments, the seed nucleic acid sequence for the gRNA comprises one or more fixed nucleotide identities. For instance, in some implementations, the one or more fixed nucleotide identities in the seed nucleic acid sequence for the gRNA comprises a guanine that is fixed at the corresponding position, in the seed nucleic acid sequence for the gRNA, opposite from the target nucleotide position in the target mRNA, upon binding of the gRNA to the target mRNA. In other words, in some embodiments, the seed nucleic acid sequence for the gRNA comprises a guanine that is fixed at the position that corresponds to (e.g., is across from) the target nucleotide position in the target mRNA in a guide-target RNA scaffold formed upon binding of the gRNA to the target mRNA. Without being limited by any one theory of operation, enrichment of guanine nucleotides at the position across from the target position have been observed in ADAR guide sequences that facilitate editing by ADAR2 but not ADAR1. For instance, FIG. 36 illustrates A-G mismatches across from the target adenosine in guide-target mRNA scaffolds that drive high ADAR2, but not ADAR1, on-target editing (dashed circle).

Referring to block 3210, in some embodiments, the seed information further comprises a plurality of structural features of a guide-target RNA scaffold formed between the gRNA and the target mRNA when the gRNA hybridizes to the target mRNA.

Referring to block 3212, in some embodiments, the plurality of structural features comprises at least 5, at least 10, at least 15, or at least 20 structural features, and the plurality of structural features comprises secondary structural features, tertiary structures, or a combination thereof.

In some embodiments, the plurality of structural features comprises at least 3, at least 5, at least 10, at least 15, at least 20, at least 30, at least 40, at least 50, at least 80, at least 100, or at least 200 structural features. In some embodiments, the plurality of structural features comprises no more than 500, no more than 200, no more than 100, no more than 50, no more than 30, no more than 20, or no more than 10 structural features. In some embodiments, the plurality of structural features consists of from 3 to 20, from 5 to 50, from 20 to 100, from 15 to 80, from 50 to 200, or from 100 to 500 structural features. In some embodiments, the plurality of structural features falls within another range starting no lower than 3 structural features and ending no higher than 500 structural features.

In some embodiments, the plurality of structural features comprises one or more structural features selected from the group consisting of: a structural motif comprising two or more structural features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding to the mRNA transcribed from the target gene; a position of an internal loop in the gRNA upon binding to the mRNA transcribed from the target gene; a size of an internal loop in the gRNA upon binding to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding to the mRNA transcribed from the target gene; a position of a hairpin in the gRNA upon binding to the mRNA transcribed from the target gene; a size of a hairpin in the gRNA upon binding to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a presence or absence of a U-deletion formed upon binding of the gRNA to the mRNA transcribed from the target gene; a position of a U-deletion formed upon binding of the gRNA to the mRNA transcribed from the target gene; a size of U-deletion formed upon binding of the gRNA to the mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features comprises a disruption to a micro-footprint in a guide-target RNA scaffold formed between the gRNA and the target mRNA when the gRNA hybridizes to the target mRNA. In some embodiments, the plurality of structural features comprises a disruption to a macro-footprint in a guide-target RNA scaffold formed between the gRNA and the target mRNA when the gRNA hybridizes to the target mRNA. In some embodiments, the plurality of structural features comprises a disruption to a barbell in a guide-target RNA scaffold formed between the gRNA and the target mRNA when the gRNA hybridizes to the target mRNA. In some embodiments, the plurality of structural features comprises a disruption to a macro-footprint, other than a barbell, in a guide-target RNA scaffold formed between the gRNA and the target mRNA when the gRNA hybridizes to the target mRNA.

In some embodiments, the plurality of structural features further comprises a U-deletion formed upon binding of the gRNA to the mRNA transcribed from the target gene.

In some embodiments, the plurality of structural features further comprises indications of a plurality of tertiary structures of the gRNA (e.g., in the guide-target RNA scaffold). In some embodiments, the plurality of tertiary structures comprises indications for at least five types of tertiary structures of the gRNA.

In some embodiments, the plurality of tertiary structures comprises indications for at least 1, at least 2, at least 3, at least 4, at least 5, at least 6, at least 7, at least 8, at least 9, at least 10, at least 15, at least 20, at least 25, at least 30, at least 40, at least 50, at least 60, at least 80, or at least 100 types of tertiary structures of the gRNA (e.g., in the guide-target RNA scaffold). In some embodiments, the plurality of tertiary structures comprises indications for no more than 100, indications for no more than 80, indications for no more than 60, indications for no more than 50, indications for no more than 40, no more than 25, no more than 15, no more than 10, or no more than 5 types of tertiary structures of the gRNA. In some embodiments, the plurality tertiary structures comprises indications for from 1 to 5, from 4 to 10, from 5 to 20, from 10 to 40, from 2 to 100, from 2 to 50, from 1 to 100, from 5 to 100, or from 10 to 100 types of tertiary structures of the gRNA. In some embodiments, the plurality of tertiary structures comprises indications that fall within another range starting no lower than 1 and ending no higher than 100 types of tertiary structures of the gRNA.

In some embodiments, the plurality of tertiary structures comprises one or more tertiary structures selected from the group consisting of a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the seed information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding. As described above, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

Referring to block 3216, in some embodiments, the method further includes inputting the seed information (3032, 3034) into a model comprising a plurality of parameters 3042 to obtain as output from the model a calculated set of the one or more metrics 3052 for the efficiency or specificity of deamination of the target nucleotide position in the target mRNA 3022 by the ADAR protein.

In some embodiments, the set of one or more metrics comprises at least 1 metric, at least 2 metrics, at least 3 metrics, at least 4 metrics, at least 5 metrics, at least 10 metrics, or at least 20 metrics. In some embodiments, the set of one or more metrics comprises no more than 50, no more than 20, no more than 10, no more than 5, or no more than 3 metrics. In some embodiments, the set of one or more metrics consists of from 1 to 5, from 1 to 10, from 3 to 15, from 5 to 20, from 8 to 30, or from 20 to 50 metrics. In some embodiments, the set of one or more metrics falls within another range starting no lower than 1 metric and ending no higher than 50 metrics.

In some embodiments, the set of one or more metrics for the efficiency or specificity of deamination of the target nucleotide position by the ADAR protein is determined using a plurality of sequence reads obtained from a plurality of target mRNAs.

Referring to block 3218, in some embodiments, the set of one or more metrics for the efficiency or specificity of deamination of the target nucleotide position by the ADAR protein comprises a metric for the efficiency of deamination of the target nucleotide position by a first ADAR protein.

In some embodiments, the metric for the efficiency of deamination of the target nucleotide position by the first ADAR protein is (i) a prevalence of deamination of the target nucleotide position in a plurality of instances of the target mRNA or (ii) a prevalence of the absence of deamination of any nucleotide position in a respective instance of a target mRNA in a plurality of instances of the target mRNA.

Referring to block 3220, in some embodiments, the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

In some embodiments, the metric for the specificity of deamination of the target nucleotide position relative to one or more nucleotide positions, other than the target nucleotide position, in the target mRNA by the first ADAR protein is (i) a comparison of (a) a prevalence of deamination of the target nucleotide position in a plurality of instances of the target mRNA and (b) a prevalence of deamination of at least one nucleotide position, other than the target nucleotide position, in a respective instance of the target mRNA in a plurality of instances of the target mRNA, (ii) a prevalence of deamination of the target nucleotide position, without coincident deamination of one or more nucleotide positions other than the target nucleotide position, in a respective instance of the target mRNA in a plurality of instances of the target mRNA, or (iii) a prevalence of deamination of at least one nucleotide position, other than the target nucleotide position, in a respective instance of the target mRNA in a plurality of instances of the target mRNA.

Referring to block 3222, in some embodiments, at each respective nucleotide position in the one or more nucleotide positions, other than the target nucleotide position, in the target mRNA, deamination results in a non-synonymous codon edit. Referring to block 3222, in some embodiments, at one or more nucleotide positions, other than the target nucleotide position, in the target mRNA, deamination results in a non-synonymous codon edit.

Referring to block 3224, in some embodiments, a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Referring to block 3226, in some embodiments, the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Referring to block 3228, in some embodiments, the first ADAR protein is ADAR1 or ADAR2. Referring to block 3228, in some embodiments, the first ADAR protein is human ADAR1 or human ADAR2.

Referring to block 3230, in some embodiments, the set of one or metrics for the efficiency or specificity of deamination of the target nucleotide position by the ADAR protein comprises a metric for the efficiency or specificity of deamination of the target nucleotide position by a plurality of different ADAR proteins.

Referring to block 3232, in some embodiments, the output from the model further comprises one or more metrics for an efficiency or specificity of deamination of the target nucleotide position by a second ADAR protein when facilitated by hybridization of the gRNA to the target mRNA.

Referring to block 3234, in some embodiments, the one or more metrics for the efficiency or specificity of deamination of the target nucleotide position by the second ADAR protein comprises a metric for the efficiency of deamination of the target nucleotide position by the second ADAR protein.

In some embodiments, the metric for the efficiency of deamination of the target nucleotide position by the second ADAR protein is (i) a prevalence of deamination of the target nucleotide position in a plurality of instances of the target mRNA or (ii) a prevalence of the absence of deamination of any nucleotide position in a respective instance of a target mRNA in a plurality of instances of the target mRNA.

Referring to block 3236, in some embodiments, the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

In some embodiments, the metric for the specificity of deamination of the target nucleotide position relative to one or more nucleotide positions, other than the target nucleotide position, in the target mRNA by the second ADAR protein is (i) a comparison of (a) a prevalence of deamination of the target nucleotide position in a plurality of instances of the target mRNA and (b) a prevalence of deamination of at least one nucleotide position, other than the target nucleotide position, in a respective instance of the target mRNA in a plurality of instances of the target mRNA, (ii) a prevalence of deamination of the target nucleotide position, without coincident deamination of one or more nucleotide positions other than the target nucleotide position, in a respective instance of the target mRNA in a plurality of instances of the target mRNA, or (iii) a prevalence of deamination of at least one nucleotide position, other than the target nucleotide position, in a respective instance of the target mRNA in a plurality of instances of the target mRNA.

Referring to block 3238, in some embodiments, at each respective nucleotide position in the one or more nucleotide positions, other than the target nucleotide position, in the target mRNA, deamination results in a non-synonymous codon edit. Referring to block 3238, in some embodiments, at one or more nucleotide positions, other than the target nucleotide position, in the target mRNA, deamination results in a non-synonymous codon edit.

Referring to block 3240, in some embodiments, the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Referring to block 3242, in some embodiments, the first ADAR protein is ADAR1 and the second ADAR protein is ADAR2. Referring to block 3242, in some embodiments, the first ADAR protein is human ADAR1 and the second ADAR protein is human ADAR2.

In some embodiments, the metric for the efficiency of deamination of the target nucleotide position by the first ADAR protein is a comparison of (a) a prevalence of deamination of the target nucleotide position by a first ADAR protein in a plurality of instances of the target mRNA and (b) a prevalence of deamination of the target nucleotide position by a second ADAR protein in the plurality of instances of the target mRNA.

In some embodiments, the specificity is determined as the target edit percentage divided by the sum of all nonsynonymous off-target edits. In some embodiments, the specificity is determined as the (sum of on-target editing of the desired nucleotide)/(sum of off-target editing). In some embodiments, the specificity is determined as 1−(# of reads with only on-target edits)−(# of reads with zero edits).

Alternatively or additionally, in some embodiments, the one or more metrics comprises any on-target editing, specificity, target-only editing, no editing, and/or normalized specificity, for one or more ADAR proteins in a plurality of different ADAR proteins. For instance, in some embodiments, any on-target editing is determined as a proportion of sequence reads with any on-target edits. In some embodiments, specificity is determined as a (proportion of sequence reads with on-target edits+1)/(proportion of sequence reads with off-target edits+1). In some embodiments, target-only editing is determined as a proportion of sequence reads with only on-target edits. In some embodiments, no editing is determined as a proportion of sequence reads without any edits. In some embodiments, normalized specificity is determined as 1−(proportion of sequence reads with any off-target edits). In some embodiments, the one or more metrics further includes a difference in editing preference between a first ADAR protein and a second ADAR protein, in the plurality of different ADAR proteins. In some embodiments, the difference in editing preference is determined as (target-only editing of the first ADAR protein)−(target-only editing of the second ADAR protein). In some embodiments, the one or more metrics are obtained for ADAR1, ADAR2, or ADAR1/2. Alternatively or additionally, in some embodiments, the one or more metrics further includes editability, where editability is a measure of central tendency of the any on-target editing and target-only editing scores. In some embodiments, editability is the average of the any on-target editing and target-only editing scores.

In some embodiments, the output from the model further comprises a determination of a free energy for the gRNA and/or for the guide-target RNA scaffold formed between the guide RNA (gRNA) and the target mRNA.

Referring to block 3244, in some embodiments, the model further generates an estimation of a minimum free energy (MFE) for the gRNA.

Referring to block 3246, in some embodiments, the model further generates an estimation of a minimum free energy (MFE) for the guide-target RNA scaffold formed between the guide RNA (gRNA) and the target mRNA.

Referring to block 3248, in some embodiments, the model is a neural network, a support vector machine, a Naive Bayes model, a nearest neighbor model, a boosted trees model, a random forest model, a decision tree, or a clustering model.

Referring to block 3250, in some embodiments, the model is an extreme gradient boost (XGBoost) model.

Referring to block 3252, in some embodiments, the model is a convolutional or graph-based neural network.

Referring to block 3254, in some embodiments, the plurality of parameters is at least 1000 parameters, at least 5000 parameters, at least 10,000 parameters, at least 100,000 parameters, at least 250,000 parameters, at least 500,000 parameters, or at least 1,000,000 parameters.

In some embodiments, the plurality of parameters comprises at least 200, at least 500, at least 1000, at least 5000, at least 10,000, at least 100,000, at least 250,000, at least 500,000, at least 1,000,000, or at least 5,000,000 parameters. In some embodiments, the plurality of parameters comprises no more than 10,000,000, no more than 5,000,000, no more than 1,000,000, no more than 100,000, no more than 10,000, or no more than 1000 parameters. In some embodiments, the plurality of parameters consists of from 200 to 10,000, from 1000 to 100,000, from 5000 from 200,000, from 100,000 to 500,000, from 500,000 to 1,000,000, from 1,000,000 to 5,000,000, or from 5,000,000 to 10,000,000 parameters. In some embodiments, the plurality of parameters falls within another range starting no lower than 200 parameters and ending no higher than 10,000,000 parameters.

Referring to block 3256, in some embodiments, the plurality of parameters reflects a first plurality of values, where each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

In some embodiments, the first plurality of values comprises at least 100, at least 1000 values, at least 5000 values, at least 10,000 values, at least 100,000 values, at least 250,000 values, at least 500,000 values, or at least 1,000,000 values. In some embodiments, the first plurality of values comprises no more than 5,000,000, no more than 1,000,000, no more than 100,000, no more than 10,000, or no more than 1000 values. In some embodiments, the first plurality of values consists of from 200 to 10,000, from 1000 to 100,000, from 5000 from 200,000, from 100,000 to 500,000, from 500,000 to 1,000,000, or from 1,000,000 to 5,000,000 values. In some embodiments, the first plurality of values falls within another range starting no lower than 100 values and ending no higher than 5,000,000 values.

In some embodiments, the first plurality of training gRNA comprises at least 1000 gRNAs, at least 5000 gRNAs, at least 10,000 gRNAs, at least 100,000 gRNAs, at least 250,000 gRNAs, at least 500,000 gRNAs, or at least 1,000,000 gRNAs. In some embodiments, the first plurality of training gRNA comprises no more than 5,000,000, no more than 1,000,000, no more than 100,000, no more than 10,000, or no more than 1000 gRNAs. In some embodiments, the first plurality of training gRNA consists of from 200 to 10,000, from 1000 to 100,000, from 5000 from 200,000, from 100,000 to 500,000, from 500,000 to 1,000,000, or from 1,000,000 to 5,000,000 gRNAs. In some embodiments, the first plurality of training gRNA falls within another range starting no lower than 100 gRNAs and ending no higher than 5,000,000 gRNAs.

Referring to block 3258, in some embodiments, the plurality of parameters further reflects a second plurality of values, where each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

In some embodiments, the second plurality of values comprises at least 100, at least 1000 values, at least 5000 values, at least 10,000 values, at least 100,000 values, at least 250,000 values, at least 500,000 values, or at least 1,000,000 values. In some embodiments, the second plurality of values comprises no more than 5,000,000, no more than 1,000,000, no more than 100,000, no more than 10,000, or no more than 1000 values. In some embodiments, the second plurality of values consists of from 200 to 10,000, from 1000 to 100,000, from 5000 from 200,000, from 100,000 to 500,000, from 500,000 to 1,000,000, or from 1,000,000 to 5,000,000 values. In some embodiments, the second plurality of values falls within another range starting no lower than 100 values and ending no higher than 5,000,000 values.

In some embodiments, the second plurality of training gRNA comprises at least 1000 gRNAs, at least 5000 gRNAs, at least 10,000 gRNAs, at least 100,000 gRNAs, at least 250,000 gRNAs, at least 500,000 gRNAs, or at least 1,000,000 gRNAs. In some embodiments, the second plurality of training gRNA comprises no more than 5,000,000, no more than 1,000,000, no more than 100,000, no more than 10,000, or no more than 1000 gRNAs. In some embodiments, the second plurality of training gRNA consists of from 200 to 10,000, from 1000 to 100,000, from 5000 from 200,000, from 100,000 to 500,000, from 500,000 to 1,000,000, or from 1,000,000 to 5,000,000 gRNAs. In some embodiments, the second plurality of training gRNA falls within another range starting no lower than 100 gRNAs and ending no higher than 5,000,000 gRNAs.

Referring to block 3260, in some embodiments, the first plurality of training gRNA and the second plurality of training gRNA are the same.

Referring to block 3262, in some embodiments, the method further includes iteratively updating the seed nucleic acid sequence 3032, while holding the plurality of parameters 3042 and the target nucleic acid sequence 3034 fixed, to reduce a difference between (i) the desired set of the one or more metrics 3024 and (ii) the calculated set of the one or metrics 3052, thereby generating the candidate sequence.

Referring to block 3264, in some embodiments, the method further includes determining, using a gRNA having the candidate sequence, an experimental set of the one or more metrics for the efficiency or specificity of deamination of the target nucleotide position in the target mRNA by an ADAR protein; and training a model using a training dataset comprising the experimental set of the one or more metrics for the efficiency or specificity of deamination of the target nucleotide position in the target mRNA by the ADAR protein.

### ADDITIONAL EMBODIMENTS

Another aspect of the present disclosure provides a computer system comprising one or more processors and a non-transitory computer-readable medium including computer-executable instructions that, when executed by the one or more processors, cause the processors to perform any of the methods and/or embodiments disclosed herein.

Yet another aspect of the present disclosure provides a non-transitory computer-readable storage medium having stored thereon program code instructions that, when executed by a processor, cause the processor to perform any of the methods and/or embodiments disclosed herein.

Still another aspect of the present disclosure provides a computer-implemented method comprising receiving a sequence of a target nucleic acid, where the target nucleic acid sequence comprises a target nucleotide. The method further includes receiving a candidate sequence of an engineered guide RNA, where the engineered guide RNA when bound to the target nucleic acid forms a guide-target RNA scaffold. A version of the target nucleic acid sequence and a version of the candidate sequence of the engineered guide RNA are inputted to a machine learning model, the machine learning model iteratively trained by a set of training samples of the target nucleic acid sequence and candidate sequences of engineered guide RNAs. The method further includes generating, by the machine learning model, a prediction associated with a percentage of on-target editing of the target nucleotide, a specificity score, or both, after formation of the ADAR substrate comprising a version of the candidate sequence of the engineered guide RNA bound to the target nucleic acid, the prediction being specific to the nucleic acid sequence inputted to the machine learning model.

In some embodiments, the target adenosine causes a genetic disease and the candidate sequence of the engineered guide agent is capable of being encoded in a vector to treat the genetic disease. In some embodiments, the target RNA, in which the protein expressed from it causes or is associated with a disease, comprises a target adenosine, and the candidate sequence of the engineered guide agent is capable of being encoded in a vector to edit the target adenosine to treat the disease.

In some embodiments, the machine learning model is a regression model, random forests, a support vector machine, or a neural network.

In some embodiments, the machine learning model is a neural network that comprises one or more convolutional layers.

In some embodiments, the version of the nucleic acid sequence and the candidate sequence of the engineered guide RNA sequence are raw sequences, encoded sequences, or a set of one or more extracted features associated with these sequences.

In some embodiments, training of the machine learning model comprises determining, in a forward propagation, predicted scores of one or more training samples in the set of training samples. The predicted scores are compared to actual scores of the one or more training samples, and an objective function of the machine learning model is determined based on comparing the predicted scores to the actual scores. Training further includes adjusting, in a backpropagation, one or more weights of the machine learning model, and repeating the forward propagation and the backpropagation for a plurality of iterations.

In some embodiments, the specificity score is a target nucleotide edit percentage divided by a sum of off-target nucleotide edits.

In some embodiments, the method further comprises identifying a list of candidate features of the candidate sequence that have impact on model outputs of the machine learning model and selecting one or more features that have strong impacts on the model outputs compared to the rest of the candidate features.

In some embodiments, the target nucleic acid sequence is a target RNA sequence.

In some embodiments, the method further comprises inputting the percentage of on-target editing of the target nucleotide, the specificity score, or both, into the machine learning model, and generating, by the machine learning model, a prediction of a version of the candidate sequence of the engineered guide RNA having the percentage of on-target editing of the target nucleotide, the specificity score, or both, after formation of the ADAR substrate comprising a version of the candidate sequence of the engineered guide RNA bound to the target nucleic acid, where the prediction is specific to the percentage of on-target editing of the target nucleotide, the specificity score, or both inputted to the machine learning model.

In some embodiments, at least one of the version of the target nucleic acid sequence and the version of the candidate sequence is one hot-encoded.

In some embodiments, the method further comprises inputting positional encodings into the machine learning model, where the positional encoding transfers coordinate information to the machine learning model.

In some embodiments, the engineered guide RNA comprises an ADAR recruiting domain, one or more latent structural features, or both.

In some embodiments, the ADAR recruiting domain comprises a recruitment hairpin.

In some embodiments, the one or more latent structural features comprises a bulge, an internal loop, a Wobble base pair, or non-recruitment hairpin.

In some embodiments, the method further comprises generating, using a second machine learning model of a different type than the machine learning model, a second prediction of the percentage of on-target editing of the target nucleotide, the specificity score, or both, after formation of the ADAR substrate comprising a version of the candidate sequence of the engineered guide RNA bound to the target nucleic acid, and identifying the candidate sequence as a good sequence responsive to the prediction and the second prediction both indicating that the percentage of on-target editing of the target nucleotide, the specificity score, or both, exceed corresponding thresholds.

Another aspect of the present disclosure provides a system comprising a processor and a memory storing instructions, which when executed by the processor, cause the processor to perform steps comprising any of the methods disclosed herein.

Yet another aspect of the present disclosure provides a non-transitory computer-readable medium storing computer code comprising instructions, when executed by one or more processors, causing the processors to perform any of the methods disclosed herein.

### Equivalents and Incorporation by Reference

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### Additional Consideration

The foregoing description of the embodiments has been presented for the purpose of illustration; it is not intended to be exhaustive or to limit the patent rights to the precise forms disclosed. Persons skilled in the relevant art will appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter, in some embodiments, includes not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein, in some embodiments, are claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines are, in some embodiments, embodied in software, firmware, hardware, or any combinations thereof.

Any of the steps, operations, or processes described herein, in some embodiments, are performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure describes, in some embodiments, a process that includes multiple steps sequentially with arrows present in a flowchart, the steps in the process do not need to be performed by the specific order claimed or described in the disclosure. In some implementations, some steps are performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, in some implementations, plural instances implement components, operations, or structures described as a single instance. Although individual operations of one or more methods are illustrated and described as separate operations, in some implementations one or more of the individual operations are performed concurrently, and nothing requires that the operations be performed in the order illustrated. Structures and functionality presented as separate components in example configurations are, in some embodiments, implemented as a combined structure or component. Similarly, in some embodiments, structures and functionality presented as a single component are implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, in some instances, the use of a singular form of a noun implies at least one element even though a plural form is not used.

Finally, the language used in the specification has been principally selected for readability and instructional purposes, rather than selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

### EXAMPLES

The following illustrative examples are representative of embodiments of the stimulation, systems, and methods described herein and are not meant to be limiting in any way.

### Example 1

### Machine Learning to Predict Percent Target Editing and Specificity Score of an Engineered Guide that Target LRRK2 G2019S mRNA

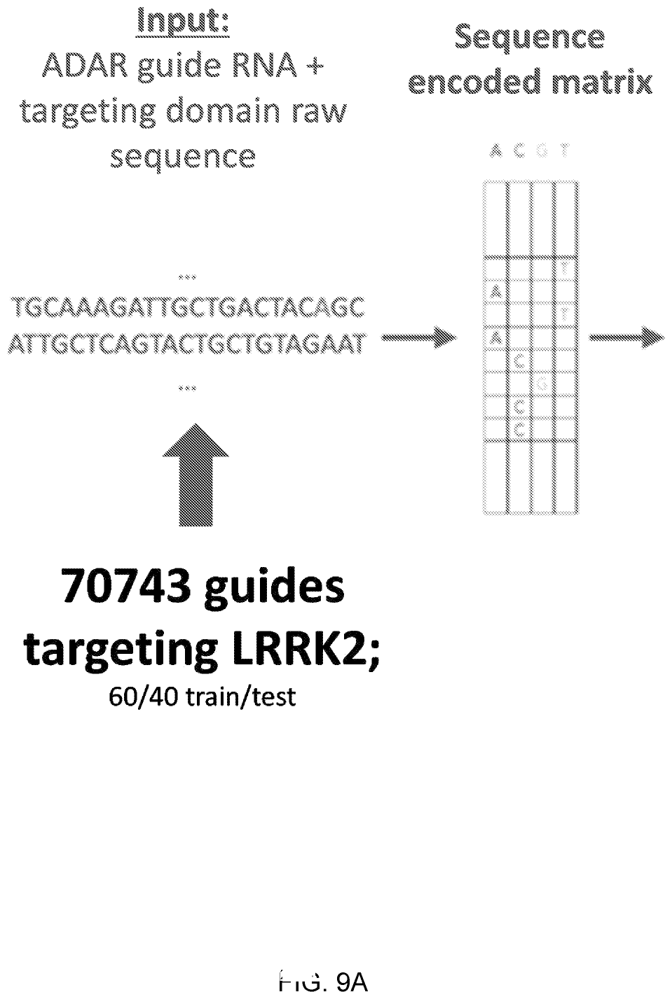

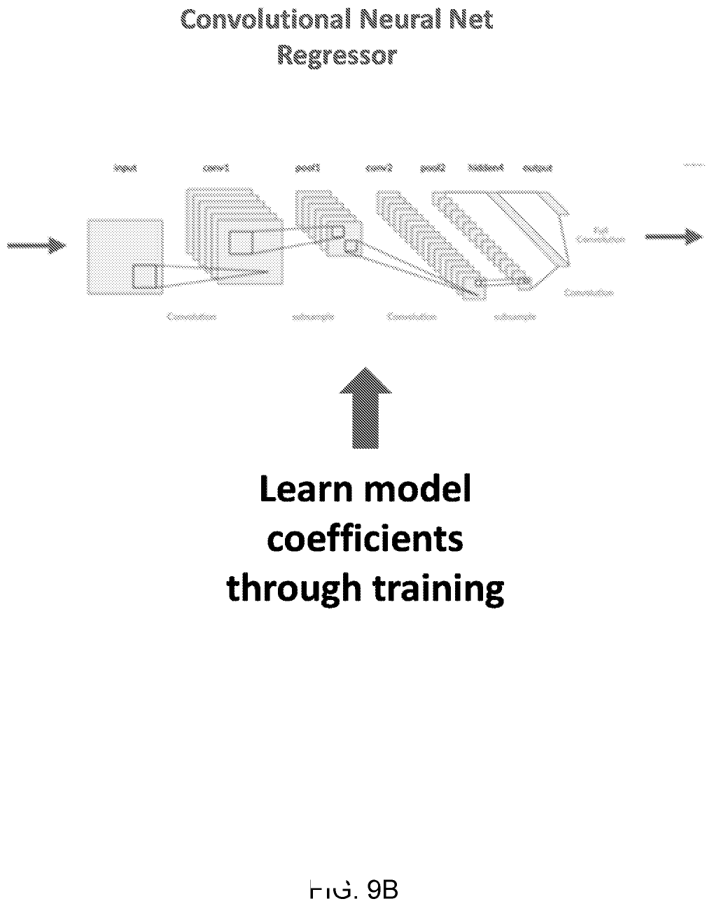

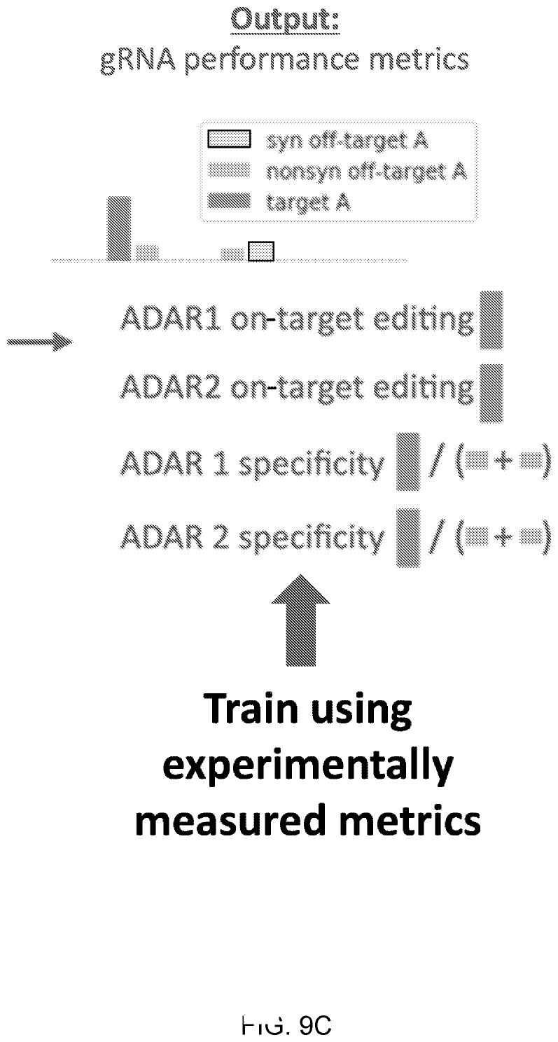

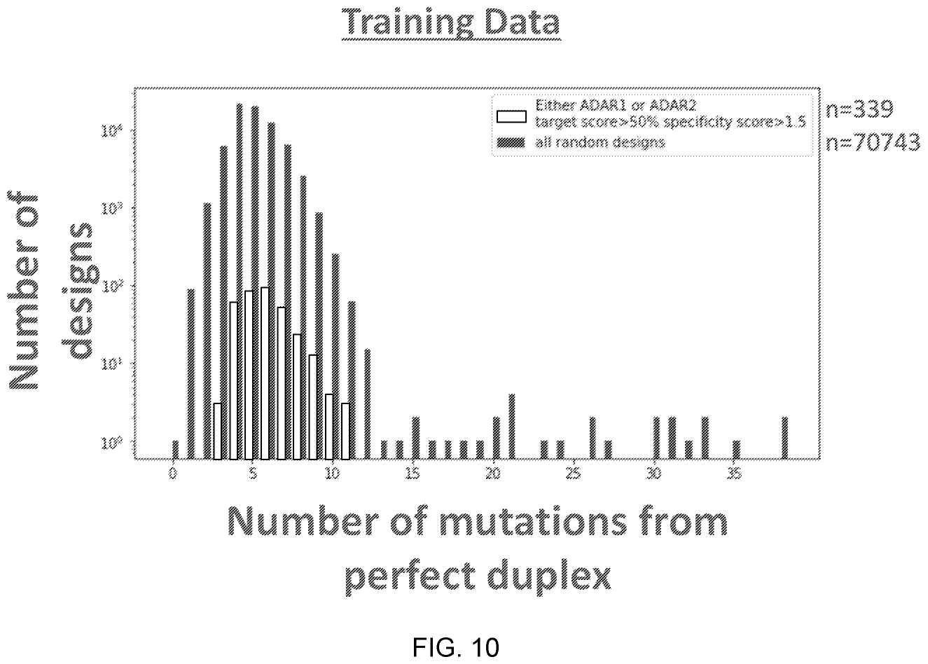

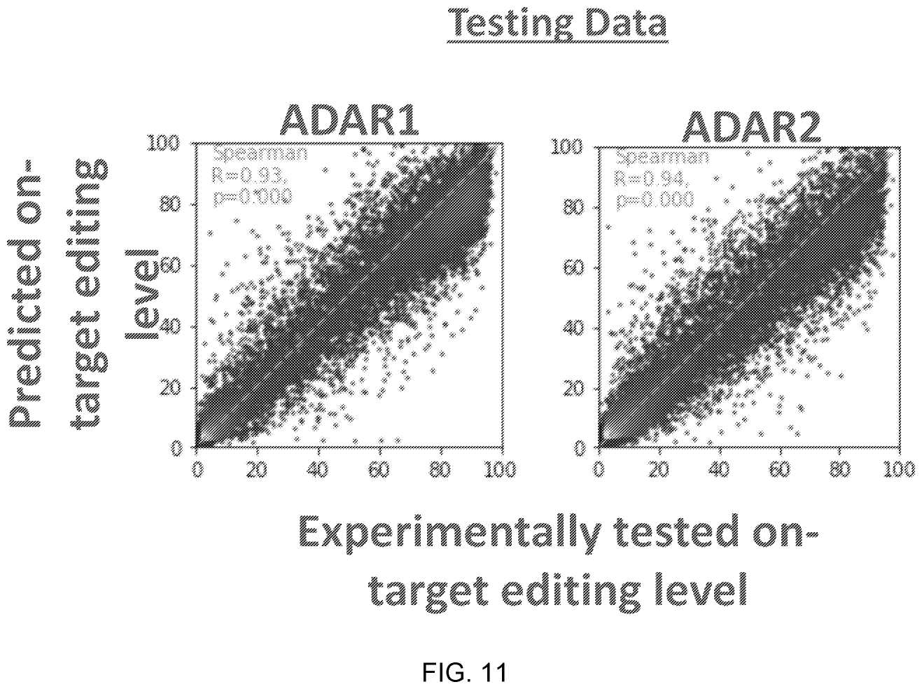

This example describes using machine learning to predict on-target editing (percentage of edited reads of the target adenosine in the LRRK2 G2019S mRNA) and a specificity score ((number of reads with on-target edits of the target adenosine in the LRRK2 G2019S mRNA)/(sum of all reads with off-target edits in the LRRK2 G2019S mRNA)) based on an engineered guide RNA sequence. A set of 70,743 guides targeting LRRK2 G2019S mRNA, in which the guide RNAs of this set form various structural features in the guide-target RNA scaffold, was used to train and test a convolutional neural network (CNN). Of this set of guides, 60% were used to train the model and 40% were used to test the accuracy of the CNN for predicting on-target editing and specificity score based on an engineered guide sequence. FIGS. 9A-C collectively show a schematic of the CNN workflow. FIG. 10 shows the number of guide RNAs with different numbers of mutations (compared to a perfect duplex) used to train the CNN, indicating that most guides with high on-target editing and specificity were centered at 5-7 mutations. FIG. 11 displays the observed high correlation (Spearman's rank order correlation coefficient=0.93 for ADAR1 and 0.94 for ADAR2) between the predicted and experimentally validated on-target editing and specificity scores, indicating that the trained CNN accurately predicts on-target editing and specificity score based on an engineered guide sequence. The experimental validation was performed in a cell-free system via high throughput screening of self-annealing guide RNAs linked to target RNAs by a hairpin and using ADAR1 and/or ADAR2 to perform the editing.

### Example 2

### Machine Learning for Engineered Guides that Target LRRK2 G2019S mRNA

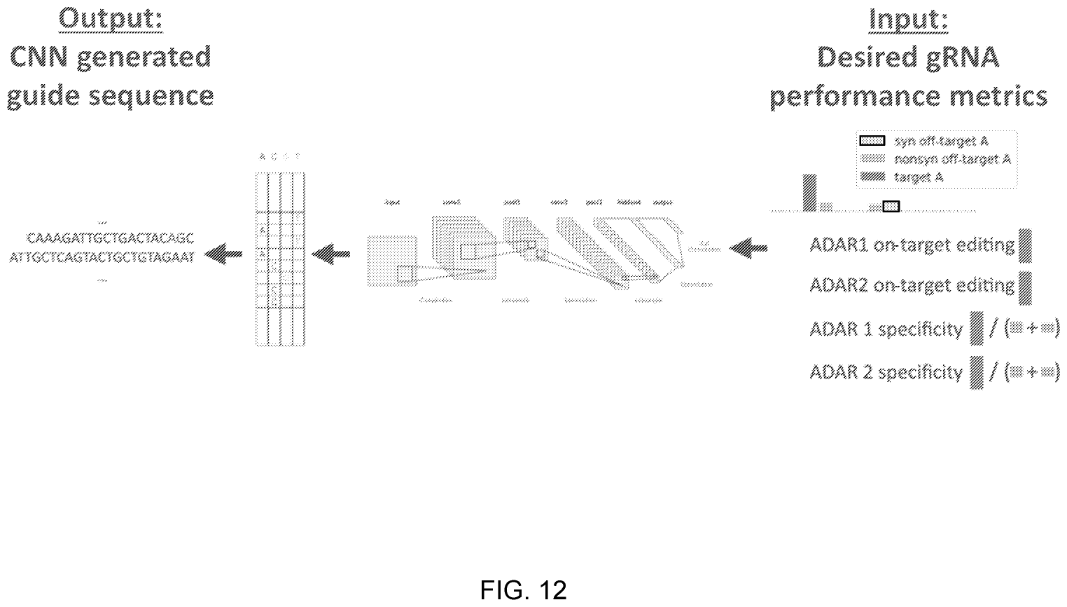

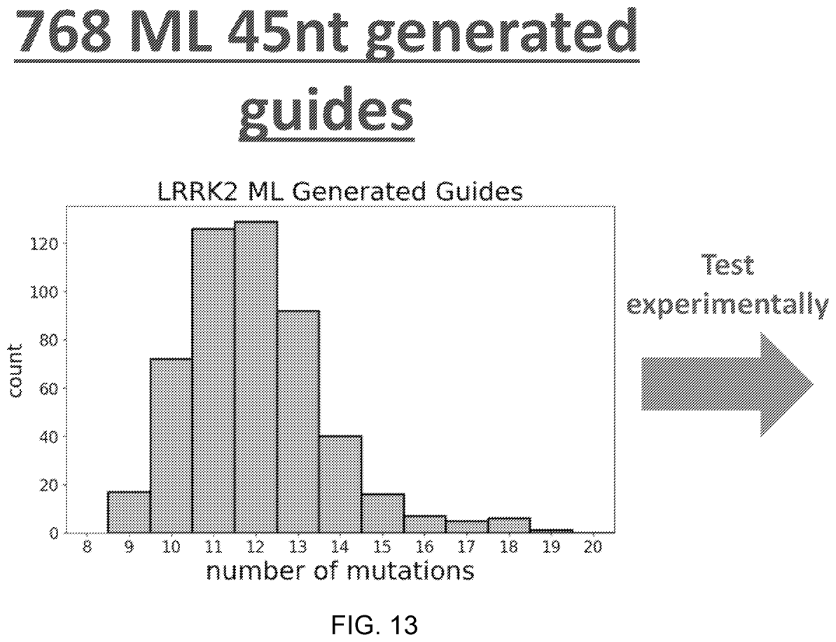

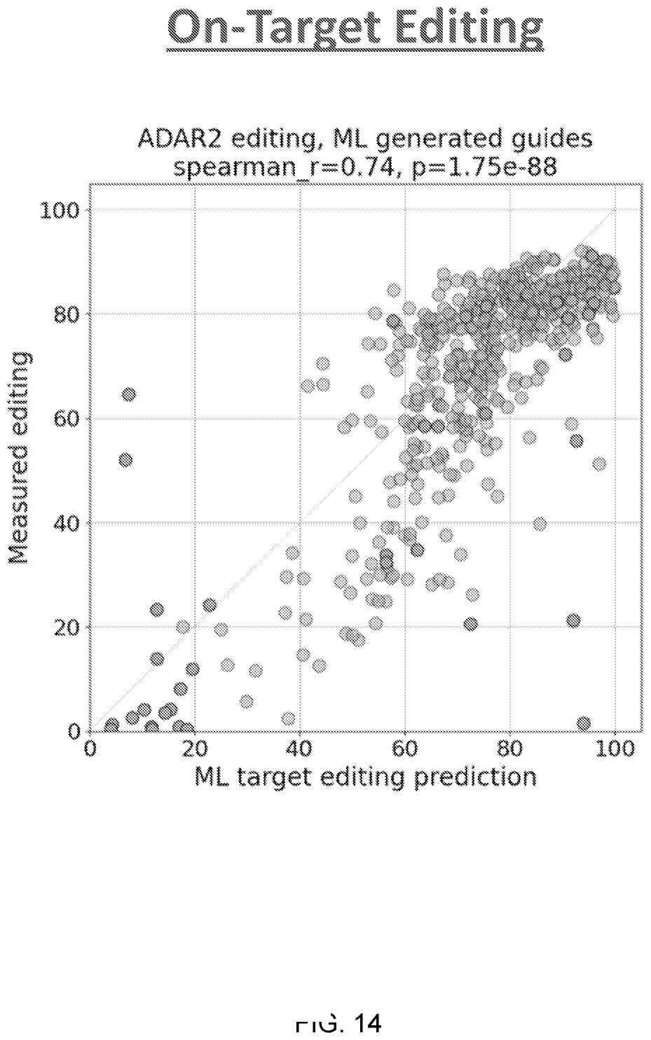

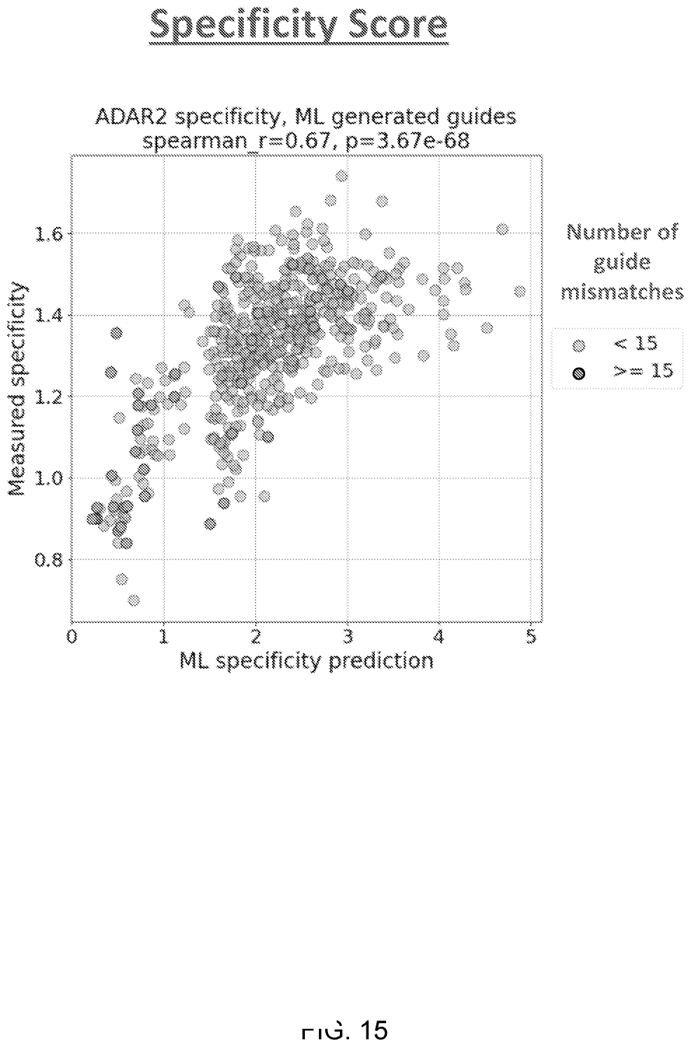

This example describes generating engineered guide RNA sequences that target LRRK2 G2019S mRNA based on a specified on-target editing and a specified specificity score using machine learning. Input optimization was used on the trained CNN of EXAMPLE 1, in which a specified on-target editing and specified specificity score was chosen and the nucleotides comprising the input sequence to the model were optimized. Following the optimization procedure, gradient descent, the resultant engineered guide sequence minimizes an L1 loss between the desired on-target and specificity scores and the values predicted by the trained CNN as shown in FIG. 12. FIG. 13 shows the number of guide RNAs with different numbers of mutations (compared to a perfect duplex) generated by the CNN, indicating that distribution of predicted top guides achieved a greater sequence diversity from the perfect duplex than the original library in FIG. 11. The generated guide RNAs on-target editing and specificity scores were then experimentally validated as described in EXAMPLE 1 by high-throughput screen. There was a high correlation between the predicted on-target editing and specificity score and the experimentally measured on-target editing and specificity score (FIG. 14 & FIG. 15), with a Spearman's rank correlation coefficient of 0.74 for on-target editing and 0.67 for the specificity score. This result indicates that the trained CNN accurately generated engineered guide sequences based on the on-target editing and specificity score inputs, many of which were over 15 mutations away from the perfect duplex.

### Example 3

### Machine Learning for Determining gRNA Features that Impact LRRK2 G2019S mRNA Editing

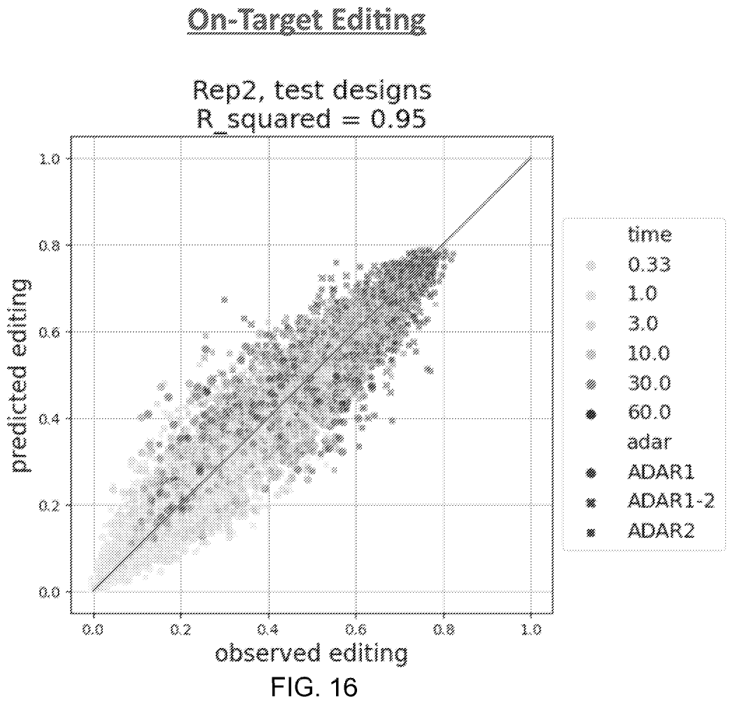

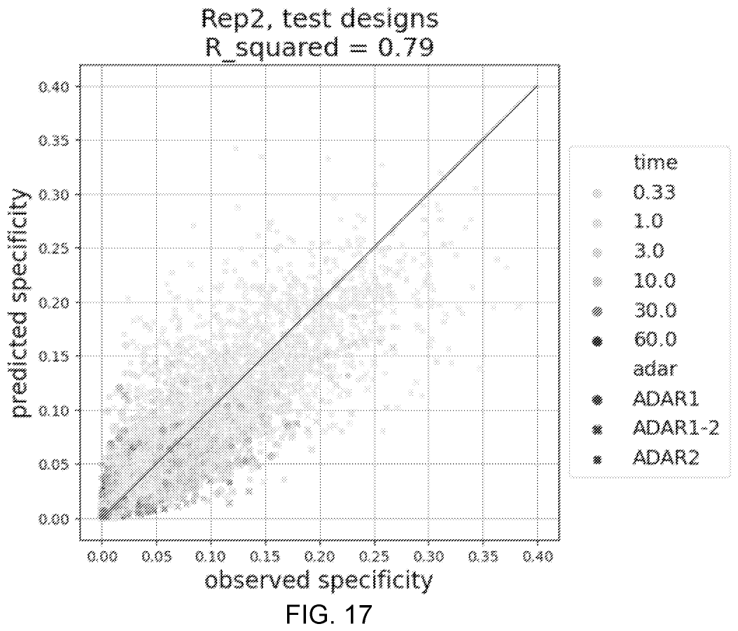

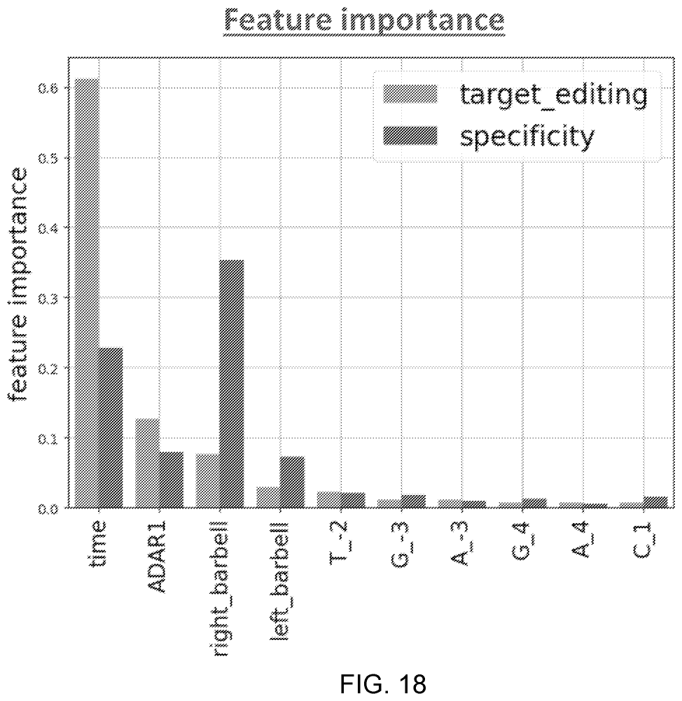

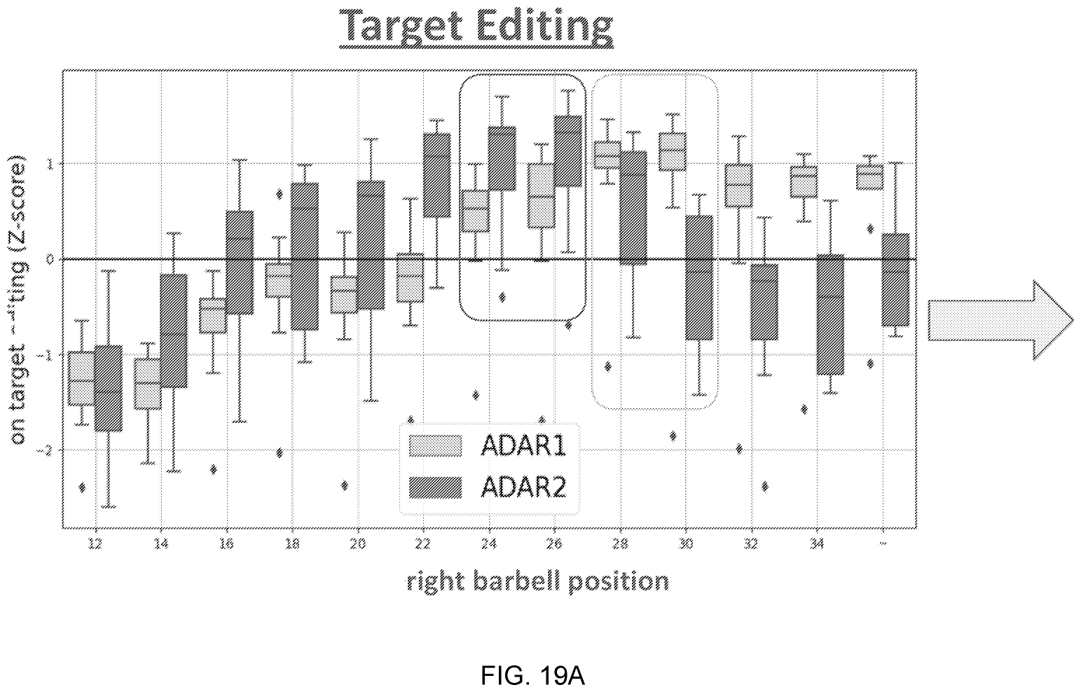

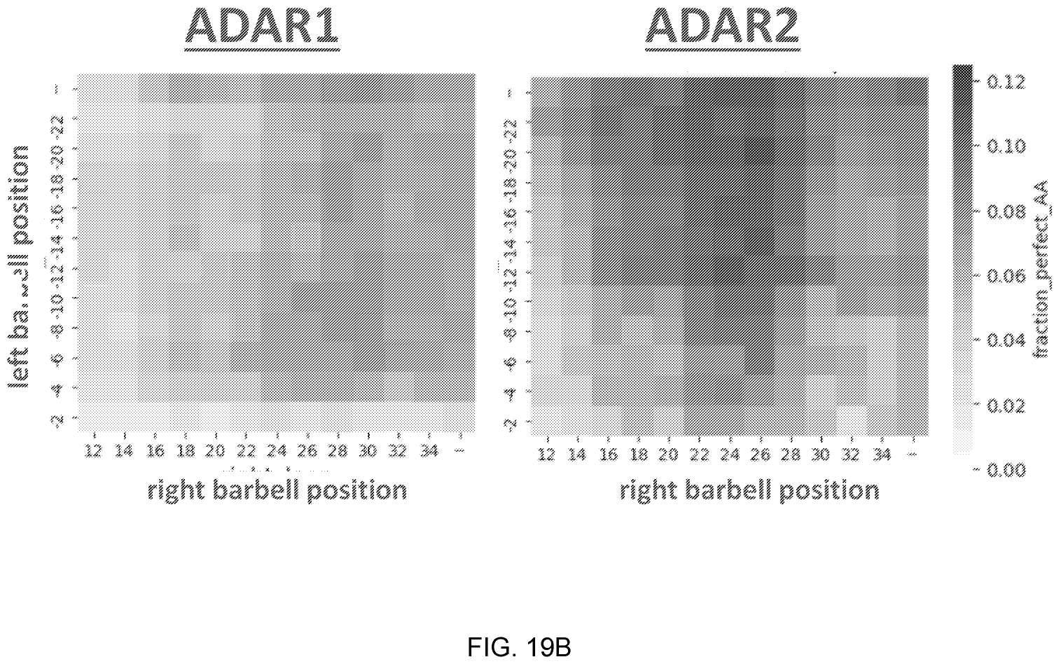

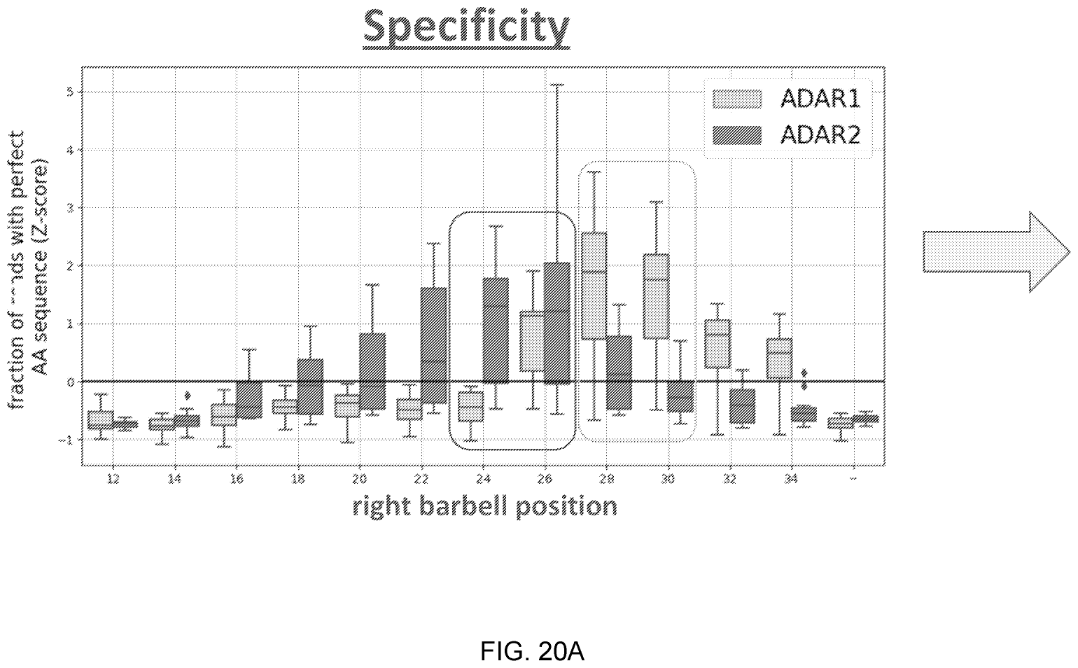

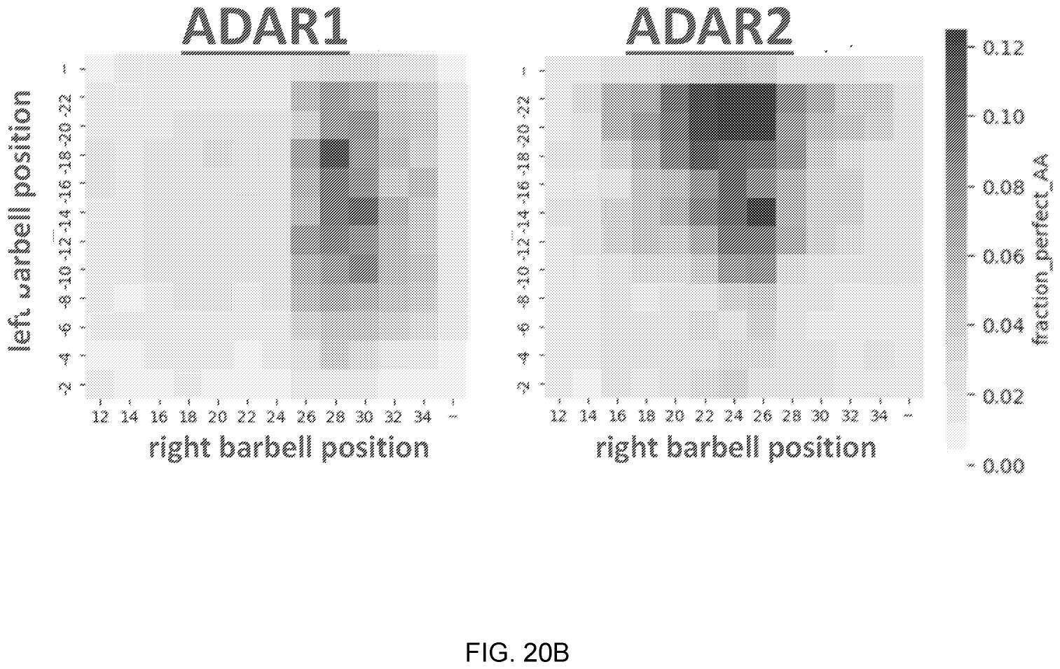

This example describes using machine learning to determine features of a guide RNA that impact on-target editing and specificity score for editing a LRRK2 G2019S mRNA. A set of 1709 engineered guide RNAs was used to train and test a random forests (RF) model. Of this set of guides, 1000 engineered guides were used to train the RF model and 709 engineered guides were used to test the accuracy of the trained RF model for predicting on-target editing and specificity score based on an engineered guide sequence. There was a high correlation between the predicted on-target editing and specificity score and the experimentally tested on-target editing and specificity score (R²=0.95 and 0.79, respectively), indicating that the trained RF model accurately predicts on-target editing and specificity score based on an engineered guide sequence. This high correlation is shown for on-target editing in FIG. 16 and for specificity score in FIG. 17. This trained RF model was then used to determine features of the guide RNAs that impact on-target editing and specificity score, such as length of time for editing (20 sec, 1 min, 3 min, 10 min, 30 min, or 60 min), the ADAR used for editing (ADAR1, ADAR2, or ADAR1 and ADAR2), positioning of a right barbell (relative to the target adenosine to be edited), positioning of a left barbell (relative to the target nucleotide to be edited), and/or nucleotide identity and relative position. The right barbell was the most important feature for predicting specificity of an engineered guide RNA and the third most important feature for predicting on-target editing, as shown in FIG. 18. For engineered guide RNAs using ADAR1 for editing, the best positioning of the right barbell to achieve a high target editing and/or a high specificity score was +28 or +30 nts, wherein the positioning is relative to the target adenosine to be edited, as shown in FIGS. 19A-B and FIGS. 20A-B. For engineered guide RNAs using ADAR2 for editing, the best positioning of the right barbell to achieve a high target editing and/or a high specificity score was +24 or +26 nts, wherein the positioning is relative to the target adenosine to be edited, as shown in FIGS. 19A-B and FIGS. 20A-B.

### Example 4

### Machine Learning for an Engineered Guide RNA that Targets LRRK2 G2019S mRNA

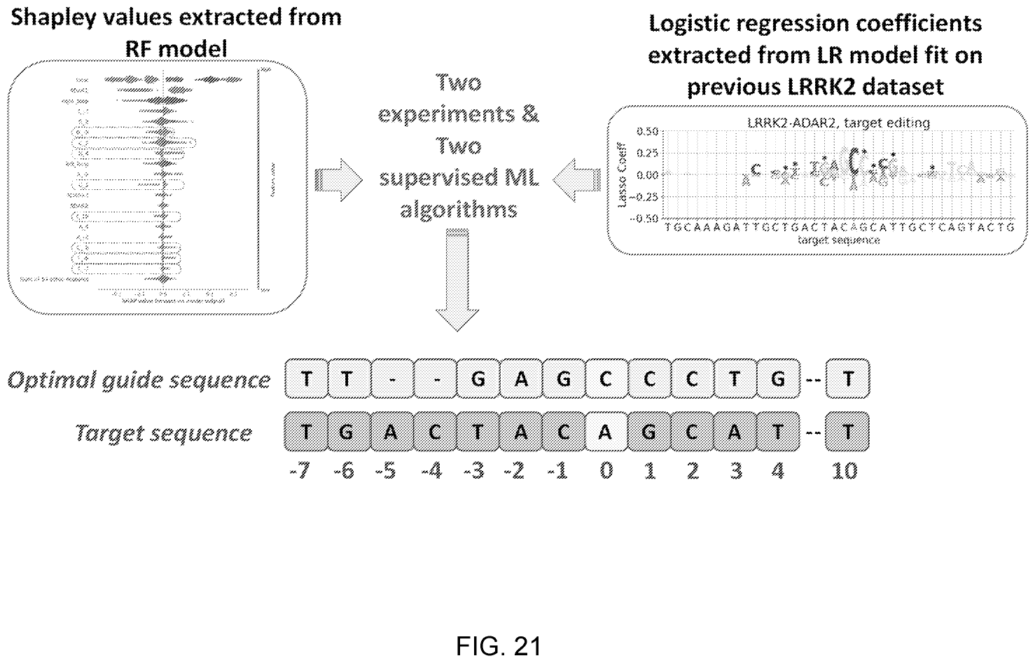

This example, shown in FIG. 21, describes using machine learning to determine identities of nucleotides at specific positions in engineered guide RNAs that target LRRK2 G2019S mRNA to achieve high on-target editing. Machine learning was performed using a logistic regression model with lasso (L1) regularization trained on a set of engineered guide RNAs. Logistic regression coefficients were extracted from the lasso regression model. The trained RF model from EXAMPLE 3 was also used. Shapley values were extracted from this trained RF model. The Shapley values and the logistic regression coefficients were then assessed for overlapping nucleotides at specific positions in the engineered guide RNAs that had high on-target editing. This overlap was used to determine the identities of nucleotides at specific positioning in engineered guide RNAs that target LRRK2 G2019S mRNA that achieve high target editing. These nucleotides and positions in the engineered guide RNA are as follows: T at position −7, T at position −6, G at position −3, A at position −2, G at position −1, C at position 1, C at position 2, G at position 4, and T at position 10, wherein these positions are relative to the target adenosine in the LRRK2 G2019S mRNA to be edited.

### Example 5

### Massively Parallel gRNA Screening and Machine Learning to Enable Efficient and Selective RNA Editing with Endogenous ADAR

RNA editing holds great promise as a therapeutic modality for correcting pathogenic single nucleotide polymorphisms (SNPs) and modulating protein function or expression. Delivery of a guide RNA (gRNA) with complementarity to a target RNA can recruit ADAR's deaminase activity, converting a target adenosine to inosine, which is read by cellular machinery as guanosine. ADAR does not naturally act on all RNA sequences with equal efficiency and specificity. However, it has been observed that a small fraction of natural ADAR substrates are edited with high selectivity and efficiency due to precise secondary structures that promote a high degree of editing specificity.

Accordingly, an experiment was designed to test the hypothesis that customizing and optimizing the secondary structures within the guide-target RNA scaffold will allow specific and efficient editing of many or all therapeutic targets of interest.

The following example demonstrates a platform for therapeutic RNA editing by identifying guide RNAs (gRNAs), in accordance with an embodiment of the present disclosure. The platform uses high-throughput screening (HTS) and machine learning (ML) approaches that enable the engineering of gRNAs that, when complexed with their various target mRNA sequences, form secondary structures that promote highly selective and efficient editing of the target adenosine by endogenously expressed ADAR enzymes.

### INTRODUCTION

ADAR enzymes promiscuously deaminate adenosine to inosine within dsRNA structures. In contrast to chemically modified gRNAs, genetically encoded gRNAs solely rely on secondary structure of the guide-target RNA scaffold to promote selective editing.

HTS and ML have enabled the identification and design of critical secondary structures in gRNAs that promoted highly efficient and selective editing. For instance, as illustrated in FIG. 23A, a workflow using a HTS and ML platform includes designing, for each novel target, a large range of structurally randomized gRNAs (e.g., in accordance with an embodiment of the present disclosure); creating a library of the variant gRNAs and binding these gRNAs to the target RNA; treating the library with ADAR enzymes (e.g., human ADAR); and sequencing the ADAR-treated library using next-generation sequencing (NGS) to identify promising gRNAs. FIG. 23B illustrates a schematic of an example gRNA design. In some implementations, secondary structures in gRNA designs promote highly selective editing for restoration or modulation of protein expression or function. In some cases, lead gRNA designs identified using a HTS and ML platform can be advanced for validation in cells and further engineering.

### Use of the HTS and ML Platform for Input Optimization.

### HTS Applied to Disease Relevant Targets.

To date, most ADAR editing studies, especially those using gene-encoded gRNAs, have focused on adenosines within a “UAG” context due to ADAR's strong preference for this motif. However, most clinically relevant targets will not be in a UAG context. A HTS platform in accordance with the present disclosure was applied to seven disease relevant targets, including adenosines with 5′ G, C, and A neighbors.

For each respective target gene, the HTS was used to generate one or more candidate sequences for a guide RNA (e.g., gRNA designs). Desired values for a set of properties were obtained, including a metric for an on-target editing fraction and a specificity score, as described herein, of deamination of a target nucleotide position in mRNA transcribed from a target gene by an ADAR protein. An example specificity score is determined as ((sum of on-target edits of the desired nucleotide)/(sum of off-target edits)).

For all targets tested, the HTS platform identified gRNA designs with diverse secondary structures that yield high editing efficiency and selectivity.

Gradient Boosted Decision Trees Predict gRNA Activity.

Unique solutions are required for each target, and basic “rules” for ADAR editing (e.g., AC mismatch at target, AG mismatch or U-deletion at off-target) often will not suffice for a therapeutic intervention. Advantageously, machine learning can be used to optimize gRNA structure and understand the principles behind ADAR-mediated editing.

As a proof-of-concept, gradient boosted XGBoost models were trained, using data from the HTS screen, that were highly predictive of both editing and specificity across targets.

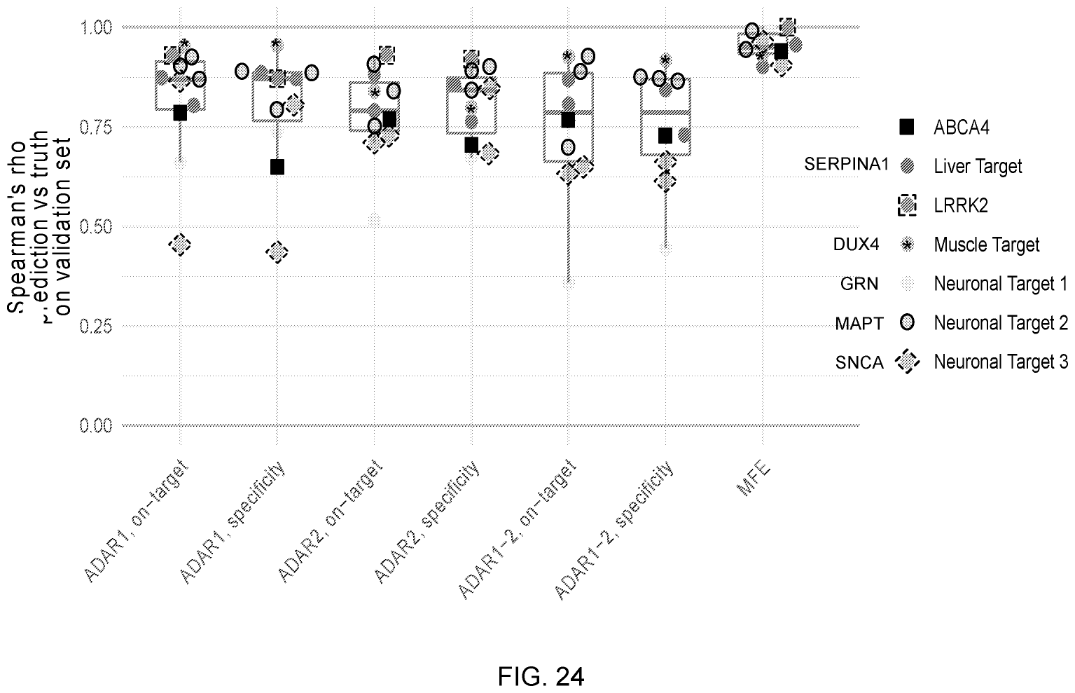

FIG. 24 illustrates example outputs from XGBoost models predicting gRNA editing and specificity. XGBoost models were trained to predict ADAR1 and ADAR2 editing efficiency, specificity, and minimum free energy (MFE) using gRNA data sets from a diverse panel of disease relevant targets. In particular, the gRNA data sets were used to train XGBoost models for the disease-relevant gene targets ABCA4, SERPINA1, LRRK2, DUX4, GRN, MAPT, and/or SNCA.

Thus, the XGBoost models were used to obtain, for each respective target gene (e.g., ABCA4, SERPINA1, LRRK2, DUX4, GRN, MAPT, and/or SNCA), for each respective gRNA in the corresponding set of gRNAs, a respective on-target percentage for ADAR1, a respective specificity for ADAR1, a respective on-target percentage for ADAR2, a respective specificity for ADAR2, a respective combined on-target percentage for ADAR1-2, a respective combined specificity for ADAR1-2, and an MFE. As illustrated in FIG. 24, Spearman's rho is plotted for each metric and disease target.

A comparison between the predictive ability of the XGBoost models and a convolutional neural network (CNN) was performed. In an example implementation, a CNN model was constructed against a library of gRNAs targeting the LRRK2 G2019S (45-mer) mutation.

Advantageously, CNN models further allow for generative design by input optimization to more efficiently explore the extremely diverse guide design sequence space. A CNN model for predicting the set of properties for gRNA-directed editing of the LRRK2 G2019S mutation target gene was trained based on data collected from a set of wet lab gRNA screens. Then, input optimization of the model was performed based on desired values for the set of properties using canonical gRNA designs as seed gRNA sequences, to generate a library of candidate gRNA sequences. Thus, the CNN model allowed for generating candidate sequences (e.g., novel gRNA designs) for gRNAs using the input optimization operation.

Millions of gRNAs targeting LRRK2 G2019S were therefore exhaustively scored by the CNN by searching sequence space within five mutations of the perfect complex as well as input optimization to design gRNAs up to 25 base pairs away.

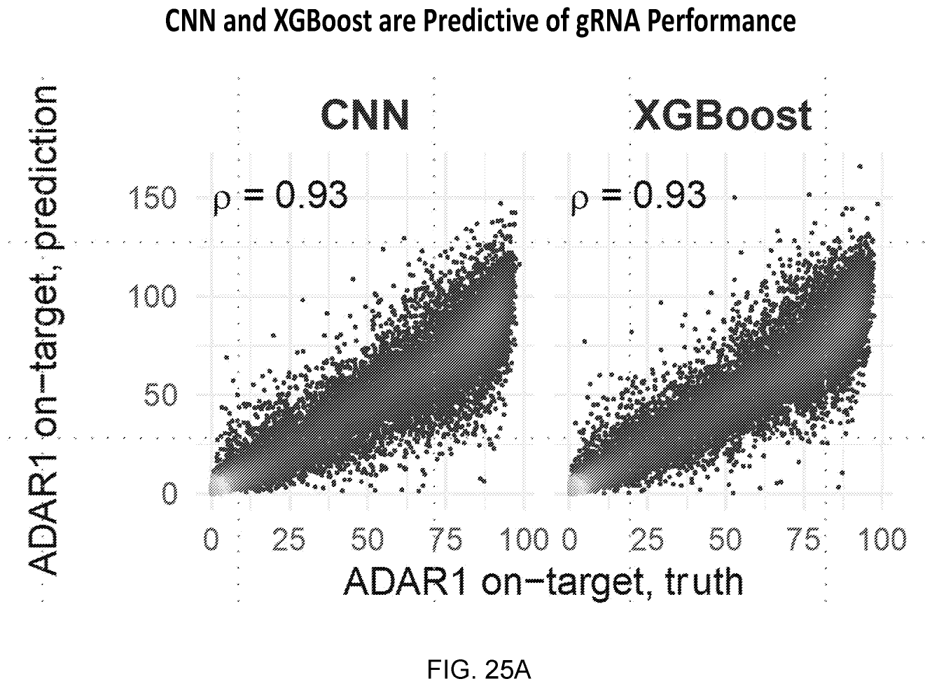

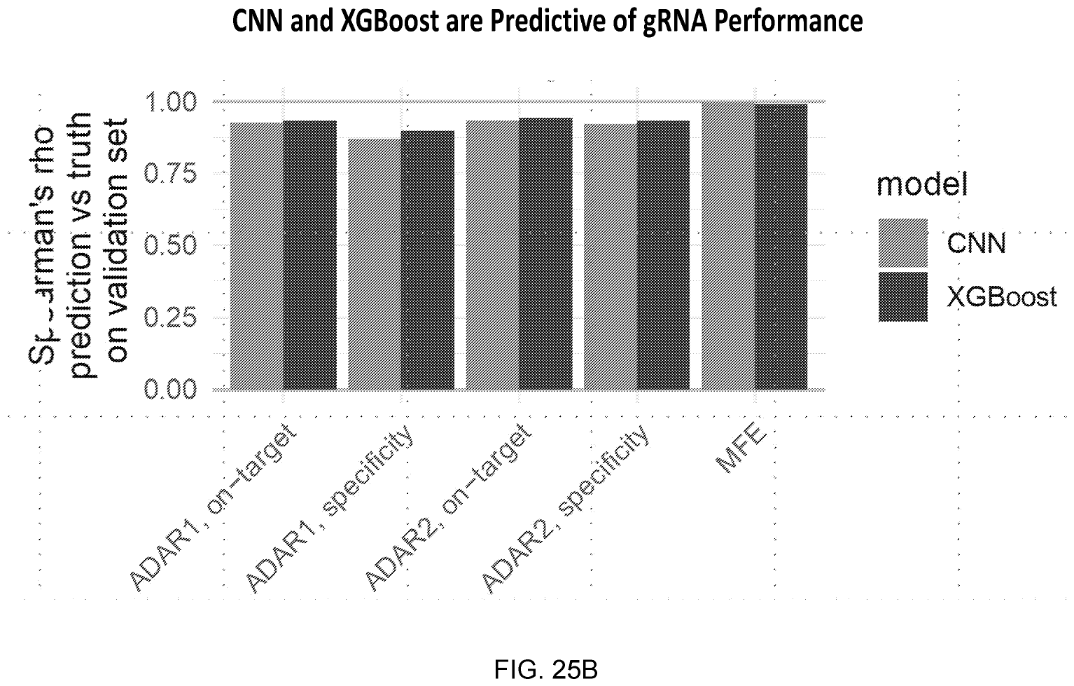

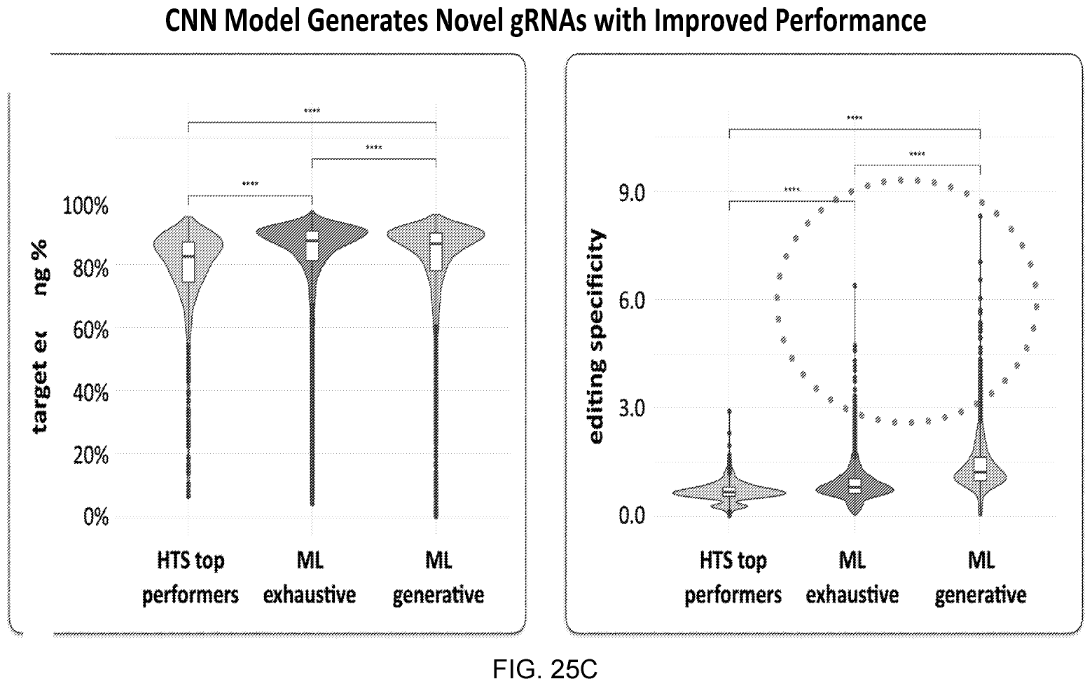

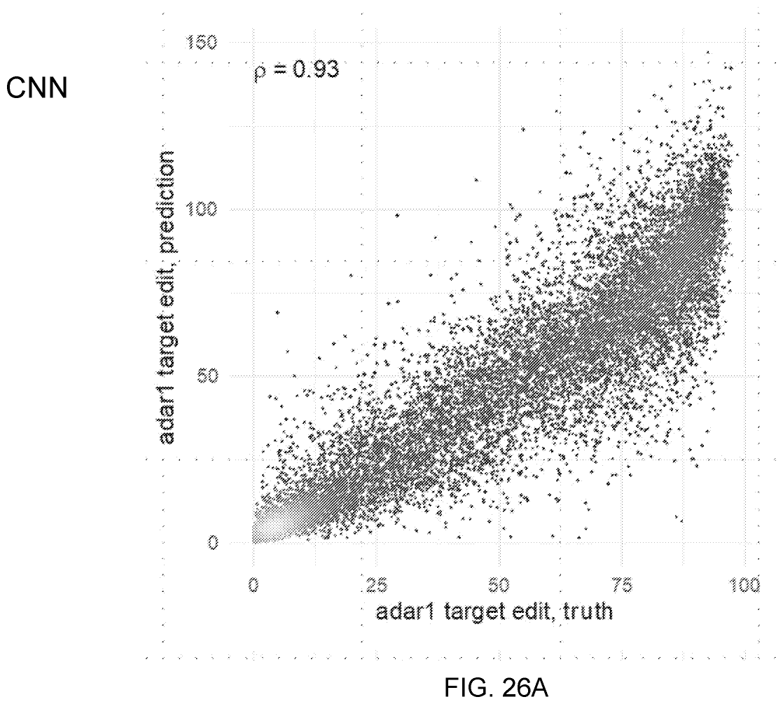

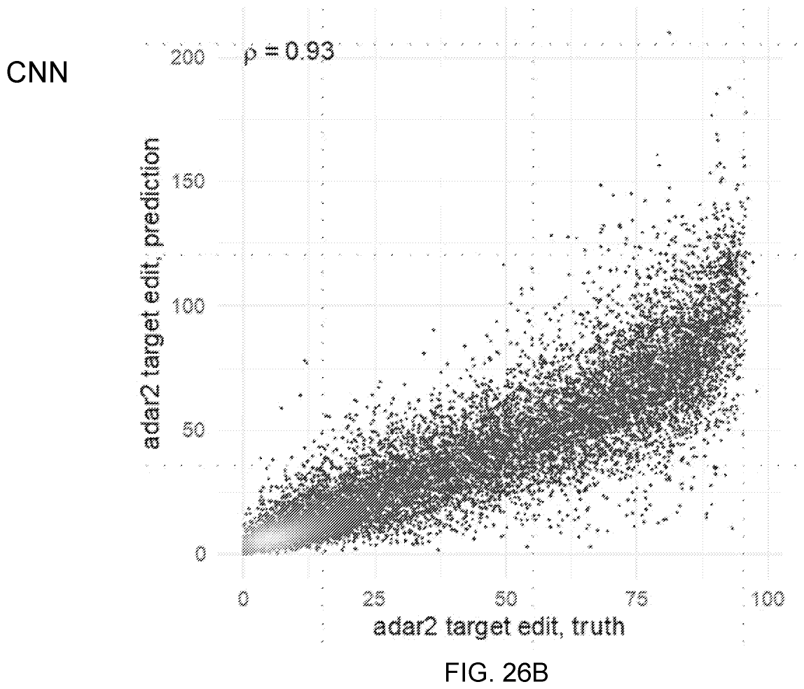

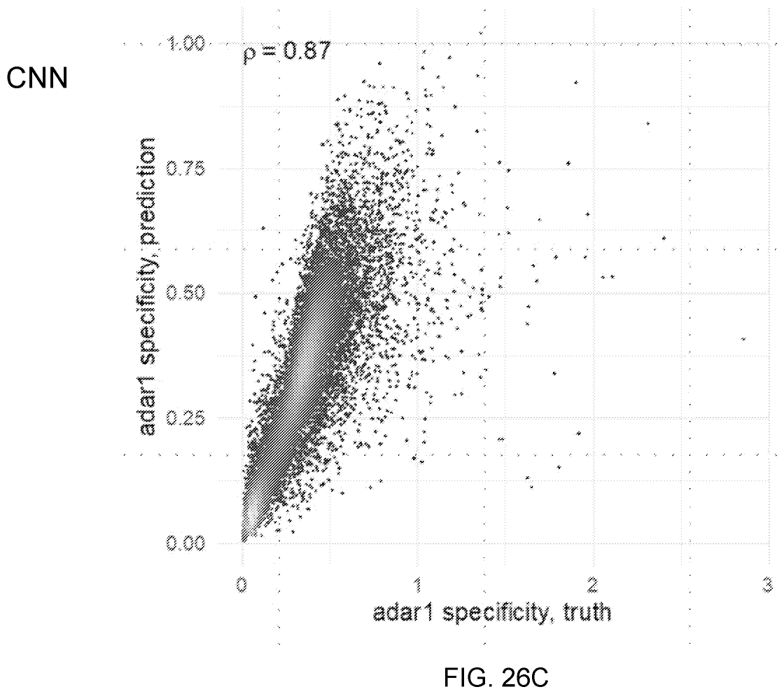

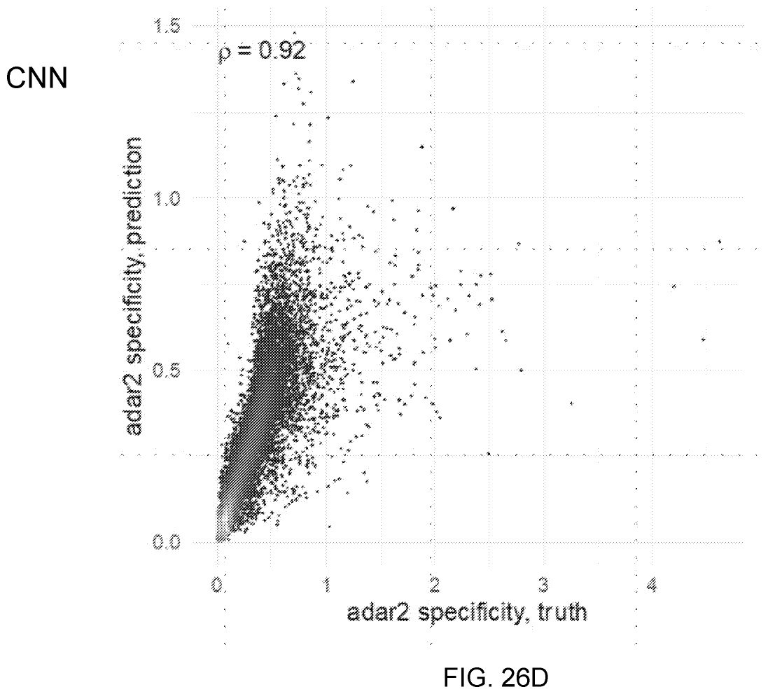

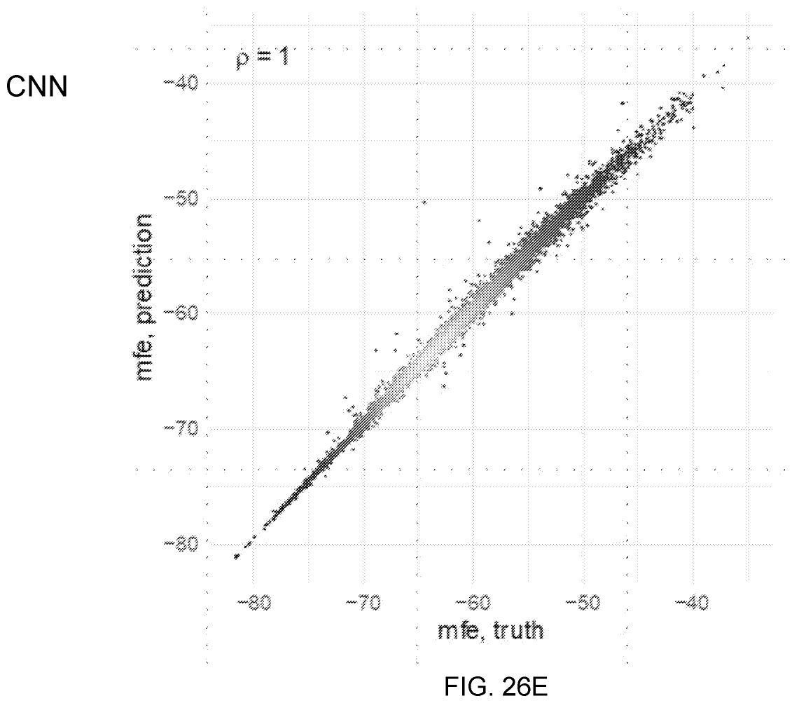

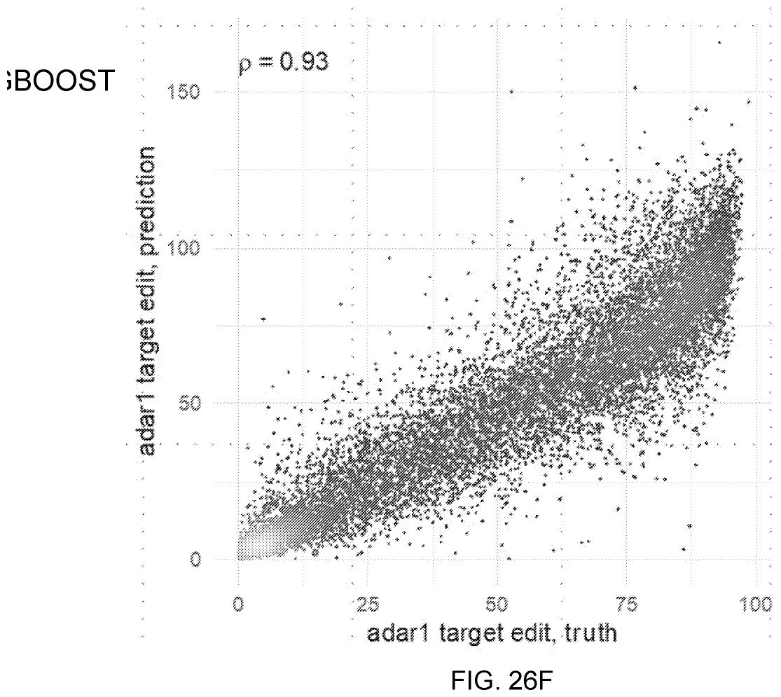

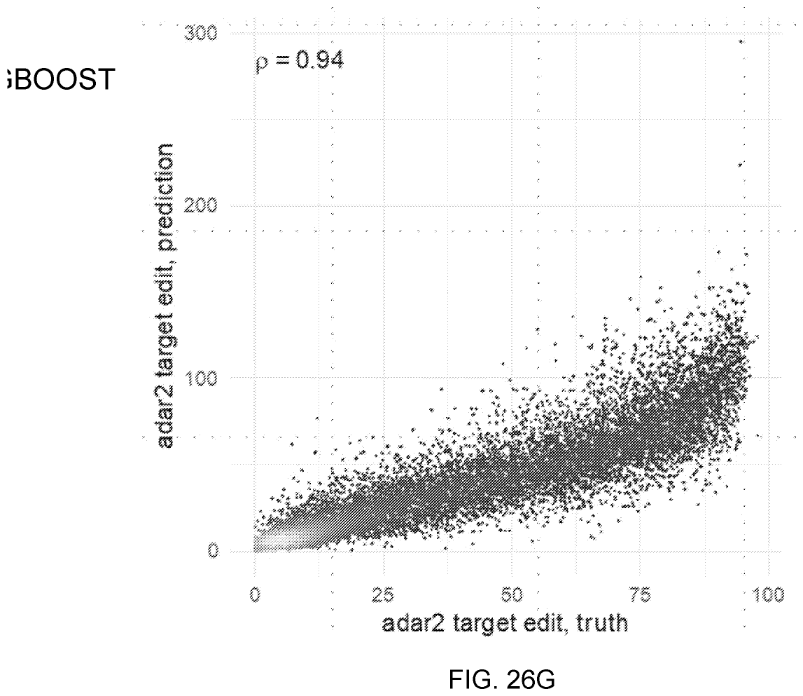

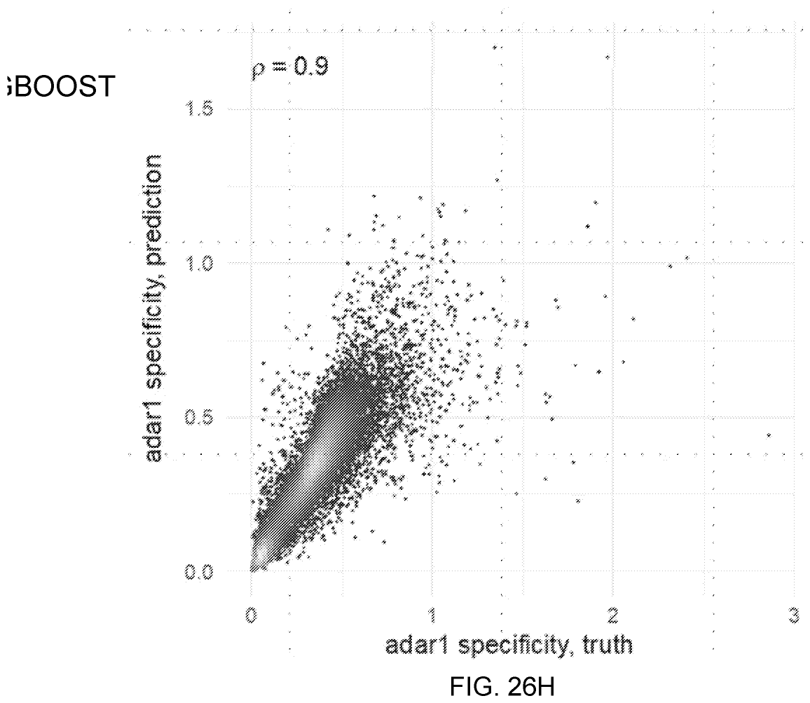

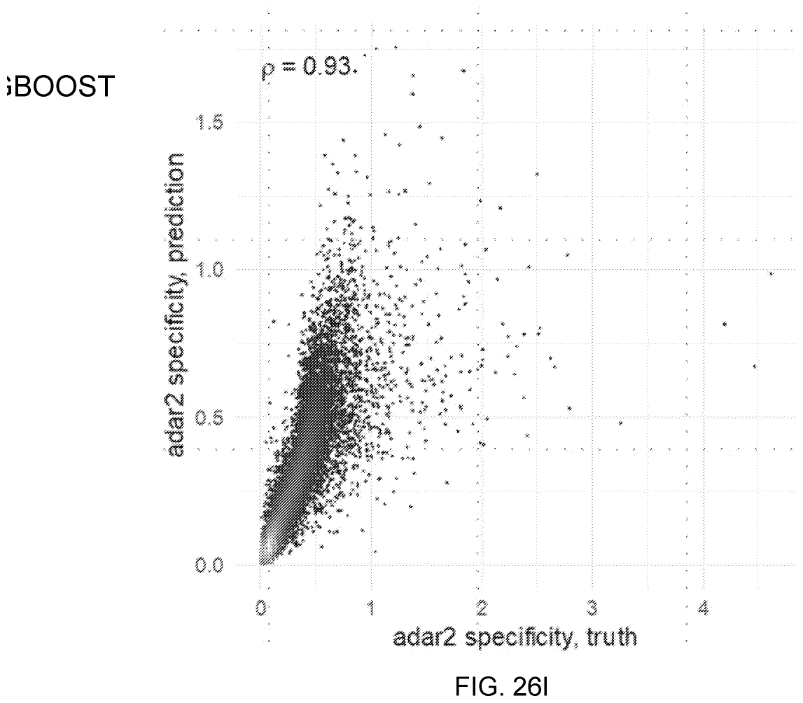

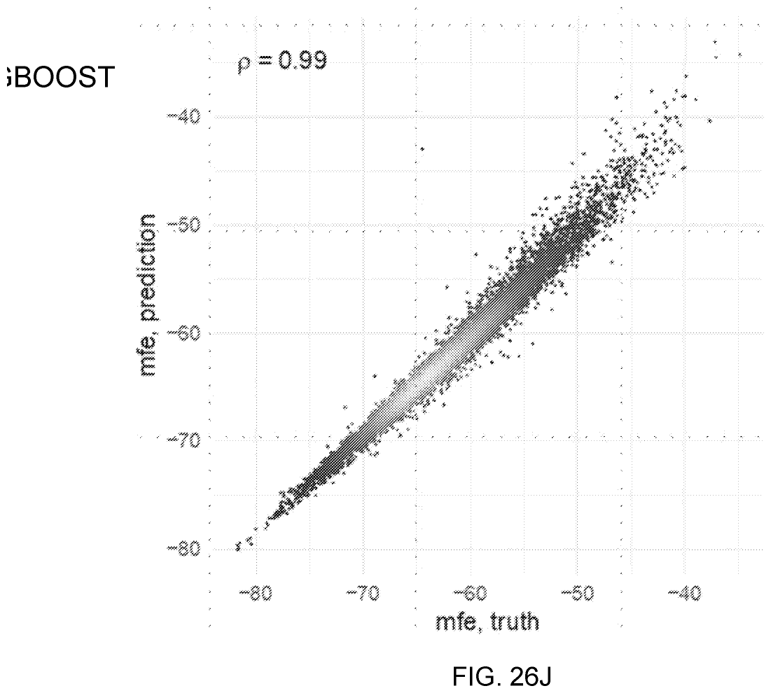

The more complex CNN framework was similarly predictive as XGBoost. For instance, FIG. 25A highlights the correlation between predicted and empirical measurements of on-target editing of the LRRK2 45-mer target for CNN and XGBoost. FIGS. 25B and 26A-J further illustrate similar predictive ability for the on-target editing, specificity, and minimum free energy (MFE) metrics for each of the two ADAR enzymes ADAR1 and ADAR2, for the CNN and XGBoost model architectures.

Machine learning (ML)-based designs from both the exhaustive and generative (e.g., input optimization) strategy achieved higher efficiency and specificity than top designs from the initial library screen when tested experimentally. Particularly, as illustrated in FIG. 25C, experimentally validated target editing and specificity was determined for a select number of top-performing guide RNAs from the HTS library (HTS top performers), guide RNAs obtained from the exhaustive machine learning strategy (ML exhaustive), and guide RNAs obtained from the generative machine learning strategy (ML generative) that were retested to confirm the predictive ability of the ML models. Guide RNAs were observed in the ML exhaustive and ML generative strategies that exhibited better target editing and specificity than the guide RNAs in the original HTS library.

### Editing of Clinical Targets.

### Parkinson's Disease: LRRK2 G2019S.

Lead candidate gRNAs derived from HTS and ML models (e.g., obtained from the exhaustive and/or generative strategies, and/or having high on-target editing and specificity metrics) were transiently expressed in HEK293 cells engineered to express the LRRK2 G2019S mutation with endogenous ADAR1.

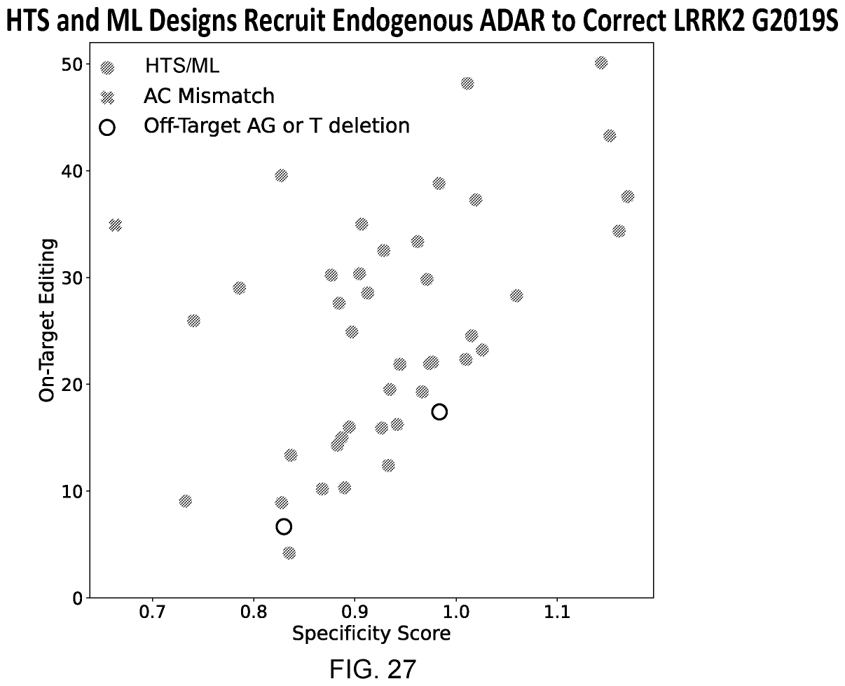

These lead designs were designated as starting scaffolds to further optimize expression, stability, efficiency, vectorization, and manufacturing. FIG. 27 is a scatter plot showing the full panel of starting scaffolds tested, with corresponding on-target editing and specificity scores. As illustrated in FIG. 27, a high proportion of candidate gRNA designs exhibited high efficiency and specificity compared to rudimentary first-generation design principles, without sacrificing on-target editing.

The data highlights the use of ML models to generate highly efficient and specific gRNAs that can recruit endogenous ADAR for the correction of pathogenic mutations.

### Example 6

### Machine Learning for Engineered Guides that Target ADAR Isoform Specific

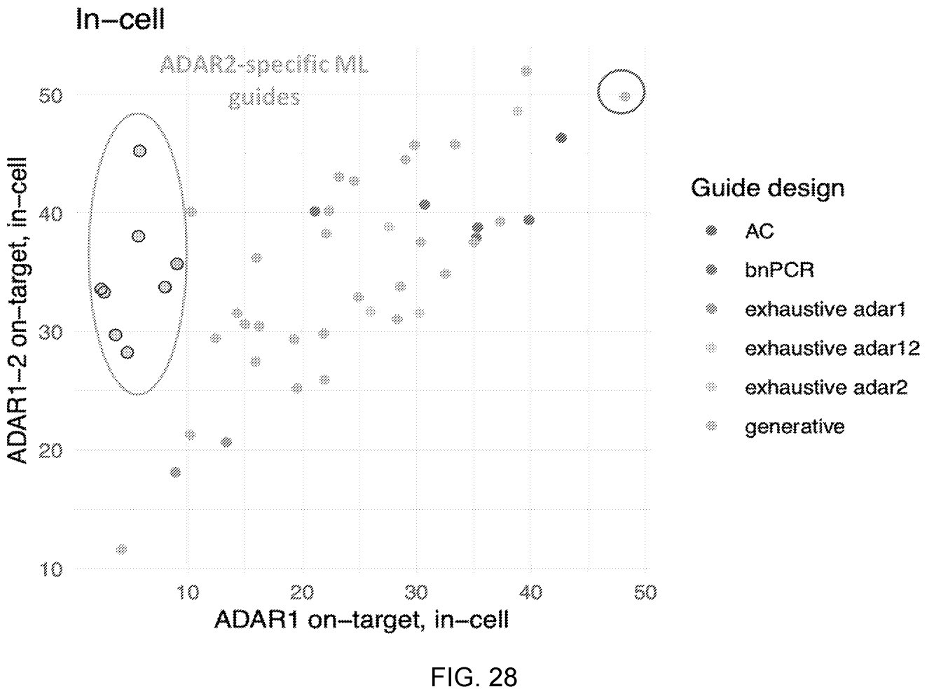

This example describes engineered guide RNA sequences that target mRNA based on a specified ADAR isoform(s) on-target editing and a specified specificity score. A group of engineered guides were tested using the same method of CNN training as described in EXAMPLE 1 to predict an ADAR isoform(s) on-target editing and an ADAR isoform(s) specificity score based on an engineered guide RNA sequence. The specified ADAR isoform(s) was either ADAR1 or two isoforms ADAR1 and ADAR 2 (ADAR1/2). A first set of gRNA sequences with predicted ADAR1 only on-target editing and an ADAR1 only specificity score was identified. A second set of gRNA sequences with predicted ADAR1/2 on-target editing and an ADAR1/2 specificity score was identified. Additionally, the trained CNN was used in reverse, in which a specified ADAR isoform(s) on-target editing and a specified ADAR isoform(s) specificity score was inputted into the trained CNN to predict an engineered guide RNA sequence having that target editing and specificity score, using the methodology shown in FIG. 12. The specified ADAR isoform(s) was either ADAR1 or ADAR1/2. A third set of gRNA sequences with predicted ADAR1 only on-target editing and an ADAR1 only specificity score was generated. A fourth set of gRNA sequences with predicted ADAR1/2 on-target editing and an ADAR1/2 specificity score was generated. All four sets of gRNAs were then experimentally tested in cells expressing ADAR1 or ADAR1/2 as shown in FIG. 28 (“AC” refers to a guide RNA having only and A/C mismatch; “bnPCR” refers to HTS top performers).

### Example 7

### Experimental in-Cell Validation of ML Model-Derived gRNAs for LRRK2 G2019S mRNA

This example describes an in-depth, in-cell testing and analysis of machine learning (ML) model-derived gRNAs for a target mRNA transcribed from a target LRRK2 G2019S gene, in accordance with an embodiment of the present disclosure.

Two machine learning model types were utilized, an exhaustive model and a generative model, to engineer LRRK2-specific gRNAs that were subsequently evaluated. Using the exhaustive model, a set of exhaustive guide RNAs was obtained by a method including receiving information comprising at least a nucleic acid sequence for a guide RNA that hybridizes to the target mRNA. The information was inputted into a machine learning (ML) model including a plurality of parameters to obtain from the model a set of metrics for a deamination efficiency or specificity by an ADAR protein of a target nucleotide position in the target mRNA when facilitated by hybridization of the gRNA to the target mRNA. Specifically, the method included using nucleic acid sequences generated for simulated guide RNAs specific to the target mRNA sequence, and inputting the simulated gRNA nucleic acid sequences through a neural network model to obtain metrics for on-target and off-target deamination (e.g., on-target efficiency and/or specificity). The method further included filtering the simulated gRNAs using the neural network model until an endpoint was reached and a set of exhaustive guides was obtained, where each exhaustive guide in the set of exhaustive guides achieved particular on-target and off-target characteristics.

Using the generative model, a set of generative guide RNAs was obtained by a method including receiving information including a desired set of metrics for an efficiency or specificity of deamination of a target nucleotide position in the target mRNA by an ADAR protein when facilitated by hybridization of the gRNA to the target mRNA. Seed information was also obtained including at least a seed nucleic acid sequence for the gRNA and a target nucleic acid sequence for the target mRNA. The seed information was inputted into a model including a plurality of parameters to obtain a calculated set of metrics. The seed nucleic acid sequence was then iteratively updated, while holding the plurality of parameters and the target nucleic acid sequence fixed, to reduce a difference between the desired set and the calculated set of metrics, thus generating a candidate sequence for a generative guide RNA. Specifically, the method included creating generative guide RNAs by establishing parameters in which machine learning was utilized to produce novel gRNA sequences as an output. The difference between the two types of machine learning approaches are depicted in FIGS. 9A-C (exhaustive model) and FIG. 12 (generative model).

Next-generation sequencing (NGS) was used to compare highly efficient and specific ML-derived gRNAs using the two approaches against gRNAs generated using in vitro high throughput screening (HTS) methods. For in-cell assays, gRNAs were dosed in HEK293 cells expressing a LRRK2 G2019S cDNA minigene.

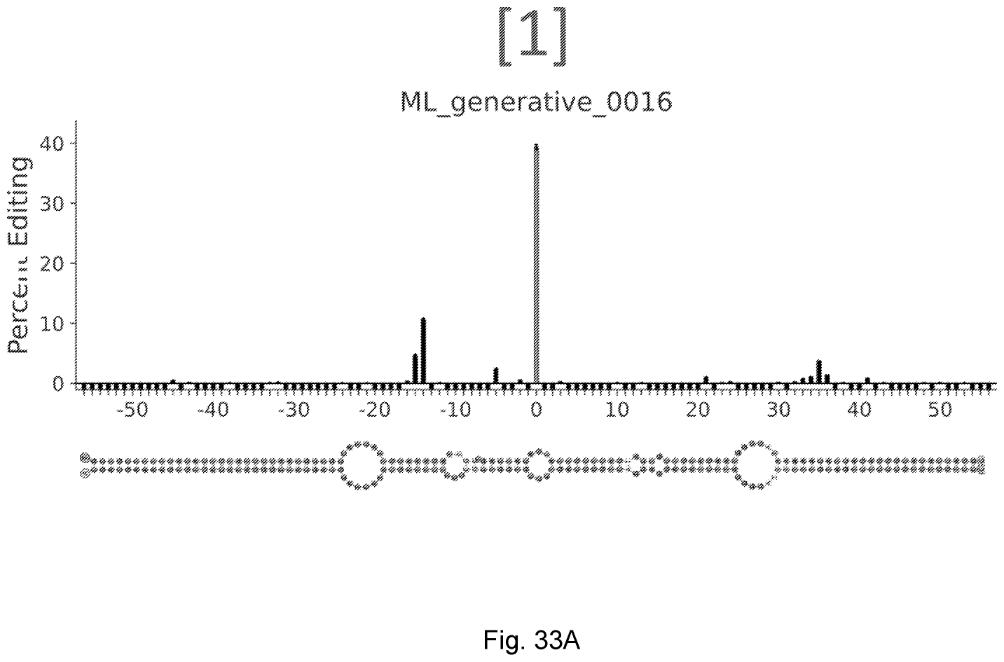

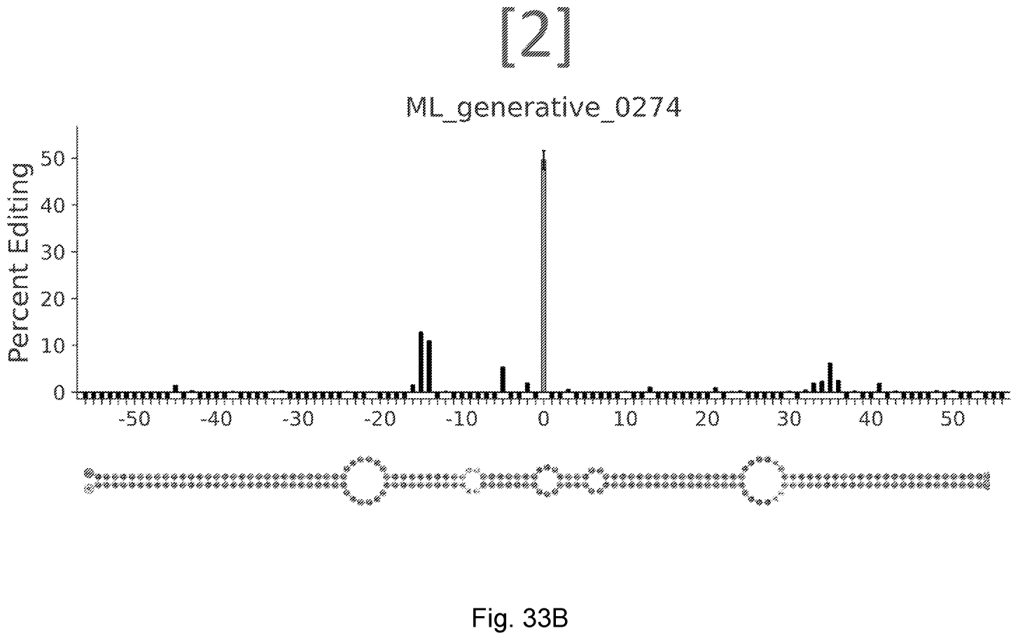

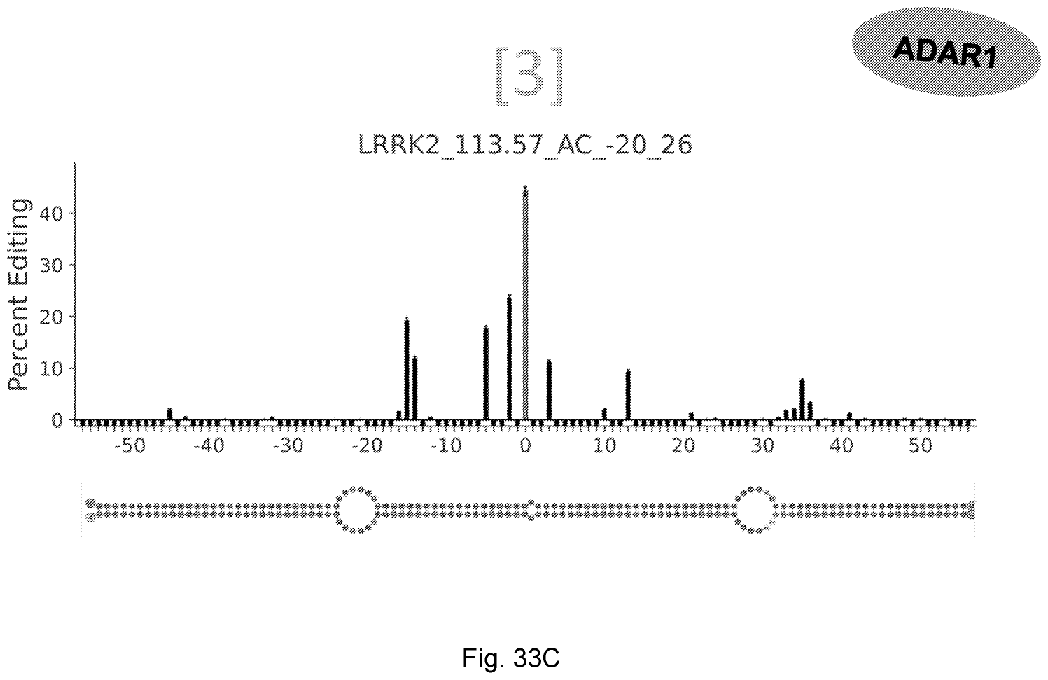

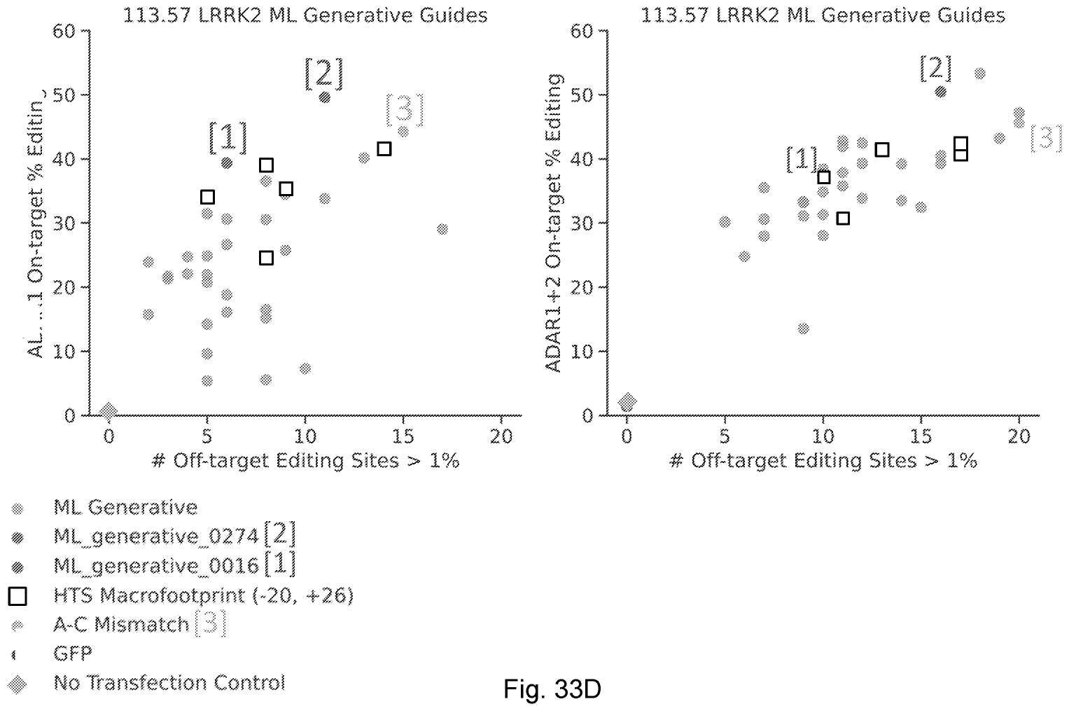

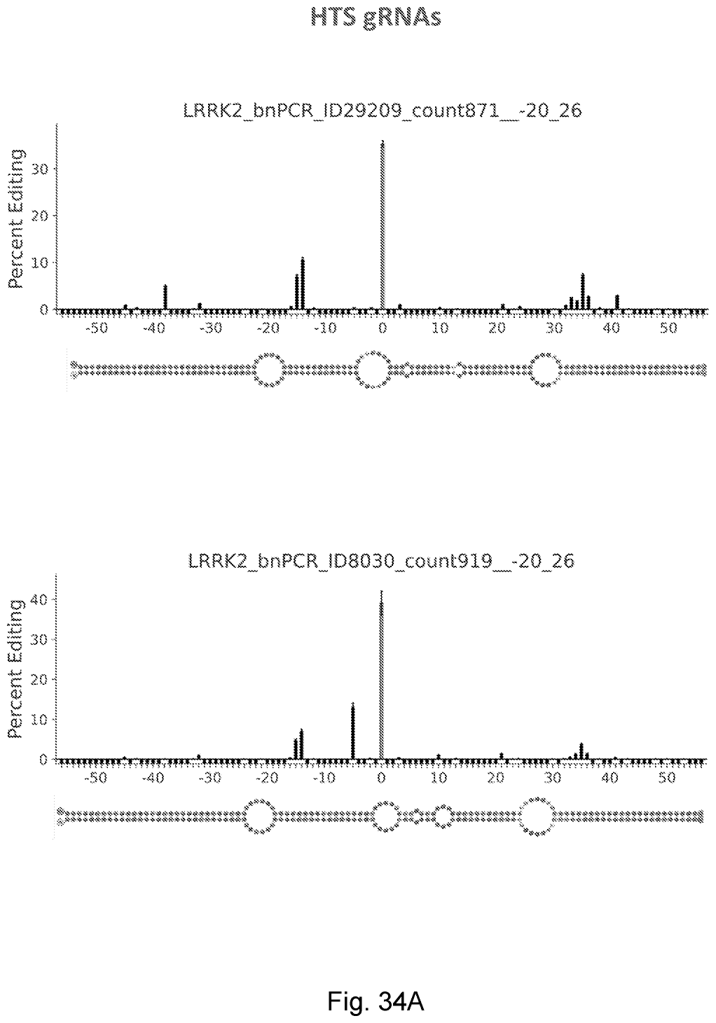

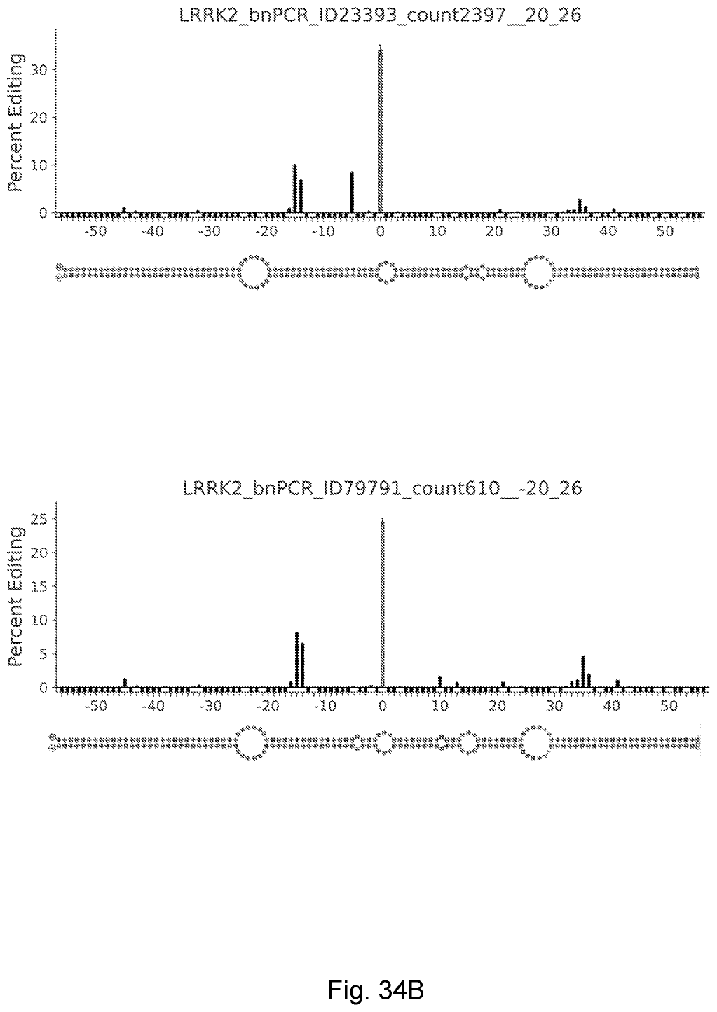

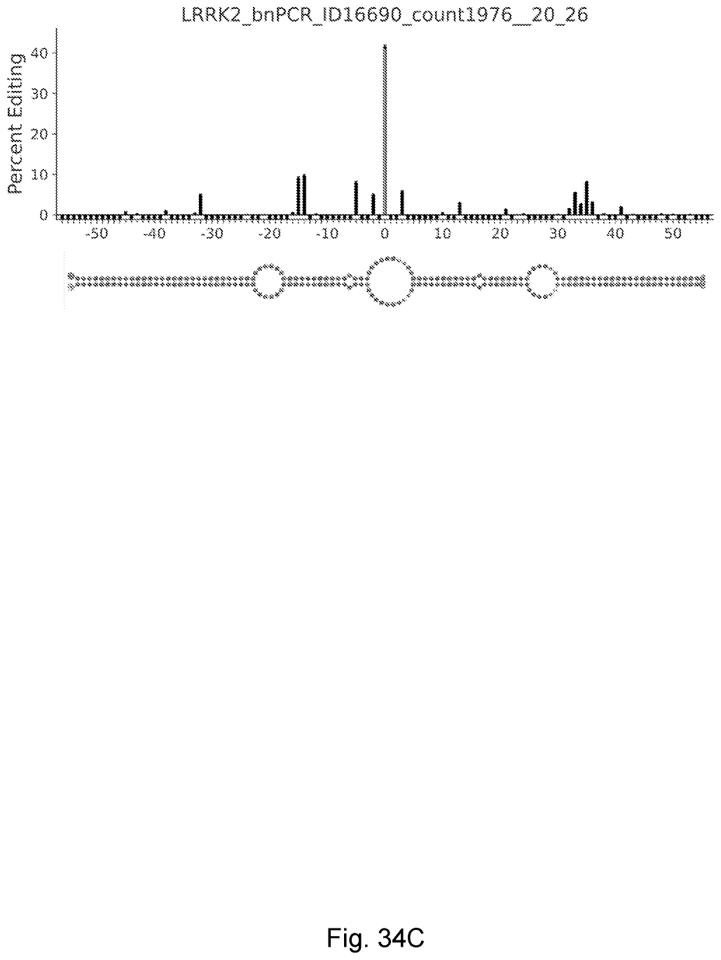

As shown in FIGS. 33A-D and 34A-C, the experimental approach identified two generative ML guides (0274 and 0016) that outperformed an A-C mismatch guide as well as the HTS guides. These ML guides showed relatively higher on-target efficiency and similar or improved specificity when compared to original HTS gRNAs. In particular, FIGS. 33A-C illustrate editing percentages by ADAR1 at the target nucleotide position (denoted by 0 on the x-axis) and at off-target positions along the target mRNA sequence (denoted as distance in nucleotides relative to the target nucleotide position) for the generative ML guide 0016 (FIG. 33A; [1]), the generative ML guide 0274 (FIG. 33B; [2]), and the A-C mismatch guide (FIG. 33C; [3]), which all have the same barbell macro-foot print (positioned at −20; +26 relative to the target nucleotide position). FIGS. 34A-C also illustrate editing percentage at the target nucleotide position and at off-target positions for a subset of the HTS guide RNAs. FIG. 33D shows a scatterplot of on-target editing percentages versus number of off-target editing sites with greater than 1% editing, for ADAR1 and ADAR1/2. Both generative ML guides were observed to have reduced off-target editing compared to the A-C mismatch guide, as well as higher on-target editing efficiency compared to HTS gRNAs, for both ADAR1 and ADAR1/2.

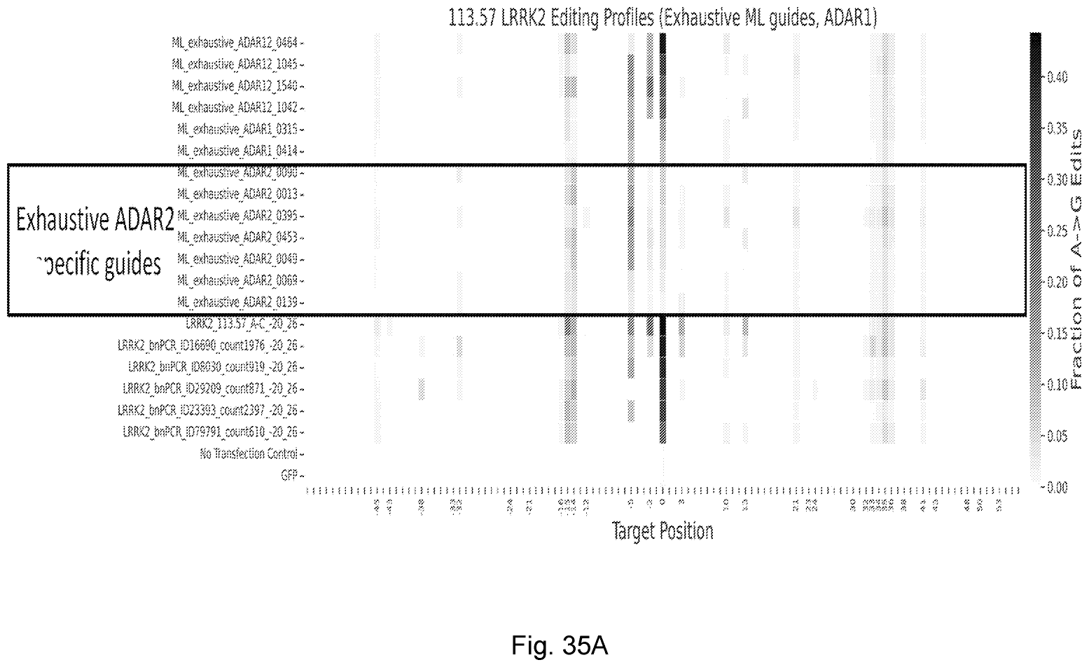

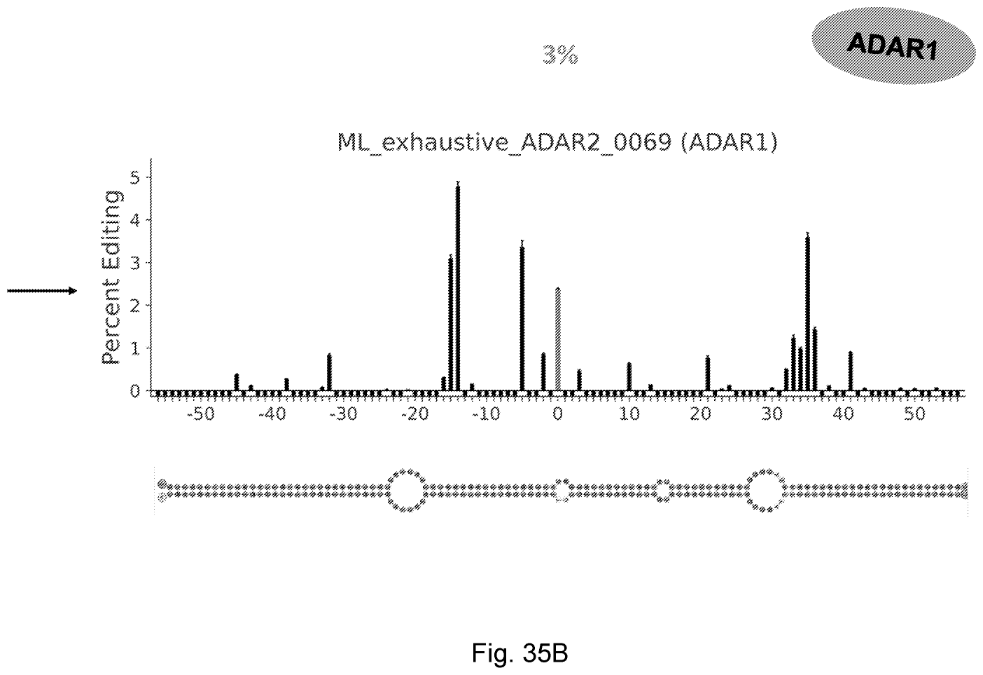

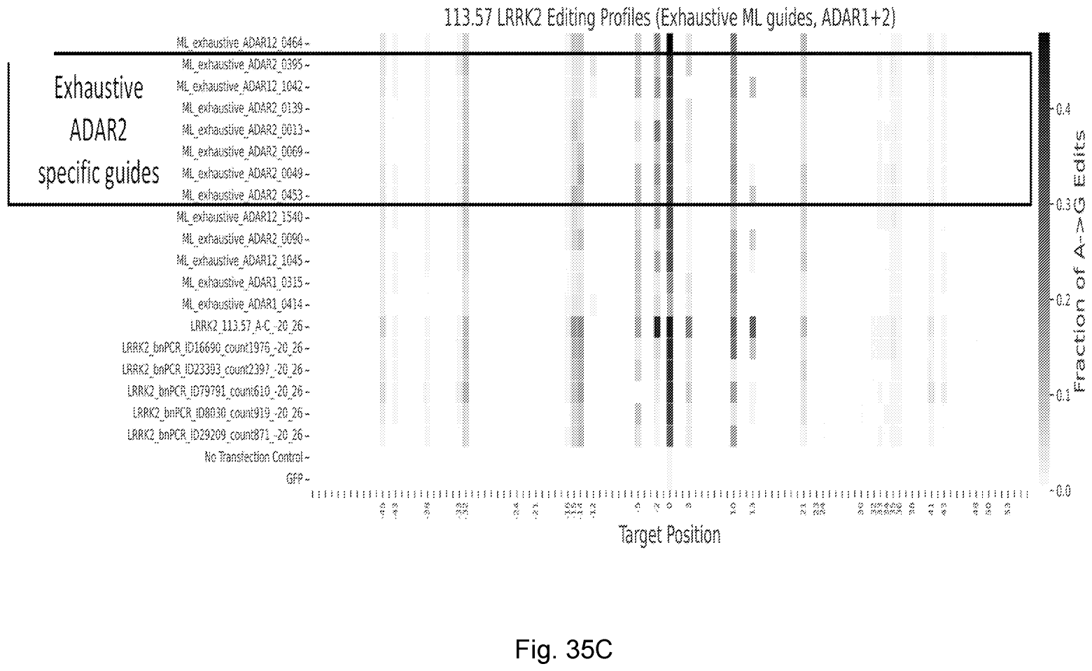

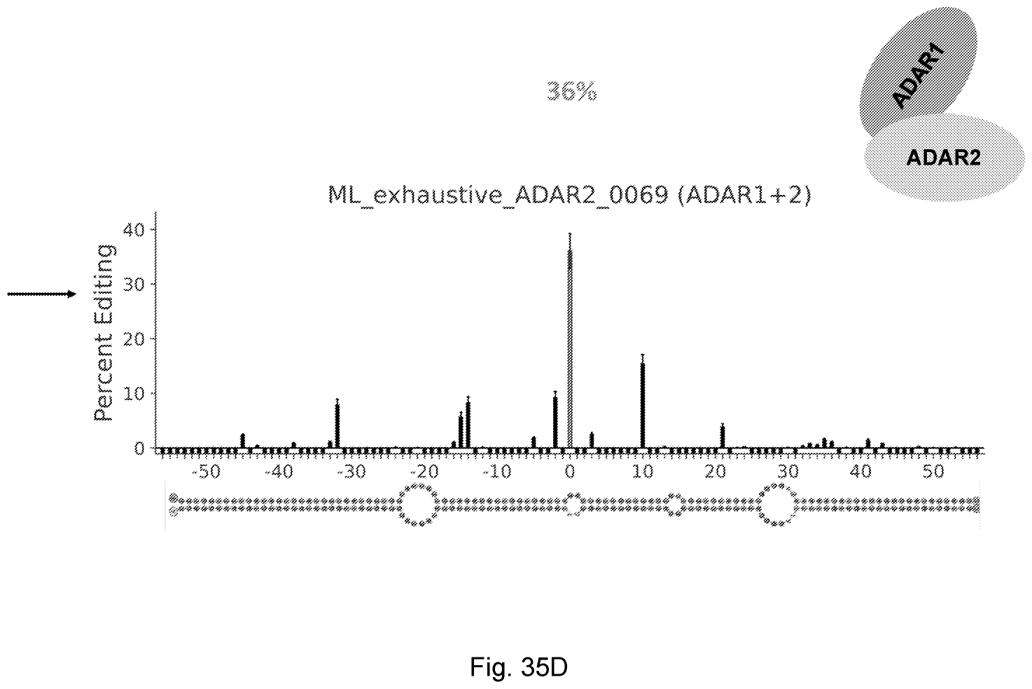

FIGS. 35A-D collectively show that, in some embodiments, exhaustive ML guides derived from the exhaustive model accurately predict ADAR preference in cells. For example, FIGS. 35A and C show the LRRK2 G2019S editing profiles of a subset of exhaustive ML guides that were selected for based on ADAR2-preferential activity (solid black box), where editing was assayed using either ADAR1 (FIG. 35A) or ADAR1/2 (FIG. 35C). Editing profiles show the fraction of adenosine to guanosine (A-to-G) edits (shown as intensity of shading) at each position in the target LRRK2 G2019S mRNA relative to the target nucleotide position (denoted by 0 on the x-axis). A comparison of the LRRK2 G2019S editing profiles for the subset of ADAR2-specific exhaustive ML guides clearly highlights a low level of editing at the target nucleotide position by ADAR1 (editing fraction of 0.10 or less) and a high level of editing at the target position by ADAR1/2 (editing fraction of at least 0.4), for all guide RNAs in the subset. These results illustrate that the exhaustive guides performed best when placed in the ADAR environment that they were selected for. To further illustrate these observations, FIGS. 35B and 35D show the LRRK2 G2019S editing profiles of a representative exhaustive ML guide selected for ADAR2-preferential activity (0069), which shows low (less than 3%) on-target editing by ADAR1 and relatively high (greater than 40%) on-target editing by ADAR1/2.

FIG. 36 illustrates a scatterplot of ADAR1 on-target editing percentage compared to ADAR1/2 on-target editing percentage for selected exhaustive ML ADAR1-preferential guides, exhaustive ML ADAR2-preferential guides, exhaustive ML ADAR1/2-preferential guides, the HTS-generated guides, the A-C mismatch guide, and controls. A cluster of ADAR2-preferential guide RNAs was observed to have low ADAR1 on-target editing and high ADAR1/2 on-target editing (shown by the dashed circle). An examination of the sequence and structural features of these ADAR2-preferential guides revealed shared A-G mismatches across from the target adenosine in the guide-target mRNA scaffold formed upon binding of the gRNA to the target mRNA. These results indicate that an A-G mismatch at the target adenosine potentially drives an ADAR2 preference.

To summarize, the testing and subsequent NGS analysis of ML derived guides gives rise to at least two conclusions. First, in some implementations, some selected generative ML derived guides outperform guides obtained from HTS. Second, in some embodiments, specific sequence characteristics underlying certain exhaustive ML derived guides are useful to predict enzymatic preference. The ADAR2 specific gRNAs containing an A-G mismatch across the target adenosine highlight the potential role of this structural feature to govern ADAR preference.

Although inventions have been particularly shown and described with reference to a preferred embodiment and various alternate embodiments, it will be understood by persons skilled in the relevant art that various changes in form and details can be made therein without departing from the spirit and scope of the invention.

1. A method for predicting a deamination efficiency or specificity comprising:
at a computer system comprising at least one processor and a memory storing at least one program for execution by the at least one processor:
A) receiving, in electronic form, information comprising a nucleic acid sequence for a guide RNA (gRNA) that hybridizes to a target mRNA; and
B) inputting the information into a model comprising a plurality of parameters to obtain as output from the model a set of one or more metrics for a deamination efficiency or specificity by an Adenosine Deaminase Acting on RNA (ADAR) protein of a target nucleotide position in the target mRNA when facilitated by hybridization of the gRNA to the target mRNA.

2-38. (canceled)

39. A method for generating a candidate sequence for a guide RNA (gRNA), comprising:
at a computer system comprising at least one processor and a memory storing at least one program for execution by the at least one processor:
A) receiving, in electronic form, information comprising a desired set of one or more metrics for an efficiency or specificity of deamination of a target nucleotide position in a target mRNA by an Adenosine Deaminase Acting on RNA (ADAR) protein when facilitated by hybridization of the gRNA to the target mRNA;
B) receiving, in electronic form, seed information comprising (i) a seed nucleic acid sequence for the gRNA and (ii) a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA;
C) inputting the seed information into a model comprising a plurality of parameters to obtain as output from the model a calculated set of the one or more metrics for the efficiency or specificity of deamination of the target nucleotide position in the target mRNA by the ADAR protein; and
D) iteratively updating the seed nucleic acid sequence, while holding the plurality of parameters and the target nucleic acid sequence fixed, to reduce a difference between (i) the desired set of the one or more metrics and (ii) the calculated set of the one or metrics, thereby generating the candidate sequence.

40. The method of claim 39, further comprising:
E) determining, using a gRNA having the candidate sequence, an experimental set of the one or more metrics for the efficiency or specificity of deamination of the target nucleotide position in the target mRNA by an ADAR protein; and
F) training a model using a training dataset comprising the experimental set of the one or more metrics for the efficiency or specificity of deamination of the target nucleotide position in the target mRNA by the ADAR protein.

41. The method of claim 39, wherein the set of one or more metrics for the efficiency or specificity of deamination of the target nucleotide position by the ADAR protein comprises a metric for the efficiency of deamination of the target nucleotide position by a first ADAR protein.

42. The method of claim 41, wherein the metric for the efficiency of deamination of the target nucleotide position by the first ADAR protein is (i) a prevalence of deamination of the target nucleotide position in a plurality of instances of the target mRNA or (ii) a prevalence of the absence of deamination of any nucleotide position in a respective instance of a target mRNA in a plurality of instances of the target mRNA.

43. The method of claim 39, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

44. The method of claim 43, wherein the metric for the specificity of deamination of the target nucleotide position relative to one or more nucleotide positions, other than the target nucleotide position, in the target mRNA by the first ADAR protein is:
(i) a comparison of (a) a prevalence of deamination of the target nucleotide position in a plurality of instances of the target mRNA and (b) a prevalence of deamination of at least one nucleotide position, other than the target nucleotide position, in a respective instance of the target mRNA in a plurality of instances of the target mRNA,
(ii) a prevalence of deamination of the target nucleotide position, without coincident deamination of one or more nucleotide positions other than the target nucleotide position, in a respective instance of the target mRNA in a plurality of instances of the target mRNA, or
(iii) a prevalence of deamination of at least one nucleotide position, other than the target nucleotide position, in a respective instance of the target mRNA in a plurality of instances of the target mRNA.

45. The method of claim 44, wherein, at the one or more nucleotide positions, other than the target nucleotide position, in the target mRNA, deamination results in a non-synonymous codon edit.

46. The method of claim 39, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

47. The method of claim 39, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

48. The method of claim 39, wherein the first ADAR protein is human ADAR1 or human ADAR2.

49. The method of claim 39, wherein the output from the model further comprises one or more metrics for an efficiency or specificity of deamination of the target nucleotide position by a second ADAR protein when facilitated by hybridization of the gRNA to the target mRNA.

50-55. (canceled)

56. The method of claim 49, wherein the first ADAR protein is human ADAR1 and the second ADAR protein is human ADAR2.

57-59. (canceled)

60. The method of claim 39, wherein the model further generates an estimation of a minimum free energy (MFE) for the guide-target RNA scaffold formed between the guide RNA (gRNA) and the target mRNA.

61. The method of claim 39, wherein the model is a neural network, a support vector machine, a Naive Bayes model, a nearest neighbor model, a boosted trees model, a random forest model, a decision tree, or a clustering model.

62-64. (canceled)

65. The method of claim 39, wherein the plurality of parameters reflects;
a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type; and
a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

66-67. (canceled)

68. The method of claim 39, wherein the seed information further comprises a plurality of structural features of a guide-target RNA scaffold formed between the gRNA and the target mRNA when the gRNA hybridizes to the target mRNA.

69. (canceled)

70. The method of claim 68, wherein the plurality of structural features comprises one or more structural features selected from the group consisting of:
a structural motif comprising two or more structural features;
a presence or absence of a mismatch formed upon binding of the gRNA to the mRNA transcribed from the target gene;
a position of a mismatch formed upon binding of the gRNA to the mRNA transcribed from the target gene;
a presence or absence of a bulge formed upon binding of the gRNA to the mRNA transcribed from the target gene;
a position of a bulge formed upon binding of the gRNA to the mRNA transcribed from the target gene;
a size of a bulge formed upon binding of the gRNA to the mRNA transcribed from the target gene;
a presence or absence of an internal loop in the gRNA upon binding to the mRNA transcribed from the target gene;
a position of an internal loop in the gRNA upon binding to the mRNA transcribed from the target gene;
a size of an internal loop in the gRNA upon binding to the mRNA transcribed from the target gene;
a presence or absence of an internal loop in the mRNA transcribed from the target gene upon binding to the gRNA;
a position of an internal loop in the mRNA transcribed from the target gene upon binding to the gRNA;
a size of an internal loop in the mRNA transcribed from the target gene upon binding to the gRNA;
a presence or absence of a hairpin in the gRNA upon binding to the mRNA transcribed from the target gene;
a position of a hairpin in the gRNA upon binding to the mRNA transcribed from the target gene;
a size of a hairpin in the gRNA upon binding to the mRNA transcribed from the target gene;
a presence or absence of a hairpin in the mRNA transcribed from the target gene upon binding to the gRNA;
a position of a hairpin in the mRNA transcribed from the target gene upon binding to the gRNA;
a size of a hairpin in the mRNA transcribed from the target gene upon binding to the gRNA;
a presence or absence of a wobble base pair formed upon binding of the gRNA to the mRNA transcribed from the target gene;
a position of a wobble base pair formed upon binding of the gRNA to the mRNA transcribed from the target gene;
a presence or absence of a barbell upon binding of the gRNA to the mRNA transcribed from the target gene;
a position of a barbell upon binding of the gRNA to the mRNA transcribed from the target gene;
a size of a barbell upon binding of the gRNA to the mRNA transcribed from the target gene;
a presence or absence of a dumbbell upon binding of the gRNA to the mRNA transcribed from the target gene;
a position of a dumbbell upon binding of the gRNA to the mRNA transcribed from the target gene;
a size of a dumbbell upon binding of the gRNA to the mRNA transcribed from the target gene;
a presence or absence of a base paired region formed upon binding of the gRNA to the mRNA transcribed from the target gene;
a position of a base paired region formed upon binding of the gRNA to the mRNA transcribed from the target gene;
a size of a base paired region formed upon binding of the gRNA to the mRNA transcribed from the target gene;
a presence or absence of a base paired region formed upon binding of the gRNA to the mRNA transcribed from the target gene;
a position of a base paired region formed upon binding of the gRNA to the mRNA transcribed from the target gene;
a size of a base paired region formed upon binding of the gRNA to the mRNA transcribed from the target gene;
a presence or absence of a U-deletion formed upon binding of the gRNA to the mRNA transcribed from the target gene;
a position of a U-deletion formed upon binding of the gRNA to the mRNA transcribed from the target gene;
a size of U-deletion formed upon binding of the gRNA to the mRNA transcribed from the target gene;
a coaxial stacking formed upon binding of the gRNA to the mRNA transcribed from the target gene;
an adenosine platform formed upon binding of the gRNA to the mRNA transcribed from the target gene;
an interhelical packing motif formed upon binding of the gRNA to the mRNA transcribed from the target gene;
a triplex formed upon binding of the gRNA to the mRNA transcribed from the target gene;
a major groove triple formed upon binding of the gRNA to the mRNA transcribed from the target gene;
a minor groove triple formed upon binding of the gRNA to the mRNA transcribed from the target gene;
a tetraloop motif formed upon binding of the gRNA to the mRNA transcribed from the target gene;
a metal-core motif formed upon binding of the gRNA to the mRNA transcribed from the target gene;
a ribose zipper formed upon binding of the gRNA to the mRNA transcribed from the target gene;
a kissing loop formed upon binding of the gRNA to the mRNA transcribed from the target gene;
and a pseudoknot formed upon binding of the gRNA to the mRNA transcribed from the target gene.

71. (canceled)

72. The method of claim 39, wherein the seed information further comprises a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA.

73. The method of claim 39, wherein the seed nucleic acid sequence for the gRNA comprises one or more fixed nucleotide identities.

74-94. (canceled)
